(19) United States
(12) Patent Application Publication
(10) Pub. No.: US 2012/0245202 A1
(43) Pub. Date: Sep. 27, 2012

(54) METHODS AND PRODUCTS FOR TREATING INFLAMMATION

(75) Inventor: Jonnie R. Williams, Bradenton, FL (US)

(73) Assignee: ROCK CREEK PHARMACEUTICALS, INC., Gloucester, MA (US)

(21) Appl. No.: 13/235,860

(22) Filed: Sep. 19, 2011

Related U.S. Application Data
(60) Provisional application No. 61/383,811, filed on Sep. 17, 2010.
(60) Provisional application No. 61/384,447, filed on Sep. 20, 2010.
(60) Provisional application No. 61/439,473, filed on Feb. 4, 2011.
(60) Provisional application No. 61/480,271, filed on Apr. 28, 2011.
(60) Provisional application No. 61/480,258, filed on Apr. 28, 2011.

(51) Int. Cl. A61K 31/444; A61P 37/00; A61P 25/28; A61P 35/00; A61P 11/00; A61P 25/16; A61P 25/08; A61P 25/00; A61P 5/16; A61P 3/10; A61P 9/10; A61P 29/00; A61P 19/02

(52) U.S. Cl. 514/334

(57) ABSTRACT

Pharmaceutical compositions comprising an isolated form of a compound of Formula I or IA (e.g., anatabine or S-(−)-anatabine) or a salt thereof can be used to treat disorders comprising an inflammatory component, including chronic, low-level inflammation. Compounds of Formula I also can be provided, for example, in other vehicles such as beverage products and consumer products such as lotions and creams.

This application claims the benefit of and incorporates by reference Ser. No. 61/383,811 filed Sep. 17, 2010; Ser. No. 61/384,447 filed Sep. 20, 2010; Ser. No. 61/439,473 filed Feb. 4, 2011; Ser. No. 61/480,271 filed Apr. 28, 2011; and Ser. No. 61/480,258 filed Apr. 28, 2011.

Each reference cited in this disclosure is incorporated herein in its entirety.

### TECHNICAL FIELD

This disclosure relates generally to methods and compositions for treating inflammation and conditions associated with inflammation.

### BACKGROUND

Inflammation is a protective response to harmful stimuli, such as oxidative stress, irritants, pathogens, and damaged cells. The inflammatory response involves the production and release of inflammatory modulators that heal injured tissue and destroy damaged cells, by directly or indirectly producing and/or signaling the release of agents that produce reactive oxygen species. Thus, an appropriate inflammatory response involves a balance between the destruction of damaged cells and the healing of injured tissue.

An unchecked inflammatory response can lead to oxidative stress and the onset of various inflammatory disease pathologies. In fact, inflammatory processes underlie a wide variety of pathologies, including immune and autoimmune diseases, gastrointestinal diseases, various types of cancer, vascular disorders, heart disease, and neurodegenerative diseases. There is a need in the art for agents that can reduce inappropriate levels of inflammation.

### BRIEF DESCRIPTION OF THE DRAWINGS

FIG. 1. Graph showing effects of anatabine on TNFα-induced NFκB activity in vitro. See Example 1.

FIG. 2. Graph showing effects of a crude extract of smokeless tobacco on TNFα-induced NFκB activity in vitro. See Example 1.

FIG. 3. Graph showing effects of nicotine and of an alkaloid extract of smokeless tobacco on TNFα-induced NFκB activity in vitro. See Example 1.

FIG. 4. Graph showing the results of a cytotoxicity assay measuring release of lactate dehydrogenase (LDH) using supernatant from the cells assayed in FIG. 1. See Example 2.

FIG. 5. Graph showing the results of a cytotoxicity assay using supernatant from the cells assayed in FIG. 2. See Example 2.

FIG. 6. Graph showing the results of a cytotoxicity assay using supernatant from the cells assayed in FIG. 3. See Example 2.

FIG. 7. Graph showing concentrations in rat plasma as a function of time of anatabine and nicotine after a single intravenous bolus injection.

FIG. 8. Graph showing concentrations of anatabine and nicotine in rat plasma as a function of time (semi-log).

FIG. 9. Graph showing AUC_{0→∞} versus dose for both anatabine and nicotine in male and female rats.

FIG. 10. Graph showing concentrations of anatabine or nicotine in rat brain extracts following a single intravenous bolus dose.

FIG. 11. Graph showing mean concentration of anatabine and nicotine in rat brain extracts 0.5 hours after a single intravenous bolus dose.

FIG. 12. Nicotine product ion scan.

FIG. 13. Nicotine sample chromatogram.

FIG. 14. Anatabine product ion scan.

FIG. 15. Anatabine sample chromatogram.

FIG. 16. Nicotine-d3 product ion scan.

FIG. 17. Nicotine-d3 sample chromatogram.

FIG. 18. Anatabine-d4 product ion scan.

FIG. 19. Anatabine-d4 sample chromatogram.

FIG. 20. Graph showing mean body weights (±Std Dev) for each treatment group and gender.

FIGS. 21A-21B. Graphs showing mean (±SEM) concentration of anatabine in plasma for male or female rats. FIG. 21A, 0.6 mg/kg body weight (BW); FIG. 21B, 6.0 mg/kg BW.

FIGS. 22A-22B. Graphs showing mean (±SEM) concentration of anatabine in plasma for male and female rats combined. FIG. 22A, 0.6 mg/kg BW; FIG. 22B, 6.0 mg/kg BW.

FIGS. 23A-23B. Graphs showing mean (±SEM), maximal (Cp, max), and minimal (Cp, min) concentrations of anatabine in plasma for male or female rats. FIG. 23A, 0.6 mg/kg BW; FIG. 23B, 6.0 mg/kg BW.

FIGS. 24A-C. Graphs showing concentration-response relationships of the positive controls on activation (Ach), potentiation (epibatidine), or inhibition (MLA) of the nAChR α3/β4 channel. FIG. 24A, Ach; FIG. 24B, epibatidine; FIG. 24C, MLA.

FIGS. 25A-B. Graphs showing concentration-response relationships of the test articles on activation of the nAChR α3/β4 channel. FIG. 25A, nicotine (−) isomer; FIG. 25B, anatabine.

FIGS. 26A-B. Graphs showing concentration-response relationships of the test articles on inhibition of the nAChR α3/β4 channel. FIG. 26A, anatabine; FIG. 26B, nicotine.

FIGS. 27A-C. Graphs showing concentration-response relationships of positive controls on activation (Ach), potentiation (epibatidine), or inhibition (MLA) of the nAChR α4/β2 channel. FIG. 27A, Ach; FIG. 27B, epibatidine and Ach; FIG. 27C, MLA, Ach, and epibatidine.

FIGS. 28A-B. Graphs showing concentration-response relationships of test articles on activation of the nAChR α4/β2 channel. FIG. 28A, anatabine; FIG. 28B, nicotine (−) isomer.

FIGS. 29A-B. Graphs showing concentration-response relationships of test articles on inhibition of the nAChR α4/β2 channel. FIG. 29A, anatabine; FIG. 29B, nicotine (−) isomer.

FIGS. 30A-B. Graphs showing concentration-response relationships of positive controls on activation (Ach) and inhibition (MLA) of the nAChR α7 channel. FIG. 30A, PNU and Ach; FIG. 30B, MLA and Ach.

FIGS. 31A-B. Graphs showing concentration-response relationships of test articles on activation of the nAChR α7 channel. FIG. 31A, anatabine; FIG. 31B, nicotine (−) isomer.

FIGS. 32A-B. Graphs showing concentration-response relationships of test articles on inhibition of the nAChR α7 channel: FIG. 32A, anatabine; FIG. 32B, nicotine (−) isomer.

FIG. 33. Graph showing effect of anatabine (“RCP006”) (30 minutes) on BACE-1 mRNA expression in human neuronal SHSY cells.

FIG. 34. Western blots and graph showing effect of anatabine (“RCP006”) (24 hours) on BACE-1 protein expression in human neuronal SHSY cells.

FIGS. 35A-B. Graphs showing the effect of anatabine (“RCP006”) on Aβ production in 7W CHO cells. FIG. 35A, Aβ1-42. FIG. 35B, Aβ1-40.

FIG. 36. Graph showing the effect of anatabine (“RCP006”) on sAPPβ/sAPPα production in 7W CHO cells.

FIG. 37. Graph demonstrating lack of observed toxicity of anatabine (“RCP006”) in 7W CHO cells.

FIG. 38. Graph demonstrating that anatabine (“RCP006”) inhibits NFκB activation in the brain of wild-type mice.

FIG. 39. Graph demonstrating the effect of anatabine (“RCP006”) on the inhibition of IL-1β release after LPS stimulation in whole human blood.

FIG. 40. Graph comparing the anti-inflammatory effects (IL-1β inhibition) of anatabine (“RCP006”) and NSAIDs after treatment of whole human blood with LPS.

FIG. 41. Graph demonstrating the anti-inflammatory effect of anatabine (“RCP006”) on LPS-induced IL-1β release in human blood over time.

FIG. 42. Graph demonstrating thyroid pathology score, expressed as percent of the thyroid area infiltrated by lymphocytes and damaged, in control mice and anatabine-treated mice. p=0.05.

FIGS. 43A-B. Photomicrographs of thyroids of control mouse (FIG. 43A) and mouse treated with anatabine (FIG. 43B).

FIG. 44. Graph demonstrating levels of antibodies to PPD (purified protein derivative) in control and anatabine-treated mice.

FIG. 45. Graph demonstrating levels of antibodies to thyroglobulin on day 7 in control and anatabine-treated mice.

FIG. 46. Graph demonstrating levels of antibodies to thyroglobulin on day 14 in control and anatabine-treated mice.

FIG. 47. Graph demonstrating levels of antibodies to thyroglobulin on day 21 in control and anatabine-treated mice.

FIG. 48. Graph demonstrating that anatabine-treated mice have fewer activated T cells than control mice.

FIG. 49. Graph demonstrating that anatabine-treated mice have fewer T regulatory cells than control mice.

FIG. 50. Graph demonstrating that anatabine-treated mice appear to have lower antigen-presentation ability than control mice.

FIG. 51. Graph demonstrating thyroid histopathology in control and anatabine-treated mice.

FIG. 52. Graph showing effect of S-(−)-anatabine on TNFα-induced NFκB activity in vitro.

### DETAILED DESCRIPTION

This disclosure describes methods of using a composition comprising an isolated form of a compound of Formula I or IA (e.g., anatabine or S-(−)-anatabine) or a salt thereof to treat disorders comprising an inflammatory component, including chronic, low-level inflammation. “Treat” as used herein refers to reducing a symptom of the inflammation or resulting disorder but does not require complete cure, either of the inflammation or the disorder. “Reduction of a symptom” of a disorder with an NFκB-mediated inflammatory component includes but is not limited to elimination of the symptom, reduction in frequency, severity, or duration of the symptom, and delaying onset of the symptom. Accordingly, compositions comprising an isolated form of a compound of Formula I or IA (e.g., anatabine or S-(−)-anatabine) or a salt thereof can be administered to individuals before or after manifestation of a symptom. Symptoms include, but are not limited to, subjective indications (e.g., pain or swelling) as well as objective indications detectable with laboratory tests (e.g., an elevated level of an inflammatory marker such as C-reactive protein). Reduction of a symptom can be recognized subjectively by the individual or an observer of the individual or can be detected or identified by clinical and/or laboratory findings. In some embodiments, compositions comprising an isolated form of a compound of Formula I or IA (e.g., anatabine or S-(−)-anatabine) or a salt thereof are used to maintain inflammation at levels that promote well-being.

Compounds of Formula I

In some embodiments, a composition comprises an isolated form of a compound of Formula I, which can be provided as a pharmaceutically acceptable or food-grade salt:

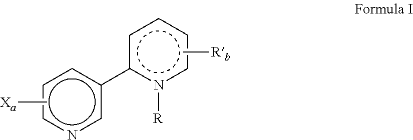

wherein:
R represents hydrogen or C₁-C₅ alkyl;
R′ represents hydrogen or C₁-C₇ alkyl; and
X represents halogen or C₁-C₇ alkyl.

In some embodiments,
R represents hydrogen or C₁-C₃ alkyl;
R′ represents hydrogen or C₁-C₄ alkyl; and
X represents halogen or C₁-C₃ alkyl.

The dotted line within the piperidine ring represents a carbon/carbon or carbon/nitrogen double bond within that ring, or two conjugated double bonds within that ring. One of the two conjugated double bonds can be a carbon/nitrogen double bond, or both of the conjugated double bonds can be carbon/carbon double bonds. When a carbon/nitrogen double bond is present, R is absent; and either (i) “a” is an integer ranging from 1-4, usually 1-2, and “b” is an integer ranging from 0-8, usually 0-4; or (ii) “a” is an integer ranging from 0-4, usually 0-2, and “b” is an integer ranging from 1-8, usually 1-4. When a carbon/nitrogen double bond is not present, R is present; “a” is an integer ranging from 0-4, usually 1-2; and “b” is an integer ranging from 0-8, usually 0-4 or 1-2. The term “alkyl,” as used herein, encompasses both straight chain and branched alkyl. The term “halogen” encompasses fluorine (F), chlorine (Cl), bromine (Br), and iodine (I).

Table 1 below illustrates non-limiting examples of compounds within Formula I:

TABLE 1

| R | R′ (position) | X (position) | a | b |
|---|---|---|---|---|
| H | CH₃ (3) | — | 0 | 1 |
| CH₃ | — | CH₃ (5) | 1 | 0 |
| H | — | CH₃CH₂ (4) | 1 | 0 |
| CH₃CH₂ | CH₃ (4) | — | 0 | 1 |
| H | CH₃ (2) | — | 0 | 2 |
| | CH₃CH₂ (5) | | | |
| H | CH₃ (3) | CH₃ (5) | 1 | 1 |
| CH₃ | — | CH₃ (2) | 2 | 0 |
| | | CH₃ (5) | | |

Compounds of Formula I may be present in the form of racemic mixtures or, in some cases, as isolated enantiomers as illustrated below in Formulas IA and IB.

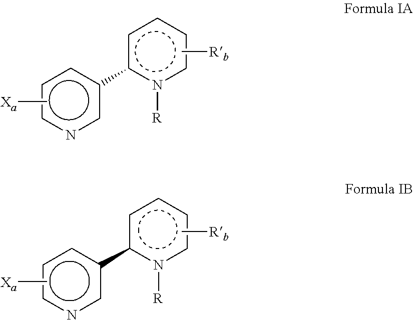

An example of a compound of Formula I is anatabine. An example of a compound of Formula IA is S-(−)-anatabine, and an example of compound of Formula IB is R-(+)-anatabine.

The chemical structure of anatabine (1,2,3,6-tetrahydro-[2,3′]bipyridinyl) is illustrated below, in which * designates an asymmetric carbon.

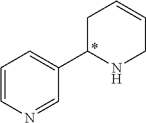

Anatabine exists in tobacco and certain foods and plants, including green tomatoes, green potatoes, ripe red peppers, tomatillos, sundried tomatoes, datura, mandrake, belladonna, capsicum, eggplant, and petunia, as a mixture of R-(+)-anatabine and S-(−)-anatabine, whose structures are illustrated below.

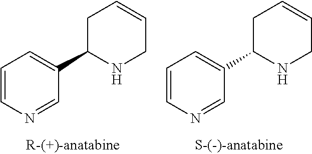

Anatabine, R-(+)-anatabine, S-(−)-anatabine, and other compounds of Formula I can be prepared synthetically. Such synthetic preparation techniques produce isolated forms of the compounds. Methods for selectively preparing the anatabine enantiomers are described, for example, in “A General Procedure for the Enantioselective Synthesis of the Minor Tobacco Alkaloids Nornicotine, Anabasine, and Anatabine,” The AAPS Journal 2005; 7(3) Article 75.

In some embodiments, anatabine is prepared via a benzophenoneimine pathway, as described in co-pending and commonly owned application Ser. No. 12/729,346, filed Mar. 23, 2010, the disclosure of which is incorporated herein by reference in its entirety.

Anatabine

In some embodiments, a compound of Formula I or IA (e.g., anatabine or S-(−)-anatabine) may be adsorbed on a cation exchange resin such as polymethacrilic acid (Amberlite IRP64 or Purolite C115HMR), as described in U.S. Pat. No. 3,901,248, the disclosure of which is hereby incorporated by reference in its entirety. Such cation exchange resins have been used commercially, for example, in nicotine replacement therapy, e.g., nicotine polacrilex.

In some embodiments, a compound of Formula I or IA (e.g., anatabine or S-(−)-anatabine) is provided in the form of a salt. “Salt,” as used herein, includes pharmaceutically acceptable and food-grade salts. In general, salts may provide improved chemical purity, stability, solubility, and/or bioavailability relative to anatabine in its native form. Non-limiting examples of possible anatabine salts are described in P. H. Stahl et al., Handbook of Pharmaceutical Salts: Properties, Selection and Use, Weinheim/Zürich:Wiley-VCH/VHCA, 2002, including salts of 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid (−L), malonic acid, mandelic acid (DL), methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid (−L), salicylic acid, sebacic acid, stearic acid, succinic acid, sulfuric acid, tartaric acid (+L), thiocyanic acid, toluenesulfonic acid (p), and undecylenic acid.

As an alternative to preparing anatabine synthetically, anatabine can be obtained by extraction from tobacco or other plants, such as members of the Solanaceae family, such as datura, mandrake, belladonna, capsicum, potato, nicotiana, eggplant, and petunia. For example, a tobacco extract may be prepared from cured tobacco stems, lamina, or both. In the extraction process, cured tobacco material is extracted with a solvent, typically water, ethanol, steam, or carbon dioxide. The resulting solution contains the soluble components of the tobacco, including anatabine. Anatabine may be purified from the other components of the tobacco using suitable techniques such as liquid chromatography.

As part of the purification process, tobacco material may be substantially denicotinized to remove a majority of other alkaloids such as nicotine, nornicotine, and anabasine. Denicotinizing is usually carried out prior to extraction of anatabine. Methods that may be used for denicotinizing tobacco materials are described, for example, in U.S. Pat. No. 5,119,835, the disclosure of which is hereby incorporated by reference. In general, tobacco alkaloids may be extracted from tobacco material with carbon dioxide under supercritical conditions. The tobacco alkaloids may then be separated from the carbon dioxide by dissolving an organic acid or a salt thereof, such as potassium monocitrate, in the carbon dioxide.

In some embodiments, an isolated form of anatabine is used. An “isolated form of anatabine,” as used herein, refers to anatabine that either has been prepared synthetically or has been substantially separated from plant materials in which it occurs naturally. The isolated form of anatabine should have a very high purity (including enantiomeric purity in the case where an enantiomer is used). In the case of synthetic anatabine, for example, purity refers to the ratio of the weight of anatabine to the weight of the end reaction product. In the case of isolating anatabine from plant material, for example, purity refers to the ratio of the weight of anatabine to the total weight of the anatabine-containing extract. Usually, the level of purity is at least about 95%, more usually at least about 96%, about 97%, about 98%, or higher. For example, the level of purity may be about 98.5%, 99.0%, 99.1%, 99.2%, 99.3%, 99.4%, 99.5%, 99.6%, 99.7%, 99.8%, 99.9%, or higher. Use of such isolated forms avoids the toxicity associated with tobacco, tobacco extracts, alkaloid extracts, and nicotine.

Anatabine and Inflammation

Without being bound by this explanation, data presented in Examples below indicate that anatabine reduces transcription mediated by nuclear factor KB (NFκB). NFκB is a transcription factor which operates in cells involved in inflammatory and immune reactions. As documented in Table IA, NFκB-mediated transcription is associated with numerous disorders, including those with an inflammatory component, an aberrant immune response, and/or inappropriate cell proliferation. Isolated forms of a compound of Formula I or IA (e.g., anatabine or S-(−)-anatabine) or salts thereof are useful for treating disorders comprising an “NFκB-mediated inflammatory component,” i.e. inflammation characterized by, caused by, resulting from, or affected by NFκB-mediated transcription.

NFκB-mediated transcription is implicated in an enormous variety of maladies. Based on anatabine's surprising efficacy in interfering with or interrupting this pivotal inflammatory-related activity, a compound of Formula I or IA (e.g., anatabine or S-(−)-anatabine) can be expected to have a wide range of therapeutic utilities. Unless otherwise clear from context, the term “anatabine” as used herein refers collectively to anatabine, either as a racemic mixture or an enantiomer, and pharmaceutically acceptable or food-grade salts of either of them.

Disorders

In some embodiments, an isolated form a compound of Formula I or IA (e.g., anatabine or S-(−)-anatabine) or a salt thereof can be administered to reduce the risk of developing a disorder comprising an NFκB-mediated inflammatory component (i.e., prophylactically). One can readily identify individuals with an increased risk or family history of a disorder. Other recognized indices of elevated risk of certain disorders can be determined by standard clinical tests or medical history.

In some embodiments, the disorder is an immune or autoimmune disorder. In some embodiments, the disorder is thyroiditis. In some embodiments, the disorder is arthritis, such as rheumatoid arthritis, primary and secondary osteoarthritis (also known as degenerative joint disease). In some embodiments, the disorder is a spondyloarthropathy, such as psoriatic arthritis, juvenile chronic arthritis with late pannus onset, and enterogenic spondyloarthropathies such as enterogenic reactive arthritis, urogenital spondyloarthropathy, and undifferentiated spondyloarthropathy. In some embodiments, the disorder is a myopathy, such as “soft tissue rheumatism” (e.g., tennis elbow, frozen shoulder, carpal tunnel syndrome, plantar fasciitis, and Achilles tendonitis).

In some embodiments, the disorder is diabetes, either type I diabetes or type II diabetes. In other embodiments the disorder is a gastrointestinal inflammatory disorder, such as an inflammatory bowel disease. Examples of inflammatory bowel disease include, but are not limited to, Crohn's disease, Barrett's syndrome, ileitis, irritable bowel syndrome, irritable colon syndrome, ulcerative colitis, pseudomembranous colitis, hemorrhagic colitis, hemolytic-uremic syndrome colitis, collagenous colitis, ischemic colitis, radiation colitis, drug and chemically induced colitis, diversion colitis, colitis in conditions such as chronic granulomatous disease, celiac disease, celiac sprue, food allergies, gastritis, infectious gastritis, enterocolitis (e.g., Helicobacter pylori-infected chronic active gastritis), and pouchitis.

In other embodiments the disorder is graft-versus-host-disease (GVHD), systemic lupus erythematosus (SLE), lupus nephritis, Addison's disease, Myasthenia gravis, vasculitis (e.g., Wegener's granulomatosis), autoimmune hepatitis, osteoporosis, and some types of infertility.

In some embodiments, the disorder is vascular inflammatory disease, associated vascular pathologies, atherosclerosis, angiopathy, inflammation-induced atherosclerotic or thromboembolic macroangiopathy, coronary artery disease, cerebrovascular disease, peripheral vascular disease, cardiovascular circulatory disease such as ischemia/reperfusion, peripheral vascular disease, restenosis following angioplasty, inflammatory aortic aneurysm, vasculitis, stroke, spinal cord injury, congestive heart failure, hemorrhagic shock, ischemic heart disease/reperfusion injury, vasospasm following subarachnoid hemorrhage, vasospasm following cerebrovascular accident, pleuritis, pericarditis, inflammation-induced myocarditis, or a cardiovascular complication of diabetes.

In some embodiments, the disorder is brain swelling or a neurodegenerative disease such as multiple sclerosis, Alzheimer's disease, or Parkinson's disease. In other embodiments the disorder is inflammation related to a kidney disease, nephritis, glomerulonephritis, dialysis, peritoneal dialysis, pericarditis, chronic prostatitis, vasculitis, gout, or pancreatitis.

In some embodiments, the disorder is an anemia. In other embodiments the disorder is an ulcer-related disease, such as peptic ulcer disease, acute pancreatitis, or aphthous ulcer. In other embodiments the disorder is related to an age-related disease, such as atherosclerosis, fibrosis, and osteoporosis, or a disorder associated with pre-maturity, such as retinopathy, chronic lung disease, arthritis, and digestive problems.

In other embodiments the disorder is preeclampsia, inflammation related to chemical or thermal trauma due to burns, acid, and alkali, chemical poisoning (MPTP/concavalin/chemical agent/pesticide poisoning), snake, spider, or other insect bites, adverse effects from drug therapy (including adverse effects from amphotericin B treatment), adverse effects from immunosuppressive therapy (e.g., interleukin-2 treatment), adverse effects from OKT3 treatment, adverse effects from GM-CSF treatment, adverse effects of cyclosporine treatment, and adverse effects of aminoglycoside treatment, stomatitis and mucositis due to immunosuppression, or exposure to ionizing radiation, such as solar ultraviolet exposure, nuclear power plant or bomb exposure, or radiation therapy exposure, such as for therapy for cancer.

In some embodiments, the disorder is a periodontal disease, such as plaque-associated gingivitis; acute necrotizing ulcerative gingivitis; hormone-induced gingival inflammation; drug-influenced gingivitis; linear gingival erythema (LGE); gingivitis due to bacterial, viral, or fungal infection; gingivitis due to blood dyscrasias or mucocutaneous diseases (e.g., lichen planus, pemphigus vulgaris, and desquamative gingivitis); plaque-associated adult periodontitis; early-onset periodontitis; prepubertal periodontitis; juvenile periodontitis; rapidly progressive periodontitis; periodontitis associated with systemic diseases; necrotizing ulcerative periodontitis; refractory periodontitis; and peri-implantitis.

In some embodiments, the disorder is a cancer, such as acute lymphoblastic leukemia, acute myeloid leukemia, adrenocortical carcinoma, AIDS-related lymphoma, anal cancer, appendix cancer, grade I (anaplastic) astrocytoma, grade II astrocytoma, grade III astrocytoma, grade IV astrocytoma, atypical teratoid/rhabdoid tumor of the central nervous system, basal cell carcinoma, bladder cancer, breast cancer, breast sarcoma, bronchial cancer, bronchoalveolar carcinoma, Burkitt lymphoma, cervical cancer, chronic lymphocytic leukemia, chronic myelogenous leukemia, colon cancer, colorectal cancer, craniopharyngioma, cutaneous T-cell lymphoma, endometrial cancer, endometrial uterine cancer, ependymoblastoma, ependymoma, esophageal cancer, esthesioneuroblastoma, Ewing's sarcoma, extracranial germ cell tumor, extragonadal germ cell tumor, extrahepatic bile duct cancer, fibrous histiocytoma, gallbladder cancer, gastric cancer, gastrointestinal carcinoid tumor, gastrointestinal stromal tumor, gestational trophoblastic tumor, gestational trophoblastic tumor, glioma, hairy cell leukemia, head and neck cancer, heart cancer, hepatocellular cancer, Hilar cholangiocarcinoma, Hodgkin's lymphoma, hypopharyngeal cancer, intraocular melanoma, islet cell tumor, Kaposi sarcoma, Langerhans cell histiocytosis, large-cell undifferentiated lung carcinoma, laryngeal cancer, lip cancer, lung adenocarcinoma, lymphoma, macroglobulinemia, malignant fibrous histiocytoma, medulloblastoma, medulloepithelioma, melanoma, Merkel cell carcinoma, mesothelioma, endocrine neoplasia, multiple myeloma, mycosis fungoides, myelodysplasia, myelodysplastic/myeloproliferative neoplasms, myeloproliferative disorders, nasal cavity cancer, nasopharyngeal cancer, neuroblastoma, non-Hodgkin's lymphoma, oral cancer, oropharyngeal cancer, osteosarcoma, ovarian clear cell carcinoma, ovarian epithelial cancer, ovarian germ cell tumor, pancreatic cancer, papillomatosis, paranasal sinus cancer, parathyroid cancer, penile cancer, pharyngeal cancer, pineal parenchymal tumor, pineoblastoma, pituitary tumor, plasma cell neoplasm, plasma cell neoplasm, pleuropulmonary blastoma, primary central nervous system lymphoma, prostate cancer, rectal cancer, renal cell cancer, respiratory tract cancer with chromosome 15 changes, retinoblastoma, rhabdomyosarcoma, salivary gland cancer, Sézary syndrome, small cell lung cancer, small intestine cancer, soft tissue sarcoma, squamous cell carcinoma, squamous non-small cell lung cancer, squamous neck cancer, supratentorial primitive neuroectodermal tumor, supratentorial primitive neuroectodermal tumor, testicular cancer, throat cancer, thymic carcinoma, thymoma, thyroid cancer, cancer of the renal pelvis, urethral cancer, uterine sarcoma, vaginal cancer, vulvar cancer, Waldenström macroglobulinemia, or Wilms tumor.

In some embodiments, the disorder is an upper respiratory tract infections (URI or URTI), such as tonsillitis, pharyngitis, laryngitis, sinusitis, otitis media, and the common cold. Infections which can be treated include, but are not limited to, rhinitis (e.g., inflammation of the nasal mucosa); rhinosinusitis or sinusitis (e.g., inflammation of the nares and paranasal sinuses, including frontal, ethmoid, maxillary, and sphenoid sinuses); nasopharyngitis (rhinopharyngitis or the common cold; e.g., inflammation of the nares, pharynx, hypopharynx, uvula, and tonsils); pharyngitis (e.g., inflammation of the pharynx, hypopharynx, uvula, and tonsils); epiglottitis (supraglottitis; e.g., inflammation of the superior portion of the larynx and supraglottic area); laryngitis (e.g., inflammation of the larynx); laryngotracheitis (e.g., inflammation of the larynx, trachea, and subglottic area); and tracheitis (e.g., inflammation of the trachea and subglottic area). By reducing underlying inflammation, symptoms which can be treated include, but are not limited to, cough, sore throat, runny nose, nasal congestion, headache, low grade fever, facial pressure, and sneezing.

In some embodiments, the disorder is a seizure disorder, i.e., any condition characterized by seizures, described in more detail below. Neuroinflammation is a well-established response to central nervous system injury (Minghetti, Curr Opin Neurol 2005; 18:315-21). Human pathologic, in vitro, and in vivo studies of Alzheimer's disease have implicated a glia-mediated neuroinflammatory response both in the pathophysiology of the disease (Mrak & Griffin, Neurobiol Aging 26:349-54, 2005) and as treatment target (Hu et al., Bioorgan Med Chem Lett 17:414-18, 2007; Ralay et al., J Neurosci 26:662-70, 2006; Craft et al., Exp Opin Therap Targets 9:887-900, 2005). Microglial activation leading to overexpression of IL-1 has been proposed as the pivotal step in initiating a self propagating cytokine cycle culminating in neurodegeneration (Mrak & Griffin, Neurobiol Aging 26:349-54, 2005; Sheng et al., Neurobiol Aging 17:761-66, 1996). As noted above, data presented in Examples 1 and 2 below indicate that anatabine reduces transcription mediated by nuclear factor κB (NFκB). IL-1β and pro-inflammatory cytokines may also function in epilepsy as pro-convulsant signaling molecules independent of such a cycle (Vezzani et al., Epilepsia 43:S30-S35, 2002), which provides a potential therapeutic target in epilepsy and other seizure disorders (Vezzani & Granata, Epilepsia 46:1724-43, 2005).

In some embodiments, an isolated form of a compound of Formula I or IA (e.g., anatabine or S-(−)-anatabine) or a salt thereof is administered to treat seizures, including the generalized and partial seizures. As described in The Pharmacological Basis of Therapeutics, 9^{th} ed., (McGraw-Hill), there are two classes of seizures: partial seizures and generalized seizures. Partial seizures consist of focal and local seizures. Partial seizures are further classified as simple partial seizures, complex partial seizures and partial seizures secondarily generalized. Generalized seizures are classified as convulsive and nonconvulsive seizures. They are further classified as absence (previously referred to as ‘petit mal’) seizures, atypical absence seizures, myoclonic seizures, clonic seizures, tonic seizures, tonic-clonic seizures, and atonic seizures.

Generalized seizures include infantile spasms, absence seizures, tonic-clonic seizures, atonic seizures, and myoclonic seizures. Abnormal motor function and a loss of consciousness are major features of these seizures. A patient may also experience an aura of sensory, autonomic, or psychic sensations. The aura may include paresthesia, a rising epigastric sensation, an abnormal smell, a sensation of fear, or a deja vu sensation. A generalized seizure is often followed by a postictal state, in which a patient may sleep deeply, be confused, and/or have a headache or muscle ache. Todd's paralysis (limb weakness contralateral to the seizure focus) may be present in the postictal state.

Infantile spasms are characterized by frequent flexion and adduction of the arms and forward flexion of the trunk, usually of short duration. They occur only in the first 5 years of life.

Typical absence seizures (also known as petit mal seizures) are characterized by a loss of consciousness with eyelid fluttering, typically for 10-30 seconds or more. There may or may not be a loss of axial muscle tone. Convulsions are absent; instead, patients abruptly stop activity, then abruptly resume it, often without realizing that a seizure has occurred. Absence seizures are genetic. They occur predominantly in children, often frequently throughout the day.

Atypical absence seizures occur as part of the Lennox-Gastaut syndrome, a severe form of epilepsy. They last longer than typical absence seizures and jerking or automatic movements are more pronounced.

Atonic seizures occur most often in children, usually as part of Lennox-Gastaut syndrome. They are characterized by a complete loss of muscle tone and consciousness.

Tonic seizures also occur most often in children, usually as part of Lennox-Gastaut syndrome. They are characterized by tonic (sustained) contraction of axial and proximal muscles, usually during sleep, and last 10 to 15 seconds. In longer tonic seizures a few, rapid clonic jerks may occur at the end of the seizure.

Tonic-clonic seizures, also known as grand mal seizures, may be primarily or secondarily generalized. A patient experiencing a primarily generalized tonic-clonic seizure will often cry out, then lose consciousness and fall. Tonic contractions then begin, followed by clonic (rapidly alternating contraction and relaxation) motion of muscles of the extremities, trunk, and head. A patient may lose urinary and fecal continence, bite his tongue, and froth at the mouth. Seizures usually last 1 to 2 min. There is no aura. Secondarily generalized tonic-clonic seizures begin with a simple partial or complex partial seizure, and then progress to a generalized seizure.

Myoclonic seizures are characterized by brief, rapid jerks of a limb, several limbs, or the trunk. They may be repetitive, leading to a tonic-clonic seizure. The jerks may be bilateral or unilateral. Consciousness is not lost unless the seizures progress into a generalized tonic-clonic seizure.

Juvenile myoclonic epilepsy is an epilepsy syndrome characterized by myoclonic, tonic-clonic, and absence seizures. Patients are usually adolescents. Seizures typically begin with bilateral, synchronous myoclonic jerks, followed in 90% by generalized tonic-clonic seizures. They often occur on rising in the morning. A third of patients may experience absence seizures.

Febrile seizures are associated with fever, but not intracranial infection. Benign febrile seizures are characterized by generalized tonic-clonic seizures of brief duration. Such seizures are common in children, affecting up to four percent of children younger than six years of age. Complicated febrile seizures are characterized by focal seizures lasting more than fifteen minutes or occurring more than twice in twenty four hours. Two percent of children with febrile seizures develop a subsequent seizure disorder. The risk is greater in children with complicated febrile seizures, preexisting neurologic abnormalities, onset before age 1 year, or a family history of seizure disorders.

Status epilepticus is a seizure disorder characterized by tonic-clonic seizure activity lasting more than five to ten minutes, or two or more seizures between which patients do not fully regain consciousness. If untreated, seizures lasting more than sixty minutes may cause brain damage or death.

Complex partial status epilepticus and absence status epilepticus are characterized by prolonged episodes of mental status changes. Generalized convulsive status epilepticus may be associated with abrupt withdrawal of anticonvulsants or head trauma.

Simple partial seizures are characterized by motor, sensory, or psychomotor symptoms without loss of consciousness. Seizures in different parts of the brain often produce distinct symptoms.

An aura often precedes complex partial seizures. Patients are usually aware of their environment but may experience impaired consciousness. Patients may also experience oral automatisms (involuntary chewing or lip smacking), hand or limb automatisms (automatic purposeless movements), utterance of unintelligible sounds, tonic or dystonic posturing of the extremity contralateral to the seizure focus, head and eye deviation, usually in a direction contralateral to the seizure focus, and bicycling or pedaling movements of the legs, especially where the seizure emanates from the medial frontal or orbitofrontal head regions. Motor symptoms subside after one or two minutes, and confusion and disorientation one to two minutes later. Postictal amnesia is common.

Epilepsy is an important example of a seizure disorder. “Epilepsy” describes a group of central nervous system disorders that are characterized by recurrent seizures that are the outward manifestation of excessive and/or hyper-synchronous abnormal electrical activity of neurons of the cerebral cortex and other regions of the brain. This abnormal electrical activity can be manifested as motor, convulsion, sensory, autonomic, or psychic symptoms.

Hundreds of epileptic syndromes have been defined as disorders characterized by specific symptoms that include epileptic seizures. These include, but are not limited to, absence epilepsy, psychomotor epilepsy, temporal lobe epilepsy, frontal lobe epilepsy, occipital lobe epilepsy, parietal lobe epilepsy, Lennox-Gastaut syndrome, Rasmussen's encephalitis, childhood absence epilepsy, Ramsay Hunt Syndrome type II, benign epilepsy syndrome, benign infantile encephalopathy, benign neonatal convulsions, early myoclonic encephalopathy, progressive epilepsy and infantile epilepsy. A patient may suffer from any combination of different types of seizures. Partial seizures are the most common, and account for approximately 60% of all seizure types.

Hence, examples of generalized seizures which may be treated include infantile spasms, typical absence seizures, atypical absence seizures, atonic seizures, tonic seizures, tonic-clonic seizures, myoclonic seizures, and febrile seizures. Examples of partial seizures which may be treated include simple partial seizures affecting the frontal lobe, contralateral frontal lobe, supplementary motor cortex, the insula, the Insular-orbital-frontal cortex, the anteromedial temporal lobe, the amygdala (including the opercular and/or other regions), the temporal lobe, the posterior temporal lobe, the amygdala, the hippocampus, the parietal lobe (including the sensory cortex and/or other regions), the occipital lobe, and/or other regions of the brain.

In some embodiments, an isolated form of a compound of Formula I or IA (e.g., anatabine or S-(−)-anatabine) or a salt thereof is administered to treat an epileptic syndrome including, but not limited to, absence epilepsy, psychomotor epilepsy, temporal lobe epilepsy, frontal lobe epilepsy, occipital lobe epilepsy, parietal lobe epilepsy, Lennox-Gastaut syndrome, Rasmussen's encephalitis, childhood absence epilepsy, Ramsay Hunt Syndrome type II, benign epilepsy syndrome, benign infantile encephalopathy, benign neonatal convulsions, early myoclonic encephalopathy, progressive epilepsy and infantile epilepsy.

An isolated form of a compound of Formula I or IA (e.g., anatabine or S-(−)-anatabine) or a salt thereof may also be useful for treating the aura that accompanies seizures. Thus, impaired consciousness, oral automatisms, hand or limb automatisms, utterance of unintelligible sounds, tonic or dystonic posturing of extremities, head and eye deviation, bicycling or pedaling movements of the legs and other symptoms that comprise the aura also may be treated.

Patients who can be treated include adults, teenagers, children, and neonates. Neonatal seizures are associated with later neurodevelopmental and cognitive deficits including mental retardation, autism, and epilepsy, and it is estimated that up to 40% of cases of autism suffer from epilepsy or have a history of or seizures earlier in life. Accordingly, important target patients are infants, particularly neonates, and persons with a personal or family a history of seizure, mental retardation or autism.

This disclosure also provides methods and compositions for treating a patient post-seizure. In one embodiment, an isolated form of a compound of Formula I or IA (e.g., anatabine or S-(−)-anatabine) or a salt thereof is administered in conjunction with a second therapeutic agent, such as a neurotransmitter receptor inhibitor (e.g., an inhibitor of an AMPA receptor, NMDA receptor GABA receptor, chloride cotransporters, or metabatropic glutamate receptor), a kinase/phosphatase inhibitor (e.g., an inhibitor of calmodulin kinase II (CamK II), protein kinase A (PKA), protein kinase C (PKC), MAP Kinase, Src kinase, ERK kinase or the phosphatase calcineurin), and/or a protein translation inhibitor.

Calmodulin kinase II (CamK II) inhibitors include KN-62, W-7, HA-1004, HA-1077, and staurosporine. Protein kinase A (PKA) inhibitors include H-89, HA-1004, H-7, H-8, HA-100, PKI, and staurosporine.

Protein kinase C (PKC) inhibitors include competitive inhibitors for the PKC ATP-binding site, including staurosporine and its bisindolylmaleimide derivatives, Ro-31-7549, Ro-31-8220, Ro-31-8425, Ro-32-0432 and Sangivamycin; drugs which interact with the PKC's regulatory domain by competing at the binding sites of diacylglycerol and phorbol esters, such as calphostin C, Safingol, D-erythro-Sphingosine; drugs which target the catalytic domain of PKC, such as chelerythrine chloride, and Melittin; drugs which inhibit PKC by covalently binding to PKC upon exposure to UV lights, such as dequalinium chloride; drugs which specifically inhibit Ca-dependent PKC such as Go6976, Go6983, Go7874 and other homologs, polymyxin B sulfate; drugs comprising competitive peptides derived from PKC sequence; and [0056]PKC inhibitors such as cardiotoxins, ellagic acid, HBDDE, 1-O-Hexadecyl-2-O-methyl-rac-glycerol, Hypercin, K-252, NGIC-I, Phloretin, piceatannol, and Tamoxifen citrate.

MAP kinase inhibitors include SB202190 and SB203580. SRC kinase inhibitors include PP1, PP2, Src Inhibitor No. 5, SU6656, and staurosporine. ERK kinase inhibitors include PD 98059, SL327, olomoucine, and 5-Iodotubercidin. Calcineurin inhibitors include tacrolimus and cyclosporine.

Protein translation inhibitors include mTOR inhibitors, such as rapamycin, CCI-779 and RAD 001.

In some embodiments, an isolated form of a compound of Formula I or IA (e.g., anatabine or S-(−)-anatabine) is administered to treat an Autism Spectrum Disorder. Autism spectrum disorders (ASDs) are pervasive neurodevelopmental disorders diagnosed in early childhood when acquired skills are lost or the acquisition of new skills becomes delayed. ASDs onset in early childhood and are associated with varying degrees of dysfunctional communication and social skills, in addition to repetitive and stereotypic behaviors. In many cases (25%-50%), a period of seemingly normal development drastically shifts directions as acquired skills are lost or the acquisition of new skills becomes delayed. Examples of Autism Spectrum Disorders include “classical” autism, Asperger's syndrome, Rett syndrome, childhood disintegrative disorder, and atypical autism otherwise known as pervasive developmental disorder not otherwise specified (PDD-NOS).

Autism is a childhood psychosis originating in infancy and characterized by a wide spectrum of psychological symptoms that progress with age (e.g., lack of responsiveness in social relationships, language abnormality, and a need for constant environmental input). It generally appears in children between the ages of two and three years and gives rise to a loss of the development previously gained by the child. Autistic individuals are at increased risk of developing seizure disorders, such as epilepsy.

Excess inflammation has been found in the colon, esophagus, and duodenum of patients with autism, and postmortem studies have also shown an increase in the expression of several markers for neuroinflammation (e.g., Wakefield et al., Lancet 351, 351-52, 1998; Wakefield et al., Lancet 351, 637-41, 1998; and Vargas et al., Ann Neurol 57, 67-81, 2004). Proinflammatory cytokines, including TNFα and IL-1, are overproduced in a subset of autistic patients, indicating that these patients had excessive innate immune responses and/or aberrant production of regulatory cytokines for T cell responses (e.g., 20030148955. Isolated forms of compounds of Formula I or IA (e.g., anatabine or S-(−)-anatabine) or salts thereof are particularly useful for treating disorders comprising an “NFκB-mediated inflammatory component,” i.e. inflammation characterized by, caused by, resulting from, or affected by NFκB-mediated transcription. Thus, a compound of Formula I or IA (e.g., anatabine or S-(−)-anatabine) in isolated form may be useful in treating or reducing a symptom of an ASD.

In some embodiments, the dose sufficient to reduce a symptom of the disorder can include a series of treatments. For example, an individual can be treated with a dose of an isolated form of anatabine or S-(−)-anatabine or a salt thereof several times per day (e.g., 2-12 or 4-10 times per day), once daily, or less frequently such as 1-6 times per week.

In some embodiments, the compound administered is an isolated form of a compound of Formula I or IA (e.g., anatabine or S-(−)-anatabine) which is administered several times per day (e.g., 2-12 or 4-10 times per day), once daily, or less frequently such as 1-6 times per week. Treatments may span between about 1 to 10 weeks (e.g., between 2 to 8 weeks, between 3 to 7 weeks, for about 1, 2, 3, 4, 5, 6, 7, 8, 9, or 10 weeks). It will also be appreciated that a dose regimen used for treatment may increase or decrease over the course of a particular treatment.

In some embodiments, an isolated form of a compound of Formula I or IA (e.g., anatabine or S-(−)-anatabine) or a salt thereof can be administered to reduce the risk of developing an ASD (i.e., prophylactically). One can readily identify individuals with an increased risk or family history of a disorder. Other recognized indices of elevated risk of certain disorders can be determined by standard clinical tests or medical history.

An isolated form of a compound of Formula I or IA (e.g., anatabine or S-(−)-anatabine) or a salt thereof can also be used to improve erectile dysfunction, either administered alone of in conjunction with other therapies such as tadalafil (e.g. CIALIS®), vardenafil (e.g., LEVITRA®, STAXYN®), and sildenafil (e.g., VIAGRA®).

In some embodiments, a therapeutically effective dose of an isolated form of a compound of Formula I or IA (e.g., anatabine or S-(−)-anatabine) or a salt thereof can be administered to an individual for treating alopecia areata or other disorders associated with hair loss.

Doses

In some embodiments an isolated form of a compound of Formula I or IA (e.g., anatabine or S-(−)-anatabine) or a salt thereof is administered to an individual in an amount sufficient to reduce NFκB-mediated transcription (“NFκB-inhibiting amounts”). In some embodiments, an isolated form of a compound of Formula I or IA (e.g., anatabine or S-(−)-anatabine) or a salt thereof is administered to an individual at a dose sufficient to reduce a symptom of a disorder with an NFκB-mediated-transcription component, such as the disorders described above. “Individual” as used herein includes warm-blooded animals, typically mammals, including humans and other primates. In some embodiments, the individual is an animal such as a companion animal, a service animal, a farm animal, or a zoo animal. Such animals include, but are not limited to, canines (including dogs, wolves), felines (including domestic cats, tigers, lions), ferrets, rabbits, rodents (e.g., rats, mice), guinea pigs, hamsters, gerbils, horses, cows, pigs, sheep, goats, giraffes, and elephants. In some embodiments, the individual is a non-human mammal.

In some embodiments an isolated form of a compound of Formula I or IA (e.g., anatabine or S-(−)-anatabine) or a salt thereof is administered to an individual to treat a disorder comprising an inflammatory component or a symptom of such a disorder. In some embodiments the inflammatory component is chronic, low-level inflammation. In some embodiments the symptom is eliminated. In some embodiments the symptom is reduced in frequency, severity, or duration. In some embodiments the onset of the symptom is delayed.

In some embodiments an isolated form of a compound of Formula I or IA (e.g., anatabine or S-(−)-anatabine) or a salt thereof is administered to an individual before manifestation of a symptom. In some embodiments the symptom is a subjective indication. In some embodiments the symptom is an objective indication. In some embodiments, the symptom is an elevated level of an inflammatory marker such as C-reactive protein.

In some embodiments, an isolated form of a compound of Formula I or IA (e.g., anatabine or S-(−)-anatabine) or a salt thereof is administered to an individual after manifestation of a symptom. In some embodiments the symptom is a subjective indication. In some embodiments the symptom is an objective indication. In some embodiments, the symptom is an elevated level of an inflammatory marker such as C-reactive protein.

In some embodiments, an isolated form of a compound of Formula I or IA (e.g., anatabine or S-(−)-anatabine) or a salt thereof is used to maintain inflammation at levels that promote well-being.

Daily doses typically range from about 1 μg/kg to about 7 mg/kg body weight, e.g.:
i. about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 μg/kg;
ii. about 0.1, 0.11, 0.12, 0.125, 0.13, 0.14, 0.145, 0.15, 0.16, 0.17, 0.175, 0.18, 0.19, 0.2, 0.3, 0.31, 0.32, 0.33, 0.34, 0.35, 0.36, 0.37, 0.38, 0.39, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 mg/kg);
iii. about 1.5 μg/kg to about 5 μg/kg, about 1 μg/kg to about 10 μg/kg, about 0.01 mg/kg to about 7 mg/kg body weight, about 0.1 mg/kg to about 5 mg/kg;
iv. about 0.1 mg/kg to about 2 mg/kg, about 1 mg/kg to about 3 mg/kg, about 0.5 mg/kg to about 2 mg/kg, about 1 mg/kg to about 2 mg/kg, about 3 mg/kg to about 5 mg/kg, about 2 mg/kg to about 4 mg/kg, about 2 mg/kg to about 5 mg/kg, or about 0.5 mg/kg to about 1.5 mg/kg;
v. about 5, 10, 15, 20, 25, 30, 35, 40, 45, 50, 55, 60, 65, 70, 71, 72, 73, 74, 75, 76, 77, 78, 79, 80, 85, 90, 100, 105, 110, 115, 117, 118, 119, 120, 125, 130, 135, 136, 140, 145, 146, 147, 148, 149, 150, 175, 200, or 205 mg/kg; or
vi. about 55 to about 120 μg/kg, about 100 to about 150 μg/kg, about 100 to about 200 μg/kg, about 110 to about 146 μg/kg, about 118 to about 150 μg/kg, about 110 to about 150 μg/kg, about 117 to about 147 μg/kg, about 70 to about 140 μg/kg, or about 125 to about 350 140 μg/kg.

Dosages described above may apply to any of the disorders disclosed herein; however, certain factors may influence the dose sufficient to reduce a symptom of a disorder (i.e., an effective dose), including the type and/or severity of the disease or disorder, previous treatments, the general health, age, and/or weight of the individual, the frequency of treatments, the rate of release from the composition, and other diseases present. This dose may vary according to factors such as the disease state, age, and weight of the subject. For example, higher doses may be administered for treatments involving conditions which are at an advanced stage and/or life-threatening. Dosage regimens also may be adjusted to provide the optimum therapeutic response.

For example, in some embodiments, a neurodegenerative disease, such as Alzheimer's disease or Parkinson's disease, is treated by administering an isolated form of a compound of Formula I or IA (e.g., anatabine or S-(−)-anatabine) in an amount that exceeds 150 μg per kg patient weight. In other embodiments, a neurodegenerative disease, such as Alzheimer's disease or Parkinson's disease, is treated by administering an isolated form of a compound of Formula I or IA (e.g., anatabine or S-(−)-anatabine) in an amount that is between about 50 μg and 100 μg or between about 100 μg and 150 μg per kg patient weight.

In some embodiments, tablets comprising about 600 μg S-(−)-anatabine citrate or about 1 mg anatabine citrate are administered from once to 25 times daily (e.g., 1, 2, 3, 4, 5, 6, 7, 8, 9, 10, 11, 12, 13, 14, 15, 16, 17, 18, 19, 20, 21, 22, 23, 24, or 25) times daily.

In some embodiments, thyroiditis is treated by administering to an individual 600 μg anatabine citrate, 20 times daily over a period of 30 days. In some embodiments, the individual is treated with approximately 0.1 mg/kg/day of anatabine or S-(−)-anatabine.

In some embodiments, the dose sufficient to reduce the symptom of the disorder being treated can include a series of treatments. For example, an individual can be treated with a dose of an isolated form of a compound of Formula I or IA (e.g., anatabine or S-(−)-anatabine) or a salt thereof several times per day (e.g., 2-12 or 4-10 times per day), once daily, or less frequently such as 1-6 times per week. Treatments may span between about 1 to 10 weeks (e.g., between 2 to 8 weeks, between 3 to 7 weeks, for about 1, 2, 3, 4, 5, 6, 7, 8, 9, or 10 weeks). It will also be appreciated that a dose regimen used for treatment may increase or decrease over the course of a particular treatment.

Use with Other Therapies

An isolated form of a compound of Formula I or IA (e.g., anatabine or S-(−)-anatabine) or a salt thereof can be used in conjunction with (i.e., before, after, or at the same time as) other therapies for any disorder with an NFκB-mediated component. In some embodiments, these therapies include other products that inhibit production of NFκB-mediated inflammatory species. These products include, but are not limited to, dexamethasone, glucocorticoids (e.g., prednisone, methyl prednisolone), cyclosporine, tacrolimus, deoxyspergualin, non-steroidal antiinflammatory drugs (NSAIDs) such as aspirin and other salicylates, tepoxalin, synthetic peptide proteosome inhibitors, antioxidants (e.g., N-acetyl-L-cysteine, vitamin A, vitamin C, vitamin E, dithiocarbamate derivatives, curcumin), IL-10, nitric oxide, cAMP, gold-containing compounds, and gliotoxin.

Pharmaceutical Compositions

Pharmaceutical compositions may be formulated together with one or more acceptable pharmaceutical or food grade carriers or excipients. As used herein, the term “acceptable pharmaceutical or food grade carrier or excipient” means a non-toxic, inert solid, semisolid or liquid filler, diluent, encapsulating material or formulation auxiliary of any type. For example, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutical compositions may be prepared by any suitable technique and is not limited by any particular method for its production. For example, a compound of Formula I or IA (e.g., anatabine or S-(−)-anatabine) can be combined with excipients and a binder, and then granulated. The granulation can be dry-blended with any remaining ingredients, and compressed into a solid form such as a tablet.

Pharmaceutical compositions may be administered by any suitable route. For example, the compositions may be administered orally, parenterally, by inhalation spray, topically, rectally, nasally, buccally, vaginally, via an implanted reservoir, or ingested as a dietary supplement or food. In some embodiments, a composition is provided in an inhaler, which may be actuated to administer a vaporized medium that is inhaled into the lungs. The term parenteral as used herein includes subcutaneous, intracutaneous, intravenous, intramuscular, and intracranial injection or infusion techniques. Most often, the pharmaceutical compositions are readily administered orally and ingested.

Pharmaceutical compositions may contain any conventional non-toxic pharmaceutically-acceptable carriers, adjuvants or vehicles. In some cases, the pH of the formulation may be adjusted with acceptable pharmaceutical or food grade acids, bases or buffers to enhance the stability of the formulated composition or its delivery form.

Liquid dosage forms for oral administration include acceptable pharmaceutical or food grade emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylsulfoxide (DMSO) dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, lozenges, pills, powders, and granules. In such solid dosage forms, the active compound is mixed with at least one inert, acceptable pharmaceutical or food grade excipient or carrier such as sodium citrate or dicalcium phosphate and/or a) fillers or extenders such as starches, lactose, sucrose, glucose, mannitol, and silicic acid, b) binders such as carboxymethylcellulose, alginates, gelatin, polyvinylpyrrolidone, sucrose and acacia, c) humectants such as glycerol, d) disintegrating agents such as agaragar, calcium carbonate, potato or tapioca starch, alginic acid, certain silicates, and sodium carbonate, e) solution retarding agents such as paraffin, f) absorption accelerators such as quaternary ammonium compounds, g) wetting agents such as cetyl alcohol and glycerol monostearate, h) absorbents such as kaolin and bentonite clay, i) lubricants such as talc, calcium stearate, magnesium stearate, solid polyethylene glycols, sodium lauryl sulfate, and mixtures thereof, and j) sweetening, flavoring, perfuming agents, and mixtures thereof. In the case of capsules, lozenges, tablets and pills, the dosage form may also comprise buffering agents.

The solid dosage forms of tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract or, optionally, in a delayed or extended manner. Examples of embedding compositions which can be used include polymeric substances and waxes. Tablet formulations for extended release are also described in U.S. Pat. No. 5,942,244.

Inflammatory Markers

An isolated form of a compound of Formula I or IA (e.g., anatabine or S-(−)-anatabine) can be used to reduce elevated blood levels of inflammatory markers such as CRP or to maintain healthy levels of such markers. Thus, in some embodiments levels of inflammatory markers can be used to aid in determining doses of an isolated form of a compound of Formula I or IA (e.g., anatabine or S-(−)-anatabine) to be administered as well as to monitor treatment of various inflammatory disorders and to assist physicians in deciding on a course of a treatment for an individual at risk of an inflammatory disorder. These markers include, but are not limited to, C-reactive protein (CRP), soluble intercellular adhesion molecule (sICAM-1), ICAM 3, BL-CAM, LFA-2, VCAM-1, NCAM, PECAM; fibrinogen, serum amyloid A (SAA), TNFα, lipoprotein associated phospholipase A2 (LpPlA2), sCD40 ligand, myeloperoxidase, interleukin-6 (IL-6), and interleukin-8 (IL-8).

The level of one or more inflammatory markers can be determined in a patient already being treated with an isolated form of a compound of Formula I or IA (e.g., anatabine or S-(−)-anatabine) or in an individual at risk for an inflammatory disorder or suspected of having an inflammatory disorder. The level is compared to a predetermined value, and the difference indicates whether the patient will benefit from administration of an isolated form of a compound of Formula I or IA (e.g., anatabine or S-(−)-anatabine) or from continued administration of an isolated form of a compound of Formula I or IA (e.g., anatabine or S-(−)-anatabine). The level of inflammatory marker can be determined by any art recognized method. Typically, the level is determined by measuring the level of the marker in a body fluid, for example, blood, lymph, saliva, or urine. The level can be determined by ELISA, or immunoassays or other conventional techniques for determining the presence of the marker. Conventional methods include sending a sample(s) of a patient's body fluid to a commercial laboratory for measurement.

The predetermined value can take a variety of forms and will vary according to the inflammatory marker. The predetermined value can be single cut-off value, such as a median or a mean, or it can be a range. The predetermined value also can depend on the individual or particular inflammatory disorder. Appropriate ranges and categories can be selected by those of ordinary skill in the art using routine methods. See US 2006/0115903; US 2004/0175754.

Markers such as CRP, sICAM-1, ICAM 3, BL-CAM, LFA-2, VCAM-1, NCAM, PECAM, fibrinogen, SAA, TNFα, lipoprotein associated phospholipase A2 (LpPlA2), sCD40 ligand, myeloperoxidase, IL-6, and IL-8 are useful markers for systemic inflammation. In some embodiments, the inflammatory marker is CRP, which is associated both with cardiovascular disease (see US 2006/0115903) and cancer, such as colon cancer (Baron et al., N. Engl. J. Med. 348, 891-99, 2003). Elevated levels of CRP are also observed in patients with insulin-resistance (Visser et al., JAMA. 1999, 282(22):2131-5). Diabetic and insulin-resistant patients also have elevated levels of TNFα, IL-6, and IL-8 (Roytblat et al., Obes Res. 2000, 8(9):673-5; Straczkowski et al., J Clin Endocrinol Metab. 2002, 87(10):4602-6; Hotamisligil et al., Science. 1996, 271(5249):665-8; Sartipy P, Loskutoff D J. Proc Natl Acad Sci USA. 2003, 100(12):7265-70; Hotamisligil et al., J Clin Invest. 1995, 95(5):2409-15).

Products Containing Anatabine

In addition to pharmaceutical compositions described above, isolated forms of a compound of Formula I or IA (e.g., anatabine or S-(−)-anatabine) or salts thereof can be provided together with other ingredients, for example, in the form of an elixir, a beverage, a chew, a tablet, a lozenge, a gum, and the like.

In some embodiments a beverage suitable for human consumption contains a liquid medium and one or more isolated forms of a compound of Formula I or IA (e.g., anatabine or S-(−)-anatabine) or salts thereof. The liquid medium may be, for example, water of sufficiently high purity, or other beverage medium such as citrus juice or the like. The liquid medium and compound(s) of Formula I or IA (e.g., anatabine or S-(−)-anatabine) or salts thereof, may be combined with other ingredients to improve product characteristics, such as flavor, taste, color/clarity, and/or stability. Other beneficial components also may be added, such as vitamins, proteinaceous ingredients, or the like.

The components may be combined using appropriate equipment, such as blenders, and packaged in conventional beverage containers, such as single-serving (or larger) glass bottles, plastic bottles, cans, or the like. A beverage container may contain, for example, from about 100 ml to about 2,000 ml purified water and from about 0.00001 to about 0.0001 wt % of an isolated form of a compound of Formula I or IA (e.g., anatabine or S-(−)-anatabine) or a salt of a compound of Formula I or IA (e.g., anatabine or S-(−)-anatabine).

In some embodiments, an isolated form of a compound of Formula I or IA (e.g., anatabine or S-(−)-anatabine) or salts thereof is provided in a fluid form (e.g., a liquid, paste, cream, lotion, etc.) for topical application. In some embodiments, the fluid form is a therapeutic product for use in treating dermatological disorders (e.g., psosriasis). In some embodiments, the fluid form is a skin care product such as a moisturizer or sunscreen. In some embodiments, the fluid form is a cosmetic product. In some embodiments the fluid form is a toothpaste or a mouthwash.

The following examples illustrate but do not limit the scope of the disclosure set forth above.

### EXAMPLE 1

### NFκB-Mediated Transcription Assays

### Cytotoxicity Assays

The effect of a range of doses of anatabine, nicotine, crude extract of smokeless tobacco, and alkaloid extract of smokeless tobacco was examined in an NFκB luciferase assay (inhibition of TNFα-induced NFκB activity). The smokeless tobacco used in these experiments was plain long-leaf Copenhagen tobacco purchased from a local vendor. Crude extract was extracted with methanol and water and clarified by centrifugation and filtration. The alkaloid extract was prepared from sodium hydroxide and methanol extraction, organic phase separation and purification. All treatment samples were prepared as a function of weight (μg/ml), and all samples were diluted in DMSO. Dilutions were made immediately before cell culture treatments and, in all cases, the final amount of DMSO did not exceed 1% in cell culture media.

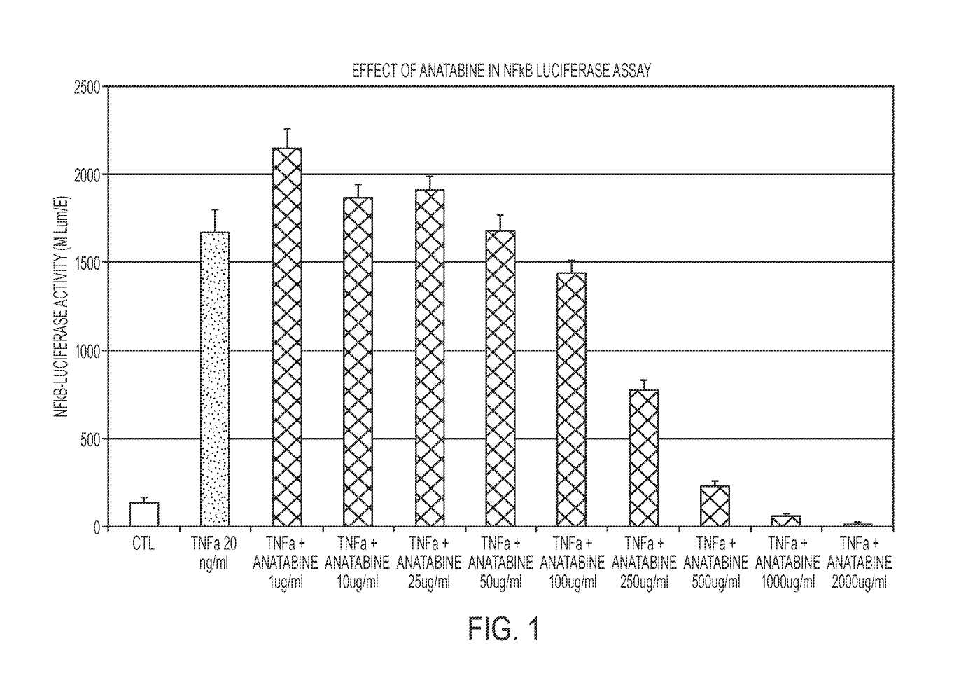

Human endothelial kidney cells (HEK293) transfected with an NFκB luciferase reporter were challenged with TNFα for three hours, then samples were applied to the challenged cells. The results are shown in FIGS. 1-3.

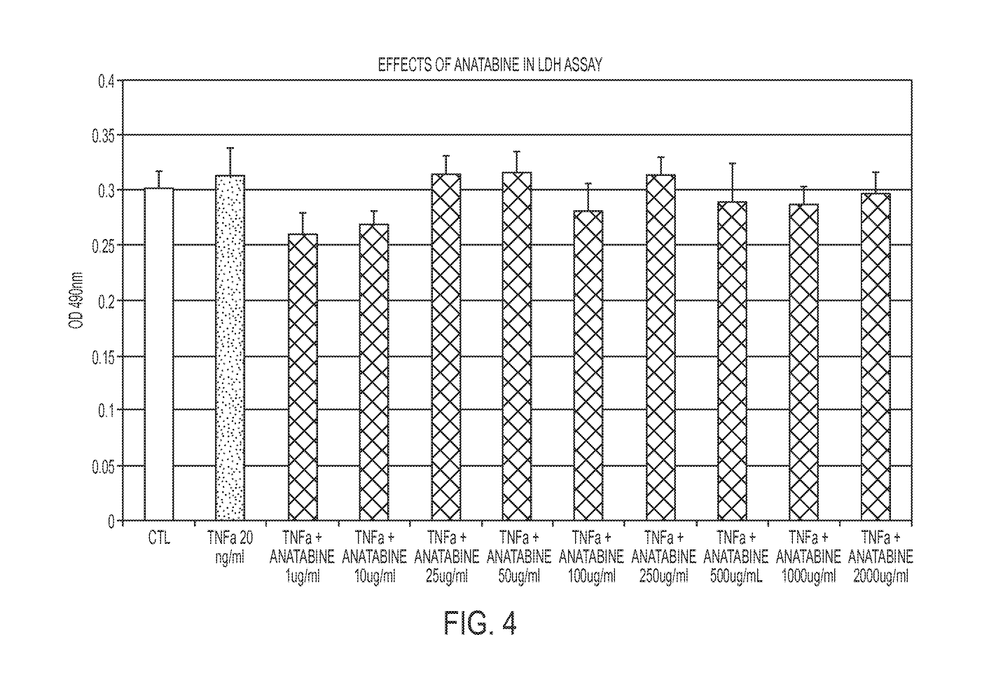

Cytotoxicity assays using the supernatants from the treated cells were conducted using an LDH Cytotoxicity Detection Kit (Roche) according to the manufacturer's instructions. The results are shown in FIGS. 4-6.

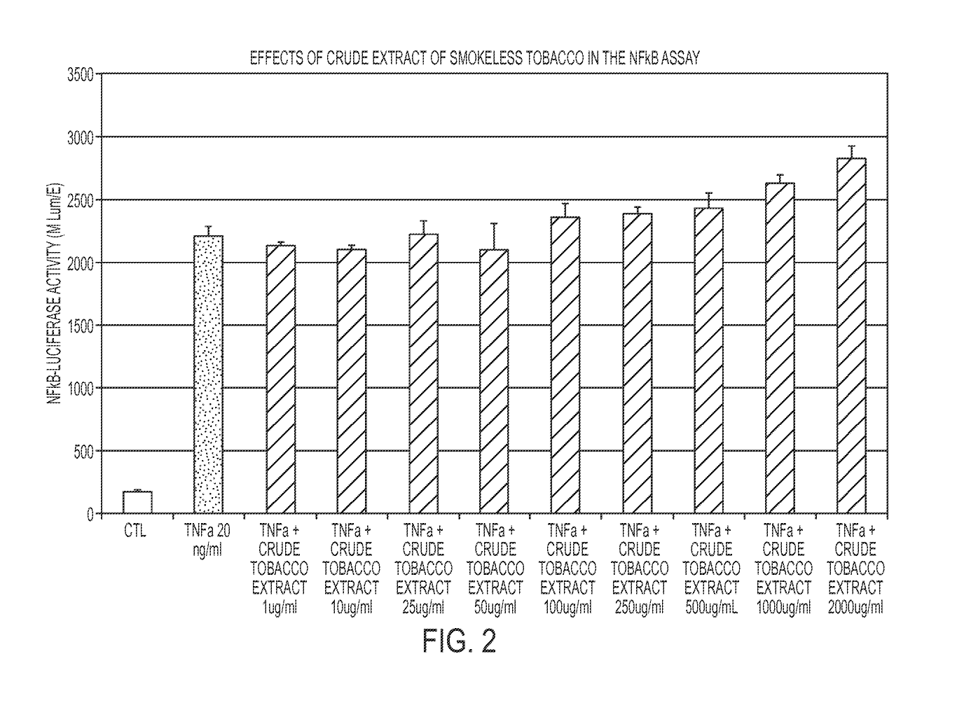

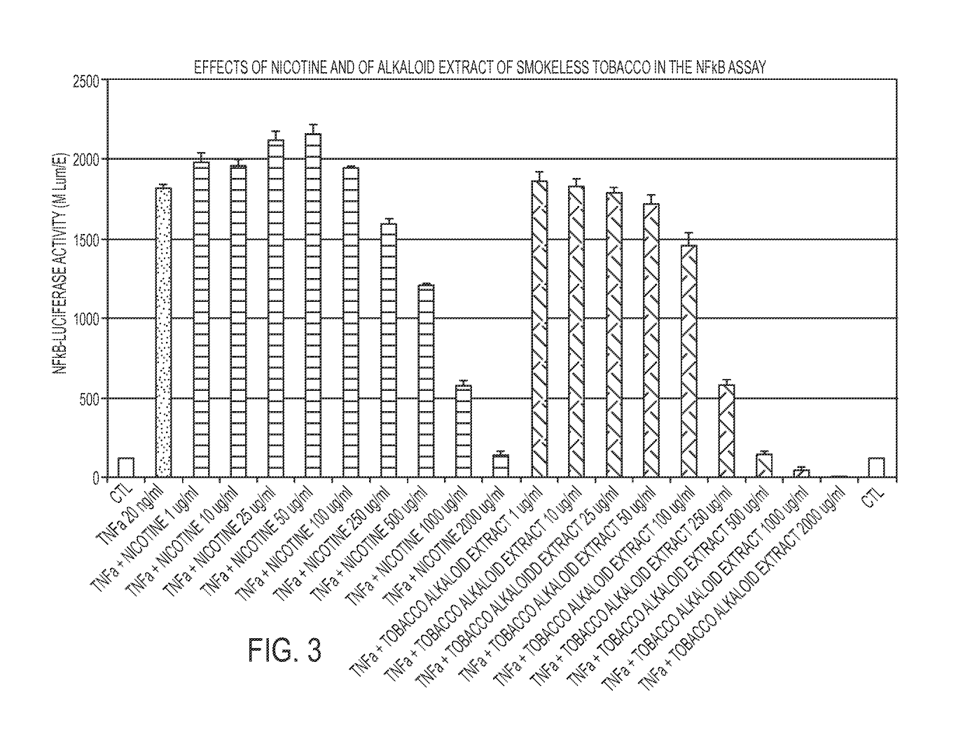

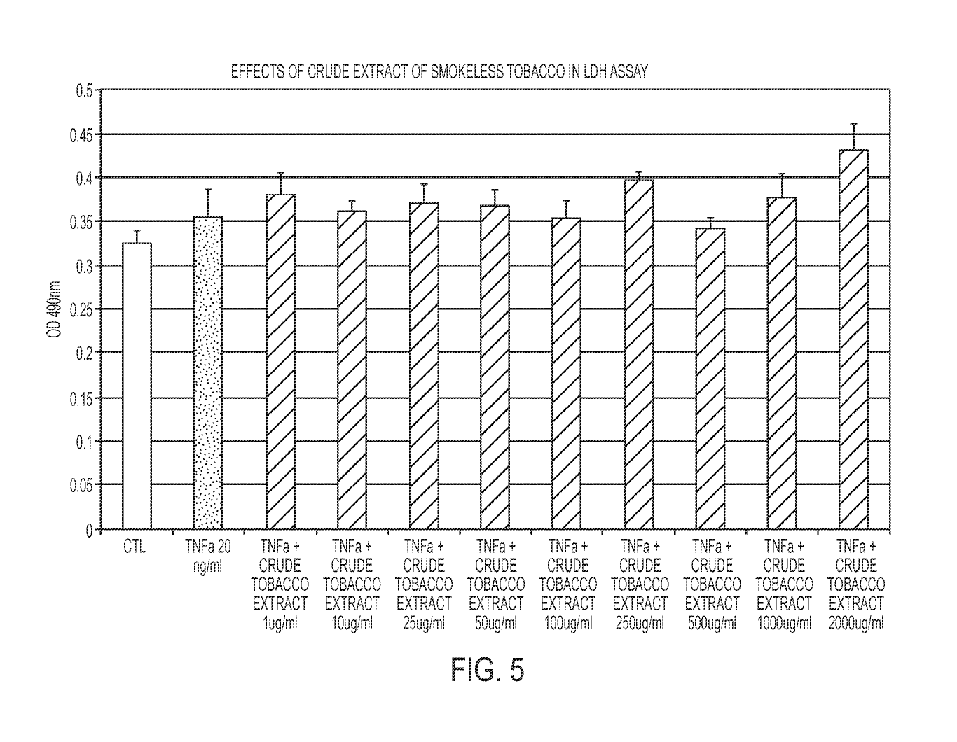

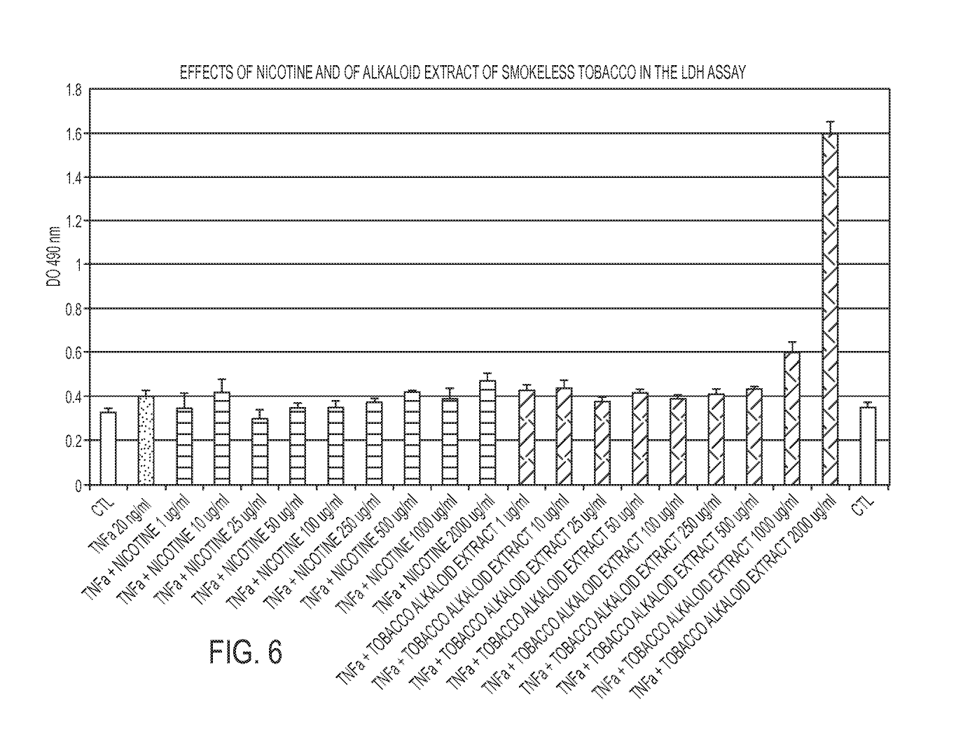

As shown in FIG. 1, TNFα induces an increase in NFκB-mediated transcription of luciferase; administration of anatabine can reduce this transcription to control levels without cellular toxicity (FIG. 4). Crude extracts of smokeless tobacco, while not toxic to cells (FIG. 5), do not reduce TNFα-induced NFκB-mediated transcription (FIG. 2). Although not suitable for administration as pharmaceuticals, both nicotine and an alkaloid extract of smokeless tobacco reduce TNFα-induced NFκB-mediated transcription (FIG. 3); at higher doses, the alkaloid extract demonstrates pronounced cytotoxicity (FIG. 6).

### EXAMPLE 2

### Materials and Methods

Animals. Male and female Sprague-Dawley rats (˜200-250 grams) were obtained from Charles River Laboratories Inc., Wilmington, Mass. and used in compliance with the Animal Welfare Act, the Guide for the Care and Use of Laboratory Animals, and the Office of Laboratory Animal Welfare. Upon receipt at the vivarium, rats were examined by trained personnel to ensure acceptable health status. Rats were acclimated for at least 5 days prior to use.

Rats were housed 3 per cage. Cage size met or exceeded the requirements set forth by the Institute for Animal Laboratory Research Guide for the Care and Use of Laboratory Animals. The rats were kept in a room maintained at 64-84° F. (22-24° C.) with humidity set at 40-70%. The room was illuminated with fluorescent lights timed to give a 12 hour-light, 12 hour-dark cycle. Standard rodent diet (PharmaServ lab diet 5001) and water were available for all rats. The feed was analyzed by the supplier, detailing nutritional information and levels of specified contaminants.

Test compounds. The following compounds were tested in the examples below:
(−) Nicotine hydrogen tartrate (Sigma Aldrich: N5260 Lot#098K0676) 35.1% (w/w) nicotine;
(R,S) Anatabine tartrate (2:3) (Toronto Research Chemicals, A637510, Lot#9-BHW-79-2) 41.6% (w/w) Anatabine;
(+/−)-nicotine-3′-d3 (Toronto Research Chemicals: N412423, Lot#9-BCC-114-2);
(R,S)-Anatabine-2,4,5,6-d4 (Toronto Research Chemicals: A637505, Lot#6-SG-82-1); and
anatabine polacrilex (Emerson Resource Inc. lot #JK02-145); purity was 5.18% as per Certificate of Analysis

Certificates of analyses for anatabine and nicotine indicated 98% and 100% purity, respectively. Anatabine was stored at 4° C. in a desiccated environment (silica), protected from light. Nicotine was stored at room temperature. Vehicle was sterile phosphate buffered saline (PBS) (Amresco lot#2819B188).

Supplies. The following were obtained from Becton Dickinson, Franklin Lakes, N.J.: MICROTAINER® Brand Tubes (K₂) EDTA (lot#9050883); serum separator blood collection tubes (lot#9104015); and sodium citrate blood collection tubes (lot#8310564). Ten percent neutral-buffered formalin was from Sigma Aldrich, St. Louis, Mo. (batch#019K4386).

### EXAMPLE 3

### Toxicokinetic Evaluation of Single Doses of Anatabine and Nicotine in Sprague-Dawley Rats

This example reports evaluation of the toxicokinetics of anatabine and nicotine following a single intravenous injection in Sprague-Dawley rats.

Summary

Anatabine was administered as a single intravenous (i.v.) injection at doses of 0.10, 0.75, or 1.0 mg/kg. Nicotine was administered as a single intravenous injection at a dose of 0.4 mg/kg. Six rats (3 males and 3 females) were dosed per dose group. Blood was collected for plasma at 15, 30, 60, 90, 120, 240, 360, 480, and 1440 minutes post i.v. administration. At the 1440 minute time point, animals were euthanized and perfused, and brains were removed and then homogenized. Plasma and brain homogenates were stored at −80° C. until analysis.

An additional 48 rats (24 males and 24 females) received a single intravenous dose via the tail vein at the same doses as mentioned above. At 30 and 360 minutes post administration, 6 rats (3 males and 3 females) per dose group, per time point were euthanized, bled via cardiac puncture, and perfused, and brains were collected. The brains were homogenized. Blood was spun, and plasma was collected. Plasma and brain homogenate were stored at −80° C.

Both anatabine and nicotine can be measured in rat plasma and brain following a single bolus i.v. dose. The concentration of anatabine in plasma is dose-related. Both compounds are also rapidly cleared from plasma; however, the elimination half-life of anatabine is approximately 2- to 2.5-fold greater than that of nicotine (t_{1/2}, 1.64 to 1.68 hr for anatabine compared to 0.67 hr for nicotine). The apparent volume of distribution (V_{D}) for anatabine is also significantly greater than that of nicotine.

At all doses of anatabine the elimination half-life (t_{1/2}), mean residence time (MRT), and exposures (AUC_{0→∞}) tended to be higher for female rats compared to male rats; however, only at the highest dose of anatabine (1.0 mg/kg) was this difference statistically significant. At this dose level, the elimination half-life (t_{1/2}) of anatabine in females was 1.84 hr compared to 1.44 hr for males; mean residence time (MRT) was 2.80 hr for females compared to 2.18 hr for males; and exposure (AUC_{0∝∞}) was 788.9 ng·hr/mL for females compared to 631.3 ng·hr/mL for males.

Anatabine and nicotine rapidly appear in brain tissue following i.v. administration, and the concentration of anatabine is dose-dependent. At each dose level the mean concentration of anatabine appeared to be higher in the brains of female animals compared to males; however, the differences were not statistically significant.

Anatabine tartrate (2:3) or nicotine bitartrate was dissolved to the appropriate concentrations in sterile PBS for the i.v. formulations (Table 2). The dosing solutions for each test compound were prepared on the basis of the relative content of the anatabine or nicotine base so that the final concentrations are reflective of the actual base concentration. Four aliquots of each dose level formulation were collected and stored at −80° C. The test compound, corresponding dose level, number of animals, and sample collection times for Phase I of the study are shown in Table 3. The test compound, corresponding dose level, number of animals, and sample collection times for Phase II of the study are shown in Table 4. The physical signs of each animal were monitored following administration of the test compound.

The animals were weighed prior to dosing and received a single i.v. dose of either test compound at a volume of 5 mL/kg. Blood was collected via the venus plexus (retro-orbital) into tubes containing (K₂) EDTA. No more than 0.5 mL was collected per time point. For the 1440-minute time point of Phase I or the 30- and 360-minute time points of Phase II, the animals were euthanized, bled via cardiac puncture, and perfused. The brain, was removed, weighed, homogenized in sterile 0.9% saline at a volume equal to its weight, and stored at −80° C.

Plasma was separated according to the instructions for MICROTAINER® brand collection tubes (3 minutes, 2000×g). Plasma was decanted into microfuge tubes and stored at −80° C. Remaining test compound was stored at −80° C.

Analytical Methods

The signal was optimized for each compound by electrospray ionization (ESI) positive or negative ionization mode. A single ion mode (SIM) scan was used to optimize the Fragmentor for the precursor ion and a product ion analysis was used to identify the best fragment for analysis and to optimize the collision energy. The fragment which gave the most sensitive and specific signal was chosen.

Sample preparation. Plasma and brain samples were treated with three volumes of methanol containing internal standard at 1 μM (either (+/−)-nicotine-3′-d₃ for nicotine or (R,S)-Anatabine-2,4,5,6-d₄ for anatabine), incubated 10 min at 4° C., and centrifuged. The amount of the test agent in the supernatant was determined by liquid chromatography tandem mass spectrometry (LC/MS/MS).

Analysis. Samples were analyzed by LC/MS/MS using an Agilent 6410 mass spectrometer coupled with an Agilent 1200 high pressure liquid chromatography (HPLC) and a CTC PAL chilled autosampler, all controlled by MassHunter software (Agilent). After separation on a hydrophilic interaction liquid chromatography (HILIC) HPLC column (Sepax) using an acetonitrile-ammonium acetate/acetic acid gradient system, peaks were analyzed by mass spectrometry (MS) using ESI ionization in multiple reaction monitoring (MRM) mode. MassHunter software was used to calculate the concentration of the test compounds in samples from the peak area using the appropriate calibration curves.

Recovery. Recovery standards were prepared by spiking blank matrix (plasma or brain homogenate) prior to deproteination or after with 23, 62, or 1667 ng/mL of test compound. Deproteination was done by adding 3 columns of methanol containing internal standard with centrifugation to pellet the precipitated protein. Recovery was calculated by dividing the area ratio (peak area of compound over internal standard of the precipitated sample over the recovery standard multiplied by 100. For example: area ratio of spiked plasma/area ratio of spiked deproteinated plasma×100.

Calibration samples. Calibration curves were determined for both rat plasma and brain homogenate. Calibration samples were prepared by diluting a 50× stock solution of the test compound in PBS with blank matrix to the appropriate concentration and these samples were prepared as described above in the sample preparation. Stock solutions were prepared by serial dilution as shown in Table 5.

Results

Physical Signs. All males and two females that received nicotine at 0.4 mg/kg experienced tremors immediately post dose and recovered within 2 to 4 minutes. One male (7C) and two females (8A and 8C) in this group also experienced labored breathing which lasted 2 to 4 minutes post dose. The same male (7C) was lethargic and recovered approximately 8 minutes post dose. All other animals in each dose group appeared normal following the administration of the test compounds.

Method Development. Table 6 shows the results of the LC/MS/MS method development for the determination of the appropriate ionization conditions and the mass to charge ratios (m/z) of the parent and product ions for anatabine and nicotine, and their deuterated analogues. The indicated product m/z ratios were used for the analysis of the relevant test samples.

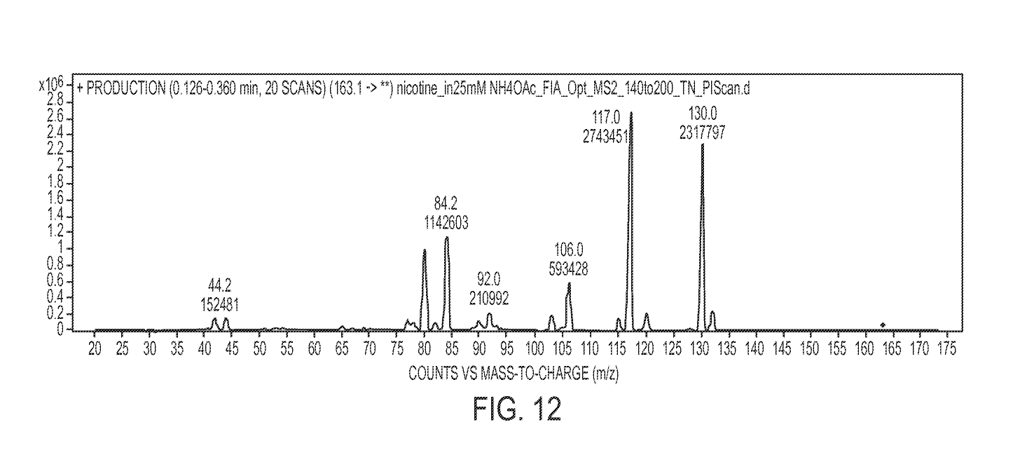

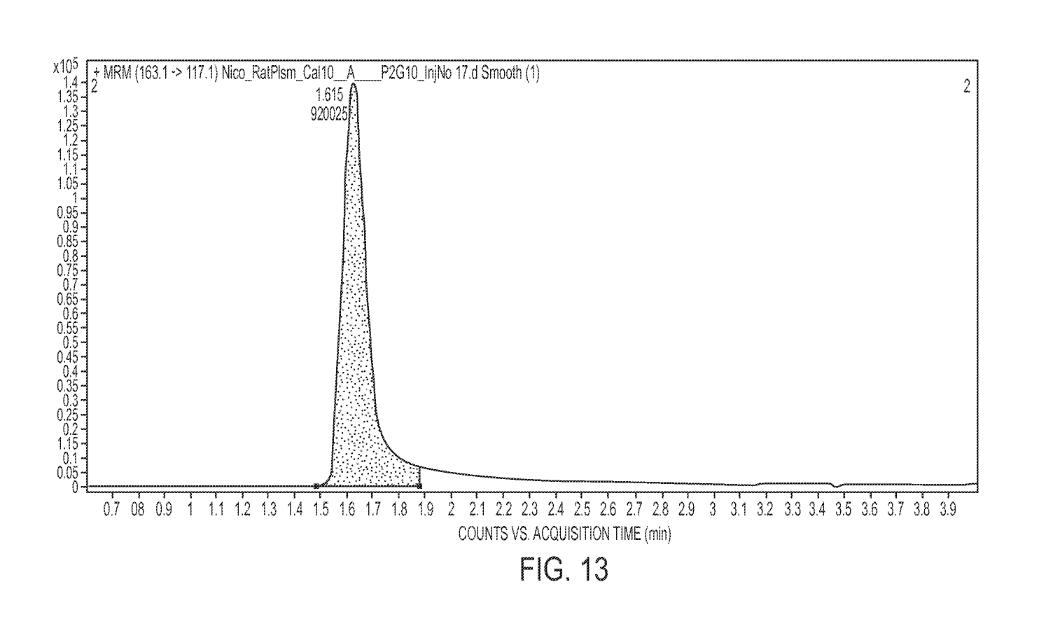

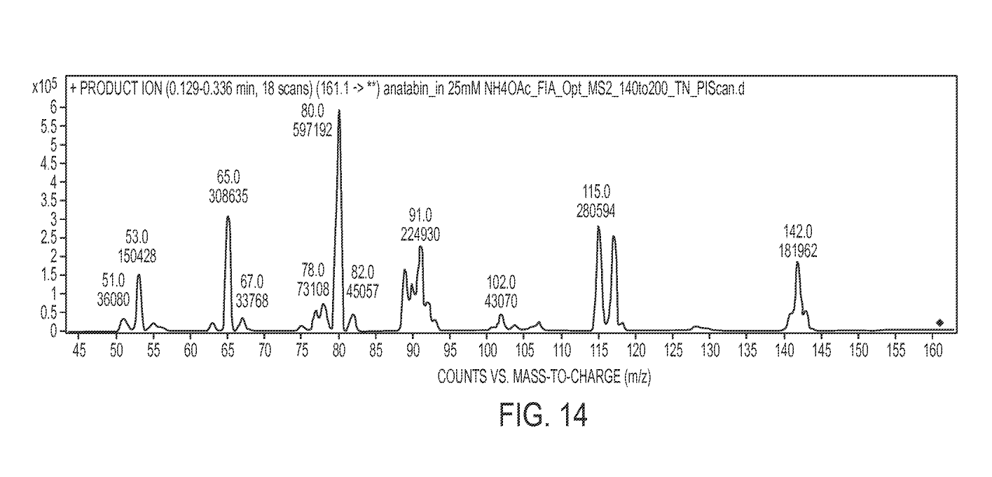

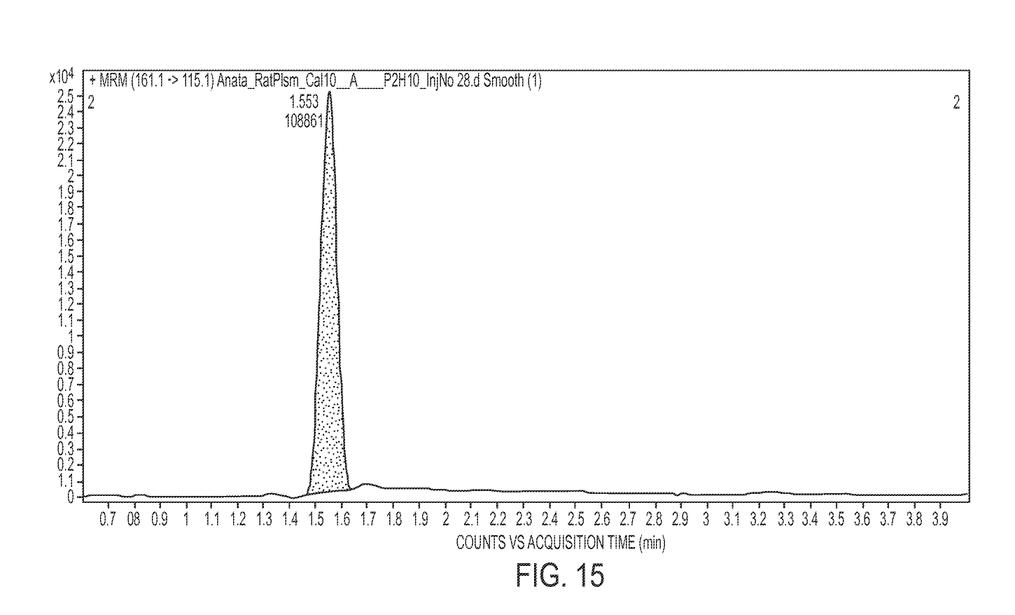

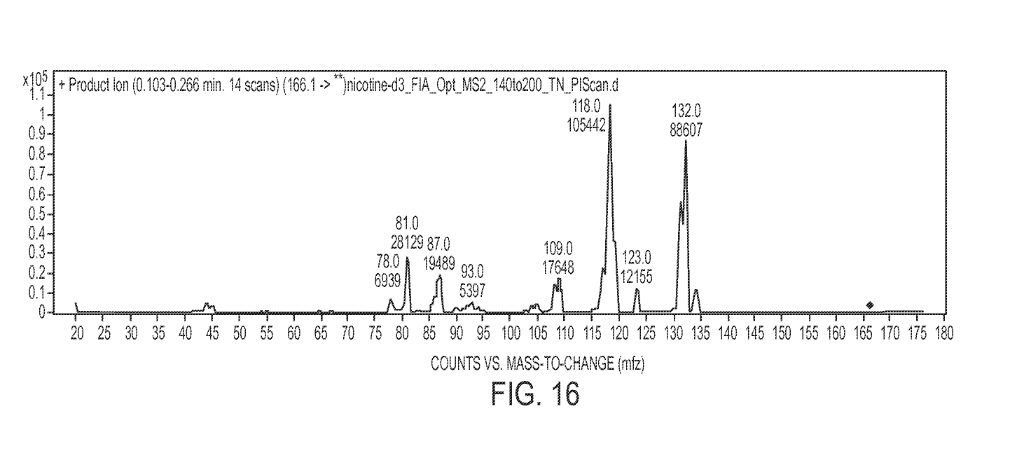

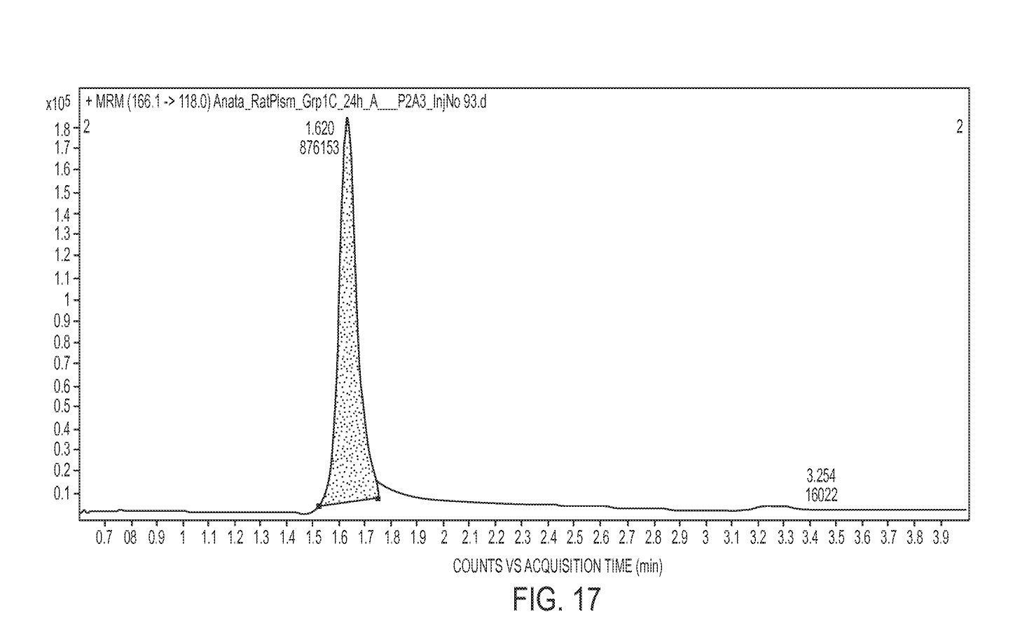

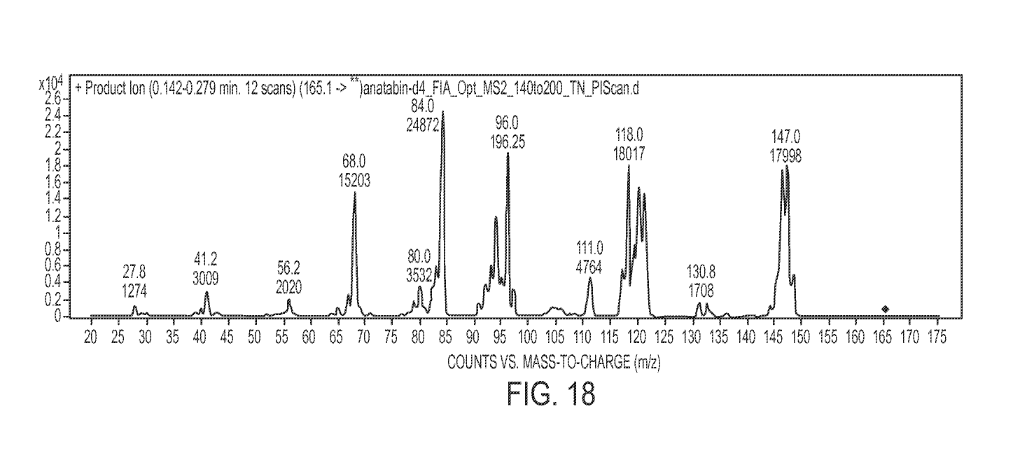

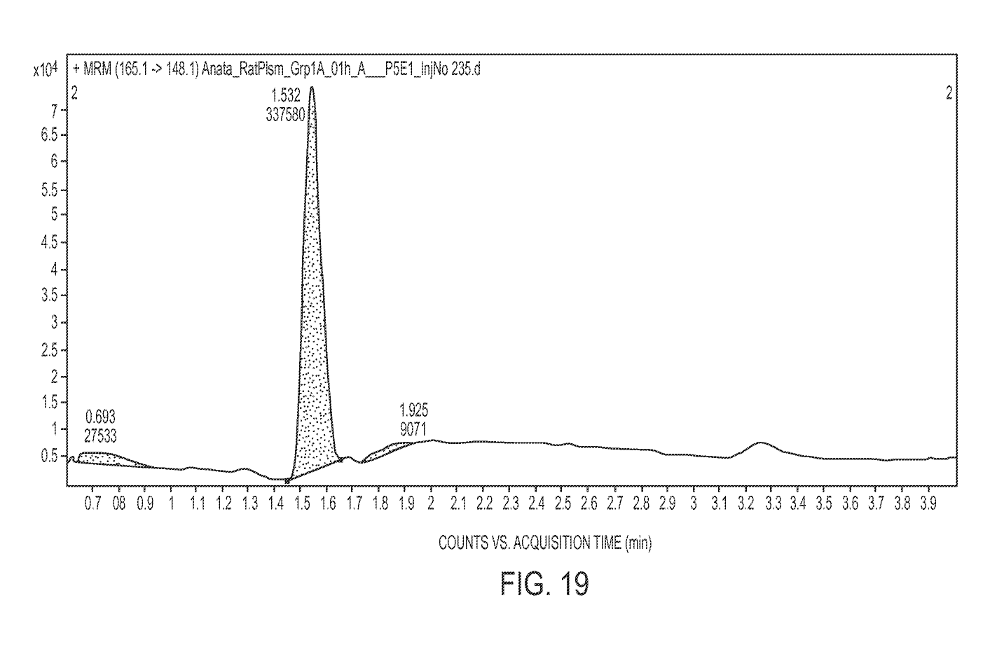

The product ion spectra and sample chromatograms for each compound in Table 6 are shown in FIGS. 12-19. The limits of detection (LOD) of anatabine and nicotine and their lower (LLQ) and upper (ULQ) limits of quantitation were derived from the appropriate calibration curves for each test compound and are shown in Table 7.

Table 8 provides data on the percent recovery of each test compound from either rat plasma or brain as a function of the given concentration. Except for the anatabine sample at the LOD and the nicotine samples in rat brain, recovery was generally greater than 90 percent.

Analysis of Dosing Solutions

Table 9 summarizes the analyses of the dosing solutions used in this study. The percent differences between the actual and expected concentrations are shown. Except for the lowest dose of anatabine, which was 70% of the expected concentration, the actual concentrations of test compounds were within 20% of the expected levels.

Plasma Pharmacokinetic Results & Analysis

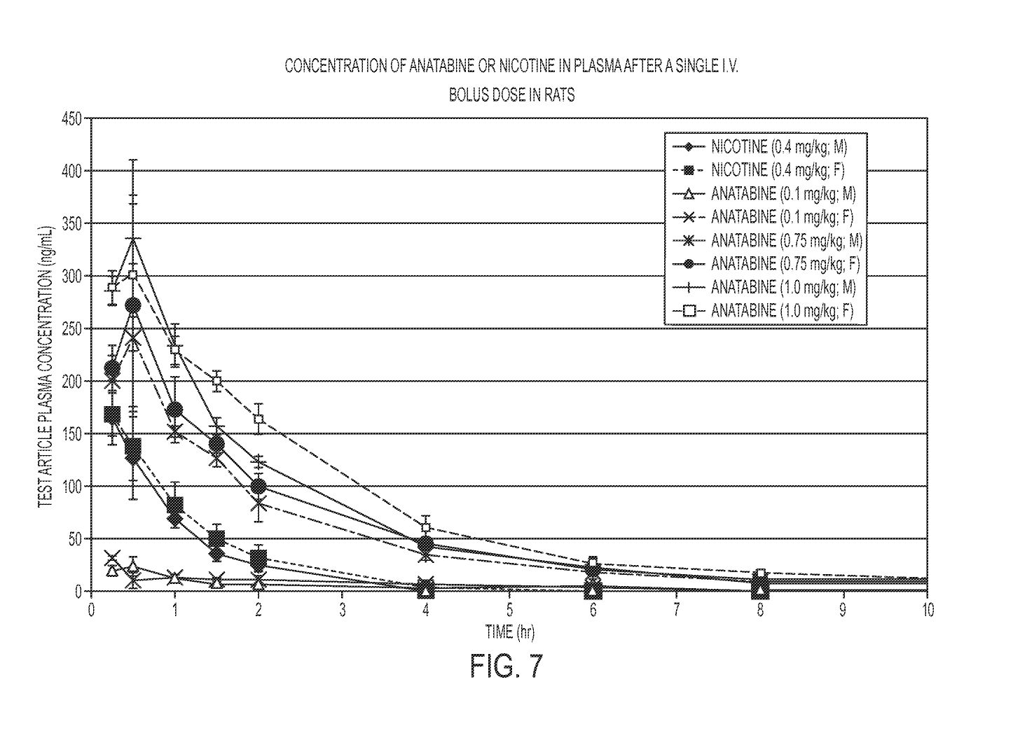

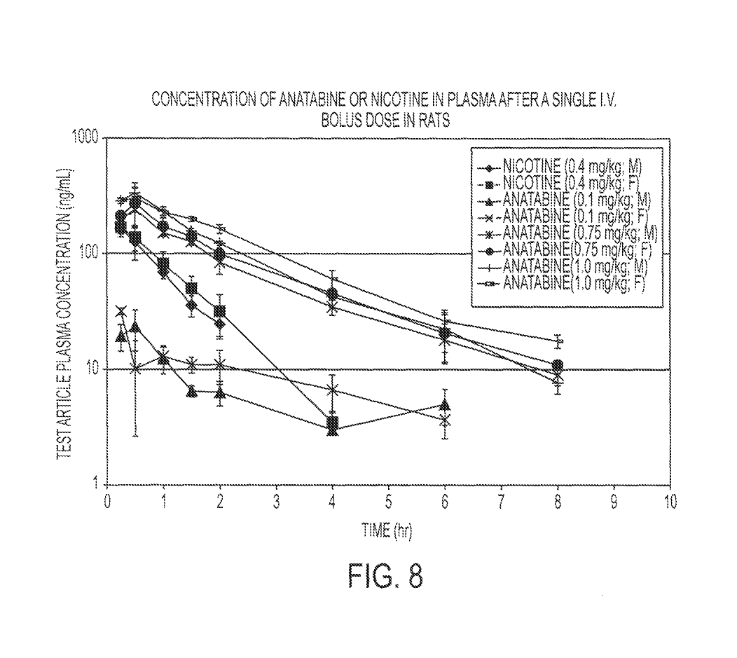

Table 14 lists the plasma concentrations of anatabine and nicotine for all animals at each time point. Table 15 summarizes this data in terms of the mean plasma concentrations of the test compound at each time point for males, females and both genders combined. This data is presented graphically in FIG. 7 and FIG. 8 (semi-log plot). The 24-hr data points from all treatment groups were below the limits of quantitation. Between approximately 6 and 8 hours the plasma concentrations of nicotine and anatabine (0.1 mg/kg) were below the limits of quantitation.

Table 10 and Table 11 provide comparisons for several pharmacokinetic parameters between the different treatment groups and between male and female animals. Both nicotine and anatabine can be measured in rat plasma following a single i.v. bolus, and their concentrations appear to be dose-related. The elimination half-life (t_{1/2}) for each of the anatabine treatment groups was significantly greater than that for the nicotine treatment group (2.1× to 2.5× greater; 0.67 hr for nicotine compared to 1.44 to 1.68 hr for anatabine). The elimination half-lives were similar among the anatabine treatment groups. The longer half-life for anatabine is reflected in the longer mean residence times (MRT), which are about 2-fold longer for anatabine compared to nicotine. Finally, the apparent volume of distribution (V_{D}) was lower for the nicotine group compared to the anatabine treatment groups. Amongst the anatabine treatment groups, V_{D} was significantly greater for the 0.1 mg/kg dose group compared to either of the two higher doses; however, it is not known whether this is a real difference or whether it is due to variability and the fewer number of measurable data points at the low dose.

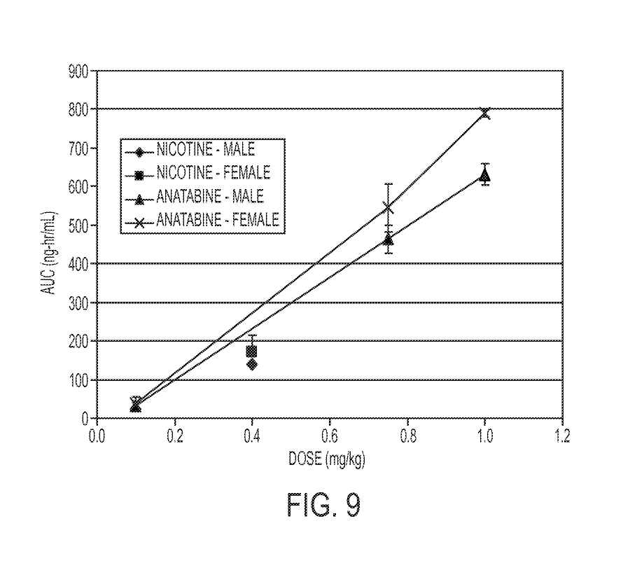

Table 11 shows a comparison of these same parameters between male and female rats within each treatment group. There were no statistically significant differences between males and females except in the highest anatabine treatment group (1.0 mg/kg) where the females exhibited a longer elimination half-life and therefore, longer mean residence time than the males (t_{1/2}, 1.84±0.16 hr and MRT, 2.80±0.24 hr, females compared to t_{1/2}, 1.44±0.08 and MRT, 2.18±0.12 hr, males). This difference is apparent for all treatment groups, although it only achieved statistical significance in the highest anatabine group. The females in this treatment group also displayed a much greater overall exposure (AUC_{0→∞}) to anatabine than the male animals. This difference is depicted in FIG. 9, which shows the dose-exposure relationship for anatabine and nicotine. Overall, there appears to be a linear response between dose and exposure for anatabine; it is not possible to determine if the female animals display a non-linear response at high doses of anatabine.

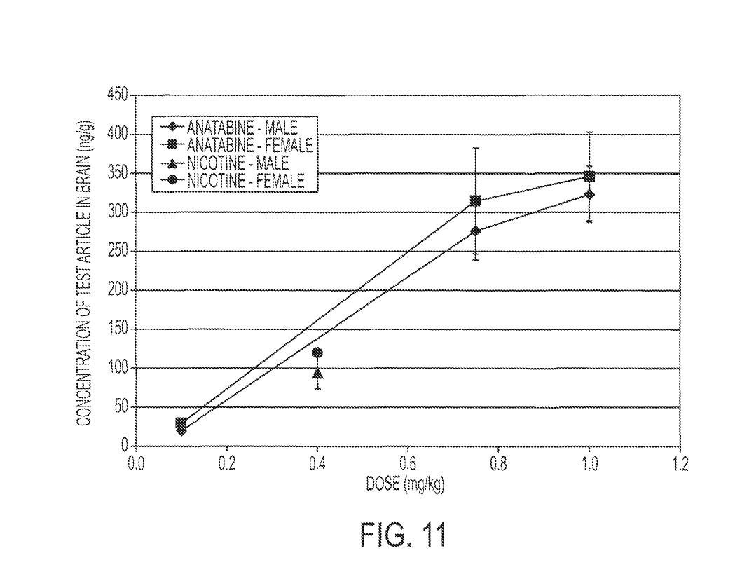

FIG. 11 shows the dose-concentration response for the 0.5-hour time point for males and females at each dose level. It appears that the brain levels of anatabine begin to level off between 0.75 mg/kg and 1.0 mg/kg.

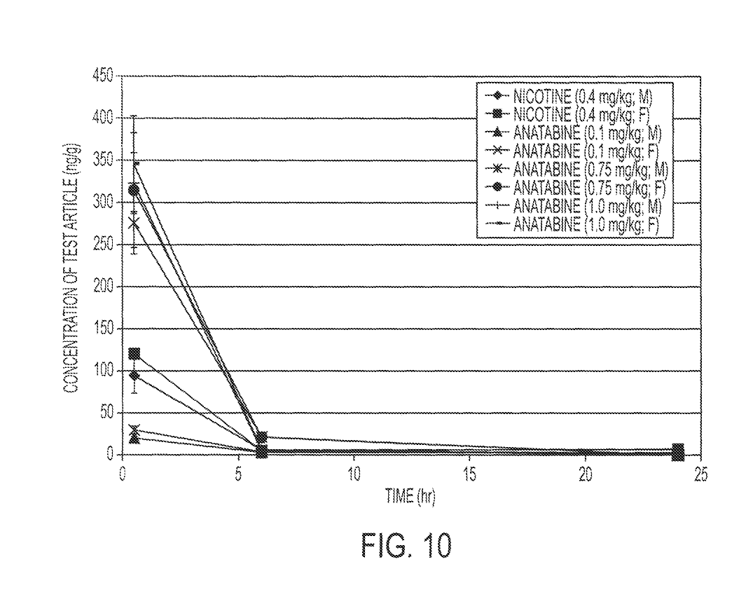

Table 14 lists the concentrations of anatabine and nicotine in the brain extracts for all animals at each time point. Table 15 summarizes this data in terms of the mean concentrations of the test compound per gram of brain tissue at each time point for males, females and both genders combined. This data is presented graphically in FIG. 10 and in tabular form in Table 12. After the 6-hour time point most concentrations were below the limits of quantitation; however, the test compound concentration was quantifiable in several samples at 24-hours.

FIG. 11 shows the dose-concentration response for the 0.5-hour time point for males and females at each dose level. It appears that the brain levels of anatabine begin to level off between 0.75 mg/kg and 1.0 mg/kg.

Discussion

All males and two females that received nicotine at 0.4 mg/kg experienced tremors immediately post dose; however they recovered within 2 to 4 minutes. One male (7C) and two females (8A and 8C) in this group also experienced labored breathing which lasted 2 to 4 minutes post dose. The same male (7C) was lethargic and recovered approximately 8 minutes post dose. All animals in each of the anatabine dose groups appeared normal immediately following administration of the test compounds and no obvious adverse signs were observed.

Both nicotine and anatabine can be measured in rat plasma following a single, bolus, i.v. dose and their concentrations appear to be dose-related. The elimination half-life of anatabine is approximately 2- to 2.5-fold greater than that of nicotine, and this is also reflected in a longer mean residence time, which is approximately twice as long as that for nicotine. The 24-hr data points from all treatment groups were below the limits of quantitation and it appears that at the doses selected, the test compounds are cleared from rat plasma between 8 and 24 hours post-administration.

The apparent volume of distribution (V_{D}) was also significantly lower for the nicotine group compared to the anatabine treatment groups. Amongst the anatabine treatment groups, V_{D} was significantly greater for the 0.1 mg/kg dose group compared to either of the two higher doses; however, it is not known whether this is a real difference or whether it is due to variability and the fewer number of measurable data points at the low dose.

When comparisons between male and female animals were conducted for these same parameters, within each treatment group, there were no statistically significant differences observed except for the highest anatabine treatment group (1.0 mg/kg) where the females exhibited a longer elimination half-life and therefore, longer mean residence time than the males (t_{1/2}, 1.84±0.16 hr and MRT, 2.80±0.24 hr, females compared to t_{1/2}, 1.44±0.08 and MRT, 2.18±0.12 hr, males). In fact, these differences between male and female animals were apparent for all treatment groups, although statistical significance was achieved only at the highest anatabine dose tested. The females in this treatment group also displayed a much greater overall exposure (AUC_{0→∞}) to anatabine than the male animals. Overall, there is a linear response between dose and plasma concentrations or exposure to anatabine in both male and female rats; although the response appears to be somewhat greater in female animals and is more pronounced at the higher dose levels. It is not possible to determine from the data if the female animals display a non-linear response at higher doses of anatabine.

Both anatabine and nicotine rapidly appear in brain tissue following i.v. administration. The concentrations of anatabine are dose-dependent but appear to level off between 0.75 mg/kg and 1.0 mg/kg. This observation is based on the levels measured only at the 0.5-hour time point and a greater number of time points are required for a more thorough evaluation. There were no statistically significant differences in the concentrations of either test compound in brain between male and female animals; however at each dose level the mean concentrations in the brains of females tended to be somewhat higher.

### EXAMPLE 4

### Toxicokinetic Evaluation of Single Doses of Anatabine and Nicotine with a 14-Day Observation Period

This example reports the evaluation of the toxicity of anatabine or nicotine for a period of fourteen days following a single intravenous injection in Sprague-Dawley rats. The toxicity of anatabine and nicotine was evaluated after a single intravenous (i.v.) injection in the rat. Anatabine was administered as a single intravenous injection at doses of 0.10, 0.75, or 1.5 mg/kg. Nicotine was administered as a single intravenous injection at a dose of 1.50 mg/kg. One control group of animals received a single i.v. dose of the vehicle at 5 mL/kg. Ten rats (5 males and 5 females) were dosed per group. Due to animal mortality in the nicotine-dosed group, the surviving animals were taken off study and a separate nicotine tolerability study was conducted. One female received a single i.v. dose of 1.25 mg/kg, and 3 females received a single i.v. dose of 1.0 mg/kg. Following the tolerability study, a group of 5 males and 5 females received a single i.v. dose of nicotine at 0.75 mg/kg.

All rats dosed with vehicle or anatabine, and the animals dosed with 0.75 mg/kg of nicotine were observed daily for 14 days. Body weight and food consumption was measured daily for 14 days. On day 15, urine was collected on all surviving animals. The animals were euthanized and bled via cardiac puncture, and blood was collected for analysis. Tissues were collected, weighed, evaluated for gross abnormalities, and stored in 10% neutral-buffered formalin.

All groups appeared normal immediately after dosing except for the animals dosed with 1.5 mg/kg of anatabine and those dosed with 1.5 mg/kg of nicotine. Both males and females dosed with 1.5 mg/kg of anatabine experienced tremors upon compound administration. The animals appeared normal by 15 minutes post dose. Upon completion of the 1.5 mg/kg dose of nicotine, tremors and rigidity were observed in all dosed animals. The tremors were more severe in the females. One male did not survive, whereas the other 4 appeared normal after 15 minutes. Three females were dosed and two died within 5 minutes of dosing; the remaining 2 females were not dosed due to the morbidity in the group. The surviving animals from this group were removed from study. These results suggest that both anatabine and nicotine affect both the peripheral and central nervous systems.

During the tolerability study, all rats (1 female dosed with 1.25 mg/kg of nicotine and 3 females dosed with 1.0 mg/kg of nicotine) experienced severe tremors upon completion of dosing, but all returned to normal by 20 minutes post dose. These animals were not included in the 14-day observation period.

Both males and females dosed with nicotine at 0.75 mg/kg experienced tremors upon compound administration but returned to normal within 15-20 minutes post dose. One male and two females died post dose. Surviving animals in all groups appeared normal throughout the 14-day observation period. The body weights for both male and female rats in the nicotine group were lower than those in the control and anatabine treatment groups; however, these were still within the study-specified range. Consequently body weight gain for this treatment group was also somewhat lower than the vehicle controls. Food consumption was similar among the groups over the 14-day period; however, consumption by males treated with 0.1 mg/kg or 1.5 mg/kg anatabine appeared to be somewhat higher than animals in the control group. This is not considered to be a treatment-related effect.

Hematology and blood chemistries for male and female animals were analyzed and evaluated for differences between the individual treatment groups and the relevant vehicle controls. All treatment groups showed no significant differences relative to the controls and/or the values were well within the normal ranges expected for this species. Similarly, no notable differences in any of the urinalysis parameters were observed between animals treated with either anatabine or nicotine, relative to the controls.

Anatabine or nicotine was dissolved to the appropriate concentrations in sterile PBS for the i.v. formulations (see Table 16). The dosing solutions for each test compound were prepared on the basis of the relative content of the anatabine or nicotine base so that the final concentrations reflect the actual base concentrations. Four aliquots of each dose formulation were collected and stored at −80° C. The test compound, corresponding dose level, number of animals, and frequency of observations are shown in Table 17.

The animals were weighed prior to dosing and received a single i.v. dose via the lateral tail vein of either test compound or vehicle at a volume of 5 mL/kg. Due to animal mortality in the nicotine-dosed group (1.5 mg/kg), the surviving animals were taken off study and a separate nicotine tolerability study was conducted.

Nicotine Tolerability Study

One female rat was dosed intravenously with 1.25 mg/kg of nicotine, and three females were received 1.0 mg/kg intravenously. Following the tolerability study, an additional group was added to the study. Five males and five females received a single intravenous dose of nicotine at 0.75 mg/kg. All animals were observed daily. Body weight and food consumption was measured daily, with any abnormal observations noted. Average daily body weights and food consumption was tabulated with standard deviation calculated.

On day 15, urine was collected on all surviving animals for urinalysis. The animals were euthanized and bled via cardiac puncture. Blood was collected for hematology, clinical chemistry, and coagulation analysis. Tissues were collected, weighed, and stored in 10% neutral-buffered formalin for possible future analysis. The tests and tissues collected are summarized in Table 18.

### Results

Dosing Solution Analysis

Table 19 summarizes the dosing solutions used during the conduct of this study. The percent differences between the actual and expected concentrations of the test compounds are shown. The actual concentrations were within 20 percent of the expected levels.

### General Observations

All groups appeared normal immediately after dosing except for the animals dosed with 1.5 mg/kg of anatabine and those dosed with 1.5 mg/kg of nicotine. Both males and females dosed with 1.5 mg/kg of anatabine experienced tremors upon compound administration. The animals appeared normal by 15 minutes post dose. Following administration of the 1.5 mg/kg dose of nicotine, tremors and rigidity were observed in all animals. The tremors were more severe in the females. One male did not survive, whereas the other 4 appeared normal after 15 minutes. Three females in this group were dosed and two died within 5 minutes of dosing; the remaining 2 females were not treated due to the observed morbidity in the group. The surviving animals from this group were removed from the study.

During the tolerability study, all rats (1 female dosed with 1.25 mg/kg of nicotine and 3 females dosed with 1.0 mg/kg of nicotine) experienced severe tremors upon completion of dosing, but all returned to normal by 20 minutes post dose. These animals were not included in the 14-day observation period.

Both males and females dosed with nicotine at 0.75 mg/kg experienced tremors upon compound administration, but returned to normal within 15-20 minutes post dose. One male and two females died post dose.

Surviving animals in all groups appeared normal throughout the 14-day observation period.

Body Weights, Growth Rates and Food Consumption

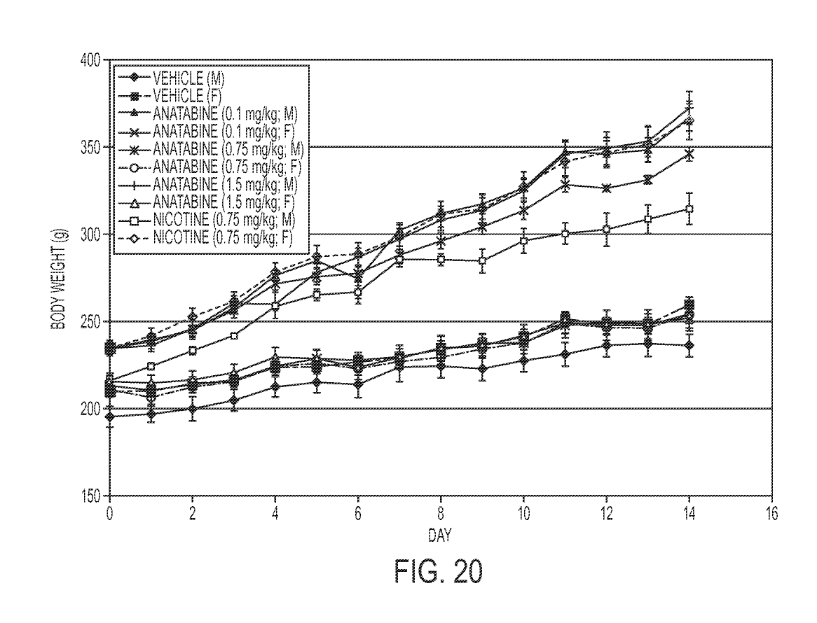

The daily measured body weights for each animal are tabulated in Tables 28A-F and the average daily food consumption is summarized in Tables 29A, B. These data are summarized in Table 20 for the average weight gain over the 14-day observation period and the average daily food consumption, by treatment group and gender. FIG. 20 shows the mean body weights of animals in each treatment group on the day of dosing (Day 0) and for each day, thereafter.

The average weight gains for animals in each treatment group over the 14-day observation period were similar to those in the vehicle control group, except for the nicotine-dosed group of male animals that exhibited weight gains that were significantly lower than the controls. The mean increase in the weight of females of the nicotine-dosed group was also lower than that of the vehicle control, though not statistically significant at the 5 percent level. It should be noted that the mean weights of the male and female animals in the nicotine-treated group at Day 0 were lower than their corresponding genders in the vehicle control. The difference for males was statistically significant (Vehicle: 234.6±9.9 g versus Nicotine: 216.0±6.2 g; p=0.014), although that for females was not (Vehicle: 209.8±7.3 g versus Nicotine: 195.3±10.4 g; p=0.058).

The average daily food consumption per animal was statistically higher in the males of the 0.1 mg/kg and 1.5 mg/kg anatabine treatment groups. This difference is not considered to be clinically significant or related to any treatment effects.

Overall, although some differences in the changes in weight and food consumption were statistically significant, they are not considered to be treatment-related.

Necropsy Observations and Organ Weights

Upon necropsy and organ collection no noticeable differences or abnormalities were observed between the vehicle-dosed animals and the test compound-dosed animals. Individual organ weights can be found in Table 36. Several statistically significant differences in organ weights were noted (see Table 21 and Table 22); however, they do not appear to be dose-related and likely due to the small sample sizes and variability in the organ collection. In general, several organ weights tended to be lower in the nicotine-treated group, although this observation is likely related to the lower animal weights in this group relative to the controls.

Hematology and Coagulation Parameters

Plasma samples collected for hematology were analyzed, and individual values for the various parameters for each animal are listed in Table 31 (normal ranges, Table 30) and these are summarized in terms of descriptive statistics in Table 23A, Table 23B, and Table 24. Also shown are statistical comparisons between the vehicle controls and the various treatment groups, subdivided by gender.

In general, there were few significant differences between the treatment groups and the vehicle control group for either gender. Female rats in 0.1 mg/kg anatabine group showed a small but statistically significant decrease in mean corpuscular hemoglobin concentration (MCHC) relative to the control; however, the values are still within the normal range for this species. Similarly, females in the 1.5 mg/kg anatabine and 0.75 mg/kg nicotine treatment groups showed small, but statistically significant decreases in mean corpuscular volume (MCV) and mean corpuscular hemoglobin (MCH), although these values were still within the normal range for this species as well.

Males and females in the 0.75 mg/kg and 1.5 mg/kg anatabine groups showed a statistically significant decrease in reticulocyte count compared to the control animals; however, these values are also well within the normal range for this parameter.

There were no notable differences in red blood cells, white blood cells, platelet counts, lymphocyte, monocyte, eosinophil and basophil counts, or neutrophil segmentation.

Individual values for the coagulation parameters activated partial thromboplastin (aPTT) and prothrombin times (PT) for each animal are listed in Table 34 (normal ranges, Table 30). These are summarized in terms of descriptive statistics in Table 25. Also shown are statistical comparisons between the vehicle controls and the various treatment groups, subdivided by gender. There were no significant differences in aPTT or PT between the vehicle control and each of the anatabine treatment groups; although the aPTT values for all these groups were outside the normal range. In both male and female animals of the nicotine group, however, aPTT was significantly lower relative to the vehicle control group, indicative of faster clotting times due to the intrinsic, contact activation pathway. The origin of this difference is not known, although the values are within the normal range for this species.

Clinical Chemistry

Plasma samples collected for blood chemistries were analyzed, and individual values for the various parameters for each animal are listed in Table 33 (normal ranges, Table 32), and these are summarized in terms of descriptive statistics in Tables 26A, 26B, 27A, and 27B. Also shown are statistical comparisons between the vehicle controls and the various treatment groups, subdivided by gender.

Values for all clinical chemistry parameters were within the respective normal ranges. There were several parameters where statistically significant differences were noted between treatment groups and controls. Specifically, males treated with anatabine at 0.75 mg/kg and 1.5 mg/kg showed slight increases in albumin levels, as did females treated with 0.1 mg/kg and 0.75 mg/kg anatabine, but not at 1.5 mg/kg. Total protein was slightly increased in males in all anatabine treatment groups and the nicotine group relative to vehicles controls. In females, total protein was somewhat higher only in the 0.1 mg/kg anatabine and nicotine groups. Finally, as with total protein, globulins were marginally higher at all anatabine dose levels and the nicotine dose group in males. Globulins were also slightly higher in females in the 0.1 mg/kg anatabine and nicotine groups. The higher globulin levels, but not albumin, in the nicotine group is reflected in slightly lower A/G ratios, for both genders. Nevertheless, all the reported values for albumin, globulins and total protein were within the normal range for this species. There were small, but statistically significant differences noted for calcium levels in males in the nicotine-treated group and for sodium levels in males at 0.75 mg/kg and 1.5 mg/kg anatabine and females in the 1.5 mg/kg anatabine treatment groups. The values are well within normal ranges and therefore, not clinically significant.

Urinalysis

Individual values of the urinalysis parameters for each animal are listed in Table 35. There were no notable differences between the active treatment groups and controls and the observations are all consistent with those expected for this species.

Discussion

The toxicity of anatabine and nicotine was evaluated after a single intravenous (i.v.) injection in the rat. Anatabine was administered at doses of 0.10, 0.75, or 1.5 mg/kg. Nicotine was administered at a dose of 1.50 mg/kg, initially; however, due to mortality and significant adverse effects observed at this dose and at lower doses of 1.0 mg/kg and 1.25 mg/kg, a separate group was included in the study and dosed with nicotine at 0.75 mg/kg. One group of animals received a single i.v. dose of the vehicle at 5 mL/kg. Ten rats (5 males and 5 females) were dosed per group.

All rats dosed with vehicle or anatabine, and the animals dosed with 0.75 mg/kg of nicotine were observed daily for 14 days. Body weight and food consumption was measured daily for 14 days. On day 15, urine was collected on all surviving animals. The animals were euthanized, bled via cardiac puncture, and blood was collected for analysis. Tissues were collected, weighed, any gross abnormalities were noted, and stored in 10% neutral-buffered formalin for possible future analysis.

All animals, at all dose levels of anatabine, survived the study; however, those in the 1.5 mg/kg anatabine group experienced tremors and shaking immediately after test compound administration, which lasted for approximately 15 minutes post-treatment. In the nicotine treatment group (0.75 mg/kg), one male animal and 2 females died following test compound administration, and all animals experienced tremors and shaking for up to 20 minutes post-administration. These results suggest that both anatabine and nicotine affect both the peripheral and central nervous systems.

The growth rates and food consumption in all anatabine treatment groups were similar to their appropriate male or female vehicle controls. Male rats in the nicotine treatment group had a slightly lower growth rate; however, this is unlikely to be related to the test compound. This group of animals began the study at a lower average weight than males in the control or anatabine treatment groups. The food consumption in males, in the 0.1 mg/kg and 1.5 mg/kg anatabine groups was somewhat higher than controls and although the result was statistically significant it is not likely to be related to an effect of the test compound.

At necropsy, no noticeable differences or gross abnormalities were observed in any of the organs collected between the vehicle-treated and the test compound-treated animals. Several statistically significant differences in organ weights were noted; however, they do not appear to be dose-related and are likely due to the small sample sizes and the inherent variability associated with organ collection. The weights of heart, liver and kidneys in males, and thymus and heart in females of the nicotine-treated group were significantly lower than those of the corresponding vehicle controls; however, this observation is likely related to the lower overall animal weights in this group relative to the controls.

The hematology parameters for all treatment groups and genders were within the normal ranges expected for this species or displayed no significant differences when compared to the vehicle controls. Activated partial thromboplastin and prothrombin times were similar for all anatabine treatment groups relative to the controls; however, they were higher than the expected normal range. Both males and females in the nicotine group displayed significantly shorter clotting times via the intrinsic or contact activation pathway (aPTT) compared to the relevant control animals; however, the values were within the normal ranges for this species. Clotting times via the extrinsic or tissue factor pathway as determined by prothrombin times (PT) were normal.

Values for all clinical chemistry parameters were within the respective normal ranges or showed no differences relative to the vehicle control group.

Evaluation of the individual urinalysis parameters for each animal showed no notable differences between the active treatment groups and controls.

### EXAMPLE 5

### Toxicokinetic Evaluation in Sprague-Dawley Rats of Oral Multi-Dose Administration of Anatabine

This example reports the results of an evaluation of the pharmacokinetics of anatabine following multiple oral doses in Sprague-Dawley rats.

Summary

The plasma pharmacokinetic profile of orally administered anatabine was investigated in the rat. This study consisted of two groups of 8 animals each, 4 males and 4 females. One group received a total of 0.6 mg anatabine per kilogram body weight (BW) and the second group received 6.0 mg anatabine per kilogram BW in three, divided, oral, doses of 0.2 mg/kg BW (0.6 mg total) or 2.0 mg/kg BW (6.0 mg total). The test compound was administered as anatabine polacrilex and each dose was administered at 0, 4, and 8 hours and was administered in a volume of 5 mL/kg BW. Blood was collected for plasma at 30, 60, 240, 270, 300, 480, 540, 600, 720 and 1440 minutes post initial dose.

All animals in both treatment groups appeared normal immediately following each administration of the test compound and no adverse signs were observed for the duration of the observation and plasma sampling period.

The mean time to maximal plasma concentration following the first two oral doses ranged from 0.50 to 0.88±0.25 hr. There were no significant differences between gender or dose group. After the third dose of test compound, the mean time to maximal plasma concentration ranged from 1.00 to 2.00±1.41 hr. Within each dose group there were no significant differences in C_{p, max} between males and females and nor was there any significant change in this parameter over time. In females of the high dose group C_{p, max} appeared to increase from 259.8±35.4 ng/mL to 374.8±122.9 ng/mL; however, the trend was not statistically significant.

There were two, observable, minima following the first two oral doses of anatabine polacrilex. In general, the minima were not significantly different from one another over time, except for females of the high dose group, which increased from 51.5±26.0 ng/mL to 180±31 ng/mL.

The total exposure, elimination half-lives, mean transit times and mean absorption times did not differ significantly between male and female rats within the two treatment groups. When these data are combined and grouped according to dose level the total exposure is significantly greater at the high dose as would be expected; however, the terminal elimination half-life is also significantly higher in the 6.0 mg/kg BW group compared to the 0.6 mg/kg BW dose group.

The overall elimination half-life of anatabine following the first oral dose was 1.93±0.73 hr, the mean transit time was 3.01±1.25 hr and the mean absorption time was 0.56±1.25 hr. The mean absorption time of 0.56 compares favorably with the calculated Tₘₐₓ values following the first two doses and indicates that the absorption of anatabine occurs within the first 30 to 60 minutes after oral administration.

Anatabine was stored at 4° C., protected from light. The vehicle was sterile phosphate buffered saline (PBS) (Amresco). The test compound was formulated in sterile phosphate buffered saline (PBS) based on the content of anatabine base in the anatabine polacrilex. Two formulations were prepared; one for each of the two treatment groups. The test compound was formulated for each treatment group just prior to the first dose administration and constantly stirred until dosing was completed (Table 37). Four aliquots of each dose level formulation were collected and stored at −80° C. The test compound, corresponding dose level, and number of animals are shown in Table 38. The sample collection times are shown in Table 39.

The physical signs of each animal were monitored following administration of the test compound.

The animals were weighed prior to dosing and received three doses p.o. of test compound at a volume of 5 mL/kg. Blood was collected via the venus plexus (retro-orbital) into tubes containing (K₂) EDTA. No more than 0.5 mL was collected per time point. For the 1440-minute time point the animals were euthanized, and bled via cardiac puncture.

Plasma was separated as per package instructions for MICROTAINER® brand collection tubes (3 minutes, 2000×g). Plasma was decanted into microfuge tubes and stored at −80° C. Remaining test compound was placed at −80° C.

Sample preparation. Plasma samples were treated with three volumes of methanol containing internal standard at 1 μM (R,S)-Anatabine-2,4,5,6-d₄), incubated 10 min at 4° C., and centrifuged. The amount of the test agent in the supernatant was determined by LC/MS/MS.

Analysis. Samples were analyzed by LC/MS/MS using an Agilent 6410 mass spectrometer coupled with an Agilent 1200 high pressure liquid chromatography (HPLC) and a CTC PAL chilled autosampler, all controlled by MassHunter software (Agilent). After separation on a Hydrophilic interaction liquid chromatography (HILIC) HPLC column (Sepax) using an acetonitrile-ammonium acetate/acetic acid gradient system, peaks were analyzed by mass spectrometry (MS) using ESI ionization in multiple reaction monitoring (MRM) mode. MassHunter software was used to calculate the concentration of the test compounds in samples from the peak area using the appropriate calibration curves.

Calibration samples. Calibration curves were determined in rat plasma. Calibration samples were prepared by diluting a 50× stock solution of the test compound in PBS with blank matrix to the appropriate concentration and these samples were prepared as described above in the sample preparation. Stock solutions were prepared by serial dilution as shown in Table 40.

Data Analysis. Descriptive statistics were calculated for all pharmacokinetic parameters. Elimination half-lives (t_{1/2}) were calculated by linear regression of logarithmically transformed plasma concentration data for each period between doses and following the final dose.

Total areas under the plasma concentration curves (AUC) and under the first moment curves (AUMC) were calculated using linear trapezoidal summation across all concentration time points as well as for intervals between each dose administration and following the final dose. For the interval following the first oral dose of anatabine polacrilex, mean transit times (MTT) were calculated from the corresponding ratio of AUMC to AUC. Mean absorption times (MAT) were calculated according to the following relation:

MAT=MTT−MRT,

where MRT represents the mean residence time. This was calculated from the mean residence times.

The statistical comparison of parameters between male and female animals was made using a two-tailed, unpaired, t-test with a 95 percent confidence interval. Repeated-measures analysis of variance (ANOVA) was used for multiple comparisons of C_{p, max} involving successive determinations on the same group of animals.

Results

Physical Signs. No adverse events were observed.

Method Development. Table 41 shows the results of the LC/MS/MS method development for the determination of the appropriate ionization conditions and the mass to charge ratios (m/z) of the parent and product ions for anatabine and its deuterated analogue as determined above. The indicated product m/z ratios were used for the analysis of the relevant test samples.

See Example 3 for the product ion spectra and sample chromatograms for each compound in Table 41. The limits of detection (LOD), lower (LLQ), and upper (ULQ) limits of quantitation was derived from the calibration curve and are shown in Table 42.

Analysis of Dosing Solutions. Table 43 provides a summary of the analyses of the dosing solutions used during the conduct of this study. The percent differences between the actual and expected concentrations are shown. The lowest dose of anatabine, which was 63% of the expected concentration and the high dose was 84% of the expected level.

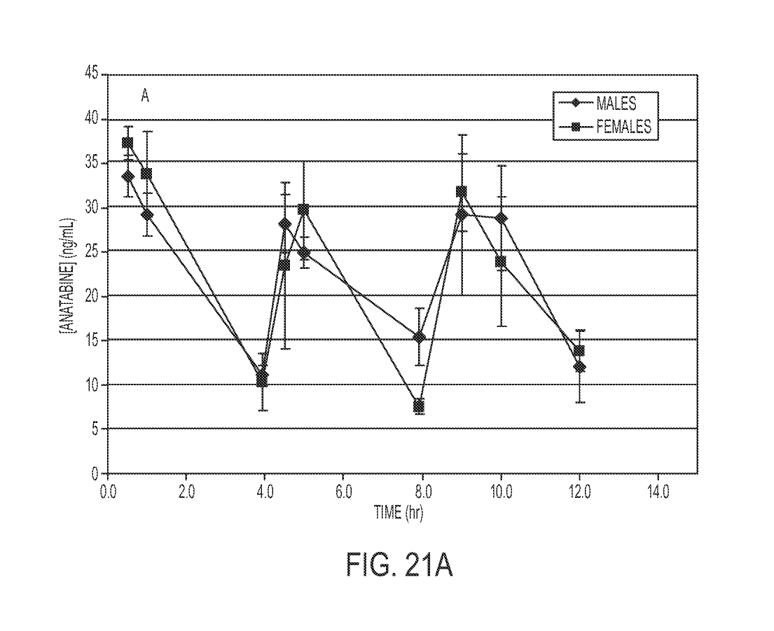

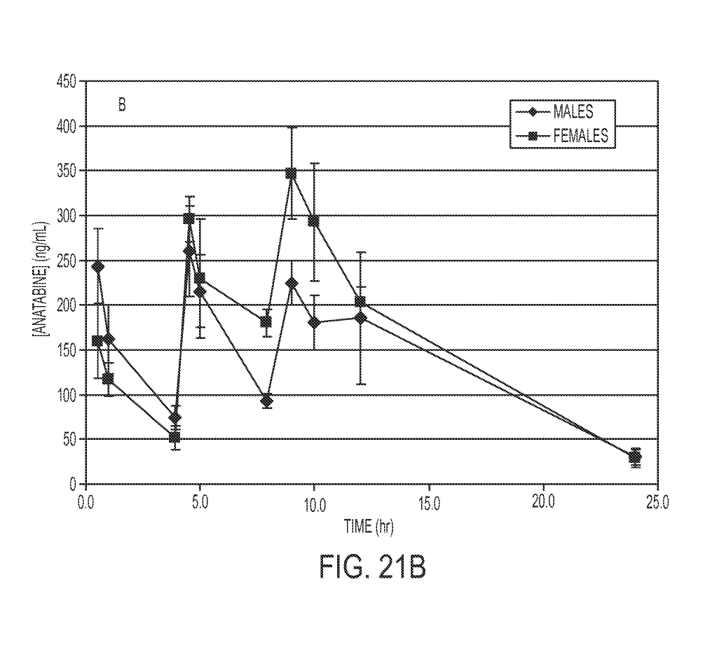

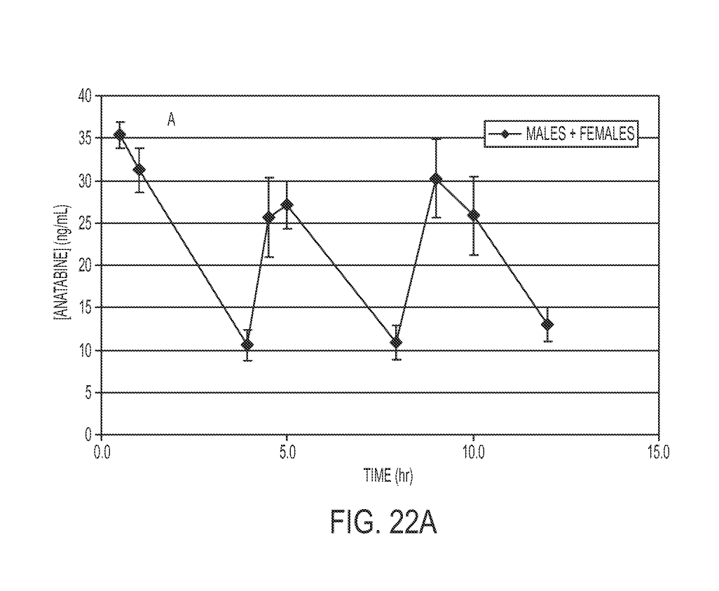

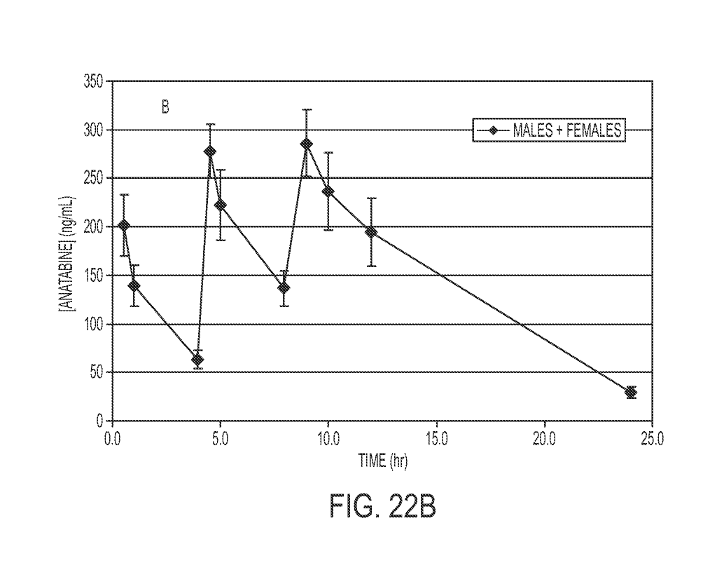

Plasma Pharmacokinetic Results & Analysis. FIG. 21A and FIG. 21B show the mean plasma anatabine concentration-time curves for male and female rats in each of the two dose groups: 0.6 mg/kg (FIG. 21A) and 6.0 mg/kg BW (FIG. 21B). FIG. 22A and FIG. 22B show the same data with the values from both males and females combined. In each instance, three plasma concentration maxima can be observed corresponding to the administration of the three divided doses of anatabine polacrilex at 0, 4 and 8 hours. Similarly, two anatabine plasma concentration minima are found prior to administration of the final dose.

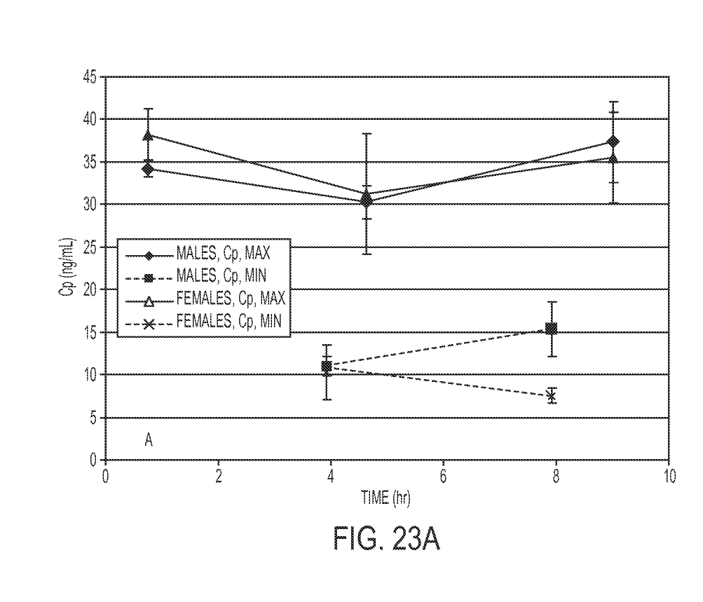

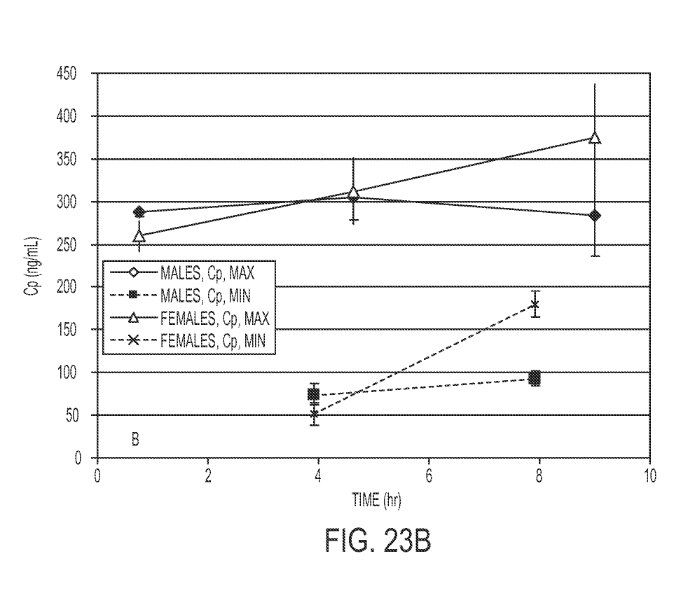

The mean maxima and minima anatabine plasma concentrations (C_{p, max}, C_{p, min}) for males and females in each dose group are recorded in Table 44 along with the mean time to maximal concentration following each of the three doses (Tₘₐₓ). Statistical comparisons between male and female animals within each dose group revealed no significant differences in any of the parameters, except for the second plasma concentration minimum (C_{p, min(2)}) in both treatment groups; 15.3±5.5 ng/mL versus 7.5±1.7 ng/mL in the 0.6 mg/kg BW treatment group, and 93±16 ng/mL versus 180±31 ng/mL in the 6.0 mg/kg BW treatment group. FIG. 23A and FIG. 23B show the data in Table 44 plotted as a function of time.

The times to reach maximal concentration generally occurred within 0.5 hr and 1.0 hr post administration in both treatment groups and for both genders, following doses one and two (see Table 45). After the third dose, tₘₐₓ₍₃₎ was generally between 1.0 and 2.0 hours post-administration; however, it should be noted that the earliest sampling point was at 1 hr following this dose.

Table 45 shows a comparison of the plasma concentration maxima and minima over time for male and female rats in both treatment groups. There were no statistically significant changes in any of these parameters except for the plasma concentration minima for female rats in the high dose group; C_{p, min} increased from 51.5±26.0 ng/mL to 180.0±30.7 ng/mL.

The mean exposures (AUC), elimination half-lives (t_{1/2}), mean transit times (MTT) and mean absorption times (MAT) are reported in Table 46 for male and female animals in the two treatment groups. There are no significant differences between the genders in any parameter, at either dose level.

When the male and female data are combined, as shown in Table 47, there is a significant difference in total exposure as would be expected as a consequence of the two different dose levels (AUC_{0→∞}; 285±77 ng·hr/mL versus 3496±559 ng·hr/mL). There is also a significant difference in the terminal elimination half-life between the two treatment groups (t_{1/2, terminal}; 1.79±0.64 hr versus 4.53±1.77 hr), where t_{1/2, terminal} refers to the elimination half-life following the final dose of anatabine polacrilex.

As there were no significant differences in the calculated elimination half-life, mean transit times and mean absorption times between treatment groups following the first dose of the test compound (t_{1/2, 0→4}, MTT_{0→4}, and MAT_{0→4}, respectively), the data at both dose levels were combined for males and females (see Table 48). There were no significant differences in these parameters between genders.

Table 49 provides animal weights and dosing times. Table 50 provides measured concentrations of anatabine in rat plasma samples at each time point. Table 51 provides mean concentration and description statistics of anatabine in plasma samples at each time point.

The data from both genders are also combined to give corresponding overall values. The calculated mean elimination half-life (t_{1/2, 0→4}) is 1.93±0.73 hr, the mean transit time (MTT_{0→4}) is 3.01±1.25 hr, and the mean absorption time (MAT_{0→4}) is 0.56±1.25 hr.

Discussion

This study evaluated the pharmacokinetics of anatabine in male and female Sprague-Dawley rats following the repeat-dose administration of anatabine polacrilex by oral gavage at two different dose levels. Anatabine was administered at 0.6 mg/kg BW in three, divided, doses of 0.2 mg/kg BW, or at 6.0 mg/kg BW in three, divided, doses of 2.0 mg/kg BW. Each dose was separated by an interval of four hours. All animals in both treatment groups appeared normal immediately following each administration of the test compound and no adverse signs were observed for the duration of the observation and plasma sampling period.

Anatabine concentrations can be measured in rat plasma following single and repeat oral dosing. The mean time to maximal plasma concentration following the first two oral doses ranged from 0.50 to 0.88±0.25 hr. There were no significant differences between gender or dose group. After the third dose of test compound, the mean time to maximal plasma concentration ranged from 1.00 to 2.00±1.41 hr; although in this instance the first time point measured was at one hour post-dose and therefore, it is possible that actual maximum occurred prior to this time. Within each dose group there were no significant differences in C_{p, max} between males and females, nor was there any significant change in this parameter over time. In females of the high dose group C_{p, max} appeared to increase from 259.8±35.4 ng/mL to 374.8±122.9 ng/mL; however, the trend was not statistically significant.

There were also two, observable, minima following the first two oral doses of anatabine polacrilex. In general, the minima were not significantly different from one another over time, except for females of the high dose group, which increased from 51.5±26.0 ng/mL to 180±31 ng/mL. Overall, these results suggest that with a 4-hour dosing interval, and after eight hours, near steady-state conditions appear have been achieved in male animals, whereas in females this may not yet be the case.

Within the two treatment groups, the total exposure, elimination half-lives, mean transit times and mean absorption times did not differ significantly between male and female rats. When these data are combined and grouped according to dose level the total exposure is significantly greater at the high dose as would be expected; however, the terminal elimination half-life is also significantly higher in the 6.0 mg/kg BW group compared to the 0.6 mg/kg BW dose group. The reason for this difference is not apparent since the mean transit times and mean absorption times did not differ significantly.

The elimination half-life, mean transit time and mean absorption time following the first oral dose of the test compound are the most reliable estimates of these parameters since the plasma concentration data are not confounded by carry-over amounts from a previous dose. The overall elimination half-life of anatabine following the first oral dose was 1.93±0.73 hr, the mean transit time was 3.01±1.25 hr and the mean absorption time was 0.56±1.25 hr. The mean absorption time (also often called mean arrival time) of 0.56 compares favorably with the calculated Tₘₐₓ values following the first two doses and indicates that the absorption of anatabine occurs within the first 30 to 60 minutes after oral administration.

TABLE 1A

| | |
|---|---|
| headaches | Reuter et al. (2006) Headache 43: 426-427 |
| pain | Tegeder et al. (2004) Journal of Neuroscience 24: 1637-1645 |
| | Niederberger et al. (2004) Neurosci. 24, 1637-45 |
| | Niederberger et al. (2008) FASEB Journal 22: 3432-3442 |
| complex regional pain | Hettne et al. (2007) Journal of Biomedical Discovery and |
| syndrome | Collaboration 2: 2 |
| cardiac hypertrophy | Purcell & Molkentin (2003) Circulation 108: 638-640 |
| | Freund et al. (2005) Circulation 111: 2319-2325 |
| | Sen & Roy (2005) American Journal of Physiology: Heart |
| | and Circulatory Physiology 289: H17-H19 |
| muscular dystrophy (type | Baghdiguian et al. (1999) Nature Medicine 5: 503-511; |
| 2a) | erratum in Nature Medicine 1999 Jul; 5(7): 849] |
| muscle wasting | Hasselgren (2007) Journal of Cellular Physiology 293: |
| | R1545-R1551 |
| catabolic disorders | Holmes-McNary (2002) Current Opinion in Clinical |
| | Nutrition and Metabolic Care 5: 255-263 |
| diabetes mellitus, type 1 | Ho & Bray (1999) Proceedings of the Society of |
| | Experimental Biology and Medicine 222: 205-213 |
| | Eldor et al. (2006) Proceedings of the National Academy |
| | of Sciences USA 103: 5072-5077 |
| diabetes mellitus, type 2 | Yuan et al. (2001) Science 293: 1673-1677 |
| | Lehrke et al. (2004) PLoS Medicine 1: e45 |
| | Chen F (2005) Biochemical and Biophysical Research |
| | Communications 332: 1-3 |
| obesity | Gil et al. (2007) British Journal of Nutition 98: S121-S126 |
| fetal growth retardation | Mammon et al. (2005) Review of Diabetic Studies 2 27-34 |
| hypercholesterolemia | Wilson et al. (2000) Atherosclerosis 148: 23-30 |
| atherosclerosis | Brand et al., J Clin Invest 1996; 97: 1715-1722. |
| | Ross et al. (2001) American Journal of Clinical Pathology |
| | 116: S97-S107 |
| | Li & Gao (2005) Medical Hypotheses 64: 694-698 |
| heart disease | Valen et al. (2001) Journal of the American College of |
| | Cardiology 38: 307-314 |
| chronic heart failure | Frantz et al. (2003) Cardiovascular Research 57: 749-756 |
| | Gong et al. (2007) International Journal of Clinical |
| | Practice 61: 611-621 |
| ischemia/reperfusion | Toledo-Pereyra et al. (2004) Experimental and Clinical |
| | Transplantation 2: 174-177 |
| | Nichols (2004) Drug News and Perspectives 17: 99-104 |
| | Ridder & Schwaninger (2008) Neuroscience 158(3): 995-1006 |
| stroke | Herrmann et al. (2005) Nature Medicine 11: 13322-1329 |
| cerebral aneurysm | Aoki et al. (2007) Circulation 116 2830-2840 |
| | Aoki et al. (2009) Arteriosclerosis, Thrombosis, and |
| | Vascular Biology 29: 1080-1086 |
| angina pectoris | Ritchie (1998) Circulation 98: 1707-1713 |
| pulmonary disease | Christman et al. (2000) Chest 117: 1482-1487 |
| cystic fibrosis | Bodas & Vij, Discovery Medicine, 9(47): 346-356, April |
| | 2010 |
| | Pollard et al. (2005) Proteomics 5: 2210-2216 |
| | Carrabino et al. (2006) Journal of Cystic Fibrosis 5: 113-119 |
| | Rottner et al. (2007) FASEB Journal 21: 2939-2948 |
| acid-induced lung injury | Madjdpour et al. (2003) Anesthesiology 99: 1323-1332 |
| pulmonary hypertension | Sawada et al. (2007) Chest 132: 1265-1274 |
| chronic obstructive | Barnes (2002) Nature Reviews Drug Discovery 1: 437-446 |
| pulmonary disease | Rahman & Kilty (2006) Current Drug Targets 7: 707-720 |
| (COPD) | |
| adult respiratory distress | Schwartz et al., Crit Care Med 1996; 24: 1285-1292. |
| syndrome | |
| hyaline membrane disease | Cheah et al. (2005) Pediatric Research 57: 616-623 |
| kidney disease | Guijarro & Egido (2001) Kidney International 59: 415-424 |
| | Camici (2007) Medical Hypotheses 68: 900-905 |
| | Guzik & Harrison (2007) Circulation Research 101: 227-229 |
| glomerular disease | Zheng et al. (2005) Virchows Archiv 448: 172-183 |
| alcoholic liver disease | Zima & Kalousova (2005) Alcoholism, Clinical and |
| | Experimental Research 29 (Supplement): 110S-115S |
| leptospirosis renal disease | Yang et al. (2001) Nephrology Dialysis Transplantation 16 |
| | Suppl 5: 73-77 |
| gut diseases | Neurath et al. (1998) Gut 43: 856-860 |
| peritoneal endometriosis | Gonzalez-Ramos et al. (2007) Molecular Human |
| | Reproduction 13(7): 503-509 |
| skin diseases | Bell et al. (2003) Cell Signaling 15: 1-7 |
| nasal sinusitis | Xu et al. (2006) Neurochemistry Research 31: 1263-1269 |
| anhidrotic ecodermal | Puel et al. (2005) Journal of Endotoxin Research 11: 220-224 |
| dysplasia-id | |
| Behcet's disease | Todaro et al. (2005) Arthritis and Rheumatism 52: 2179-2191 |
| incontinentia pigmenti | Courtois & Israel (2000) Sci STKE Nov 14 (58): PE1 |
| tuberculosis | Zea et al. (2006) Journal of Infectious Diseases 194: 1385-1393 |
| asthma | Barnes, Am J Respir Crit Care Med 1996; 154: S21-S27 |
| | Stacey et al., Biochem Biophys Res Commun |
| | 1997; 236: 522-526 |
| | Pahl & Szelenyi (2002) Inflammation Research 51: 273-282 |
| arthritis | Roshak et al. (2002) Current Opinion in Pharmacology 2: |
| | 316-321 |
| | Roman-Blas & Jimenez (2006) Osteoarthritis and |
| | Cartilage 14 839-848 |
| | Aud & Peng (2006) Nature Clinical Practice: |
| | Rheumatology 2: 434-442 |
| | Okamoto (2006) Endocrine, Metabolic & Immune |
| | Disorders: Drug Targets 6: 359-372 |
| rheumatoid arthritis | Handel et al., Arthritis Rheum 1995; 38: 1762-1770 |
| | Sakurada et al., Int Immunol 1996; 8: 1483-1493 |
| | Roshak et al., J Biol Chem 1996; 271: 31496-31501. |
| Crohn's disease | Pena & Penate (2002) Revista Espanola d Enfermedades |
| | Digestivas 94: 351-360 |
| colitis | Chen et al. (2005) World Journal of Gastroenterology 11: |
| | 1508-1514 |
| ulcerative colitis | Barnes & Karin, NEJM 336, 1066-71, 1997 |
| ocular allergy | Bielory et al. (2002) Current Opinion in Allergy and |
| | Clinical Immunology 2: 435-445 |
| glaucoma | Zhou et al. (2005) Journal of Biological Chemistry 280: |
| | 31240-31248 |
| appendicitis | Pennington et al. (2000) American Surgery 66: 914-918 |
| Paget's disease | Lin et al. (2007) Journal of Dermatological Science 45: |
| | 187-192 |
| pancreatitis | Weber & Adler (2001) Pancreatology 1: 356-362 |
| | Gray et al. (2006) Pancreas 33: 260-267 |
| periodonitis | Nichols et al. (2001) Annals of Periodontology 6: 20-29; |
| | Ambili et al. (2005) Journal of Periodontology 76: 1148-1153 |
| endometriosis | Guo (2006) Gynecology and Obstetrics Investigations 63: |
| | 71-97 |
| | Celik et al. (2008) Human Reproduction 23: 2458-2465 |
| inflammatory bowel | Neurath et al., Nature Med 1996; 2: 998-1004 |
| disease | Dijkstra et al. (2002) Scandinavian Journal of |
| | Gastroenterology Supplement 236: 37-41 |
| | Atreya et al. (2008) Journal of Internal Medicine 263: 591-596 |
| inflammatory lung disease | Park & Christman (2006) Current Drug Targets 7: 661-668 |
| sepsis | Wratten et al. (2001) Contributions to Nephrology 132: |
| | 400-414 |
| septic shock | Mukaida et al., J Leukoc Res 1996; 59: 145-151 |
| | Bohrer et al., J Clin Invest 1997; 100: 972-985. |
| silica-induced diseases | Chen & Shi 2002) Molecular and Cellular Biochemistry |
| | 234-235: 169-176 |
| sleep apnea | Lavie (2003) Sleep Medicine Review 7: 35-51 |
| viral infection (e.g., | Barnes & Karin, NEJM 336, 1066-71, 1997 |
| rhinovirus, influenza | |
| virus) | |
| aids (HIV-1) | Hiscott et al. (2001) Journal of Clinical Investigation 107: |
| | 143-151 |
| allograft rejection | Lee & Burckart, J. Clin. Pharmacol. 38, 981-93, 1998 |
| | Bach et al., Nature Med 13, 944-48, 1997A |
| | Bach et al., Transplant Proc 29, 56-58, 1997B |
| autoimmunity | Hayashi & Faustman (2002) Diabetes Technology & |
| | Therapeutics 2: 415-428 |
| antiphospholipid | Lopez-Pedrera et al. (2005) Arthritis and Rheumatism 54: |
| syndrome | 301-311 |
| lupus | Kammer & Tsokos (2002) Current Directions in |
| | Autoimmunity 5: 131-150 |
| lupus nephritis | Zheng et al. (2006) Human Pathology 37: 637-647 |
| chronic disease syndrome | Maes et al. (2007) Neuro Endocinol Letters 28: 456-462 |
| familial Mediterranean | Onen (2006) Rheumatology International 26: 489-496 |
| fever | |
| hereditary periodic fever | Jeru et al. (2008) Proceedings of the National Academy of |
| syndrome | Sciences USA 105, 1614-1619 |
| psychosocial stress | Bierhaus et al. (2004) Pediatic Nephrology 19: 1189-1191 |
| diseases | |
| neuropathological diseases | Cechetto (2001) Progress in Brain Research 132: 391-404 |
| familial amyloidotic | Mazzeo et al. (2004) Archives of Neurology 61: 1097-1102 |
| polyneuropathy | |
| traumatic brain injury | Hang et al. (2005) World Journal of Gastroenterology 11: |
| | 1149-1154 |
| spinal cord injury | Brambilla et al. (2005) Journal of Experimental Medicine |
| | 202: 145-156 |
| Parkinson's disease | Soos et al. (2004) Neuroreport 15 1715-1718 |
| multiple sclerosis | Satoh et al. (2007) Neuroscience Letters 422: 30-33 |
| rheumatic disease | Okamoto T (2006) Endocrine, Metabolic & Immune |
| | Disorders: Drug Targets 6: 359-372 |
| Alzheimer's disease | Mattson & Camandola (2001) Journal of Clinical |
| | Investigation 107: 247-254 |
| | Kaltschmidt et al., Proc. Natl. Acad. Sci. USA 94: 2642-2647, |
| | 1997 |
| | Lukiw, J. Biol. Chem. 2008 Nov 14; 283(46): 31315-22. |
| | Epub 2008 Sep 18 |
| amyotrophic lateral | Xu et al. (2006) Neurochemistry Research 31: 1263-1269 |
| sclerosis | |
| Huntington's disease | Khoshnan et al. (2004) Journal of Neuroscience 24: 7999-8008 |
| retinal disease | Kitaoka et al. (2004) Brain Research Molecular Brain |
| | Research 131: 8-16 |
| cataracts | Yang et al. (2006) Chinese Journal of Traumatology 9: 86-90 |
| hearing loss | Merchant et al. (2005) Otology and Neurotology 26: 151-160 |
| cancer | Gilmore et al. (2002) Cancer Letters 181: 1-9 |
| solid tumors (generally) | Pacifico & Leonardi (2006) Biochemical Pharmacology |
| | 72: 1142-1152 |
| breast cancer | Akshatri et al. (1997) Molecular and Cellular Biology 17: |
| | 3629-3639 |
| cervical cancer | Nair et al. (2003) Oncogene 22: 50-58 |
| ovarian cancer | Lin et al., Clin. Cancer Res. 13, 3423-30, 2007 |
| | Samanta et al., J. Biol. Chem. 279, 7576-83, 2004 |
| adenocarcinoma | Dejardin et al. (1999) Oncogene 18: 2567-2577 |
| vulvar cancer | Seppanen & Vihko (2000) Immunology Letters 74: 103-109 |
| prostate cancer | Huang et al. (2001) Oncogene 20: 4188-4197 |
| kidney cancer | Oya et al. (2001) Oncogene 20: 3888-3896 |
| | Oya et al. (2003) Carcinogenesis 24: 377-384 |
| bladder cancer | Horiguchi et al. (2003) Expert Reviews in Anticancer |
| | Therapy 3: 793-798 |
| lung cancer | Tichelaar et al. (2004) Chest 125: 153S |
| mesothelioma | Bertino et al. (2007) International Journal of Cancer 121: |
| | 2766-2774 |
| non small-cell lung cancer | Zhang et al. (2006) Annals of Thoracic Surgery 82 243-248 |
| liver cancer | Tai et al. (2000) Cancer 89: 2274-2281 |
| pancreatic cancer | Wang et al. (1999) Clinical Cancer Research 5: 119-127 |
| | Zhang & Rigas (2006) International Journal of Oncology |
| | 29: 185-192 |
| esophageal/gastric cancer | Sutter et al. (2004) Onkologie 27: 17-21 |
| | Jackson & Evers (2006) Cancer Treatment and Research |
| | 130: 39-65 |
| laryngeal cancer | Zhu et al. (2004) Lin Chuang Er Bi Yan Hou Ke Za Zhi |
| | 18: 745-6, 766 |
| stomach cancer | Sasaki et al. (2001) Clinical Cancer Research 7: 4136-4142 |
| colon cancer | Lind et al. (2001) Surgery 130: 363-369 |
| thyroid cancer | Visconti et al. (1997) Oncogene 15: 1987-1994 |
| parathyroid cancer | Corbetta et al. (2005) Endocrine Related Cancer 12: 929-937 |
| melanoma | Yang & Richmond (2001) Cancer Research 61: 4901-4909 |
| | Amiri KI and Richmond A (2005) Cancer Metastasis |
| | Reviews 24: 301-313 |
| squamous cell carcinoma | Loercher et al. (2004) Cancer Research 64: 6511-6523 |
| head and neck cancer | Ondrey et al. (1999) Molecular Carcinogenesis 26: 119-129 |
| | Jackson-Bernitsas et al. (2007) Oncogene 26: 1385-1397 |
| endometrial (uterine) | Pallares et al. (2004) Journal of Pathology 204: 595-577 |
| cancer | |
| cylindromatosis | Kovalenko et al. (2003) Nature 424: 801-805 |
| trichoepithelioma | Almeida et al. (2008) Dermatol. 127, 587-93 |
| Hilar cholangiocarcinoma | Chen et al. (2005) World Journal of Gastroenterology 11: |
| | 726-728 |
| oral carcinoma | Nakayama et al. (2001) Cancer 92: 3037-3044 |
| | Ruan et al. (2007) Phytotherapy Research 22, 407-15 |
| astrocytoma/glioblastoma | Hayashi et al. (2001) Neurologia Medico-Chirufica 41: |
| | 187-195 |
| | Smith et al. (2007) Molecular and Cellular Biochemistry |
| | 307: 1-2, 141-147 |
| neuroblastoma | Bian et al. (2002) Journal of Biological Chemistry 277: |
| | 42144-42150 |
| glioblastoma | Raychaudhuri et al. (2007) Journal of Neurooncology 85: |
| | 39-47 |
| Hodgkin's lymphoma | Bargou et al. (1996) Blood 87: 4340-4347 |
| | Bargou et al. (1997) Journal of Clinical Investigation 100: |
| | 2961-2969 |
| acute lymphoblastic | Kordes et al. (2000) Leukemia 14: 399-402 |
| leukemia | |
| acute myelogenous | Guzman et al. (2001) Blood 98: 2301-2307 |
| leukemia | |
| acute T-cell leukemia (+/− | Arima & Tei (2001) Leukemia and Lymphoma 40: 267-278 |
| HTLV-1) | |
| acute non-lymphocytic | Lei & Zhao (2007) Zhongguo Shi Yan Xue Za Zhi 15 253-257 |
| leukemia | |
| chronic lymphocytic | Furman et al. (2000) Journal of Immunology 164: 2200-2206 |
| leukemia | |
| Burkitt lymphoma (EBV) | Knecht et al. (2001) Oncology 60: 289-302 |
| mantle cell lymphoma | Martinez (2003) Cancer Research 63: 8226-8232 |
| myelodysplastic syndrome | Fabre et al. (2007) Oncogene 26: 4071-4083 |
| multiple myeloma | Gilmore (2007) Cancer Cell 12 95-97 |
| | Berenson et al.(2001) Seminars in Oncology 28: 626-633 |
| diffuse large b-cell | Davis et al. (2001) Journal of Experimental Medicine 194: |
| lymphoma | 1861-1874 |
| MALT lymphoma | Sagaert et al. (2007) Leukemia 21: 389-396 |
| Waldenstrom | Leleu et al. (2008) Blood 111: 5068-5077 |
| macroglobulinemia | |
| osteoporosis | Ray & Cohn, J. Clin. Invest. 104, 985-993, 1999; |
| | Christman et al., Chest 117, 1482-1487, 2000 |

TABLE 2
Dosing solutions

| | | | Test | Test | |
|---|---|---|---|---|---|
| | | | compound | compound | |
| | Percentage | | concen- | concen- | |
| | content of | | tration | tration | Injection |
| Test | anatabine or | Dose level | (total) | (base) | volume |
| compound | nicotine base | (mg/kg) | (mg/mL) | (mg/mL) | (mL/kg) |
| Anatabine | 41.6 | 0.10 | 0.048 | 0.020 | 5 |
| Anatabine | 41.6 | 0.75 | 0.36 | 0.15 | 5 |
| Anatabine | 41.6 | 1.0 | 0.48 | 0.20 | 5 |
| Nicotine | 35.1 | 0.4 | 0.23 | 0.081 | 5 |

TABLE 3
Phase I

| | | | | Collection |
|---|---|---|---|---|
| | | Dose level | Number of | times |
| Test compound | Route | (mg/kg) | animals (M/F) | (minutes)^{a} |
| Anatabine | i.v. | 0.10 | 3/3 | 15, 30, 60, 90, |
| | | | | 120, 240, 360, |
| | | | | 480, 1440 |
| Anatabine | i.v. | 0.75 | 3/3 | 15, 30, 60, 90, |
| | | | | 120, 240, 360, |
| | | | | 480, 1440 |
| Anatabine | i.v. | 1.0 | 3/3 | 15, 30, 60, 90, |
| | | | | 120, 240, 360, |
| | | | | 480, 1440 |
| Nicotine | i.v. | 0.4 | 3/3 | 15, 30, 60, 90, |
| | | | | 120, 240, 360, |
| | | | | 480, 1440 |

^{a}Plasma samples were collected at all time points,. Brain tissue was collected at 1440 minutes.

TABLE 4
Phase II

| | | | Number of animals | Collection |
|---|---|---|---|---|
| | | Dose level | per time point | times |
| Test compound | Route | (mg/kg) | (M/F) | (minutes)^{a} |
| Anatabine | i.v. | 0.10 | 3/3 | 30, 360 |
| Anatabine | i.v. | 0.75 | 3/3 | 30, 360 |
| Anatabine | i.v. | 1.0 | 3/3 | 30, 360 |
| Nicotine | i.v. | 0.4 | 3/3 | 30, 360 |

^{a}Plasma samples and brain tissue were collected at all time points.

TABLE 5
Calibration Curve Concentrations

| nominal concentration | stock concentration |
|---|---|
| (ng/mL) | (μg/mL) |
| 5000 | 250 |
| 1667 | 83.3 |
| 555.5 | 27.8 |
| 185.2 | 9.3 |
| 61.7 | 3.1 |
| 20.6 | 1.0 |
| 6.9 | 0.34 |
| 2.3 | 0.11 |
| 0.76 | 0.038 |
| 0.25 | 0.013 |

TABLE 6
LC/MS/MS ionization conditions and identity of parent and product ions

| | | | | | Collision |
|---|---|---|---|---|---|
| | | | Precursor | Product | energy |
| Compound | MW | Polarization | m/z | m/z | (V) |
| Anatabine | 160.2 | Positive | 161.1 | 115.1 | 28 |
| Nicotine | 162.3 | Positive | 163.1 | 117.1 | 28 |
| (+/−)-nicotine- | 165.25 | Positive | 166.1 | 118 | 28 |
| 3′-d₃ | | | | | |
| (R,S)- | 164.24 | Positive | 165.1 | 148.1 | 20 |
| Antabine- | | | | | |
| 2,4,5,6-d₄ | | | | | |

TABLE 7
Limits of Detection and Calibration Curves

| | | Lower Limit of | Upper Limit of |
|---|---|---|---|
| | | Quantitation | Quantitation |
| | Limit of Detection | (LLQ) | (ULQ) |
| Sample | (LOD) (ng/mL) | (ng/mL) | (ng/mL) |
| Anatabine in rat | 0.76 | 2.3 | 5000 |
| plasma | | | |
| Anatabine in rat | 0.76 | 2.3 | 5000 |
| brain | | | |
| Nicotine in rat | 0.76 | 2.3 | 5000 |
| plasma | | | |
| Nicotine in rat | 0.76 | 2.3 | 5000 |
| brain | | | |

TABLE 8
Recovery from Plasma

| | % Recovery at Given Concentrations | | |
|---|---|---|---|
| Sample | 2.3 ng/mL | 62 ng/mL | 1667 ng/mL |

| | | | |
|---|---|---|---|
| Anatabine in rat plasma | 74 | 96 | ND^{a} |
| Anatabine in rat brain | ND | 96 | 90 |
| Nicotine in rat plasma | ND | 105 | 104 |
| Nicotine in rat brain | ND | 78 | 84 |

^{a}ND—not determined; two points per condition were evaluated for measuring recovery.

TABLE 9
Dosing Solution Analysis

| | | | | Actual |
|---|---|---|---|---|
| | | | | Concentration |
| | | Expected | Actual | relative to |
| | Dose | Concentration | Concentration | Expected |
| Compound | (mg/kg) | (mg/mL) | (mg/mL) | (%) |
| Nicotine | 0.4 | 0.081 | 0.096 | 118.5 |
| Nicotine | 0.4 | 0.081 | 0.096 | 118.5 |
| Anatabine | 0.1 | 0.02 | 0.014 | 70 |
| Anatabine | 0.75 | 0.15 | 0.135 | 90 |
| Anatabine | 1 | 0.2 | 0.177 | 88.5 |
| Anatabine | 0.1 | 0.02 | 0.015 | 75 |
| Anatabine | 0.75 | 0.15 | 0.133 | 88.7 |
| Anatabine | 1 | 0.2 | 0.162 | 81 |

TABLE 10
Statistical Comparison of the Pharmacokinetic Parameters

| | Nicotine (0.4 mg/kg) | | Anatabine (0.1 mg/kg) | | Anatabine (0.75 mg/kg) | | Anatabine (1.0 mg/kg) |
|---|---|---|---|---|---|---|---|
| Parameter | M + F | p | M + F | p | M + F | p | M + F |
| AUC_{0→∞} | 156.5 ± 32.6 | | 35.0 ± 11.6 | | 504.4 ± 63.5 | | 710.07 ± 88.4 |
| (ng · hr/mL) | | | | | | | |
| t_{1/2} (hr) | 0.67 ± 0.07 | 0.003^{a} | 1.44 ± 0.48 | 0.25^{d} | 1.68 ± 0.09 | 0.69^{f} | 1.64 ± 0.25 |
| | | <0.001^{b} | | 0.39^{e} | | | |
| | | <0.001^{c} | | | | | |
| MRT (hr) | 1.22 ± 0.13 | 0.004^{a} | 2.29 ± 0.07 | 0.35^{d} | 2.58 ± 0.14 | 0.63^{f} | 2.49 ± 0.38 |
| | | <0.001^{b} | | 0.55^{e} | | | |
| | | <0.001^{c} | | | | | |
| V_{D} (L/kg) | 2.06 ± 0.13 | <0.001^{a} | 6.08 ± 0.45 | <0.001^{d} | 3.33 ± 0.13 | 0.15^{f} | 3.08 ± 0.16 |
| | | <0.001^{b} | | <0.001^{e} | | | |
| | | <0.001^{c} | | | | | |

^{a}Nicotine vs. Anatabine (0.1 mg/kg)
^{b}Nicotine vs. Anatabine (0.75 mg/kg)
^{c}Nicotine vs. Anatabine (1.0 mg/kg)
^{d}Anatabine (0.1 mg/kg) vs. Anatabine (0.75 mg/kg)
^{e}Anatabine (0.1 mg/kg) vs. Anatabine (1.0 mg/kg)
^{f}Anatabine (0.75 mg/kg) vs. Anatabine (1.0 mg/kg)

TABLE 11
Comparison of Pharmacokinetic Parameters (±Std Dev) between
male and female animals in each Treatment Group

| | | | | | | | Anatabine |
|---|---|---|---|---|---|---|---|
| | Nicotine (0.4 mg/kg) | | Anatabine (0.1 mg/kg) | | | | (0.75 mg/kg) |
| Parameter | male | female | p | male | female | p | male |
| AUC_{0→∞} | 140.5 ± 5.8 | 172.6 ± 43.1 | 0.27 | 32.0 ± 7.7 | 37.9 ± 15.9 | 0.59 | 464.4 ± 36.2 |
| (ng · hr/mL) | | | | | | | |
| t_{1/2} (hr) | 0.66 ± 0.11 | 0.68 ± 0.02 | 0.81 | 1.25 ± 0.32 | 1.64 ± 0.59 | 0.37 | 1.64 ± 0.12 |
| MRT (hr) | 1.21 ± 0.20 | 1.22 ± 0.05 | 0.90 | 2.03 ± 0.41 | 2.55 ± 0.93 | 0.42 | 2.50 ± 0.19 |
| V_{D} (L/kg) | 2.10 ± 0.20 | 1.91 ± 0.17 | 0.54 | 5.29 ± 0.49 | 6.52 ± 0.66 | 0.22 | 3.48 ± 0.18 |

| | Anatabine | | | | |
|---|---|---|---|---|---|
| | (0.75 mg/kg) | | Anatabine (1.0 mg/kg) | | |
| Parameter | female | p | male | female | p |
| AUC_{0→∞} | 544.5 ± 62.7 | 0.13 | 631.3 ± 28.2 | 788.9 ± 9.3 | <0.001 |
| (ng · hr/mL) | | | | | |
| t_{1/2} (hr) | 1.73 ± 0.03 | 0.28 | 1.44 ± 0.08 | 1.84 ± 0.16 | 0.02 |
| MRT (hr) | 2.65 ± 0.01 | 0.25 | 2.18 ± 0.12 | 2.80 ± 0.24 | 0.02 |
| V_{D} (L/kg) | 3.19 ± 0.15 | 0.38 | 3.01 ± 0.12 | 3.16 ± 0.15 | 0.29 |

TABLE 12
Mean (ng/g ± Std Dev) concentrations of nicotine and anatabine
in rat brain extracts following a single, bolus, i.v. dose

| | Treatment Group | | | | | | | |
|---|---|---|---|---|---|---|---|---|
| | Nicotine | | Anatabine | | Anatabine | | Anatabine | |
| | 0.4 mg/kg | | 0.1 mg/kg | | 0.75 mg/kg | | 1.0 mg/kg | |
| Time (hr) | Male | Female | Male | Female | Male | Female | Male | Female |
| 0.5 | 94.3 ± 20.6 | 120.0 ± 3.0 | 20.0 ± 1.4 | 29.7 ± 4.5 | 276.0 ± 37.0 | 314.7 ± 67.9 | 323.0 ± 35.9 | 346.0 ± 56.7 |
| 6 | 6.0 ± 1.4 | 5.0 | 3.0 | 3.7 ± 1.5 | 21.3 ± 5.1 | 21.0 ± 6.2 | 5.0 ± 2.6 | 6.0 ± 2.8 |
| 24 | 6.3 ± 2.3 | 7.0 | 3.0 | 2.0 | <LLQ | <LLQ | 2.5 ± 0.7 | <LLQ |

TABLE 13A
Rat PK i.v. dose-Brain Collection
Compound: Anatabine, Nicoti
Dose: 5 mL/kg
Route: i.v. PBS

| | | | Dose | | 24 hr | | |
|---|---|---|---|---|---|---|---|
| Cmpnd | Rat | B.W. (g) | volume (mL) | time | time | Brain wt (g) | Volume (mL) |
| 1 | A | 245 | 1.23 | 8:17 | 8:17 | 1.74 | 1.74 |
| MALE | B | 247 | 1.23 | 8:19 | 8:19 | 1.88 | 1.88 |
| Anatabine | C | 238 | 1.20 | 8:21 | 8:21 | 1.80 | 1.80 |
| 0.1 mg/kg | | | | | | | |
| 2 | A | 205 | 1.03 | 8:23 | 8:23 | 1.68 | 1.68 |
| FEMALE | B | 211 | 1.05 | 8:25 | 8:25 | 1.83 | 1.83 |
| Anatabine | C | 202 | 1.00 | 8:27 | 8:27 | 1.66 | 1.66 |
| 0.1 mg/kg | | | | | | | |
| 3 | A | 255 | 1.28 | 8:29 | 8:29 | 1.90 | 1.90 |
| MALE | B | 223 | 1.13 | 8:31 | 8:31 | 1.82 | 1.82 |
| Anatabine | C | 242 | 1.20 | 8:33 | 8:33 | 1.84 | 1.84 |
| 0.75 mg/kg | | | | | | | |
| 4 | A | 204 | 1.03 | 8:35 | 8:35 | 1.82 | 1.82 |
| FEMALE | B | 205 | 1.03 | 8:37 | 8:37 | 1.82 | 1.82 |
| Anatabine | C | 210 | 1.05 | 8:39 | 8:39 | 1.71 | 1.71 |
| 0.75 mg/kg | | | | | | | |
| 5 | A | 242 | 1.20 | 8:41 | 8:41 | 1.83 | 1.83 |
| MALE | B | 251 | 1.25 | 8:43 | 8:43 | 1.85 | 1.85 |
| Anatabine | C | 246 | 1.23 | 8:45 | 8:45 | 1.90 | 1.90 |
| 1.0 mg/kg | | | | | | | |
| 6 | A | 213 | 1.08 | 8:47 | 8:47 | 1.95 | 1.95 |
| FEMALE | B | 218 | 1.10 | 8:49 | 8:49 | 1.75 | 1.75 |
| Anatabine | C | 219 | 1.10 | 8:51 | 8:51 | 1.91 | 1.91 |
| 1.0 mg/kg | | | | | | | |
| 7 | A | 242 | 1.20 | 8:54 | 8:54 | 2.03 | 2.03 |
| MALE | B | 241 | 1.20 | 8:56 | 8:56 | 1.98 | 1.98 |
| Nicotine | C | 252 | 1.25 | 8:58 | 8:58 | 1.86 | 1.86 |
| 0.4 mg/kg | | | | | | | |
| 8 | A | 219 | 1.10 | 9:00 | 9:00 | 1.82 | 1.82 |
| FEMALE | B | 215 | 1.08 | 9:02 | 9:02 | 1.87 | 1.87 |
| Nicotine | C | 220 | 1.10 | 9:04 | 9:04 | 1.87 | 1.87 |
| 0.4 mg/kg | | | | | | | |
| Control | 1 | N/A | N/A | N/A | N/A | 1.83 | 1.83 |
| Male | 2 | N/A | N/A | N/A | N/A | 1.94 | 1.94 |
| | 3 | N/A | N/A | N/A | N/A | 1.82 | 1.82 |

TABLE 13B
Animal Weights and Dosing Times
Rat PK i.v. dose-Brain Collection
Dose: 5 mL/kg
Compound: Anatabine, Nicotine
Route: i.v., PBS

| | | | Dose | | 0.5 hour | | |
|---|---|---|---|---|---|---|---|
| Cmpnd | Rat | B.W.(g) | volume (mL) | time | time | Brain wt (g) | Volume (mL) |
| 1 | D | 264 | 1.30 | 12:38 | 13:08 | 1.58 | 1.58 |
| MALE | E | 270 | 1.35 | 12:41 | 13:11 | 1.54 | 1.54 |
| Anatabine 0.1 mg/kg | F | 252 | 1.26 | 12:45 | 13:15 | 1.69 | 1.69 |
| 2 | D | 220 | 1.10 | 12:46 | 13:16 | 1.78 | 1.78 |
| FEMALE | E | 206 | 1.03 | 12:50 | 13:20 | 1.54 | 1.54 |
| Anatabine 0.1 mg/kg | F | 214 | 1.08 | 12:52 | 13:22 | 1.69 | 1.69 |
| 3 | D | 259 | 1.30 | 12:56 | 13:26 | 1.70 | 1.70 |
| MALE | E | 266 | 1.33 | 12:58 | 13:28 | 1.66 | 1.66 |
| Anatabine 0.75 mg/kg | F | 264 | 1.33 | 13:03 | 13:33 | 1.46 | 1.46 |
| 4 | D | 208 | 1.05 | 13:05 | 13:35 | 1.81 | 1.81 |
| FEMALE | E | 219 | 1.10 | 13:09 | 13:39 | 1.84 | 1.84 |
| Anatabine 0.75 mg/kg | F | 223 | 1.13 | 13:12 | 13:42 | 1.69 | 1.69 |
| 5 | D | 276 | 1.38 | 13:16 | 13:46 | 2.00 | 2.00 |
| MALE | E | 252 | 1.25 | 13:19 | 13:49 | 1.84 | 1.84 |
| Anatabine 1.0 mg/kg | F | 255 | 1.28 | 13:22 | 13:52 | 1.68 | 1.68 |
| 6 | D | 212 | 1.05 | 13:25 | 13:55 | 1.66 | 1.66 |
| FEMALE | E | 225 | 1.13 | 13:29 | 13:59 | 1.56 | 1.56 |
| Anatabine 1.0 mg/kg | F | 236 | 1.18 | 13:32 | 14:02 | 1.79 | 1.79 |
| 7 | D | 273 | 1.38 | 13:36 | 14:06 | 1.76 | 1.76 |
| MALE | E | 256 | 1.28 | 13:39 | 14:09 | 1.72 | 1.72 |
| Nicotine 0.4 mg/kg | F | 263 | 1.33 | 13:44 | 14:14 | 1.77 | 1.77 |
| 8 | D | 212 | 1.05 | 13:48 | 14:18 | 1.75 | 1.75 |
| FEMALE | E | 213 | 1.05 | 13:52 | 14:22 | 1.81 | 1.81 |
| Nicotine 0.4 mg/kg | F | 231 | 1.15 | 13:59 | 14:29 | 1.90 | 1.90 |

TABLE 14
Measured Concentrations of Anatabine and Nicotine in Rat Brain Extracts
and Plasma Samples at Each Time Point

| | | | | Time | |
|---|---|---|---|---|---|
| | Dose | | | Point | Concentration |
| Compound | Group | Tissue | Rat ID | (hr) | (ng/mL) |
| Nicotine | Nicotine | Rat Brain | 7D | 0.5 | 110 |
| | 0.4 mg/kg | | | | |
| Nicotine | Nicotine | Rat Brain | 7E | 0.5 | 71 |
| | 0.4 mg/kg | | | | |
| Nicotine | Nicotine | Rat Brain | 7F | 0.5 | 102 |
| | 0.4 mg/kg | | | | |
| Nicotine | Nicotine | Rat Brain | 7G | 6 | 5 |
| | 0.4 mg/kg | | | | |
| Nicotine | Nicotine | Rat Brain | 7H | 6 | <LLQ |
| | 0.4 mg/kg | | | | |
| Nicotine | Nicotine | Rat Brain | 7I | 6 | 7 |
| | 0.4 mg/kg | | | | |
| Nicotine | Nicotine | Rat Brain | 7A | 24 | 5 |
| | 0.4 mg/kg | | | | |
| Nicotine | Nicotine | Rat Brain | 7B | 24 | 5 |
| | 0.4 mg/kg | | | | |
| Nicotine | Nicotine | Rat Brain | 7C | 24 | 9 |
| | 0.4 mg/kg | | | | |
| Nicotine | Nicotine | Rat Brain | 8D | 0.5 | 117 |
| | 0.4 mg/kg | | | | |
| Nicotine | Nicotine | Rat Brain | 8E | 0.5 | 120 |
| | 0.4 mg/kg | | | | |
| Nicotine | Nicotine | Rat Brain | 8F | 0.5 | 123 |
| | 0.4 mg/kg | | | | |
| Nicotine | Nicotine | Rat Brain | 8G | 6 | <LLQ |
| | 0.4 mg/kg | | | | |
| Nicotine | Nicotine | Rat Brain | 8H | 6 | <LLQ |
| | 0.4 mg/kg | | | | |
| Nicotine | Nicotine | Rat Brain | 8I | 6 | 5 |
| | 0.4 mg/kg | | | | |
| Nicotine | Nicotine | Rat Brain | 8A | 24 | <LLQ |
| | 0.4 mg/kg | | | | |
| Nicotine | Nicotine | Rat Brain | 8B | 24 | 7 |
| | 0.4 mg/kg | | | | |
| Nicotine | Nicotine | Rat Brain | 8C | 24 | <LLQ |
| | 0.4 mg/kg | | | | |
| Anatabine | Anatabine | Rat Brain | 1D | 0.5 | <LLQ |
| | 0.1 mg/kg | | | | |
| Anatabine | Anatabine | Rat Brain | 1E | 0.5 | 19 |
| | 0.1 mg/kg | | | | |
| Anatabine | Anatabine | Rat Brain | 1F | 0.5 | 21 |
| | 0.1 mg/kg | | | | |
| Anatabine | Anatabine | Rat Brain | 1G | 6 | 3 |
| | 0.1 mg/kg | | | | |
| Anatabine | Anatabine | Rat Brain | 1H | 6 | <LLQ |
| | 0.1 mg/kg | | | | |
| Anatabine | Anatabine | Rat Brain | 1I | 6 | <LLQ |
| | 0.1 mg/kg | | | | |
| Anatabine | Anatabine | Rat Brain | 1A | 24 | 3 |
| | 0.1 mg/kg | | | | |
| Anatabine | Anatabine | Rat Brain | 1B | 24 | <LLQ |
| | 0.1 mg/kg | | | | |
| Anatabine | Anatabine | Rat Brain | 1C | 24 | <LLQ |
| | 0.1 mg/kg | | | | |
| Anatabine | Anatabine | Rat Brain | 2D | 0.5 | 34 |
| | 0.1 mg/kg | | | | |
| Anatabine | Anatabine | Rat Brain | 2E | 0.5 | 25 |
| | 0.1 mg/kg | | | | |
| Anatabine | Anatabine | Rat Brain | 2F | 0.5 | 30 |
| | 0.1 mg/kg | | | | |
| Anatabine | Anatabine | Rat Brain | 2G | 6 | 5 |
| | 0.1 mg/kg | | | | |
| Anatabine | Anatabine | Rat Brain | 2H | 6 | 4 |
| | 0.1 mg/kg | | | | |
| Anatabine | Anatabine | Rat Brain | 2I | 6 | 2 |
| | 0.1 mg/kg | | | | |
| Anatabine | Anatabine | Rat Brain | 2A | 24 | 2 |
| | 0.1 mg/kg | | | | |
| Anatabine | Anatabine | Rat Brain | 2B | 24 | <LLQ |
| | 0.1 mg/kg | | | | |
| Anatabine | Anatabine | Rat Brain | 2C | 24 | <LLQ |
| | 0.1 mg/kg | | | | |
| Anatabine | Anatabine | Rat Brain | 3D | 0.5 | 266 |
| | 0.75 mg/kg | | | | |
| Anatabine | Anatabine | Rat Brain | 3E | 0.5 | 317 |
| | 0.75 mg/kg | | | | |
| Anatabine | Anatabine | Rat Brain | 3F | 0.5 | 245 |
| | 0.75 mg/kg | | | | |
| Anatabine | Anatabine | Rat Brain | 3G | 6 | 17 |
| | 0.75 mg/kg | | | | |
| Anatabine | Anatabine | Rat Brain | 3H | 6 | 27 |
| | 0.75 mg/kg | | | | |
| Anatabine | Anatabine | Rat Brain | 3I | 6 | 20 |
| | 0.75 mg/kg | | | | |
| Anatabine | Anatabine | Rat Brain | 3A | 24 | <LLQ |
| | 0.75 mg/kg | | | | |
| Anatabine | Anatabine | Rat Brain | 3B | 24 | <LLQ |
| | 0.75 mg/kg | | | | |
| Anatabine | Anatabine | Rat Brain | 3C | 24 | <LLQ |
| | 0.75 mg/kg | | | | |
| Anatabine | Anatabine | Rat Brain | 4D | 0.5 | 393 |
| | 0.75 mg/kg | | | | |
| Anatabine | Anatabine | Rat Brain | 4E | 0.5 | 272 |
| | 0.75 mg/kg | | | | |
| Anatabine | Anatabine | Rat Brain | 4F | 0.5 | 279 |
| | 0.75 mg/kg | | | | |
| Anatabine | Anatabine | Rat Brain | 4G | 6 | 16 |
| | 0.75 mg/kg | | | | |
| Anatabine | Anatabine | Rat Brain | 4H | 6 | 28 |
| | 0.75 mg/kg | | | | |
| Anatabine | Anatabine | Rat Brain | 4I | 6 | 19 |
| | 0.75 mg/kg | | | | |
| Anatabine | Anatabine | Rat Brain | 4A | 24 | <LLQ |
| | 0.75 mg/kg | | | | |
| Anatabine | Anatabine | Rat Brain | 4B | 24 | <LLQ |
| | 0.75 mg/kg | | | | |
| Anatabine | Anatabine | Rat Brain | 4C | 24 | <LLQ |
| | 0.75 mg/kg | | | | |
| Anatabine | Anatabine | Rat Brain | 5D | 0.5 | 349 |
| | 1.0 mg/kg | | | | |
| Anatabine | Anatabine | Rat Brain | 5E | 0.5 | 338 |
| | 1.0 mg/kg | | | | |
| Anatabine | Anatabine | Rat Brain | 5F | 0.5 | 282 |
| | 1.0 mg/kg | | | | |
| Anatabine | Anatabine | Rat Brain | 5G | 6 | 3 |
| | 1.0 mg/kg | | | | |
| Anatabine | Anatabine | Rat Brain | 5H | 6 | 4 |
| | 1.0 mg/kg | | | | |
| Anatabine | Anatabine | Rat Brain | 5I | 6 | 8 |
| | 1.0 mg/kg | | | | |
| Anatabine | Anatabine | Rat Brain | 5A | 24 | 3 |
| | 1.0 mg/kg | | | | |
| Anatabine | Anatabine | Rat Brain | 5B | 24 | 2 |
| | 1.0 mg/kg | | | | |
| Anatabine | Anatabine | Rat Brain | 5C | 24 | <LLQ |
| | 1.0 mg/kg | | | | |
| Anatabine | Anatabine | Rat Brain | 6D | 0.5 | 362 |
| | 1.0 mg/kg | | | | |
| Anatabine | Anatabine | Rat Brain | 6E | 0.5 | 393 |
| | 1.0 mg/kg | | | | |
| Anatabine | Anatabine | Rat Brain | 6F | 0.5 | 283 |
| | 1.0 mg/kg | | | | |
| Anatabine | Anatabine | Rat Brain | 6G | 6 | <LLQ |
| | 1.0 mg/kg | | | | |
| Anatabine | Anatabine | Rat Brain | 6H | 6 | 8 |
| | 1.0 mg/kg | | | | |
| Anatabine | Anatabine | Rat Brain | 6I | 6 | 4 |
| | 1.0 mg/kg | | | | |
| Anatabine | Anatabine | Rat Brain | 6A | 24 | <LLQ |
| | 1.0 mg/kg | | | | |
| Anatabine | Anatabine | Rat Brain | 6B | 24 | <LLQ |
| | 1.0 mg/kg | | | | |
| Anatabine | Anatabine | Rat Brain | 6C | 24 | <LLQ |
| | 1.0 mg/kg | | | | |
| Nicotine | Nicotine | Rat | 7A | 0.25 | 194 |
| | 0.4 mg/kg | Plasma | | | |
| Nicotine | Nicotine | Rat | 7B | 0.25 | 156 |
| | 0.4 mg/kg | Plasma | | | |
| Nicotine | Nicotine | Rat | 7C | 0.25 | 145 |
| | 0.4 mg/kg | Plasma | | | |
| Nicotine | Nicotine | Rat | 7A | 0.5 | 123 |
| | 0.4 mg/kg | Plasma | | | |
| Nicotine | Nicotine | Rat | 7B | 0.5 | 85 |
| | 0.4 mg/kg | Plasma | | | |
| Nicotine | Nicotine | Rat | 7C | 0.5 | 90 |
| | 0.4 mg/kg | Plasma | | | |
| Nicotine | Nicotine | Rat | 7D | 0.5 | 187 |
| | 0.4 mg/kg | Plasma | | | |
| Nicotine | Nicotine | Rat | 7E | 0.5 | 118 |
| | 0.4 mg/kg | Plasma | | | |
| Nicotine | Nicotine | Rat | 7F | 0.5 | 157 |
| | 0.4 mg/kg | Plasma | | | |
| Nicotine | Nicotine | Rat | 7A | 1 | 72 |
| | 0.4 mg/kg | Plasma | | | |
| Nicotine | Nicotine | Rat | 7B | 1 | 67 |
| | 0.4 mg/kg | Plasma | | | |
| Nicotine | Nicotine | Rat | 7C | 1 | 68 |
| | 0.4 mg/kg | Plasma | | | |
| Nicotine | Nicotine | Rat | 7A | 1.5 | 33 |
| | 0.4 mg/kg | Plasma | | | |
| Nicotine | Nicotine | Rat | 7B | 1.5 | 30 |
| | 0.4 mg/kg | Plasma | | | |
| Nicotine | Nicotine | Rat | 7C | 1.5 | 44 |
| | 0.4 mg/kg | Plasma | | | |
| Nicotine | Nicotine | Rat | 7A | 2 | 21 |
| | 0.4 mg/kg | Plasma | | | |
| Nicotine | Nicotine | Rat | 7B | 2 | 32 |
| | 0.4 mg/kg | Plasma | | | |
| Nicotine | Nicotine | Rat | 7C | 2 | 21 |
| | 0.4 mg/kg | Plasma | | | |
| Nicotine | Nicotine | Rat | 7A | 4 | <LLQ |
| | 0.4 mg/kg | Plasma | | | |
| Nicotine | Nicotine | Rat | 7B | 4 | <LLQ |
| | 0.4 mg/kg | Plasma | | | |
| Nicotine | Nicotine | Rat | 7C | 4 | <LLQ |
| | 0.4 mg/kg | Plasma | | | |
| Nicotine | Nicotine | Rat | 7A | 6 | <LLQ |
| | 0.4 mg/kg | Plasma | | | |
| Nicotine | Nicotine | Rat | 7B | 6 | <LLQ |
| | 0.4 mg/kg | Plasma | | | |
| Nicotine | Nicotine | Rat | 7C | 6 | <LLQ |
| | 0.4 mg/kg | Plasma | | | |
| Nicotine | Nicotine | Rat | 7G | 6 | <LLQ |
| | 0.4 mg/kg | Plasma | | | |
| Nicotine | Nicotine | Rat | 7H | 6 | <LLQ |
| | 0.4 mg/kg | Plasma | | | |
| Nicotine | Nicotine | Rat | 7I | 6 | <LLQ |
| | 0.4 mg/kg | Plasma | | | |
| Nicotine | Nicotine | Rat | 7A | 8 | <LLQ |
| | 0.4 mg/kg | Plasma | | | |
| Nicotine | Nicotine | Rat | 7B | 8 | <LLQ |
| | 0.4 mg/kg | Plasma | | | |
| Nicotine | Nicotine | Rat | 7C | 8 | <LLQ |
| | 0.4 mg/kg | Plasma | | | |
| Nicotine | Nicotine | Rat | 7A | 24 | <LLQ |
| | 0.4 mg/kg | Plasma | | | |
| Nicotine | Nicotine | Rat | 7B | 24 | <LLQ |
| | 0.4 mg/kg | Plasma | | | |
| Nicotine | Nicotine | Rat | 7C | 24 | <LLQ |
| | 0.4 mg/kg | Plasma | | | |
| Nicotine | Nicotine | Rat | 8A | 0.25 | 175 |
| | 0.4 mg/kg | Plasma | | | |
| Nicotine | Nicotine | Rat | 8B | 0.25 | 145 |
| | 0.4 mg/kg | Plasma | | | |
| Nicotine | Nicotine | Rat | 8C | 0.25 | 184 |
| | 0.4 mg/kg | Plasma | | | |
| Nicotine | Nicotine | Rat | 8A | 0.5 | 111 |
| | 0.4 mg/kg | Plasma | | | |
| Nicotine | Nicotine | Rat | 8B | 0.5 | 95 |
| | 0.4 mg/kg | Plasma | | | |
| Nicotine | Nicotine | Rat | 8C | 0.5 | 125 |
| | 0.4 mg/kg | Plasma | | | |
| Nicotine | Nicotine | Rat | 8D | 0.5 | 180 |
| | 0.4 mg/kg | Plasma | | | |
| Nicotine | Nicotine | Rat | 8E | 0.5 | 157 |
| | 0.4 mg/kg | Plasma | | | |
| Nicotine | Nicotine | Rat | 8F | 0.5 | 160 |
| | 0.4 mg/kg | Plasma | | | |
| Nicotine | Nicotine | Rat | 8A | 1 | 67 |
| | 0.4 mg/kg | Plasma | | | |
| Nicotine | Nicotine | Rat | 8B | 1 | 72 |
| | 0.4 mg/kg | Plasma | | | |
| Nicotine | Nicotine | Rat | 8C | 1 | 107 |
| | 0.4 mg/kg | Plasma | | | |
| Nicotine | Nicotine | Rat | 8A | 1.5 | 49 |
| | 0.4 mg/kg | Plasma | | | |
| Nicotine | Nicotine | Rat | 8B | 1.5 | 37 |
| | 0.4 mg/kg | Plasma | | | |
| Nicotine | Nicotine | Rat | 8C | 1.5 | 64 |
| | 0.4 mg/kg | Plasma | | | |
| Nicotine | Nicotine | Rat | 8A | 2 | 25 |
| | 0.4 mg/kg | Plasma | | | |
| Nicotine | Nicotine | Rat | 8B | 2 | 24 |
| | 0.4 mg/kg | Plasma | | | |
| Nicotine | Nicotine | Rat | 8C | 2 | 46 |
| | 0.4 mg/kg | Plasma | | | |
| Nicotine | Nicotine | Rat | 8A | 4 | 3 |
| | 0.4 mg/kg | Plasma | | | |
| Nicotine | Nicotine | Rat | 8B | 4 | <LLQ |
| | 0.4 mg/kg | Plasma | | | |
| Nicotine | Nicotine | Rat | 8C | 4 | 4 |
| | 0.4 mg/kg | Plasma | | | |
| Nicotine | Nicotine | Rat | 8A | 6 | <LLQ |
| | 0.4 mg/kg | Plasma | | | |
| Nicotine | Nicotine | Rat | 8B | 6 | <LLQ |
| | 0.4 mg/kg | Plasma | | | |
| Nicotine | Nicotine | Rat | 8C | 6 | <LLQ |
| | 0.4 mg/kg | Plasma | | | |
| Nicotine | Nicotine | Rat | 8G | 6 | <LLQ |
| | 0.4 mg/kg | Plasma | | | |
| Nicotine | Nicotine | Rat | 8H | 6 | <LLQ |
| | 0.4 mg/kg | Plasma | | | |
| Nicotine | Nicotine | Rat | 8I | 6 | <LLQ |
| | 0.4 mg/kg | Plasma | | | |
| Nicotine | Nicotine | Rat | 8A | 8 | <LLQ |
| | 0.4 mg/kg | Plasma | | | |
| Nicotine | Nicotine | Rat | 8B | 8 | <LLQ |
| | 0.4 mg/kg | Plasma | | | |
| Nicotine | Nicotine | Rat | 8C | 8 | <LLQ |
| | 0.4 mg/kg | Plasma | | | |
| Nicotine | Nicotine | Rat | 8A | 24 | <LLQ |
| | 0.4 mg/kg | Plasma | | | |
| Nicotine | Nicotine | Rat | 8B | 24 | <LLQ |
| | 0.4 mg/kg | Plasma | | | |
| Nicotine | Nicotine | Rat | 8C | 24 | <LLQ |
| | 0.4 mg/kg | Plasma | | | |
| Anatabine | Anatabine | Rat | 1A | 0.25 | 24 |
| | 0.1 mg/kg | Plasma | | | |
| Anatabine | Anatabine | Rat | 1B | 0.25 | 14 |
| | 0.1 mg/kg | Plasma | | | |
| Anatabine | Anatabine | Rat | 1C | 0.25 | 20 |
| | 0.1 mg/kg | Plasma | | | |
| Anatabine | Anatabine | Rat | 1A | 0.5 | 16 |
| | 0.1 mg/kg | Plasma | | | |
| Anatabine | Anatabine | Rat | 1B | 0.5 | 17 |
| | 0.1 mg/kg | Plasma | | | |
| Anatabine | Anatabine | Rat | 1C | 0.5 | 13 |
| | 0.1 mg/kg | Plasma | | | |
| Anatabine | Anatabine | Rat | 1D | 0.5 | 30 |
| | 0.1 mg/kg | Plasma | | | |
| Anatabine | Anatabine | Rat | 1E | 0.5 | 32 |
| | 0.1 mg/kg | Plasma | | | |
| Anatabine | Anatabine | Rat | 1F | 0.5 | 33 |
| | 0.1 mg/kg | Plasma | | | |
| Anatabine | Anatabine | Rat | 1A | 1 | 10 |
| | 0.1 mg/kg | Plasma | | | |
| Anatabine | Anatabine | Rat | 1B | 1 | 16 |
| | 0.1 mg/kg | Plasma | | | |
| Anatabine | Anatabine | Rat | 1C | 1 | 11 |
| | 0.1 mg/kg | Plasma | | | |
| Anatabine | Anatabine | Rat | 1A | 1.5 | 7 |
| | 0.1 mg/kg | Plasma | | | |
| Anatabine | Anatabine | Rat | 1B | 1.5 | <LLQ |
| | 0.1 mg/kg | Plasma | | | |
| Anatabine | Anatabine | Rat | 1C | 1.5 | 6 |
| | 0.1 mg/kg | Plasma | | | |
| Anatabine | Anatabine | Rat | 1A | 2 | 6 |
| | 0.1 mg/kg | Plasma | | | |
| Anatabine | Anatabine | Rat | 1B | 2 | 8 |
| | 0.1 mg/kg | Plasma | | | |
| Anatabine | Anatabine | Rat | 1C | 2 | 5 |
| | 0.1 mg/kg | Plasma | | | |
| Anatabine | Anatabine | Rat | 1A | 4 | 3 |
| | 0.1 mg/kg | Plasma | | | |
| Anatabine | Anatabine | Rat | 1B | 4 | 3 |
| | 0.1 mg/kg | Plasma | | | |
| Anatabine | Anatabine | Rat | 1C | 4 | <LLQ |
| | 0.1 mg/kg | Plasma | | | |
| Anatabine | Anatabine | Rat | 1A | 6 | <LLQ |
| | 0.1 mg/kg | Plasma | | | |
| Anatabine | Anatabine | Rat | 1B | 6 | <LLQ |
| | 0.1 mg/kg | Plasma | | | |
| Anatabine | Anatabine | Rat | 1C | 6 | <LLQ |
| | 0.1 mg/kg | Plasma | | | |
| Anatabine | Anatabine | Rat | 1G | 6 | 7 |
| | 0.1 mg/kg | Plasma | | | |
| Anatabine | Anatabine | Rat | 1H | 6 | 4 |
| | 0.1 mg/kg | Plasma | | | |
| Anatabine | Anatabine | Rat | 1I | 6 | 4 |
| | 0.1 mg/kg | Plasma | | | |
| Anatabine | Anatabine | Rat | 1A | 8 | <LLQ |
| | 0.1 mg/kg | Plasma | | | |
| Anatabine | Anatabine | Rat | 1B | 8 | <LLQ |
| | 0.1 mg/kg | Plasma | | | |
| Anatabine | Anatabine | Rat | 1C | 8 | <LLQ |
| | 0.1 mg/kg | Plasma | | | |
| Anatabine | Anatabine | Rat | 1A | 24 | <LLQ |
| | 0.1 mg/kg | Plasma | | | |
| Anatabine | Anatabine | Rat | 1B | 24 | <LLQ |
| | 0.1 mg/kg | Plasma | | | |
| Anatabine | Anatabine | Rat | 1C | 24 | <LLQ |
| | 0.1 mg/kg | Plasma | | | |
| Anatabine | Anatabine | Rat | 2D | 0.5 | 31 |
| | 0.1 mg/kg | Plasma | | | |
| Anatabine | Anatabine | Rat | 2E | 0.5 | 30 |
| | 0.1 mg/kg | Plasma | | | |
| Anatabine | Anatabine | Rat | 2F | 0.5 | 34 |
| | 0.1 mg/kg | Plasma | | | |
| Anatabine | Anatabine | Rat | 2G | 6 | 3 |
| | 0.1 mg/kg | Plasma | | | |
| Anatabine | Anatabine | Rat | 2H | 6 | 4 |
| | 0.1 mg/kg | Plasma | | | |
| Anatabine | Anatabine | Rat | 2I | 6 | 4 |
| | 0.1 mg/kg | Plasma | | | |
| Anatabine | Anatabine | Rat | 2A | 0.25 | 15 |
| | 0.1 mg/kg | Plasma | | | |
| Anatabine | Anatabine | Rat | 2B | 0.25 | 21 |
| | 0.1 mg/kg | Plasma | | | |
| Anatabine | Anatabine | Rat | 2C | 0.25 | 14 |
| | 0.1 mg/kg | Plasma | | | |
| Anatabine | Anatabine | Rat | 2A | 0.5 | 16 |
| | 0.1 mg/kg | Plasma | | | |
| Anatabine | Anatabine | Rat | 2B | 0.5 | 13 |
| | 0.1 mg/kg | Plasma | | | |
| Anatabine | Anatabine | Rat | 2C | 0.5 | 10 |
| | 0.1 mg/kg | Plasma | | | |
| Anatabine | Anatabine | Rat | 2A | 1 | 12 |
| | 0.1 mg/kg | Plasma | | | |
| Anatabine | Anatabine | Rat | 2B | 1 | 12 |
| | 0.1 mg/kg | Plasma | | | |
| Anatabine | Anatabine | Rat | 2C | 1 | 9 |
| | 0.1 mg/kg | Plasma | | | |
| Anatabine | Anatabine | Rat | 2A | 1.5 | 14 |
| | 0.1 mg/kg | Plasma | | | |
| Anatabine | Anatabine | Rat | 2B | 1.5 | 12 |
| | 0.1 mg/kg | Plasma | | | |
| Anatabine | Anatabine | Rat | 2C | 1.5 | 7 |
| | 0.1 mg/kg | Plasma | | | |
| Anatabine | Anatabine | Rat | 2A | 2 | 8 |
| | 0.1 mg/kg | Plasma | | | |
| Anatabine | Anatabine | Rat | 2B | 2 | 8 |
| | 0.1 mg/kg | Plasma | | | |
| Anatabine | Anatabine | Rat | 2C | 2 | 4 |
| | 0.1 mg/kg | Plasma | | | |
| Anatabine | Anatabine | Rat | 2A | 4 | 3 |
| | 0.1 mg/kg | Plasma | | | |
| Anatabine | Anatabine | Rat | 2B | 4 | 5 |
| | 0.1 mg/kg | Plasma | | | |
| Anatabine | Anatabine | Rat | 2C | 4 | <LLQ |
| | 0.1 mg/kg | Plasma | | | |
| Anatabine | Anatabine | Rat | 2A | 6 | <LLQ |
| | 0.1 mg/kg | Plasma | | | |
| Anatabine | Anatabine | Rat | 2B | 6 | 3 |
| | 0.1 mg/kg | Plasma | | | |
| Anatabine | Anatabine | Rat | 2C | 6 | <LLQ |
| | 0.1 mg/kg | Plasma | | | |
| Anatabine | Anatabine | Rat | 2A | 8 | <LLQ |
| | 0.1 mg/kg | Plasma | | | |
| Anatabine | Anatabine | Rat | 2B | 8 | <LLQ |
| | 0.1 mg/kg | Plasma | | | |
| Anatabine | Anatabine | Rat | 2C | 8 | <LLQ |
| | 0.1 mg/kg | Plasma | | | |
| Anatabine | Anatabine | Rat | 2A | 24 | <LLQ |
| | 0.1 mg/kg | Plasma | | | |
| Anatabine | Anatabine | Rat | 2B | 24 | <LLQ |
| | 0.1 mg/kg | Plasma | | | |
| Anatabine | Anatabine | Rat | 2C | 24 | <LLQ |
| | 0.1 mg/kg | Plasma | | | |
| Anatabine | Anatabine | Rat | 3A | 0.25 | 216 |
| | 0.75 mg/kg | Plasma | | | |
| Anatabine | Anatabine | Rat | 3B | 0.25 | 162 |
| | 0.75 mg/kg | Plasma | | | |
| Anatabine | Anatabine | Rat | 3C | 0.25 | 223 |
| | 0.75 mg/kg | Plasma | | | |
| Anatabine | Anatabine | Rat | 3A | 0.5 | 176 |
| | 0.75 mg/kg | Plasma | | | |
| Anatabine | Anatabine | Rat | 3B | 0.5 | 176 |
| | 0.75 mg/kg | Plasma | | | |
| Anatabine | Anatabine | Rat | 3C | 0.5 | 190 |
| | 0.75 mg/kg | Plasma | | | |
| Anatabine | Anatabine | Rat | 3D | 0.5 | 342 |
| | 0.75 mg/kg | Plasma | | | |
| Anatabine | Anatabine | Rat | 3E | 0.5 | 271 |
| | 0.75 mg/kg | Plasma | | | |
| Anatabine | Anatabine | Rat | 3F | 0.5 | 292 |
| | 0.75 mg/kg | Plasma | | | |
| Anatabine | Anatabine | Rat | 3A | 1 | 153 |
| | 0.75 mg/kg | Plasma | | | |
| Anatabine | Anatabine | Rat | 3B | 1 | 146 |
| | 0.75 mg/kg | Plasma | | | |
| Anatabine | Anatabine | Rat | 3C | 1 | 156 |
| | 0.75 mg/kg | Plasma | | | |
| Anatabine | Anatabine | Rat | 3A | 1.5 | 120 |
| | 0.75 mg/kg | Plasma | | | |
| Anatabine | Anatabine | Rat | 3B | 1.5 | 124 |
| | 0.75 mg/kg | Plasma | | | |
| Anatabine | Anatabine | Rat | 3C | 1.5 | 136 |
| | 0.75 mg/kg | Plasma | | | |
| Anatabine | Anatabine | Rat | 3A | 2 | 73 |
| | 0.75 mg/kg | Plasma | | | |
| Anatabine | Anatabine | Rat | 3B | 2 | 74 |
| | 0.75 mg/kg | Plasma | | | |
| Anatabine | Anatabine | Rat | 3C | 2 | 104 |
| | 0.75 mg/kg | Plasma | | | |
| Anatabine | Anatabine | Rat | 3A | 4 | 29 |
| | 0.75 mg/kg | Plasma | | | |
| Anatabine | Anatabine | Rat | 3B | 4 | 36 |
| | 0.75 mg/kg | Plasma | | | |
| Anatabine | Anatabine | Rat | 3C | 4 | 39 |
| | 0.75 mg/kg | Plasma | | | |
| Anatabine | Anatabine | Rat | 3A | 6 | 15 |
| | 0.75 mg/kg | Plasma | | | |
| Anatabine | Anatabine | Rat | 3B | 6 | 14 |
| | 0.75 mg/kg | Plasma | | | |
| Anatabine | Anatabine | Rat | 3C | 6 | 13 |
| | 0.75 mg/kg | Plasma | | | |
| Anatabine | Anatabine | Rat | 3G | 6 | 15 |
| | 0.75 mg/kg | Plasma | | | |
| Anatabine | Anatabine | Rat | 3H | 6 | 31 |
| | 0.75 mg/kg | Plasma | | | |
| Anatabine | Anatabine | Rat | 3I | 6 | 20 |
| | 0.75 mg/kg | Plasma | | | |
| Anatabine | Anatabine | Rat | 3A | 8 | 8 |
| | 0.75 mg/kg | Plasma | | | |
| Anatabine | Anatabine | Rat | 3B | 8 | 11 |
| | 0.75 mg/kg | Plasma | | | |
| Anatabine | Anatabine | Rat | 3C | 8 | 8 |
| | 0.75 mg/kg | Plasma | | | |
| Anatabine | Anatabine | Rat | 3A | 24 | <LLQ |
| | 0.75 mg/kg | Plasma | | | |
| Anatabine | Anatabine | Rat | 3B | 24 | <LLQ |
| | 0.75 mg/kg | Plasma | | | |
| Anatabine | Anatabine | Rat | 3C | 24 | <LLQ |
| | 0.75 mg/kg | Plasma | | | |
| Anatabine | Anatabine | Rat | 4A | 0.25 | 204 |
| | 0.75 mg/kg | Plasma | | | |
| Anatabine | Anatabine | Rat | 4B | 0.25 | 226 |
| | 0.75 mg/kg | Plasma | | | |
| Anatabine | Anatabine | Rat | 4C | 0.25 | 207 |
| | 0.75 mg/kg | Plasma | | | |
| Anatabine | Anatabine | Rat | 4A | 0.5 | 166 |
| | 0.75 mg/kg | Plasma | | | |
| Anatabine | Anatabine | Rat | 4B | 0.5 | 229 |
| | 0.75 mg/kg | Plasma | | | |
| Anatabine | Anatabine | Rat | 4C | 0.5 | 175 |
| | 0.75 mg/kg | Plasma | | | |
| Anatabine | Anatabine | Rat | 4D | 0.5 | 307 |
| | 0.75 mg/kg | Plasma | | | |
| Anatabine | Anatabine | Rat | 4E | 0.5 | 359 |
| | 0.75 mg/kg | Plasma | | | |
| Anatabine | Anatabine | Rat | 4F | 0.5 | 396 |
| | 0.75 mg/kg | Plasma | | | |
| Anatabine | Anatabine | Rat | 4A | 1 | 146 |
| | 0.75 mg/kg | Plasma | | | |
| Anatabine | Anatabine | Rat | 4B | 1 | 207 |
| | 0.75 mg/kg | Plasma | | | |
| Anatabine | Anatabine | Rat | 4C | 1 | 165 |
| | 0.75 mg/kg | Plasma | | | |
| Anatabine | Anatabine | Rat | 4A | 1.5 | 136 |
| | 0.75 mg/kg | Plasma | | | |
| Anatabine | Anatabine | Rat | 4B | 1.5 | 134 |
| | 0.75 mg/kg | Plasma | | | |
| Anatabine | Anatabine | Rat | 4C | 1.5 | 150 |
| | 0.75 mg/kg | Plasma | | | |
| Anatabine | Anatabine | Rat | 4A | 2 | 89 |
| | 0.75 mg/kg | Plasma | | | |
| Anatabine | Anatabine | Rat | 4B | 2 | 113 |
| | 0.75 mg/kg | Plasma | | | |
| Anatabine | Anatabine | Rat | 4C | 2 | 97 |
| | 0.75 mg/kg | Plasma | | | |
| Anatabine | Anatabine | Rat | 4A | 4 | 45 |
| | 0.75 mg/kg | Plasma | | | |
| Anatabine | Anatabine | Rat | 4B | 4 | 50 |
| | 0.75 mg/kg | Plasma | | | |
| Anatabine | Anatabine | Rat | 4C | 4 | 41 |
| | 0.75 mg/kg | Plasma | | | |
| Anatabine | Anatabine | Rat | 4A | 6 | 14 |
| | 0.75 mg/kg | Plasma | | | |
| Anatabine | Anatabine | Rat | 4B | 6 | 23 |
| | 0.75 mg/kg | Plasma | | | |
| Anatabine | Anatabine | Rat | 4C | 6 | 18 |
| | 0.75 mg/kg | Plasma | | | |
| Anatabine | Anatabine | Rat | 4G | 6 | 15 |
| | 0.75 mg/kg | Plasma | | | |
| Anatabine | Anatabine | Rat | 4H | 6 | 38 |
| | 0.75 mg/kg | Plasma | | | |
| Anatabine | Anatabine | Rat | 4I | 6 | 16 |
| | 0.75 mg/kg | Plasma | | | |
| Anatabine | Anatabine | Rat | 4A | 8 | 10 |
| | 0.75 mg/kg | Plasma | | | |
| Anatabine | Anatabine | Rat | 4B | 8 | 12 |
| | 0.75 mg/kg | Plasma | | | |
| Anatabine | Anatabine | Rat | 4C | 8 | 11 |
| | 0.75 mg/kg | Plasma | | | |
| Anatabine | Anatabine | Rat | 4A | 24 | <LLQ |
| | 0.75 mg/kg | Plasma | | | |
| Anatabine | Anatabine | Rat | 4B | 24 | <LLQ |
| | 0.75 mg/kg | Plasma | | | |
| Anatabine | Anatabine | Rat | 4C | 24 | <LLQ |
| | 0.75 mg/kg | Plasma | | | |
| Anatabine | Anatabine | Rat | 5A | 0.25 | 296 |
| | 1.0 mg/kg | Plasma | | | |
| Anatabine | Anatabine | Rat | 5B | 0.25 | 291 |
| | 1.0 mg/kg | Plasma | | | |
| Anatabine | Anatabine | Rat | 5C | 0.25 | 270 |
| | 1.0 mg/kg | Plasma | | | |
| Anatabine | Anatabine | Rat | 5A | 0.5 | 288 |
| | 1.0 mg/kg | Plasma | | | |
| Anatabine | Anatabine | Rat | 5B | 0.5 | 264 |
| | 1.0 mg/kg | Plasma | | | |
| Anatabine | Anatabine | Rat | 5C | 0.5 | 265 |
| | 1.0 mg/kg | Plasma | | | |
| Anatabine | Anatabine | Rat | 5D | 0.5 | 447 |
| | 1.0 mg/kg | Plasma | | | |
| Anatabine | Anatabine | Rat | 5E | 0.5 | 384 |
| | 1.0 mg/kg | Plasma | | | |
| Anatabine | Anatabine | Rat | 5F | 0.5 | 366 |
| | 1.0 mg/kg | Plasma | | | |
| Anatabine | Anatabine | Rat | 5A | 1 | 257 |
| | 1.0 mg/kg | Plasma | | | |
| Anatabine | Anatabine | Rat | 5B | 1 | 225 |
| | 1.0 mg/kg | Plasma | | | |
| Anatabine | Anatabine | Rat | 5C | 1 | 219 |
| | 1.0 mg/kg | Plasma | | | |
| Anatabine | Anatabine | Rat | 5A | 1.5 | 163 |
| | 1.0 mg/kg | Plasma | | | |
| Anatabine | Anatabine | Rat | 5B | 1.5 | 148 |
| | 1.0 mg/kg | Plasma | | | |
| Anatabine | Anatabine | Rat | 5C | 1.5 | 160 |
| | 1.0 mg/kg | Plasma | | | |
| Anatabine | Anatabine | Rat | 5A | 2 | 121 |
| | 1.0 mg/kg | Plasma | | | |
| Anatabine | Anatabine | Rat | 5B | 2 | 129 |
| | 1.0 mg/kg | Plasma | | | |
| Anatabine | Anatabine | Rat | 5C | 2 | 119 |
| | 1.0 mg/kg | Plasma | | | |
| Anatabine | Anatabine | Rat | 5A | 4 | 36 |
| | 1.0 mg/kg | Plasma | | | |
| Anatabine | Anatabine | Rat | 5B | 4 | 51 |
| | 1.0 mg/kg | Plasma | | | |
| Anatabine | Anatabine | Rat | 5C | 4 | 40 |
| | 1.0 mg/kg | Plasma | | | |
| Anatabine | Anatabine | Rat | 5A | 6 | 20 |
| | 1.0 mg/kg | Plasma | | | |
| Anatabine | Anatabine | Rat | 5B | 6 | 19 |
| | 1.0 mg/kg | Plasma | | | |
| Anatabine | Anatabine | Rat | 5C | 6 | 15 |
| | 1.0 mg/kg | Plasma | | | |
| Anatabine | Anatabine | Rat | 5G | 6 | 26 |
| | 1.0 mg/kg | Plasma | | | |
| Anatabine | Anatabine | Rat | 5H | 6 | 38 |
| | 1.0 mg/kg | Plasma | | | |
| Anatabine | Anatabine | Rat | 5I | 6 | 17 |
| | 1.0 mg/kg | Plasma | | | |
| Anatabine | Anatabine | Rat | 5A | 8 | 8 |
| | 1.0 mg/kg | Plasma | | | |
| Anatabine | Anatabine | Rat | 5B | 8 | 9 |
| | 1.0 mg/kg | Plasma | | | |
| Anatabine | Anatabine | Rat | 5C | 8 | 6 |
| | 1.0 mg/kg | Plasma | | | |
| Anatabine | Anatabine | Rat | 5A | 24 | <LLQ |
| | 1.0 mg/kg | Plasma | | | |
| Anatabine | Anatabine | Rat | 5B | 24 | <LLQ |
| | 1.0 mg/kg | Plasma | | | |
| Anatabine | Anatabine | Rat | 5C | 24 | <LLQ |
| | 1.0 mg/kg | Plasma | | | |
| Anatabine | Anatabine | Rat | 6A | 0.25 | 293 |
| | 1.0 mg/kg | Plasma | | | |
| Anatabine | Anatabine | Rat | 6B | 0.25 | 271 |
| | 1.0 mg/kg | Plasma | | | |
| Anatabine | Anatabine | Rat | 6C | 0.25 | 302 |
| | 1.0 mg/kg | Plasma | | | |
| Anatabine | Anatabine | Rat | 6A | 0.5 | 222 |
| | 1.0 mg/kg | Plasma | | | |
| Anatabine | Anatabine | Rat | 6B | 0.5 | 253 |
| | 1.0 mg/kg | Plasma | | | |
| Anatabine | Anatabine | Rat | 6C | 0.5 | 236 |
| | 1.0 mg/kg | Plasma | | | |
| Anatabine | Anatabine | Rat | 6D | 0.5 | 347 |
| | 1.0 mg/kg | Plasma | | | |
| Anatabine | Anatabine | Rat | 6E | 0.5 | 362 |
| | 1.0 mg/kg | Plasma | | | |
| Anatabine | Anatabine | Rat | 6F | 0.5 | 395 |
| | 1.0 mg/kg | Plasma | | | |
| Anatabine | Anatabine | Rat | 6A | 1 | 218 |
| | 1.0 mg/kg | Plasma | | | |
| Anatabine | Anatabine | Rat | 6B | 1 | 225 |
| | 1.0 mg/kg | Plasma | | | |
| Anatabine | Anatabine | Rat | 6C | 1 | 244 |
| | 1.0 mg/kg | Plasma | | | |
| Anatabine | Anatabine | Rat | 6A | 1.5 | 196 |
| | 1.0 mg/kg | Plasma | | | |
| Anatabine | Anatabine | Rat | 6B | 1.5 | 192 |
| | 1.0 mg/kg | Plasma | | | |
| Anatabine | Anatabine | Rat | 6C | 1.5 | 211 |
| | 1.0 mg/kg | Plasma | | | |
| Anatabine | Anatabine | Rat | 6A | 2 | 147 |
| | 1.0 mg/kg | Plasma | | | |
| Anatabine | Anatabine | Rat | 6B | 2 | 170 |
| | 1.0 mg/kg | Plasma | | | |
| Anatabine | Anatabine | Rat | 6C | 2 | 174 |
| | 1.0 mg/kg | Plasma | | | |
| Anatabine | Anatabine | Rat | 6A | 4 | 73 |
| | 1.0 mg/kg | Plasma | | | |
| Anatabine | Anatabine | Rat | 6B | 4 | 52 |
| | 1.0 mg/kg | Plasma | | | |
| Anatabine | Anatabine | Rat | 6C | 4 | 57 |
| | 1.0 mg/kg | Plasma | | | |
| Anatabine | Anatabine | Rat | 6A | 6 | 33 |
| | 1.0 mg/kg | Plasma | | | |
| Anatabine | Anatabine | Rat | 6B | 6 | 34 |
| | 1.0 mg/kg | Plasma | | | |
| Anatabine | Anatabine | Rat | 6C | 6 | 21 |
| | 1.0 mg/kg | Plasma | | | |
| Anatabine | Anatabine | Rat | 6G | 6 | 27 |
| | 1.0 mg/kg | Plasma | | | |
| Anatabine | Anatabine | Rat | 6H | 6 | 24 |
| | 1.0 mg/kg | Plasma | | | |
| Anatabine | Anatabine | Rat | 6I | 6 | 18 |
| | 1.0 mg/kg | Plasma | | | |
| Anatabine | Anatabine | Rat | 6A | 8 | 19 |
| | 1.0 mg/kg | Plasma | | | |
| Anatabine | Anatabine | Rat | 6B | 8 | 19 |
| | 1.0 mg/kg | Plasma | | | |
| Anatabine | Anatabine | Rat | 6C | 8 | 15 |
| | 1.0 mg/kg | Plasma | | | |
| Anatabine | Anatabine | Rat | 6A | 24 | <LLQ |
| | 1.0 mg/kg | Plasma | | | |
| Anatabine | Anatabine | Rat | 6B | 24 | <LLQ |
| | 1.0 mg/kg | Plasma | | | |
| Anatabine | Anatabine | Rat | 6C | 24 | <LLQ |
| | 1.0 mg/kg | Plasma | | | |

TABLE 15
Mean Concentrations and Descriptive Statistics of Anatabine and Nicotine
in Rat Brain Extracts and Plasma Samples at Each Time Point

| | | | | | | | | | Combined Male and Female | | | |
|---|---|---|---|---|---|---|---|---|---|---|---|---|
| | | | | | | | | | Male and | | | |
| | | | | | Male or Female | | | | Female | | | |
| | | | | | | Avg. Conc. | | | Avg. Conc. | | | |
| | | | | | | (ng/ml for | | | (ng/ml for | | | |
| | | | | Time | | plasma and | | | plasma and | | | |
| | | | | Point | | ng/g for | | | ng/g for | | | |
| Compound | Dose Group | Tissue | Sex | (hr) | N = | brain) | ±STDEV | ±SEM | brain) | n = | stdev | sem |
| Nicotine | Nicotine | Rat Brain | male | 0.5 | 3 | 94.3 | 20.6 | 11.9 | 107.2 | 6 | 19.3 | 7.9 |
| | 0.4 mg/kg | | | | | | | | | | | |
| Nicotine | Nicotine | Rat Brain | male | 6 | 2 | 6.0 | 1.4 | 1.0 | 5.7 | 3 | 1.2 | 0.7 |
| | 0.4 mg/kg | | | | | | | | | | | |
| Nicotine | Nicotine | Rat Brain | male | 24 | 3 | 6.3 | 2.3 | 1.3 | 6.5 | 4 | 1.9 | 1.0 |
| | 0.4 mg/kg | | | | | | | | | | | |
| Nicotine | Nicotine | Rat Brain | female | 0.5 | 3 | 120.0 | 3.0 | 1.7 | | | | |
| | 0.4 mg/kg | | | | | | | | | | | |
| Nicotine | Nicotine | Rat Brain | female | 6 | 1 | 5.0 | ND | ND | | | | |
| | 0.4 mg/kg | | | | | | | | | | | |
| Nicotine | Nicotine | Rat Brain | female | 24 | 1 | 7.0 | ND | ND | | | | |
| | 0.4 mg/kg | | | | | | | | | | | |
| Anatabine | Anatabine | Rat Brain | male | 0.5 | 2 | 20.0 | 1.4 | 1.0 | 25.8 | 5 | 6.2 | 2.8 |
| | 0.1 mg/kg | | | | | | | | | | | |
| Anatabine | Anatabine | Rat Brain | male | 6 | 1 | 3.0 | ND | ND | 3.5 | 4 | 1.3 | 0.6 |
| | 0.1 mg/kg | | | | | | | | | | | |
| Anatabine | Anatabine | Rat Brain | male | 24 | 1 | 3.0 | ND | ND | 2.5 | 2 | 0.7 | 0.5 |
| | 0.1 mg/kg | | | | | | | | | | | |
| Anatabine | Anatabine | Rat Brain | female | 0.5 | 3 | 29.7 | 4.5 | 2.6 | | | | |
| | 0.1 mg/kg | | | | | | | | | | | |
| Anatabine | Anatabine | Rat Brain | female | 6 | 3 | 3.7 | 1.5 | 0.9 | | | | |
| | 0.1 mg/kg | | | | | | | | | | | |
| Anatabine | Anatabine | Rat Brain | female | 24 | 1 | 2.0 | ND | ND | | | | |
| | 0.1 mg/kg | | | | | | | | | | | |
| Anatabine | Anatabine | Rat Brain | male | 0.5 | 3 | 276.0 | 37.0 | 21.4 | 295.3 | 6 | 53.3 | 21.8 |
| | 0.75 mg/kg | | | | | | | | | | | |
| Anatabine | Anatabine | Rat Brain | male | 6 | 3 | 21.3 | 5.1 | 3.0 | 21.2 | 6 | 5.1 | 2.1 |
| | 0.75 mg/kg | | | | | | | | | | | |
| Anatabine | Anatabine | Rat Brain | male | 24 | 0 | <LLQ | ND | ND | <LLQ | 0 | ND | ND |
| | 0.75 mg/kg | | | | | | | | | | | |
| Anatabine | Anatabine | Rat Brain | female | 0.5 | 3 | 314.7 | 67.9 | 39.2 | | | | |
| | 0.75 mg/kg | | | | | | | | | | | |
| Anatabine | Anatabine | Rat Brain | female | 6 | 3 | 21.0 | 6.2 | 3.6 | | | | |
| | 0.75 mg/kg | | | | | | | | | | | |
| Anatabine | Anatabine | Rat Brain | female | 24 | 0 | <LLQ | ND | ND | | | | |
| | 0.75 mg/kg | | | | | | | | | | | |
| Anatabine | Anatabine | Rat Brain | male | 0.5 | 3 | 323.0 | 35.9 | 20.7 | 334.5 | 6 | 44.3 | 18.1 |
| | 1.0 mg/kg | | | | | | | | | | | |
| Anatabine | Anatabine | Rat Brain | male | 6 | 3 | 5.0 | 2.6 | 1.5 | 5.4 | 5 | 2.4 | 1.1 |
| | 1.0 mg/kg | | | | | | | | | | | |
| Anatabine | Anatabine | Rat Brain | male | 24 | 2 | 2.5 | 0.7 | 0.5 | 2.5 | 2 | 0.7 | 0.5 |
| | 1.0 mg/kg | | | | | | | | | | | |
| Anatabine | Anatabine | Rat Brain | female | 0.5 | 3 | 346.0 | 56.7 | 32.7 | | | | |
| | 1.0 mg/kg | | | | | | | | | | | |
| Anatabine | Anatabine | Rat Brain | female | 6 | 2 | 6.0 | 2.8 | 2.0 | | | | |
| | 1.0 mg/kg | | | | | | | | | | | |
| Anatabine | Anatabine | Rat Brain | female | 24 | 0 | <LLQ | ND | ND | | | | |
| | 1.0 mg/kg | | | | | | | | | | | |
| Nicotine | Nicotine | Rat | male | 0.25 | 3 | 165.0 | 25.7 | 14.8 | 166.5 | 6 | 20.8 | 8.5 |
| | 0.4 mg/kg | Plasma | | | | | | | | | | |
| Nicotine | Nicotine | Rat | male | 0.5 | 3 | 99.3 | 20.6 | 11.9 | 104.8 | 6 | 17.2 | 7.0 |
| | 0.4 mg/kg | Plasma | | | | | | | | | | |
| Nicotine | Nicotine | Rat | male | 0.5 | 3 | 154.0 | 34.6 | 20.0 | 159.8 | 6 | 24.1 | 9.9 |
| | 0.4 mg/kg | Plasma | | | | | | | | | | |
| Nicotine | Nicotine | Rat | male | 1 | 3 | 69.0 | 2.6 | 1.5 | 75.5 | 6 | 15.6 | 6.4 |
| | 0.4 mg/kg | Plasma | | | | | | | | | | |
| Nicotine | Nicotine | Rat | male | 1.5 | 3 | 35.7 | 7.4 | 4.3 | 42.8 | 6 | 12.5 | 5.1 |
| | 0.4 mg/kg | Plasma | | | | | | | | | | |
| Nicotine | Nicotine | Rat | male | 2 | 3 | 24.7 | 6.4 | 3.7 | 28.2 | 6 | 9.6 | 3.9 |
| | 0.4 mg/kg | Plasma | | | | | | | | | | |
| Nicotine | Nicotine | Rat | male | 4 | 0 | <LLQ | ND | ND | 3.5 | 2 | 0.7 | 0.5 |
| | 0.4 mg/kg | Plasma | | | | | | | | | | |
| Nicotine | Nicotine | Rat | male | 6 | 0 | <LLQ | ND | ND | <LLQ | 0 | ND | ND |
| | 0.4 mg/kg | Plasma | | | | | | | | | | |
| Nicotine | Nicotine | Rat | male | 8 | 0 | <LLQ | ND | ND | <LLQ | 0 | ND | ND! |
| | 0.4 mg/kg | Plasma | | | | | | | | | | |
| Nicotine | Nicotine | Rat | male | 24 | 0 | <LLQ | ND | ND | <LLQ | 0 | ND | ND |
| | 0.4 mg/kg | Plasma | | | | | | | | | | |
| Nicotine | Nicotine | Rat | female | 0.25 | 3 | 168.0 | 20.4 | 11.8 | 108.4 | 5 | 83.0 | 37.1 |
| | 0.4 mg/kg | Plasma | | | | | | | | | | |
| Nicotine | Nicotine | Rat | female | 0.5 | 3 | 110.3 | 15.0 | 8.7 | 79.7 | 12 | 65.0 | 18.8 |
| | 0.4 mg/kg | Plasma | | | | | | | | | | |
| Nicotine | Nicotine | Rat | female | 0.5 | 3 | 165.7 | 12.5 | 7.2 | 95.3 | 6 | 77.7 | 31.7 |
| | 0.4 mg/kg | Plasma | | | | | | | | | | |
| Nicotine | Nicotine | Rat | female | 1 | 3 | 82.0 | 21.8 | 12.6 | 50.8 | 6 | 37.6 | 15.3 |
| | 0.4 mg/kg | Plasma | | | | | | | | | | |
| Nicotine | Nicotine | Rat | female | 1.5 | 3 | 50.0 | 13.5 | 7.8 | 33.6 | 5 | 24.4 | 10.9 |
| | 0.4 mg/kg | Plasma | | | | | | | | | | |
| Nicotine | Nicotine | Rat | female | 2 | 3 | 31.7 | 12.4 | 7.2 | 19.2 | 6 | 15.8 | 6.5 |
| | 0.4 mg/kg | Plasma | | | | | | | | | | |
| Nicotine | Nicotine | Rat | female | 4 | 2 | 3.5 | 0.7 | 0.5 | 3.6 | 5 | 0.9 | 0.4 |
| | 0.4 mg/kg | Plasma | | | | | | | | | | |
| Nicotine | Nicotine | Rat | female | 6 | 0 | <LLQ | ND | ND | 5.5 | 2 | 2.1 | 1.5 |
| | 0.4 mg/kg | Plasma | | | | | | | | | | |
| Nicotine | Nicotine | Rat | female | 6 | 0 | <LLQ | ND | ND | 5.5 | 2 | 2.1 | 1.5 |
| | 0.4 mg/kg | Plasma | | | | | | | | | | |
| Nicotine | Nicotine | Rat | female | 8 | 0 | <LLQ | ND | ND | 4.0 | 1 | ND | ND |
| | 0.4 mg/kg | Plasma | | | | | | | | | | |
| Nicotine | Nicotine | Rat | female | 24 | 0 | <LLQ | ND | ND | <LLQ | 0 | ND | ND |
| | 0.4 mg/kg | Plasma | | | | | | | | | | |
| Anatabine | Anatabine | Rat | male | 0.25 | 3 | 19.3 | 5.0 | 2.9 | 18.0 | 6 | 4.2 | 1.7 |
| | 0.1 mg/kg | Plasma | | | | | | | | | | |
| Anatabine | Anatabine | Rat | male | 0.5 | 3 | 15.3 | 2.1 | 1.2 | 22.9 | 12 | 9.4 | 2.7 |
| | 0.1 mg/kg | Plasma | | | | | | | | | | |
| Anatabine | Anatabine | Rat | male | 0.5 | 3 | 31.7 | 1.5 | 0.9 | 31.7 | 6 | 1.6 | 0.7 |
| | 0.1 mg/kg | Plasma | | | | | | | | | | |
| Anatabine | Anatabine | Rat | male | 1 | 3 | 12.3 | 3.2 | 1.9 | 8.0 | 6 | 5.2 | 2.1 |
| | 0.1 mg/kg | Plasma | | | | | | | | | | |
| Anatabine | Anatabine | Rat | male | 1.5 | 2 | 6.5 | 0.7 | 0.5 | 9.2 | 5 | 2.8 | 1.2 |
| | 0.1 mg/kg | Plasma | | | | | | | | | | |
| Anatabine | Anatabine | Rat | male | 2 | 3 | 6.3 | 1.5 | 0.9 | 8.7 | 6 | 3.6 | 1.5 |
| | 0.1 mg/kg | Plasma | | | | | | | | | | |
| Anatabine | Anatabine | Rat | male | 4 | 2 | 3.0 | 0.0 | 0.0 | 5.2 | 5 | 2.6 | 1.2 |
| | 0.1 mg/kg | Plasma | | | | | | | | | | |
| Anatabine | Anatabine | Rat | male | 6 | 0 | <LLQ | ND | ND | 4.3 | 6 | 1.5 | 0.6 |
| | 0.1 mg/kg | Plasma | | | | | | | | | | |
| Anatabine | Anatabine | Rat | male | 6 | 3 | 5.0 | 1.7 | 1.0 | 4.5 | 4 | 1.7 | 0.9 |
| | 0.1 mg/kg | Plasma | | | | | | | | | | |
| Anatabine | Anatabine | Rat | male | 8 | 0 | <LLQ | ND | ND | <LLQ | 0 | ND | ND |
| | 0.1 mg/kg | Plasma | | | | | | | | | | |
| Anatabine | Anatabine | Rat | male | 24 | 0 | <LLQ | ND | ND | <LLQ | 0 | ND | ND |
| | 0.1 mg/kg | Plasma | | | | | | | | | | |
| Anatabine | Anatabine | Rat | female | 0.25 | 3 | 16.7 | 3.8 | 2.2 | | | | |
| | 0.1 mg/kg | Plasma | | | | | | | | | | |
| Anatabine | Anatabine | Rat | female | 0.5 | 3 | 13.0 | 3.0 | 1.7 | | | | |
| | 0.1 mg/kg | Plasma | | | | | | | | | | |
| Anatabine | Anatabine | Rat | female | 0.5 | 3 | 31.7 | 2.1 | 1.2 | | | | |
| | 0.1 mg/kg | Plasma | | | | | | | | | | |
| Anatabine | Anatabine | Rat | female | 6 | 3 | 3.7 | 0.6 | 0.3 | | | | |
| | 0.1 mg/kg | Plasma | | | | | | | | | | |
| Anatabine | Anatabine | Rat | female | 1 | 3 | 11.0 | 1.7 | 1.0 | | | | |
| | 0.1 mg/kg | Plasma | | | | | | | | | | |
| Anatabine | Anatabine | Rat | female | 1.5 | 3 | 11.0 | 3.6 | 2.1 | | | | |
| | 0.1 mg/kg | Plasma | | | | | | | | | | |
| Anatabine | Anatabine | Rat | female | 2 | 3 | 6.7 | 2.3 | 1.3 | | | | |
| | 0.1 mg/kg | Plasma | | | | | | | | | | |
| Anatabine | Anatabine | Rat | female | 4 | 2 | 4.0 | 1.4 | 1.0 | | | | |
| | 0.1 mg/kg | Plasma | | | | | | | | | | |
| Anatabine | Anatabine | Rat | female | 6 | 1 | 3.0 | ND | ND | | | | |
| | 0.1 mg/kg | Plasma | | | | | | | | | | |
| Anatabine | Anatabine | Rat | female | 8 | 0 | <LLQ | ND | ND | | | | |
| | 0.1 mg/kg | Plasma | | | | | | | | | | |
| Anatabine | Anatabine | Rat | female | 24 | 0 | <LLQ | ND | ND | | | | |
| | 0.1 mg/kg | Plasma | | | | | | | | | | |
| Anatabine | Anatabine | Rat | male | 0.25 | 3 | 200.3 | 33.4 | 19.3 | 206.3 | 6 | 23.4 | 9.5 |
| | 0.75 mg/kg | Plasma | | | | | | | | | | |
| Anatabine | Anatabine | Rat | male | 0.5 | 3 | 180.7 | 8.1 | 4.7 | 256.6 | 12 | 82.2 | 23.7 |
| | 0.75 mg/kg | Plasma | | | | | | | | | | |
| Anatabine | Anatabine | Rat | male | 0.5 | 3 | 301.7 | 36.5 | 21.1 | 327.8 | 6 | 46.4 | 18.9 |
| | 0.75 mg/kg | Plasma | | | | | | | | | | |
| Anatabine | Anatabine | Rat | male | 1 | 3 | 151.7 | 5.1 | 3.0 | 162.2 | 6 | 23.1 | 9.4 |
| | 0.75 mg/kg | Plasma | | | | | | | | | | |
| Anatabine | Anatabine | Rat | male | 1.5 | 3 | 126.7 | 8.3 | 4.8 | 133.3 | 6 | 10.6 | 4.3 |
| | 0.75 mg/kg | Plasma | | | | | | | | | | |
| Anatabine | Anatabine | Rat | male | 2 | 3 | 83.7 | 17.6 | 10.2 | 91.7 | 6 | 16.1 | 6.6 |
| | 0.75 mg/kg | Plasma | | | | | | | | | | |
| Anatabine | Anatabine | Rat | male | 4 | 3 | 34.7 | 5.1 | 3.0 | 40.0 | 6 | 7.3 | 3.0 |
| | 0.75 mg/kg | Plasma | | | | | | | | | | |
| Anatabine | Anatabine | Rat | male | 6 | 3 | 14.0 | 1.0 | 0.6 | 19.3 | 12 | 7.8 | 2.2 |
| | 0.75 mg/kg | Plasma | | | | | | | | | | |
| Anatabine | Anatabine | Rat | male | 6 | 3 | 22.0 | 8.2 | 4.7 | 22.5 | 6 | 9.7 | 4.0 |
| | 0.75 mg/kg | Plasma | | | | | | | | | | |
| Anatabine | Anatabine | Rat | male | 8 | 3 | 9.0 | 1.7 | 1.0 | 10.0 | 6 | 1.7 | 0.7 |
| | 0.75 mg/kg | Plasma | | | | | | | | | | |
| Anatabine | Anatabine | Rat | male | 24 | 0 | <LLQ | ND | ND | <LLQ | 0 | ND | ND |
| | 0.75 mg/kg | Plasma | | | | | | | | | | |
| Anatabine | Anatabine | Rat | female | 0.25 | 3 | 212.3 | 11.9 | 6.9 | | | | |
| | 0.75 mg/kg | Plasma | | | | | | | | | | |
| Anatabine | Anatabine | Rat | female | 0.5 | 3 | 190.0 | 34.1 | 19.7 | | | | |
| | 0.75 mg/kg | Plasma | | | | | | | | | | |
| Anatabine | Anatabine | Rat | female | 0.5 | 3 | 354.0 | 44.7 | 25.8 | | | | |
| | 0.75 mg/kg | Plasma | | | | | | | | | | |
| Anatabine | Anatabine | Rat | female | 1 | 3 | 172.7 | 31.2 | 18.0 | | | | |
| | 0.75 mg/kg | Plasma | | | | | | | | | | |
| Anatabine | Anatabine | Rat | female | 1.5 | 3 | 140.0 | 8.7 | 5.0 | | | | |
| | 0.75 mg/kg | Plasma | | | | | | | | | | |
| Anatabine | Anatabine | Rat | female | 2 | 3 | 99.7 | 12.2 | 7.1 | | | | |
| | 0.75 mg/kg | Plasma | | | | | | | | | | |
| Anatabine | Anatabine | Rat | female | 4 | 3 | 45.3 | 4.5 | 2.6 | | | | |
| | 0.75 mg/kg | Plasma | | | | | | | | | | |
| Anatabine | Anatabine | Rat | female | 6 | 3 | 18.3 | 4.5 | 2.6 | | | | |
| | 0.75 mg/kg | Plasma | | | | | | | | | | |
| Anatabine | Anatabine | Rat | female | 6 | 3 | 23.0 | 13.0 | 7.5 | | | | |
| | 0.75 mg/kg | Plasma | | | | | | | | | | |
| Anatabine | Anatabine | Rat | female | 8 | 3 | 11.0 | 1.0 | 0.6 | | | | |
| | 0.75 mg/kg | Plasma | | | | | | | | | | |
| Anatabine | Anatabine | Rat | female | 24 | 0 | <LLQ | ND | ND | | | | |
| | 0.75 mg/kg | Plasma | | | | | | | | | | |
| Anatabine | Anatabine | Rat | male | 0.25 | 3 | 285.7 | 13.8 | 8.0 | 287.2 | 6 | 13.4 | 5.5 |
| | 1.0 mg/kg | Plasma | | | | | | | | | | |
| Anatabine | Anatabine | Rat | male | 0.5 | 3 | 272.3 | 13.6 | 7.8 | 319.1 | 12 | 73.1 | 21.1 |
| | 1.0 mg/kg | Plasma | | | | | | | | | | |
| Anatabine | Anatabine | Rat | male | 0.5 | 3 | 399.0 | 42.5 | 24.6 | 383.5 | 6 | 35.4 | 14.5 |
| | 1.0 mg/kg | Plasma | | | | | | | | | | |
| Anatabine | Anatabine | Rat | male | 1 | 3 | 233.7 | 20.4 | 11.8 | 231.3 | 6 | 15.7 | 6.4 |
| | 1.0 mg/kg | Plasma | | | | | | | | | | |
| Anatabine | Anatabine | Rat | male | 1.5 | 3 | 157.0 | 7.9 | 4.6 | 178.3 | 6 | 24.7 | 10.1 |
| | 1.0 mg/kg | Plasma | | | | | | | | | | |
| Anatabine | Anatabine | Rat | male | 2 | 3 | 123.0 | 5.3 | 3.1 | 143.3 | 6 | 24.3 | 9.9 |
| | 1.0 mg/kg | Plasma | | | | | | | | | | |
| Anatabine | Anatabine | Rat | male | 4 | 3 | 42.3 | 7.8 | 4.5 | 51.5 | 6 | 13.2 | 5.4 |
| | 1.0 mg/kg | Plasma | | | | | | | | | | |
| Anatabine | Anatabine | Rat | male | 6 | 3 | 18.0 | 2.6 | 1.5 | 24.3 | 12 | 7.4 | 2.1 |
| | 1.0 mg/kg | Plasma | | | | | | | | | | |
| Anatabine | Anatabine | Rat | male | 6 | 3 | 27.0 | 10.5 | 6.1 | 25.0 | 6 | 7.6 | 3.1 |
| | 1.0 mg/kg | Plasma | | | | | | | | | | |
| Anatabine | Anatabine | Rat | male | 8 | 3 | 7.7 | 1.5 | 0.9 | 12.7 | 6 | 5.8 | 2.3 |
| | 1.0 mg/kg | Plasma | | | | | | | | | | |
| Anatabine | Anatabine | Rat | male | 24 | 0 | <LLQ | ND | ND | <LLQ | 0 | ND | ND |
| | 1.0 mg/kg | Plasma | | | | | | | | | | |
| Anatabine | Anatabine | Rat | female | 0.25 | 3 | 288.7 | 15.9 | 9.2 | | | | |
| | 1.0 mg/kg | Plasma | | | | | | | | | | |
| Anatabine | Anatabine | Rat | female | 0.5 | 3 | 237.0 | 15.5 | 9.0 | | | | |
| | 1.0 mg/kg | Plasma | | | | | | | | | | |
| Anatabine | Anatabine | Rat | female | 0.5 | 3 | 368.0 | 24.6 | 14.2 | | | | |
| | 1.0 mg/kg | Plasma | | | | | | | | | | |
| Anatabine | Anatabine | Rat | female | 1 | 3 | 229.0 | 13.5 | 7.8 | | | | |
| | 1.0 mg/kg | Plasma | | | | | | | | | | |
| Anatabine | Anatabine | Rat | female | 1.5 | 3 | 199.7 | 10.0 | 5.8 | | | | |
| | 1.0 mg/kg | Plasma | | | | | | | | | | |
| Anatabine | Anatabine | Rat | female | 2 | 3 | 163.7 | 14.6 | 8.4 | | | | |
| | 1.0 mg/kg | Plasma | | | | | | | | | | |
| Anatabine | Anatabine | Rat | female | 4 | 3 | 60.7 | 11.0 | 6.3 | | | | |
| | 1.0 mg/kg | Plasma | | | | | | | | | | |
| Anatabine | Anatabine | Rat | female | 6 | 3 | 29.3 | 7.2 | 4.2 | | | | |
| | 1.0 mg/kg | Plasma | | | | | | | | | | |
| Anatabine | Anatabine | Rat | female | 6 | 3 | 23.0 | 4.6 | 2.6 | | | | |
| | 1.0 mg/kg | Plasma | | | | | | | | | | |
| Anatabine | Anatabine | Rat | female | 8 | 3 | 17.7 | 2.3 | 1.3 | | | | |
| | 1.0 mg/kg | Plasma | | | | | | | | | | |
| Anatabine | Anatabine | Rat | female | 24 | 0 | <LLQ | ND | ND | | | | |
| | 1.0 mg/kg | Plasma | | | | | | | | | | |

TABLE 16
Dosing solutions

| | Percentage | | | |
|---|---|---|---|---|
| | content of | | Test compound base | Injection |
| Test | anatabine or | Dose level | concentration | volume |
| compound | nicotine base | (mg/kg) | (mg/mL) | (mL/kg) |
| Anatabine | 41.6 | 0.10 | 0.048 | 5 |
| Anatabine | 41.6 | 0.75 | 0.36 | 5 |
| Anatabine | 41.6 | 1.5 | 0.72 | 5 |
| Nicotine | 35.1 | 0.75 | 0.36 | 5 |

TABLE 17
Study Outline

| | | | | Observations, body |
|---|---|---|---|---|
| | | | | weight and food |
| | | Dose level | Number of | consumption |
| Test compound | Route | (mg/kg) | animals (M/F) | frequency |
| Vehicle | i.v. | 0.0 | 5/5 | Daily for 14 days |
| Anatabine | i.v. | 0.10 | 5/5 | Daily for 14 days |
| Anatabine | i.v. | 0.75 | 5/5 | Daily for 14 days |
| Anatabine | i.v. | 1.5 | 5/5 | Daily for 14 days |
| Nicotine | i.v. | 1.5 | 5/5 | Not applicable |
| Nicotine¹ | i.v. | 0.75 | 5/5 | Daily for 14 days |

TABLE 18
Test Performed and Tissues Collected

| Parameter | Tests performed/tissues collected |
|---|---|
| Hematology | Hematocrit, Hemoglobin, MCH, MCH Concentration, |
| | MCV, RBC, Reticulocyte count, Platelet count, WBC, |
| | WBC differential, blood smear evaluation |
| Clinical | A/G ratio (calculated), ALT, Albumin, Alkaline |
| Chemistry | phosphatase, AST, Bilirubin, Calcium, Chloride, |
| | Cholesterol (total), Creatinine, Globulin (calculated), |
| | Glucose, Phosphorus (inorganic), Potassium, Sodium, |
| | Total protein, BUN |
| Coagulation | Activated partial thromboplastin time, Prothrombin time |
| Urinalysis | Bilirubin, Blood, color and appearance, Glucose, |
| | Ketones, pH, Protein, Specific gravity, Urobilinogen, |
| | Volume |
| Necropsy | Adrenal glands¹, Brain, Heart, Kidneys, Liver, Lungs, |
| | Ovaries and Uterus, Pituitary gland¹, Prostate gland, |
| | Spleen, Testes, Thymus, Thyroid and Parathyroid |
| | glands¹, section of small intestines |

¹Not weighed; placed in cassettes.

TABLE 19
Dosing Solution Analysis

| | | | Ex- | Actual | Actual |
|---|---|---|---|---|---|
| | | | pected | Concen- | Concentration |
| | Dosing | Dose | Conc. | tration | relative to |
| Compound | Date | (mg/kg) | (mg/ml) | (mg/mL) | Expected % |
| Nicotine | Jun 16 | 0.4 | 0.081 | 0.096 | 118.5 |
| Anatabine | Jun 09 | 0.1 | 0.02 | 0.016 | 80 |
| Anatabine | Jun 09 | 0.75 | 0.15 | 0.127 | 85 |
| Anatabine | Jun 09 | 1.5 | 0.2 | 0.239 | 119.5 |

TABLE 20
Mean Increase in Body Weights and Daily Food Consumption (descriptive statistics)

| | | | Number | Mean weight | | p | Mean food | | p |
|---|---|---|---|---|---|---|---|---|---|
| Test | Dose | | of | increase by day | Standard | (comparison | consumption/ | Standard | (comparison |
| Compound | (mg/kg) | Gender | animals | 14 (g) | Deviation | to Vehicle) | day (g) | Deviation | to Vehicle) |
| Vehicle | — | Male | 5 | 130.6 | 16.8 | | 27.7 | 2.8 | |
| | | Female | 5 | 49.7 | 6.4 | | 20.3 | 2.7 | |
| Anatabine | 0.1 | Male | 5 | 132.5 | 17.1 | 0.864 | 31.0 | 4.0 | 0.001 |
| | | Female | 5 | 39.0 | 10.3 | 0.083 | 20.8 | 3.6 | 0.535 |
| Anatabine | 0.75 | Male | 5 | 111.6 | 8.9 | 0.055 | 27.8 | 3.2 | 0.936 |
| | | Female | 5 | 43.1 | 9.3 | 0.229 | 19.7 | 2.8 | 0.434 |
| Anatabine | 1.5 | Male | 5 | 137.9 | 14.0 | 0.475 | 30.8 | 4.0 | 0.001 |
| | | Female | 5 | 38.6 | 13.0 | 0.125 | 19.8 | 3.0 | 0.504 |
| Nicotine | 0.75 | Male | 4 | 98.5 | 15.8 | 0.0001 | 26.4 | 3.2 | 0.098 |
| | | Female | 3 | 40.8 | 3.4 | 0.071 | 20.6 | 2.6 | 0.598 |

TABLE 21
Mean Organ Weights (descriptive statistics) for Male Animals by Treatment Group

| | | | Organ Weight (g) | | | | | | | | | |
|---|---|---|---|---|---|---|---|---|---|---|---|---|
| Test | Dose | | | | | | | | Small | | | |
| Compound | (mg/kg) | | Thymus | Heart | Lungs | Liver | Kidneys | Spleen | intestine | Prostate | Testes | Brain |
| Vehicle | — | Mean | 0.77 | 1.66 | 1.90 | 15.81 | 3.53 | 1.03 | 0.64 | 0.49 | 3.39 | 2.02 |
| | n = 5 | Std | 0.04 | 0.18 | 0.21 | 1.44 | 0.30 | 0.24 | 0.43 | 0.11 | 0.07 | 0.12 |
| | | Dev | | | | | | | | | | |
| Anatabine | 0.1 | Mean | 0.75 | 1.74 | 1.98 | 16.81 | 3.57 | 0.82 | 0.66 | 0.59 | 3.42 | 2.15 |
| | n = 5 | Std | 0.04 | 0.16 | 0.22 | 0.96 | 0.29 | 0.11 | 0.26 | 0.12 | 0.24 | 0.18 |
| | | Dev | | | | | | | | | | |
| | | p^{a} | ns^{b} | ns | ns | ns | ns | ns | ns | ns | ns | ns |
| Anatabine | 0.75 | Mean | 0.67 | 1.43 | 1.82 | 14.50 | 3.15 | 0.74 | 0.68 | 0.51 | 3.21 | 2.07 |
| | n = 5 | Std | 0.22 | 0.11 | 0.16 | 0.50 | 0.32 | 0.12 | 0.41 | 0.08 | 0.42 | 0.08 |
| | | Dev | | | | | | | | | | |
| | | p | ns | 0.04 | ns | ns | ns | 0.04 | ns | ns | ns | ns |
| Anatabine | 1.5 | Mean | 0.72 | 1.52 | 1.94 | 16.55 | 3.45 | 0.86 | 0.68 | 0.47 | 3.24 | 1.93 |
| | n = 5 | Std | 0.14 | 0.06 | 0.11 | 1.55 | 0.28 | 0.10 | 0.22 | 0.03 | 0.28 | 0.14 |
| | | Dev | | | | | | | | | | |
| | | p | ns | ns | ns | ns | ns | ns | ns | ns | ns | ns |
| Nicotine | 0.75 | Mean | 0.58 | 1.25 | 1.65 | 12.69 | 3.03 | 0.77 | 0.55 | 0.36 | 3.03 | 1.94 |
| | n = 4 | Std | 0.13 | 0.11 | 0.14 | 1.04 | 0.13 | 0.10 | 0.13 | 0.09 | 0.09 | 0.09 |
| | | Dev | | | | | | | | | | |
| | | p | ns | 0.006 | ns | 0.008 | 0.017 | ns | ns | ns | ns | ns |

^{a}p, probability relative to Vehicle control;
^{b}ns, not significant

TABLE 22
Mean Organ Weights (descriptive statistics) for Female Animals by Treatment Group

| | | | Organ Weight (g) | | | | | | | | |
|---|---|---|---|---|---|---|---|---|---|---|---|
| Test | Dose | | | | | | | | Small | Ovaries/ | |
| Compound | (mg/kg) | | Thymus | Heart | Lungs | Liver | Kidneys | Spleen | intestine | uterus | Brain |
| Vehicle | — | Mean | 0.69 | 1.27 | 1.52 | 10.94 | 2.34 | 0.61 | 0.82 | 1.10 | 1.93 |
| | n = 5 | Std Dev | 0.10 | 0.09 | 0.13 | 1.18 | 0.40 | 0.09 | 0.39 | 0.53 | 0.15 |
| Anatabine | 0.1 | Mean | 0.64 | 1.13 | 1.41 | 10.84 | 2.18 | 0.65 | 0.65 | 1.14 | 1.86 |
| | n = 5 | Std Dev | 0.10 | 0.14 | 0.17 | 1.38 | 0.18 | 0.10 | 0.15 | 0.45 | 0.06 |
| | | p^{a} | ns^{b} | ns | ns | ns | ns | ns | ns | ns | ns |
| Anatabine | 0.75 | Mean | 0.56 | 1.11 | 1.45 | 10.42 | 2.30 | 0.60 | 0.75 | 1.58 | 1.90 |
| | n = 5 | Std Dev | 0.04 | 0.11 | 0.12 | 1.09 | ns | 0.09 | 0.38 | 1.20 | 0.13 |
| | | p | 0.026 | 0.033 | ns | ns | ns | ns | ns | ns | ns |
| Anatabine | 1.5 | Mean | 0.57 | 1.00 | 1.46 | 10.04 | 2.28 | 0.60 | 0.70 | 1.03 | 1.95 |
| | n = 5 | Std Dev | 0.05 | 0.09 | 0.14 | 1.16 | 0.29 | 0.08 | 0.23 | 0.24 | 0.08 |
| | | p | ns | 0.001 | ns | ns | ns | ns | ns | ns | ns |
| Nicotine | 0.75 | Mean | 0.50 | 0.99 | 1.32 | 9.59 | 2.31 | 0.56 | 0.36 | 1.28 | 1.86 |
| | n = 3 | Std Dev | 0.03 | 0.14 | 0.14 | 0.47 | 0.03 | 0.17 | 0.07 | 0.34 | 0.08 |
| | | p | 0.022 | 0.013 | ns | ns | ns | ns | ns | ns | ns |

^{a}p, probability relative to Vehicle control
^{b}ns, not significant

TABLE 23A
Hematology Parameters (descriptive statistics) (Part 1) by Treatment Group and Gender

| | | | | | | | | | | | PLATELET |
|---|---|---|---|---|---|---|---|---|---|---|---|
| Treatment | | | WBC × | RBC × | HGB | | MCV | MCH | | RETICULOCYTE | COUNT × |
| Group | Gender | | 10³/μL | 10⁶/μL | g/dL | HCT % | fL | pg | MCHC % | COUNT % | 10³/μL |
| Vehicle | M | n | 5 | 5 | 5 | 5 | 5 | 5 | 5 | 5 | 4 |
| | | Mean | 10.5 | 7.3 | 15.2 | 45.4 | 62.2 | 20.7 | 33.4 | 6.1 | 1287.3 |
| | | Std | 1.4 | 0.2 | 0.5 | 1.5 | 1.5 | 0.6 | 0.4 | 1.0 | 210.8 |
| | | Dev | | | | | | | | | |
| | F | N | 5 | 5 | 5 | 5 | 5 | 5 | 5 | 5 | 5 |
| | | Mean | 9.3 | 7.8 | 16.4 | 47.3 | 61.0 | 21.0 | 34.5 | 5.0 | 1198.4 |
| | | Std | 2.8 | 0.7 | 1.7 | 4.7 | 1.2 | 0.6 | 0.8 | 1.1 | 200.2 |
| | | Dev | | | | | | | | | |
| Anatabine | M | n | 5 | 5 | 5 | 5 | 5 | 5 | 5 | 5 | 5 |
| 0.1 mg/kg | | Mean | 15.6 | 7.4 | 15.8 | 47.1 | 63.4 | 21.2 | 33.5 | 5.5 | 1248.6 |
| | | Std | 4.7 | 0.4 | 1.2 | 2.9 | 1.8 | 0.6 | 0.9 | 0.5 | 358.1 |
| | | Dev | | | | | | | | | |
| | | p^{a} | ns^{b} | ns | ns | ns | ns | ns | ns | ns | ns |
| | F | n | 5 | 5 | 5 | 5 | 5 | 5 | 5 | 5 | 3 |
| | | Mean | 9.3 | 7.8 | 15.6 | 46.4 | 60.0 | 20.2 | 33.7 | 4.5 | 997.3 |
| | | Std | 3.4 | 0.7 | 1.0 | 3.2 | 2.0 | 0.8 | 0.3 | 1.5 | 165.8 |
| | | Dev | | | | | | | | | |
| | | p | ns | ns | ns | ns | ns | ns | 0.042^{c} | ns | ns |
| Anatabine | M | n | 5 | 5 | 5 | 5 | 5 | 5 | 5 | 5 | 4 |
| 0.75 mg/kg | | Mean | 12.1 | 7.5 | 15.6 | 45.8 | 61.4 | 20.9 | 34.0 | 4.4 | 1325.8 |
| | | Std | 5.7 | 0.7 | 1.5 | 4.0 | 1.5 | 0.8 | 0.7 | 0.6 | 193.7 |
| | | Dev | | | | | | | | | |
| | | p | ns | ns | ns | ns | ns | ns | ns | 0.012^{c} | ns |
| | F | n | 5 | 5 | 5 | 5 | 5 | 5 | 5 | 5 | 5 |
| | | Mean | 10.3 | 7.5 | 15.4 | 45.2 | 59.8 | 20.4 | 34.1 | 3.3 | 1269.6 |
| | | Std | 4.0 | 0.3 | 0.5 | 1.5 | 2.3 | 0.8 | 0.3 | 0.9 | 356.4 |
| | | Dev | | | | | | | | | |
| | | p | ns | ns | ns | ns | ns | ns | ns | 0.022^{c} | ns |

TABLE 23B
Hematology Parameters (descriptive statistics) (Part 1) by Treatment Group and Gender - cont'd

| | | | | | | | | | | | PLATELET |
|---|---|---|---|---|---|---|---|---|---|---|---|
| Treatment | | | WBC × | RBC × | HGB | | MCV | MCH | | RETICULOCYTE | COUNT × |
| Group | Gender | | 10³/μL | 10⁶/μL | g/dL | HCT % | fL | pg | MCHC % | COUNT % | 10³/μL |
| Anatabine | M | n | 5 | 5 | 5 | 5 | 5 | 5 | 5 | 5 | 5 |
| 1.5 mg/kg | | Mean | 13.1 | 7.5 | 15.4 | 46.1 | 62.0 | 20.6 | 33.4 | 4.2 | 1222.5 |
| | | Std | 3.1 | 0.2 | 0.6 | 2.2 | 1.9 | 0.5 | 0.4 | 0.8 | 248.4 |
| | | Dev | | | | | | | | | |
| | | p | ns | ns | ns | ns | ns | ns | ns | 0.011^{c} | ns |
| | F | n | 5 | 5 | 5 | 5 | 5 | 5 | 5 | 5 | 5 |
| | | Mean | 9.2 | 7.7 | 15.2 | 45.4 | 58.8 | 19.6 | 33.4 | 3.2 | 1127.4 |
| | | Std | 2.5 | 0.4 | 0.7 | 2.7 | 0.8 | 0.4 | 0.6 | 0.9 | 503.7 |
| | | Dev | | | | | | | | | |
| | | p | ns | ns | ns | ns | 0.011^{c} | 0.002^{c} | 0.036^{c} | 0.021^{c} | ns |
| Nicotine | M | n | 4 | 4 | 4 | 4 | 4 | 4 | 4 | 4 | 4 |
| 0.75 mg/kg | | Mean | 11.4 | 7.6 | 15.8 | 46.6 | 61.5 | 20.9 | 34.0 | 5.2 | 1355.8 |
| | | Std | 3.2 | 0.5 | 0.7 | 2.4 | 1.0 | 0.6 | 0.5 | 0.8 | 83.0 |
| | | Dev | | | | | | | | | |
| | | p | ns | ns | ns | ns | ns | ns | ns | ns | ns |
| | F | n | 3 | 3 | 3 | 3 | 3 | 3 | 3 | 3 | 2 |
| | | Mean | 10.2 | 8.0 | 16.0 | 46.7 | 58.7 | 19.9 | 34.2 | 5.1 | 1363.0 |
| | | Std | 3.1 | 0.5 | 0.7 | 2.2 | 1.2 | 0.5 | 0.2 | 0.7 | 248.9 |
| | | Dev | | | | | | | | | |
| | | p | ns | ns | ns | ns | 0.038^{c} | 0.038^{c} | ns | ns | ns |

^{a}p, probability relative to Vehicle control
^{b}ns, not significant
^{c}Mean within normal range

TABLE 24
Hematology Parameters (descriptive statistics) (Part 2) by Treatment
Group and Gender

| Treatment | | | NEUTROPHIL | | | | |
|---|---|---|---|---|---|---|---|
| Group | Gender | | SEG % | LYMPHOCYTE % | MONOCYTE % | EOSINOPHIL % | B [symbol] |
| Vehicle | M | n | 5 | 5 | 5 | 5 | [symbol] |
| | | Mean | 8.8 | 89.6 | 1.6 | 0.0 | [symbol] |
| | | Std Dev | 2.2 | 2.3 | 1.5 | 0.0 | [symbol] |
| | F | N | 5 | 5 | 5 | 5 | [symbol] |
| | | Mean | 12.6 | 85.2 | 2.2 | 0.0 | [symbol] |
| | | Std | 2.6 | 2.9 | 0.4 | 0.0 | [symbol] |
| Anatabine | M | n | 5 | 5 | 5 | 5 | [symbol] |
| 0.1 mg/kg | | Mean | 8.6 | 89.2 | 2.0 | 0.2 | [symbol] |
| | | Std Dev | 3.6 | 2.9 | 1.0 | 0.4 | [symbol] |
| | | p^{a} | ns^{b} | ns | ns | ns | [symbol] |
| | F | n | 5 | 5 | 5 | 5 | [symbol] |
| | | Mean | 8.0 | 90.2 | 1.8 | 0.0 | [symbol] |
| | | Std Dev | 1.9 | 1.9 | 0.8 | 0.0 | [symbol] |
| | | p | ns | ns | ns | ns | [symbol] |
| Anatabine | M | n | 5 | 5 | 5 | 5 | [symbol] |
| 0.75 mg/kg | | Mean | 9.8 | 88.8 | 1.4 | 0.0 | [symbol] |
| | | Std Dev | 2.5 | 2.2 | 0.5 | 0.0 | [symbol] |
| | | p | ns | ns | ns | ns | [symbol] |
| | F | n | 5 | 5 | 5 | 5 | [symbol] |
| | | Mean | 8.6 | 90.6 | 0.8 | 0.0 | [symbol] |
| | | Std Dev | 2.3 | 2.6 | 0.8 | 0.0 | [symbol] |
| | | p | ns | ns | ns | ns | [symbol] |
| Anatabine | M | n | 5 | 5 | 5 | 5 | [symbol] |
| 1.5 mg/kg | | Mean | 14.6 | 83.8 | 1.6 | 0.0 | [symbol] |
| | | Std Dev | 7.7 | 8.0 | 0.9 | 0.0 | [symbol] |
| | | p | ns | ns | ns | ns | [symbol] |
| | F | n | 5 | 5 | 5 | 5 | [symbol] |
| | | Mean | 12.6 | 86.6 | 0.8 | 0.0 | [symbol] |
| | | Std Dev | 4.4 | 3.8 | 0.8 | 0.0 | [symbol] |
| | | p | ns | ns | ns | ns | [symbol] |
| Nicotine | M | n | 4 | 4 | 4 | 4 | [symbol] |
| 0.75 mg/kg | | Mean | 11.8 | 85.8 | 2.5 | 0.0 | [symbol] |
| | | Std Dev | 3.3 | 4.2 | 1.0 | 0.0 | [symbol] |
| | | p | ns | ns | ns | ns | [symbol] |
| | F | n | 3 | 3 | 3 | 3 | [symbol] |
| | | Mean | 12.7 | 86.3 | 1.0 | 0.0 | [symbol] |
| | | Std Dev | 2.1 | 3.1 | 1.0 | 0.0 | [symbol] |
| | | p | ns | ns | ns | ns | |

^{a}p, probability relative to Vehicle control
^{b}ns, not significant [symbol]
indicates data missing or illegible when filed

TABLE 25
Coagulation Parameters (descriptive statistics)
by Treatment Group and Gender

| | | | ACTIVATED | |
|---|---|---|---|---|
| | | | PARTIAL THROM- | PROTHROMBIN |
| Treatment | | | BOPLASTIN TIME | TIME |
| Group | Gender | | (seconds) | (seconds) |
| Vehicle | M | n | 5 | 5 |
| | | Mean | 32.0 | 12.9 |
| | | Std Dev | 2.2 | 0.5 |
| | F | n | 5 | 5 |
| | | Mean | 35.4 | 12.7 |
| | | Std Dev | 4.1 | 0.3 |
| Anatabine | M | n | 5 | 5 |
| 0.1 | | Mean | 32.4 | 12.8 |
| mg/kg | | Std Dev | 3.3 | 0.7 |
| | | p^{a} | ns^{b} | ns |
| | F | n | 5 | 5 |
| | | Mean | 29.4 | 12.1 |
| | | Std Dev | 3.4 | 0.4 |
| | | p | ns | ns |
| Anatabine | M | n | 5 | 5 |
| 0.75 | | Mean | 38.7 | 13.3 |
| mg/kg | | Std Dev | 9.1 | 2.6 |
| | | p | ns | ns |
| | F | n | 5 | 5 |
| | | Mean | 33.3 | 14.1 |
| | | Std Dev | 2.8 | 5.5 |
| | | p | ns | ns |
| Anatabine | M | n | 5 | 5 |
| 1.5 | | Mean | 30.6 | 13.2 |
| mg/kg | | Std Dev | 3.1 | 0.4 |
| | | p | ns | ns |
| | F | n | 5 | 5 |
| | | Mean | 32.5 | 13.3 |
| | | Std Dev | 2.9 | 0.2 |
| | | p | ns | ns |
| Nicotine | M | n | 4 | 4 |
| 0.75 | | Mean | 16.3 | 13.6 |
| mg/kg | | Std Dev | 1.1 | 0.6 |
| | | p | <0.001^{c} | ns |
| | F | n | 2 | 2 |
| | | Mean | 16.9 | 14.1 |
| | | Std Dev | 2.6 | 0.2 |
| | | p | 0.002^{c} | ns |

^{a}p, probability relative to Vehicle control
^{b}ns, not significant
^{c}Mean within normal range

TABLE 26A
Clinical Chemistry Parameters (descriptive statistics) (Part 1) by Treatment Group and Gender

| | | | ALK | | | | TOT | | TOT | DIR | |
|---|---|---|---|---|---|---|---|---|---|---|---|
| Treatment | | | PHOSPHATASE | ALT | AST | ALBUMIN | PROTEIN | GLOBULIN | BILIRUBIN | BILIRUBIN | BUN |
| Group | Gender | | IU/L | IU/L | IU/L | g/dL | g/dL | g/dL | mg/dL | mg/dL | mg/dL |
| Vehicle | M | n | 5 | 5 | 5 | 5 | 5 | 5 | 5 | 5 | 5 |
| | | Mean | 370.80 | 59.40 | 99.00 | 3.12 | 5.62 | 2.50 | 0.00 | 0.00 | 18.60 |
| | | Std | 72.47 | 2.07 | 22.86 | 0.04 | 0.04 | 0.00 | 0.00 | 0.00 | 1.14 |
| | | Dev | | | | | | | | | |
| | F | N | 5 | 5 | 5 | 5 | 5 | 5 | 5 | 5 | 5 |
| | | Mean | 230.20 | 58.20 | 80.80 | 3.40 | 6.24 | 2.84 | 0.00 | 0.00 | 18.20 |
| | | Std | 54.56 | 4.44 | 11.32 | 0.07 | 0.09 | 0.11 | 0.00 | 0.00 | 4.44 |
| | | Dev | | | | | | | | | |
| Anatabine | M | n | 5 | 5 | 5 | 5 | 5 | 5 | 5 | 5 | 5 |
| 0.1 mg/kg | | Mean | 407.40 | 70.60 | 96.00 | 3.24 | 6.10 | 2.86 | 0.00 | 0.00 | 18.00 |
| | | Std | 145.89 | 17.77 | 7.75 | 0.15 | 0.23 | 0.18 | 0.00 | 0.00 | 2.55 |
| | | Dev | | | | | | | | | |
| | | p^{a} | ns^{b} | ns | ns | ns | 0.002^{c} | 0.002^{c} | ns | ns | ns |
| | F | n | 5 | 5 | 5 | 5 | 5 | 5 | 5 | 5 | 5 |
| | | Mean | 248.40 | 49.60 | 77.00 | 3.62 | 6.64 | 3.02 | 0.00 | 0.00 | 19.80 |
| | | Std | 101.28 | 9.63 | 7.42 | 0.11 | 0.22 | 0.13 | 0.00 | 0.00 | 1.10 |
| | | Dev | | | | | | | | | |
| | | p | ns | ns | ns | 0.005^{c} | 0.005^{c} | 0.049^{c} | ns | ns | ns |
| Anatabine | M | n | 5 | 5 | 5 | 5 | 5 | 5 | 5 | 5 | 5 |
| 0.75 mg/kg | | Mean | 370.80 | 66.80 | 89.60 | 3.28 | 6.04 | 2.76 | 0.02 | 0.02 | 16.60 |
| | | Std | 74.89 | 14.64 | 14.12 | 0.13 | 0.24 | 0.11 | 0.04 | 0.04 | 3.36 |
| | | Dev | | | | | | | | | |
| | | p | ns | ns | ns | 0.032^{c} | 0.005^{c} | 0.001^{c} | ns | ns | ns |
| | F | n | 5 | 5 | 5 | 5 | 5 | 5 | 5 | 5 | 5 |
| | | Mean | 158.00 | 54.60 | 73.20 | 3.52 | 6.38 | 2.86 | 0.00 | 0.00 | 15.40 |
| | | Std | 45.27 | 5.90 | 14.75 | 0.08 | 0.16 | 0.13 | 0.00 | 0.00 | 0.55 |
| | | Dev | | | | | | | | | |
| | | p | ns | ns | ns | 0.040^{c} | ns | ns | ns | ns | ns |

TABLE 26B
Clinical Chemistry Parameters (descriptive statistics) (Part 1) by Treatment Group and Gender - cont'd

| | | | ALK | | | | TOT | | TOT | DIR | |
|---|---|---|---|---|---|---|---|---|---|---|---|
| Treatment | | | PHOSPHATASE | ALT | AST | ALBUMIN | PROTEIN | GLOBULIN | BILIRUBIN | BILIRUBIN | |
| Group | Gender | | IU/L | IU/L | IU/L | g/dL | g/dL | g/dL | mg/dL | mg/dL | BUN mg/dL |
| Anatabine | M | n | 5 | 5 | 5 | 5 | 5 | 5 | 5 | 5 | 5 |
| 1.5 mg/kg | | Mean | 411.40 | 70.40 | 97.00 | 3.32 | 6.00 | 2.68 | 0.00 | 0.00 | 17.20 |
| | | Std | 133.64 | 20.61 | 26.88 | 0.13 | 0.25 | 0.13 | 0.00 | 0.00 | 1.30 |
| | | Dev | | | | | | | | | |
| | | p | ns | ns | ns | 0.012^{c} | 0.011^{c} | 0.015^{c} | ns | ns | ns |
| | F | n | 5 | 5 | 5 | 5 | 5 | 5 | 5 | 5 | 5 |
| | | Mean | 194.00 | 49.20 | 76.60 | 3.48 | 6.36 | 2.88 | 0.02 | 0.02 | 16.20 |
| | | Std | 58.97 | 6.18 | 9.21 | 0.15 | 0.28 | 0.16 | 0.04 | 0.04 | 2.17 |
| | | Dev | | | | | | | | | |
| | | p | ns | ns | ns | ns | ns | ns | ns | ns | ns |
| Nicotine | M | n | 4 | 4 | 4 | 4 | 4 | 4 | 4 | 4 | 4 |
| 0.75 mg/kg | | Mean | 291.00 | 55.75 | 82.25 | 3.08 | 6.00 | 2.93 | 0.05 | 0.00 | 17.50 |
| | | Std | 51.59 | 8.46 | 7.63 | 0.05 | 0.16 | 0.17 | 0.06 | 0.00 | 1.73 |
| | | Dev | | | | | | | | | |
| | | p | ns | ns | ns | ns | 0.001^{c} | 0.001^{c} | ns | ns | ns |
| | F | n | 3 | 3 | 3 | 3 | 3 | 3 | 3 | 3 | 3 |
| | | Mean | 188.67 | 68.33 | 103.00 | 3.30 | 6.50 | 3.20 | 0.10 | 0.03 | 19.00 |
| | | Std | 74.33 | 24.83 | 29.05 | 0.00 | 0.10 | 0.10 | 0.00 | 0.06 | 1.73 |
| | | Dev | | | | | | | | | |
| | | p | ns | ns | ns | ns | 0.009^{c} | 0.004^{c} | ns | ns | ns |

^{a}p, probability relative to Vehicle control
^{b}ns, not significant
^{c}Mean within normal range

TABLE 27A
Clinical Chemistry Parameters (descriptive statistics) (Part 2) by Treatment Group and Gender

| | | | CREAT- | CHOLES- | | | PHOSPHO- | | | | |
|---|---|---|---|---|---|---|---|---|---|---|---|
| Treatment | | | ININE | TEROL | GLUCOSE | CALCIUM | RUS | CHLORIDE | POTASSIUM | SODIUM | A/G |
| Group | Gender | | mg/dL | mg/dL | mg/dL | mg/dL | mg/dL | mEq/L | mEq/L | mEq/L | RATIO |
| Vehicle | M | n | 5 | 5 | 5 | 5 | 5 | 5 | 5 | 5 | 5 |
| | | Mean | 0.36 | 60.00 | 222.00 | 12.12 | 10.82 | 98.60 | 6.30 | 145.40 | 1.22 |
| | | Std | 0.05 | 4.06 | 47.00 | 0.31 | 0.51 | 1.52 | 0.29 | 1.14 | 0.04 |
| | | Dev | | | | | | | | | |
| | F | n | 5 | 5 | 5 | 5 | 5 | 5 | 5 | 5 | 5 |
| | | Mean | 0.42 | 67.40 | 198.20 | 11.66 | 9.06 | 99.80 | 5.92 | 145.60 | 1.18 |
| | | Std | 0.04 | 11.48 | 13.03 | 0.34 | 0.61 | 0.45 | 0.50 | 0.55 | 0.08 |
| | | Dev | | | | | | | | | |
| Anatabine | M | n | 5 | 5 | 5 | 5 | 5 | 5 | 5 | 5 | 5 |
| 0.1 mg/kg | | Mean | 0.36 | 67.20 | 219.00 | 12.00 | 10.82 | 98.00 | 6.40 | 146.20 | 1.14 |
| | | Std | 0.05 | 8.35 | 23.73 | 0.39 | 0.54 | 1.00 | 0.40 | 1.48 | 0.09 |
| | | Dev | | | | | | | | | |
| | | p^{a} | ns^{b} | ns | ns | ns | ns | ns | ns | ns | ns |
| | F | n | 5 | 5 | 5 | 5 | 5 | 5 | 5 | 5 | 5 |
| | | Mean | 0.44 | 76.40 | 207.60 | 11.72 | 8.08 | 100.20 | 5.82 | 146.80 | 1.22 |
| | | Std | 0.05 | 11.63 | 24.11 | 0.58 | 0.48 | 2.17 | 0.65 | 1.30 | 0.04 |
| | | Dev | | | | | | | | | |
| | | p | ns | ns | ns | ns | ns | ns | ns | ns | ns |
| Anatabine | M | n | 5 | 5 | 5 | 5 | 5 | 5 | 5 | 5 | 5 |
| 0.75 mg/kg | | Mean | 0.36 | 62.80 | 202.00 | 12.04 | 10.92 | 98.60 | 6.12 | 147.80 | 1.20 |
| | | Std | 0.05 | 6.38 | 25.25 | 0.38 | 0.85 | 1.14 | 0.45 | 1.48 | 0.00 |
| | | Dev | | | | | | | | | |
| | | p | ns | ns | ns | ns | ns | ns | ns | 0.021^{c} | ns |
| | F | n | 5 | 5 | 5 | 5 | 5 | 5 | 5 | 5 | 5 |
| | | Mean | 0.44 | 69.80 | 220.80 | 11.68 | 8.64 | 98.00 | 5.88 | 145.00 | 1.24 |
| | | Std | 0.05 | 9.88 | 21.73 | 0.29 | 1.22 | 2.00 | 0.30 | 1.22 | 0.05 |
| | | Dev | | | | | | | | | |
| | | p | ns | ns | ns | ns | ns | ns | ns | ns | ns |

TABLE 27B
Clinical Chemistry Parameters (descriptive statistics) (Part 2) by Treatment Group and Gender - cont'd

| | | | CREAT- | CHOLES- | | | PHOSPHO- | | | | |
|---|---|---|---|---|---|---|---|---|---|---|---|
| Treatment | | | ININE | TEROL | GLUCOSE | CALCIUM | RUS | CHLORIDE | POTASSIUM | SODIUM | A/G |
| Group | Gender | | mg/dL | mg/dL | mg/dL | mg/dL | mg/dL | mEq/L | mEq/L | mEq/L | RATIO |
| Anatabine | M | n | 5 | 5 | 5 | 5 | 5 | 5 | 5 | 5 | 5 |
| 1.5 mg/kg | | Mean | 0.36 | 62.40 | 205.60 | 11.88 | 11.00 | 99.80 | 6.02 | 147.80 | 1.24 |
| | | Std | 0.05 | 4.39 | 6.11 | 0.51 | 0.83 | 0.84 | 0.77 | 1.30 | 0.05 |
| | | Dev | | | | | | | | | |
| | | p | ns | ns | ns | ns | ns | ns | ns | 0.015^{c} | ns |
| | F | n | 5 | 5 | 5 | 5 | 5 | 5 | 5 | 5 | 5 |
| | | Mean | 0.40 | 70.00 | 188.40 | 11.36 | 8.24 | 100.80 | 5.74 | 147.20 | 1.24 |
| | | Std | 0.00 | 9.92 | 9.07 | 0.35 | 0.59 | 1.92 | 0.59 | 0.84 | 0.05 |
| | | Dev | | | | | | | | | |
| | | p | ns | ns | ns | ns | ns | ns | ns | 0.007^{c} | ns |
| Nicotine | M | n | 4 | 4 | 4 | 4 | 4 | 4 | 4 | 4 | 4 |
| 0.75 mg/kg | | Mean | 0.35 | 61.00 | 201.00 | 11.05 | 10.38 | 99.75 | 6.20 | 146.75 | 1.05 |
| | | Std | 0.06 | 8.49 | 10.42 | 0.06 | 0.58 | 0.96 | 0.50 | 0.96 | 0.06 |
| | | Dev | | | | | | | | | |
| | | p | ns | ns | ns | <0.001^{c} | ns | ns | ns | ns | 0.002^{c} |
| | F | n | 3 | 3 | 3 | 3 | 3 | 3 | 3 | 3 | 3 |
| | | Mean | 0.43 | 65.00 | 238.67 | 11.03 | 8.37 | 98.67 | 6.27 | 145.00 | 1.03 |
| | | Std | 0.06 | 14.93 | 53.72 | 0.21 | 1.02 | 1.53 | 0.50 | 1.73 | 0.06 |
| | | Dev | | | | | | | | | |
| | | p | ns | ns | ns | ns | ns | ns | ns | ns | 0.038^{c} |

^{a}p, probability relative to Vehicle control
^{b}ns, not significant
^{c}Mean within normal range

TABLE 28A
Dosing Calculations and Body Weights, days 1-5 (anatabine)

| | | | | Dose | | day 1 | day 2 | day 3 | day 4 | day 5 |
|---|---|---|---|---|---|---|---|---|---|---|
| | | | B.W. | volume | time | B.W. | B.W. | B.W. | B.W. | B.W. |
| Group | Rat | M/F | (g) | (mL) | of dosing | (g) | (g) | (g) | (g) | (g) |
| A-1 | 1 | M | 250 | 1.25 | 10:39 | 258.0 | 270.0 | 280.3 | 296.0 | 306.6 |
| vehicle | 2 | M | 228 | 1.15 | 10:41 | 238.0 | 251.0 | 257.8 | 273.7 | 286.0 |
| 5 mL/kg | 3 | M | 235 | 1.18 | 10:43 | 241.0 | 250.0 | 260.2 | 278.0 | 287.0 |
| A-2 | 4 | M | 224 | 1.13 | 10:44 | 230.0 | 239.0 | 247.6 | 262.6 | 267.0 |
| | 5 | M | 236 | 1.18 | 10:45 | 241.0 | 253.1 | 261.7 | 281.0 | 289.0 |
| A-3 | 6 | F | 207 | 1.03 | 10:49 | 210.2 | 220.5 | 218.1 | 224.2 | 227.0 |
| | 7 | F | 221 | 1.1 | 10:50 | 221.9 | 222.0 | 225.1 | 229.2 | 235.7 |
| | 8 | F | 209 | 1.05 | 10:51 | 210.0 | 214.0 | 216.9 | 219.5 | 220.0 |
| A-4 | 9 | F | 201 | 1 | 10:51 | 200.0 | 200.1 | 205.0 | 216.7 | 212.0 |
| | 10 | F | 211 | 1.05 | 10:52 | 208.4 | 215.0 | 216.1 | 227.9 | 225.0 |
| B-1 | 1 | M | 237 | 1.18 | 10:48 | 245.0 | 257.0 | 270.0 | 286.0 | 296.5 |
| Anatabine | 2 | M | 227 | 1.13 | 10:49 | 233.7 | 235.1 | 251.0 | 270.0 | 273.8 |
| 0.1 mg/kg | 3 | M | 230 | 1.15 | 10:50 | 235.0 | 231.0 | 252.0 | 271.2 | 278.5 |
| B-2 | 4 | M | 243 | 1.23 | 10:52 | 243.5 | 253.5 | 259.4 | 280.5 | 288.3 |
| | 5 | M | 235 | 1.18 | 10:55 | 239.9 | 247.3 | 256.6 | 273.7 | 286.6 |
| B-3 | 6 | F | 226 | 1.13 | 10:56 | 223.0 | 223.9 | 230.8 | 242.3 | 246.9 |
| | 7 | F | 212 | 1.05 | 10:57 | 211.1 | 215.1 | 213.2 | 222.4 | 226.4 |
| | 8 | F | 207 | 1.03 | 10:58 | 207.0 | 208.1 | 209.1 | 220.7 | 219.3 |
| B-4 | 9 | F | 205 | 1.03 | 10:58 | 201.0 | 208.8 | 209.1 | 211.4 | 221.0 |
| | 10 | F | 215 | 1.08 | 10:59 | 210.9 | 212.8 | 219.3 | 224.7 | 229.5 |

| | | | | Dose | | day 1 | day 2 | day 3 | day 4 | day 4 |
|---|---|---|---|---|---|---|---|---|---|---|
| | | | B.W. | volume | | B.W. | B.W. | B.W. | B.W. | B.W. |
| Group | Rat | M/F | (g) | (mL) | time | (g) | (g) | (g) | (g) | (g) |
| C-1 | 1 | M | 239 | 1.2 | 10:58 | 242.7 | 248.9 | 261.1 | 275.7 | 282.3 |
| Anatabine | 2 | M | 241 | 1.2 | 11:00 | 251.4 | 259.7 | 269.1 | 286.0 | 287.6 |
| 0.75 mg/kg | 3 | M | 229 | 1.15 | 11:02 | 231.9 | 240.0 | 252.0 | 268.0 | 269.9 |
| C-2 | 4 | M | 223 | 1.13 | 11:04 | 226.8 | 232.1 | 243.0 | 256.3 | 262.0 |
| | 5 | M | 240 | 1.2 | 11:06 | 239.8 | 247.2 | 256.1 | 271.2 | 275.7 |
| C-3 | 6 | F | 214 | 1.08 | 11:01 | 208.9 | 206.2 | 214.0 | 230.0 | 225.4 |
| | 7 | F | 206 | 1.03 | 11:02 | 207.7 | 210.0 | 214.0 | 214.7 | 219.3 |
| | 8 | F | 215 | 1.08 | 11:03 | 219.1 | 225.5 | 224.8 | 232.4 | 237.1 |
| C-4 | 9 | F | 207 | 1.03 | 11:05 | 196.7 | 210.0 | 213.6 | 222.1 | 220.8 |
| | 10 | F | 212 | 1.05 | 11:06 | 199.5 | 210.1 | 210.8 | 220.0 | 227.6 |
| D-1 | 1 | M | 230 | 1.15 | 11:07 | 234.0 | 240.4 | 258.4 | 244.4 | 286.3 |
| Anatabine | 2 | M | 248 | 1.25 | 11:21 | 242.6 | 254.0 | 267.9 | 255.1 | 268.1 |
| 1.5 mg/kg | 3 | M | 228 | 1.15 | 11:23 | 231.0 | 240.3 | 252.5 | 247.3 | 269.8 |
| D-2 | 4 | M | 239 | 1.2 | 11:26 | 245.3 | 257.0 | 272.5 | 290.2 | 295.8 |
| | 5 | M | 227 | 1.15 | 11:28 | 227.8 | 237.3 | 250.1 | 262.0 | 270.5 |
| D-3 | 6 | F | 216 | 1.08 | 11:08 | 213.9 | 212.0 | 220.0 | 226.2 | 228.0 |
| | 7 | F | 206 | 1.03 | 11:09 | 204.0 | 206.2 | 210.3 | 216.8 | 217.5 |
| | 8 | F | 219 | 1.1 | 11:20 | 219.6 | 221.6 | 224.3 | 234.7 | 230.9 |
| D-4 | 9 | F | 231 | 1.15 | 11:22 | 229.5 | 233.9 | 237.0 | 248.1 | 247.5 |
| | 10 | F | 206 | 1.03 | 11:23 | 206.1 | 208.6 | 211.4 | 221.8 | 218.9 |

TABLE 28B
Dosing Calculations and Body Weights, days 6-12 (anatabine)

| | | | | | | | day | day | day |
|---|---|---|---|---|---|---|---|---|---|
| | | | day 6 | day 7 | day 8 | day 9 | 10 | 11 | 12 |
| | | M/ | B.W. | B.W. | B.W. | B.W. | B.W. | B.W. | B.W. |
| Group | Rat | F | (g) | (g) | (g) | (g) | (g) | (g) | (g) |
| A-1 | 1 | M | 308 | 317.0 | 332.0 | 338.0 | 348.7 | 364.2 | 369.5 |
| vehicle | 2 | M | 283.7 | 300.0 | 310.0 | 315.0 | 327.2 | 342.6 | 342.6 |
| 5 mL/kg | 3 | M | 287.9 | 300.0 | 314.0 | 317.0 | 332.0 | 349.1 | 354.1 |
| A-2 | 4 | M | 268.8 | 273.5 | 286.0 | 286.9 | 296.1 | 312.7 | 316.7 |
| | 5 | M | 294.6 | 301.1 | 315.0 | 315.2 | 332.3 | 339.7 | 351.0 |
| A-3 | 6 | F | 228.2 | 231.7 | 235.5 | 237.4 | 246.7 | 253.8 | 248.9 |
| | 7 | F | 235.3 | 236.8 | 260.8 | 249.8 | 252.8 | 261.3 | 256.3 |
| | 8 | F | 221.9 | 225.9 | 226.4 | 230.1 | 236.4 | 248.0 | 240.9 |
| A-4 | 9 | F | 216 | 217.4 | 218.0 | 224.8 | 228.5 | 237.1 | 238.8 |
| | 10 | F | 232.4 | 232.5 | 233.0 | 238.3 | 241.9 | 256.7 | 256.9 |
| B-1 | 1 | M | 301.8 | 318.2 | 327.0 | 336.8 | 346.4 | 365.7 | 359.9 |
| Anatabine | 2 | M | 278 | 293.8 | 302.0 | 303.4 | 312.9 | 331.0 | 320.0 |
| 0.1 mg/kg | 3 | M | 284.3 | 297.6 | 309.0 | 310.0 | 319.9 | 343.8 | 341.9 |
| B-2 | 4 | M | 288.3 | 301.2 | 310.0 | 318.7 | 322.3 | 346.9 | 347.3 |
| | 5 | M | 219.6 | 300.7 | 311.0 | 317.4 | 330.4 | 348.8 | 360.9 |
| B-3 | 6 | F | 241.1 | 246.5 | 252.0 | 255.1 | 261.9 | 274.8 | 277.2 |
| | 7 | F | 235.5 | 226.4 | 232.0 | 239.9 | 234.5 | 238.5 | 246.3 |
| | 8 | F | 220.1 | 221.7 | 227.0 | 224.1 | 226.0 | 233.9 | 242.0 |
| B-4 | 9 | F | 218.8 | 222.1 | 223.0 | 236.0 | 232.5 | 245.6 | 240.0 |
| | 10 | F | 222.9 | 232.1 | 235.0 | 232.0 | 233.7 | 245.4 | 241.6 |
| C-1 | 1 | M | 282.4 | 294.5 | 300.0 | 307.6 | 317.2 | 334.6 | 327.6 |
| Anatabine | 2 | M | 287.6 | 302.9 | 310.6 | 319.8 | 331.9 | 339.8 | 325.4 |
| 0.75 mg/kg | 3 | M | 268.2 | 281.8 | 293.4 | 298.8 | 307.9 | 329.4 | 322.5 |
| C-2 | 4 | M | 268.2 | 274.1 | 284.2 | 296.2 | 303.8 | 316.1 | 324.8 |
| | 5 | M | 281.3 | 287.1 | 292.0 | 298.5 | 306.8 | 321.9 | 331.1 |
| C-3 | 6 | F | 224.4 | 227.4 | 232.0 | 237.3 | 239.3 | 250.9 | 249.7 |
| | 7 | F | 217.8 | 223 | 220.0 | 226.3 | 232.2 | 239.1 | 233.5 |
| | 8 | F | 236.2 | 243.2 | 243.0 | 247.6 | 252.7 | 268.8 | 259.6 |
| C-4 | 9 | F | 214.7 | 222 | 224.6 | 229.1 | 230.9 | 240.9 | 241.6 |
| | 10 | F | 221.4 | 219.3 | 227.4 | 230.6 | 233.5 | 244.1 | 247.9 |
| D-1 | 1 | M | 279.1 | 292 | 300.0 | 302.1 | 317.1 | 333.0 | 341.1 |
| Anatabine | 2 | M | 296.9 | 305.6 | 319.0 | 323.1 | 331.8 | 359.6 | 360.3 |
| 1.5 mg/kg | 3 | M | 280.4 | 288.2 | 298.4 | 303.8 | 318.3 | 335.4 | 327.9 |
| D-2 | 4 | M | 302.3 | 316.7 | 326.0 | 337.3 | 347.0 | 369.5 | 380.4 |
| | 5 | M | 274.8 | 283.5 | 297.0 | 301.9 | 310.0 | 332.6 | 336.3 |
| D-3 | 6 | F | 221.2 | 229 | 229.6 | 232.8 | 232.8 | 242.1 | 236.9 |
| | 7 | F | 204.6 | 208.1 | 216.4 | 218.7 | 229.0 | 233.0 | 227.4 |
| | 8 | F | 229.3 | 235.7 | 244.0 | 243.9 | 252.1 | 259.7 | 257.1 |
| D-4 | 9 | F | 246 | 249.9 | 256.7 | 258.3 | 260.9 | 266.9 | 276.3 |
| | 10 | F | 216.9 | 224 | 225.0 | 225.8 | 234.5 | 243.5 | 240.9 |

TABLE 28C
Dosing Calculations and Body Weights, days 13-14 (anatabine)

| | | | day 13 | day 14 |
|---|---|---|---|---|
| Group | Rat | M/F | B.W. (g) | B.W. (g) |
| A-1 | 1 | M | 379.1 | 389.0 |
| vehicle | 2 | M | 349 | 363.0 |
| 5 mL/kg | 3 | M | 356 | 377.0 |
| A-2 | 4 | M | 317 | 325.0 |
| | 5 | M | 356 | 371.9 |
| A-3 | 6 | F | 258 | 265.2 |
| | 7 | F | 249.8 | 268.2 |
| | 8 | F | 245.6 | 252.7 |
| A-4 | 9 | F | 235 | 245.8 |
| | 10 | F | 253 | 265.7 |
| B-1 | 1 | M | 367 | 391.0 |
| Anatabine | 2 | M | 331 | 347.3 |
| 0.1 mg/kg | 3 | M | 349 | 368.8 |
| B-2 | 4 | M | 334.4 | 353.5 |
| | 5 | M | 360 | 373.8 |
| B-3 | 6 | F | 271.8 | 272.7 |
| | 7 | F | 244 | 248.5 |
| | 8 | F | 241 | 243.9 |
| B-4 | 9 | F | 242.1 | 255.6 |
| | 10 | F | 244 | 239.2 |
| C-1 | 1 | M | 335.6 | 351.9 |
| Anatabine | 2 | M | 338 | 357.9 |
| 0.75 mg/kg | 3 | M | 330 | 345.1 |
| C-2 | 4 | M | 326 | 339 |
| | 5 | M | 326 | 335.9 |
| C-3 | 6 | F | 248 | 251.5 |
| | 7 | F | 237.2 | 242.8 |
| | 8 | F | 268 | 274.1 |
| C-4 | 9 | F | 234 | 250.7 |
| | 10 | F | 243 | 250.6 |
| D-1 | 1 | M | 339.8 | 359.8 |
| Anatabine | 2 | M | 370 | 389.7 |
| 1.5 mg/kg | 3 | M | 340 | 357.1 |
| D-2 | 4 | M | 379 | 399.9 |
| | 5 | M | 337 | 355 |
| D-3 | 6 | F | 242.4 | 249.9 |
| | 7 | F | 223 | 224.4 |
| | 8 | F | 257 | 264.7 |
| D-4 | 9 | F | 275.8 | 283.1 |
| | 10 | F | 240.3 | 248.8 |

TABLE 28D
Dosing Calculations and Body Weights, days 1-5 (nicotine)

| | | | | Dose | | day 1 | day 2 | day 3 | day 4 | day 5 |
|---|---|---|---|---|---|---|---|---|---|---|
| | | | | volume | | B.W. | B.W. | B.W. | B.W. | B.W. |
| Group | Rat | M/F | B.W. (g) | (mL) | time | (g) | (g) | (g) | (g) | (g) |
| E-1 | 1 | M | 218 | 1.1 | 8:45 | 228 | 239.1 | 245.1 | 263.4 | 269.1 |
| Nicotine | 2 | M | 218 | 1.1 | 8:48 | 223 | 229.5 | 241.1 | 257.6 | 260.7 |
| 0.75 mg/kg | 3 | M | 207 | 1.03 | 8:50 | 219 | 229.1 | 238.4 | 252.9 | 258.7 |
| E-2 | 4 | M | 214 | 1.08 | 8:51 | | | | | |
| | 5 | M | 221 | 1.1 | 8:53 | 227 | 234.9 | 243 | 260.5 | 272.1 |
| E-3 | 6 | F | 187 | 0.93 | 8:55 | 191.6 | 190.5 | 198 | 207.8 | 212.3 |
| | 7 | F | 195 | 0.98 | 8:57 | | | | | |
| | 8 | F | 193 | 0.98 | 8:59 | | | | | |
| E-4 | 9 | F | 207 | 1.05 | 9:01 | 206.1 | 213.4 | 217.1 | 223.8 | 226.3 |
| | 10 | F | 192 | 0.95 | 9:03 | 192.8 | 195.7 | 199.1 | 205.6 | 206.4 |

TABLE 28E
Dosing Calculations and Body Weights, days 6-12 (nicotine)

| | | | | | | | day | day | day |
|---|---|---|---|---|---|---|---|---|---|
| | | | day 6 | day 7 | day 8 | day 9 | 10 | 11 | 12 |
| | | M/ | B.W. | B.W. | B.W. | B.W. | B.W. | B.W. | B.W. |
| Group | Rat | F | (g) | (g) | (g) | (g) | (g) | (g) | (g) |
| E-1 | 1 | M | 270 | 288 | 285.6 | 285.6 | 301 | 300.1 | 303 |
| Nicotine | 2 | M | 262.8 | 277.8 | 278 | 274.6 | 283.4 | 291.5 | 287.3 |
| 0.75 mg/kg | 3 | M | 259 | 279.9 | 284 | 274.9 | 286.1 | 292.2 | 290.7 |
| E-2 | 4 | M | | | | | | | |
| | 5 | M | 275.4 | 295.8 | 293.7 | 303.5 | 314.2 | 317.7 | 329.5 |
| E-3 | 6 | F | 207 | 213 | 218 | 219.1 | 221.8 | 222.8 | 232.1 |
| | 7 | F | | | | | | | |
| | 8 | F | | | | | | | |
| E-4 | 9 | F | 228.9 | 240.1 | 237.4 | 236.4 | 240.1 | 244.5 | 248.9 |
| | 10 | F | 205.5 | 218.3 | 217.4 | 213.2 | 220.5 | 226 | 227.7 |

TABLE 28F
Dosing Calculations and Body Weights, days 13-14 (nicotine)

| | | | day 13 | day 14 |
|---|---|---|---|---|
| Group | Rat | M/F | B.W. (g) | B.W. (g) |
| E-1 | 1 | M | 312.3 | 316.1 |
| Nicotine | 2 | M | 292.4 | 297.6 |
| 0.75 mg/kg | 3 | M | 299.4 | 305 |
| E-2 | 4 | M | | |
| | 5 | M | 330 | 339.2 |
| E-3 | 6 | F | 232.6 | 230.4 |
| | 7 | F | | |
| | 8 | F | | |
| E-4 | 9 | F | 251.8 | 249 |
| | 10 | F | 227.3 | 229 |

TABLE 29A
Average Daily Food Consumption per Rat (grams)

| | | | | | | | Anatabine | | | |
|---|---|---|---|---|---|---|---|---|---|---|
| | | | A - Vehicle | | B - 0.1 mg/kg | | C - 0.75 mg/kg | | D - 1.5 mg/kg | |
| Date | cage# | # of rats | Male | Female | Male | Female | Male | Female | Male | Female |
| day 1 | 1 | 3 | 25.1 | 14.1 | 25.8 | 16.4 | 23.9 | 17.1 | 22.7 | 16.1 |
| | 2 | 2 | 21.1 | 15.4 | 28.0 | 14.4 | 19.2 | 17.1 | 21.4 | 13.6 |
| day 2 | 1 | 3 | 27.4 | 16.3 | 33.8 | 20.2 | 26.3 | 22.5 | 24.2 | 20.5 |
| | 2 | 2 | 24.5 | 20.4 | 29.1 | 20.4 | 29.5 | 11.3 | 30.2 | 19.8 |
| day 3 | 1 | 3 | 25.4 | 16.8 | 24.8 | 19.4 | 28.1 | 21.0 | 30.1 | 19.6 |
| | 2 | 2 | 25.3 | 20.3 | 22.0 | 18.2 | 26.3 | 17.4 | 31.2 | 20.9 |
| day 4 | 1 | 3 | 26.8 | 17.6 | 34.1 | 23.2 | 28.3 | 21.0 | 22.1 | 20.8 |
| | 2 | 2 | 26.4 | 20.2 | 28.4 | 18.0 | 29.8 | 18.7 | 30.0 | 21.6 |
| day 5 | 1 | 3 | 28.5 | 17.2 | 30.6 | 22.4 | 24.4 | 19.1 | 30.0 | 18.5 |
| | 2 | 2 | 28.3 | 22.4 | 28.6 | 21.4 | 27.0 | 21.7 | 29.0 | 18.5 |
| day 6 | 1 | 3 | 26.7 | 20.6 | 33.6 | 26.8 | 25.6 | 18.8 | 33.6 | 14.2 |
| | 2 | 2 | 26.0 | 19.2 | 28.0 | 18.2 | 29.5 | 16.4 | 35.4 | 18.4 |
| day 7 | 1 | 3 | 30.1 | 21.6 | 35.7 | 21.7 | 29.0 | 24.0 | 34.1 | 21.2 |
| | 2 | 2 | 27.9 | 20.5 | 31.8 | 22.4 | 31.5 | 17.4 | 33.5 | 28.8 |
| day 8 | 1 | 3 | 33.2 | 23.7 | 33.2 | 26.5 | 31.3 | 23.2 | 31.7 | 22.7 |
| | 2 | 2 | 31.5 | 23.8 | 31.9 | 21.3 | 32.6 | 22.8 | 34.4 | 19.8 |
| day 9 | 1 | 3 | 29.1 | 24.1 | 35.5 | 21.1 | 29.1 | 23.7 | 33.9 | 21.7 |
| | 2 | 2 | 27.7 | 24.9 | 36.8 | 23.6 | 30.3 | 23.0 | 34.5 | 23.1 |
| day 10 | 1 | 3 | 30.4 | 22.3 | 34.7 | 20.4 | 28.7 | 20.0 | 33.5 | 21.7 |
| | 2 | 2 | 29.4 | 20.5 | 26.7 | 16.9 | 28.0 | 21.0 | 32.4 | 21.7 |
| day 11 | 1 | 3 | 28.0 | 19.9 | 29.1 | 19.0 | 24.4 | 20.5 | 28.3 | 15.6 |
| | 2 | 2 | 24.4 | 22.9 | 30.6 | 19.1 | 26.7 | 18.6 | 29.9 | 19.0 |
| day 12 | 1 | 3 | 27.4 | 20.3 | 31.6 | 27.9 | 21.7 | 16.6 | 30.1 | 18.4 |
| | 2 | 2 | 25.6 | 18.8 | 34.9 | 16.7 | 31.0 | 20.4 | 33.1 | 20.8 |
| day 13 | 1 | 3 | 33.6 | 22.4 | 39.9 | 29.6 | 33.9 | 22.0 | 36.8 | 19.6 |
| | 2 | 2 | 30.8 | 20.9 | 27.1 | 19.3 | 27.2 | 17.7 | 34.7 | 20.5 |
| day 14 | 1 | 3 | 28.7 | 20.9 | 32.3 | 18.8 | 27.8 | 17.6 | 29.9 | 17.1 |
| | 2 | 2 | 26.4 | 19.6 | 28.8 | 19.1 | 26.4 | 20.7 | 31.4 | 19.2 |

TABLE 29B
Average Daily Food Consumption per Rat (grams)

| E- Nicotine 0.75 mg/kg | |
|---|---|
| Males | Female |

| Date | cage# | # of rats | grams | # of rats | grams |
|---|---|---|---|---|---|
| day 1 | 1 | 3 | 20.8 | 1 | 23.7 |
| | 2 | 1 | 19.3 | 2 | 14.8 |
| day 2 | 1 | 3 | 23.5 | 1 | 24.9 |
| | 2 | 1 | 26.2 | 2 | 18.4 |
| day 3 | 1 | 3 | 26.6 | 1 | 22.2 |
| | 2 | 1 | 26.2 | 2 | 22.3 |
| day 4 | 1 | 3 | 26.2 | 1 | 19.4 |
| | 2 | 1 | 23.7 | 2 | 15.8 |
| day 5 | 1 | 3 | 25.1 | 1 | 18.7 |
| | 2 | 1 | 21.9 | 2 | 16.2 |
| day 6 | 1 | 3 | 23.8 | 1 | 20.0 |
| | 2 | 1 | 25.9 | 2 | 17.2 |
| day 7 | 1 | 3 | 26.5 | 1 | 19.4 |
| | 2 | 1 | 27.9 | 2 | 21.7 |
| day 8 | 1 | 3 | 24.0 | 1 | 20.1 |
| | 2 | 1 | 28.2 | 2 | 20.6 |
| day 9 | 1 | 3 | 27.3 | 1 | 24.4 |
| | 2 | 1 | 26.1 | 2 | 21.5 |
| day 10 | 1 | 3 | 29.6 | 1 | 22.6 |
| | 2 | 1 | 30.9 | 2 | 20.4 |
| day 11 | 1 | 3 | 30.1 | 1 | 20.4 |
| | 2 | 1 | 29.1 | 2 | 20.3 |
| day 12 | 1 | 3 | 26.5 | 1 | 22.5 |
| | 2 | 1 | 23.4 | 2 | 21.7 |
| day 13 | 1 | 3 | 31.9 | 1 | 23.7 |
| | 2 | 1 | 28.4 | 2 | 21.2 |
| day 14 | 1 | 3 | 32.7 | 1 | 24.4 |
| | 2 | 1 | 26.4 | 2 | 19.6 |

TABLE 30
Hematology/Coagulation Parameters: Normal Ranges in the Rat

| | Unit of Measure | Range |
|---|---|---|
| WBC | ×10³/μL | 3.0-17.0 |
| RBC | ×10⁶/μL | 5-10 |
| RGB | g/dL | 11-19 |
| HCT | % | 35-57 |
| MCV | fL | 46-65 |
| MCH | pg | 18-23 |
| MCHC | g/dL | 31-40 |
| RETICULOCYTE | % | 0-25 |
| COUNT | | |
| NEUTROPHIL SEG | % | 7-15 |
| LYMPHOCYTE | % | 77-89 |
| MONOCYTE | % | 0-5 |
| EOSINOPHIL | % | 0-4 |
| BASOPHIL | % | 0-1 |
| PLATELET COUNT | ×10³/μL | 200-1500 |
| aPTT | sec | 13.2-22.4 |
| PT | sec | 11.0-15.6 |

TABLE 31
Hematology Parameters

| | | | Hematology Parameters | | | | | | | | |
|---|---|---|---|---|---|---|---|---|---|---|---|
| | | Anatabine | | | | | | | | RETICULOCYTE | PLATELET |
| Animal | | Dose | WBC | RBC | HGB | HCT | MCV | MCH | MCHC | COUNT | COUNT |
| ID | Sex | (mg/kg) | ×10³/μL | ×10⁶/μL | gm/dL | % | U³ | UUG | % | % | ×10³/μL |
| A1 | M | 0 | 9.4 | 7.33 | 14.5 | 43.6 | 60 | 19.7 | 33.1 | 4.6 | 1494 |
| A2 | M | 0 | 11.8 | 7.22 | 15.1 | 44.9 | 62 | 20.9 | 33.6 | 6.1 | 1124 |
| A3 | M | 0 | 12.2 | 7.26 | 15.4 | 45.5 | 63 | 21.2 | 33.9 | 6.8 | 1088 |
| A4 | M | 0 | 9.9 | 7.69 | 15.8 | 47.8 | 62 | 20.5 | 33 | 5.9 | TNP¹ |
| A5 | M | 0 | 9.4 | 7.13 | 15.2 | 45.4 | 64 | 21.3 | 33.4 | 7.3 | 1443 |
| A6 | F | 0 | 5.8 | 8.4 | 18.3 | 51 | 61 | 21.7 | 35.8 | 5.6 | 1500 |
| A7 | F | 0 | 11.6 | 7.2 | 15.6 | 45 | 63 | 21.6 | 34.6 | 3.4 | 1057 |
| A8 | F | 0 | 12 | 7.35 | 15.2 | 44.3 | 60 | 20.6 | 34.2 | 6.3 | 1263 |
| A9 | F | 0 | 7 | 8.71 | 18.1 | 53.5 | 61 | 20.8 | 33.8 | 4.7 | 986 |
| A10 | F | 0 | 10.1 | 7.16 | 14.6 | 42.7 | 60 | 20.4 | 34.3 | 5.2 | 1186 |
| B1 | M | 0.1 | 23.5 | 8.05 | 17.6 | 52.1 | 65 | 21.9 | 33.8 | 6.1 | 701 |
| B2 | M | 0.1 | 15 | 7.38 | 15.5 | 45.7 | 62 | 21.1 | 34 | 5.4 | 1339 |
| B3 | M | 0.1 | 11.1 | 7.4 | 16.1 | 47.5 | 64 | 21.8 | 34 | 4.7 | 1601 |
| B4 | M | 0.1 | 13.2 | 7.42 | 15.2 | 45 | 61 | 20.5 | 33.8 | 5.6 | 1107 |
| B5 | M | 0.1 | 15.4 | 6.96 | 14.5 | 45.3 | 65 | 20.8 | 32 | 5.9 | 1495 |
| B6 | F | 0.1 | 4.7 | 7.7 | 15.8 | 46.7 | 61 | 20.5 | 33.8 | 4.8 | 1145 |
| B7 | F | 0.1 | 10.7 | 8.71 | 17 | 51.3 | 59 | 19.6 | 33.2 | 5.4 | TNP¹ |
| B8 | F | 0.1 | 8 | 7.1 | 14.8 | 44 | 62 | 20.9 | 33.6 | 6.1 | TNP¹ |
| B9 | F | 0.1 | 14 | 7.06 | 14.6 | 43.1 | 61 | 20.8 | 34 | 3.7 | 1029 |
| B10 | F | 0.1 | 9.2 | 8.22 | 15.8 | 46.8 | 57 | 19.2 | 33.7 | 2.4 | 818 |
| C1 | M | 0.75 | 9.9 | 8.23 | 16.8 | 49.6 | 60 | 20.3 | 33.8 | 3.6 | 1446 |
| C2 | M | 0.75 | 11.1 | 7.51 | 15.8 | 45.5 | 61 | 21 | 34.7 | 4.2 | 1478 |
| C3 | M | 0.75 | 22.1 | 7.63 | 16.8 | 48.4 | 63 | 22 | 34.7 | 4.1 | 1327 |
| C4 | M | 0.75 | 7.5 | 6.23 | 13.1 | 39.2 | 63 | 21.1 | 33.5 | 4.7 | TNP¹ |
| C5 | M | 0.75 | 9.9 | 7.73 | 15.4 | 46.3 | 60 | 19.9 | 33.2 | 5.3 | 1052 |
| C6 | F | 0.75 | 13.5 | 7.42 | 15 | 44.7 | 60 | 20.2 | 33.6 | 3.4 | 653 |
| C7 | F | 0.75 | 14.5 | 7.78 | 14.9 | 43.9 | 56 | 19.2 | 34.1 | 1.8 | 1475 |
| C8 | F | 0.75 | 10.9 | 7.11 | 15.1 | 44.2 | 62 | 21.2 | 34.2 | 3.2 | 1304 |
| C9 | F | 0.75 | 8 | 7.58 | 15.6 | 45.4 | 60 | 20.5 | 34.3 | 3.8 | 1540 |
| C10 | F | 0.75 | 4.8 | 7.81 | 16.2 | 47.6 | 61 | 20.8 | 34.1 | 4.1 | 1376 |
| D1 | M | 1.5 | 12.4 | 7.82 | 15.8 | 48 | 62 | 20.2 | 32.9 | 4.4 | TNP2 |
| D2 | M | 1.5 | 15.3 | 7.43 | 15.4 | 46.1 | 62 | 20.7 | 33.4 | 3.3 | 1350 |
| D3 | M | 1.5 | 8.4 | 7.49 | 16.1 | 48.5 | 65 | 21.5 | 33.1 | 3.6 | 1407 |
| D4 | M | 1.5 | 13 | 7.16 | 14.6 | 43.1 | 60 | 20.4 | 33.8 | 5.1 | 1274 |
| D5 | M | 1.5 | 16.4 | 7.37 | 15 | 44.8 | 61 | 20.4 | 33.6 | 4.8 | 859 |
| D6 | F | 1.5 | 6.4 | 8.17 | 15.8 | 47.1 | 58 | 19.3 | 33.6 | 3.2 | 1399 |
| D7 | F | 1.5 | 7 | 7.77 | 15.1 | 45.9 | 59 | 19.4 | 32.8 | 2.8 | 1346 |
| D8 | F | 1.5 | 12.1 | 7.65 | 14.8 | 44.3 | 58 | 19.3 | 33.4 | 4.6 | 1312 |
| D9 | F | 1.5 | 10.9 | 8.01 | 15.9 | 48.2 | 60 | 19.8 | 32.9 | 3.4 | 228 |
| D10 | F | 1.5 | 9.5 | 7.05 | 14.2 | 41.4 | 59 | 20.2 | 34.4 | 2.1 | 1352 |
| E1 | M | 0.75¹ | 9.2 | 7.43 | 15.2 | 45.6 | 61 | 20.5 | 33.4 | 4.3 | 1458 |
| E2 | M | 0.75¹ | 13.6 | 7.61 | 15.9 | 46.2 | 61 | 20.8 | 34.4 | 4.8 | 1343 |
| E3 | M | 0.75¹ | 8.2 | 8.26 | 16.8 | 50 | 61 | 20.4 | 33.7 | 5.6 | 1366 |
| E5 | M | 0.75¹ | 14.6 | 7.04 | 15.3 | 44.5 | 63 | 21.7 | 34.3 | 6.1 | 1256 |
| E6 | F | 0.75¹ | 11.2 | 8.25 | 16.2 | 47.7 | 58 | 19.6 | 34 | 5.8 | 1539 |
| E9 | F | 0.75¹ | 6.7 | 7.41 | 15.2 | 44.2 | 60 | 20.5 | 34.4 | 4.9 | 1187 |
| E10 | F | 0.75¹ | 12.7 | 8.36 | 16.5 | 48.3 | 58 | 19.7 | 34.1 | 4.5 | TNP² |

| | | | Hematology Parameters | | | | | | | |
|---|---|---|---|---|---|---|---|---|---|---|
| Ani- | | Anatabine | NEUTROPHIL | LYM- | | | | | | |
| mal | | Dose | SEG | PHOCYTE | MONOCYTE | EOSINOPHIL | BASOPHIL | PLATELET | POLY- | ANISO- |
| ID | Sex | (mg/kg) | % | % | % | % | % | EST | CHROMASIA | CYTOSIS |
| A1 | M | 0 | 9 | 90 | 1 | 0 | 0 | ADEQUATE | SLIGHT | SLIGHT |
| A2 | M | 0 | 9 | 87 | 4 | 0 | 0 | ADEQUATE | SLIGHT | SLIGHT |
| A3 | M | 0 | 12 | 88 | 0 | 0 | 0 | ADEQUATE | SLIGHT | SLIGHT |
| A4 | M | 0 | 6 | 93 | 1 | 0 | 0 | DECREASED | SLIGHT | SLIGHT |
| A5 | M | 0 | 8 | 90 | 2 | 0 | 0 | ADEQUATE | SLIGHT | SLIGHT |
| A6 | F | 0 | 9 | 89 | 2 | 0 | 0 | INCREASED | SLIGHT | SLIGHT |
| A7 | F | 0 | 12 | 86 | 2 | 0 | 0 | ADEQUATE | SLIGHT | SLIGHT |
| A8 | F | 0 | 14 | 84 | 2 | 0 | 0 | ADEQUATE | DNR | DNR |
| A9 | F | 0 | 12 | 86 | 2 | 0 | 0 | ADEQUATE | SLIGHT | SLIGHT |
| A10 | F | 0 | 16 | 81 | 3 | 0 | 0 | ADEQUATE | SLIGHT | SLIGHT |
| B1 | M | 0.1 | 12 | 87 | 1 | 0 | 0 | ADEQUATE | SLIGHT | SLIGHT |
| B2 | M | 0.1 | 3 | 94 | 3 | 0 | 0 | ADEQUATE | SLIGHT | SLIGHT |
| B3 | M | 0.1 | 7 | 90 | 3 | 0 | 0 | INCREASED | SLIGHT | SLIGHT |
| B4 | M | 0.1 | 10 | 87 | 2 | 1 | 0 | ADEQUATE | SLIGHT | SLIGHT |
| B5 | M | 0.1 | 11 | 88 | 1 | 0 | 0 | ADEQUATE | SLIGHT | SLIGHT |
| B6 | F | 0.1 | 8 | 89 | 3 | 0 | 0 | ADEQUATE | SLIGHT | SLIGHT |
| B7 | F | 0.1 | 8 | 90 | 2 | 0 | 0 | ADEQUATE | SLIGHT | SLIGHT |
| B8 | F | 0.1 | 7 | 91 | 2 | 0 | 0 | DECREASED | SLIGHT | SLIGHT |
| B9 | F | 0.1 | 6 | 93 | 1 | 0 | 0 | ADEQUATE | SLIGHT | SLIGHT |
| B10 | F | 0.1 | 11 | 88 | 1 | 0 | 0 | ADEQUATE | SLIGHT | SLIGHT |
| C1 | M | 0.75 | 13 | 86 | 1 | 0 | 0 | ADEQUATE | SLIGHT | SLIGHT |
| C2 | M | 0.75 | 10 | 89 | 1 | 0 | 0 | ADEQUATE | SLIGHT | SLIGHT |
| C3 | M | 0.75 | 6 | 92 | 2 | 0 | 0 | ADEQUATE | SLIGHT | SLIGHT |
| C4 | M | 0.75 | 10 | 89 | 1 | 0 | 0 | DECREASED | SLIGHT | SLIGHT |
| C5 | M | 0.75 | 10 | 88 | 2 | 0 | 0 | ADEQUATE | DNR | DNR |
| C6 | F | 0.75 | 6 | 93 | 1 | 0 | 0 | ADEQUATE | SLIGHT | SLIGHT |
| C7 | F | 0.75 | 12 | 87 | 1 | 0 | 0 | ADEQUATE | SLIGHT | SLIGHT |
| C8 | F | 0.75 | 9 | 91 | 0 | 0 | 0 | ADEQUATE | SLIGHT | SLIGHT |
| C9 | F | 0.75 | 9 | 89 | 2 | 0 | 0 | INCREASED | SLIGHT | SLIGHT |
| C10 | F | 0.75 | 7 | 93 | 0 | 0 | 0 | ADEQUATE | SLIGHT | SLIGHT |
| D1 | M | 1.5 | 20 | 77 | 3 | 0 | 0 | DECREASED | SLIGHT | SLIGHT |
| D2 | M | 1.5 | 6 | 93 | 1 | 0 | 0 | ADEQUATE | SLIGHT | SLIGHT |
| D3 | M | 1.5 | 12 | 86 | 2 | 0 | 0 | ADEQUATE | SLIGHT | SLIGHT |
| D4 | M | 1.5 | 25 | 74 | 1 | 0 | 0 | ADEQUATE | SLIGHT | SLIGHT |
| D5 | M | 1.5 | 10 | 89 | 1 | 0 | 0 | ADEQUATE | SLIGHT | SLIGHT |
| D6 | F | 1.5 | 11 | 88 | 1 | 0 | 0 | ADEQUATE | SLIGHT | SLIGHT |
| D7 | F | 1.5 | 13 | 87 | 0 | 0 | 0 | ADEQUATE | SLIGHT | SLIGHT |
| D8 | F | 1.5 | 20 | 80 | 0 | 0 | 0 | ADEQUATE | SLIGHT | SLIGHT |
| D9 | F | 1.5 | 10 | 89 | 1 | 0 | 0 | ADEQUATE | SLIGHT | SLIGHT |
| D10 | F | 1.5 | 9 | 89 | 2 | 0 | 0 | ADEQUATE | SLIGHT | SLIGHT |
| E1 | M | 0.75¹ | 12 | 86 | 2 | 0 | 0 | ADEQUATE | SLIGHT | SLIGHT |
| E2 | M | 0.75¹ | 11 | 87 | 2 | 0 | 0 | ADEQUATE | SLIGHT | SLIGHT |
| E3 | M | 0.75¹ | 16 | 80 | 4 | 0 | 0 | ADEQUATE | SLIGHT | SLIGHT |
| E5 | M | 0.75¹ | 8 | 90 | 2 | 0 | 0 | ADEQUATE | SLIGHT | SLIGHT |
| E6 | F | 0.75¹ | 15 | 83 | 2 | 0 | 0 | INCREASED | SLIGHT | SLIGHT |
| E9 | F | 0.75¹ | 12 | 87 | 1 | 0 | 0 | ADEQUATE | SLIGHT | SLIGHT |
| E10 | F | 0.75¹ | 11 | 89 | 0 | 0 | 0 | DECREASED | SLIGHT | SLIGHT |

¹Dose is 0.75 mg/kg nicotine
²TNP: Test not performed due to clot in EDTA tube
DNR: Did not report - insufficient sample

TABLE 32
Clinical Chemistry Parameters: Normal Ranges in the Rat

| | Unit of Measure | Range |
|---|---|---|
| ALKALINE | IU/L | 160-500 |
| PHOSPHATASE | | |
| ALT (SGPT) | IU/L | 35-80 |
| AST (SGOT) | IU/L | 33-53 |
| GLOBULIN | g/dL | 1.4-5.0 |
| ALBUMIN | g/dL | 2.9-5.9 |
| TOTAL PROTEIN | g/dL | 4.5-8.4 |
| TOTAL BILIRUBIN | mg/dL | 0-0.64 |
| BLOOD UREA | mg/dL | 11-23 |
| NITROGEN (BUN) | | |
| CREATININE | mg/dL | 0.4-3.8 |
| CHOLESTEROL | mg/dL | 35-75 |
| GLUCOSE | mg/dL | 80-300 |
| CALCIUM | mg/dL | 9.1-15.1 |
| PHOSPHORUS | mg/dL | 4.7-16.0 |
| CHLORIDE | mEq/L | 79-111 |
| POTASSIUM | mEq/L | 3.6-9.2 |
| SODIUM | mEq/L | 142-154 |

TABLE 33
Clinical Chemistry Parameters

| | | | Clinical Chemistry Parameters | | | | | | | | |
|---|---|---|---|---|---|---|---|---|---|---|---|
| Ani- | | Anatabine | ALK | | | | TOT | | TOT | DIR | |
| mal | | Dose | PHOSPHATASE | ALT | AST | ALBUMIN | PROTEIN | GLOBULIN | BILIRUBIN | BILIRUBIN | BUN |
| ID | Sex | (mg/kg) | IU/L | IU/L | IU/L | g/dL | g/dL | g/dL | mg/dL | mg/dL | mg/dL |
| A1 | M | 0 | 463 | 62 | 85 | 3.1 | 5.6 | 2.5 | 0 | 0 | 19 |
| A2 | M | 0 | 398 | 59 | 78 | 3.1 | 5.6 | 2.5 | 0 | 0 | 17 |
| A3 | M | 0 | 275 | 61 | 96 | 3.1 | 5.6 | 2.5 | 0 | 0 | 19 |
| A4 | M | 0 | 393 | 57 | 137 | 3.2 | 5.7 | 2.5 | 0 | 0 | 18 |
| A5 | M | 0 | 325 | 58 | 99 | 3.1 | 5.6 | 2.5 | 0 | 0 | 20 |
| A6 | F | 0 | 248 | 55 | 71 | 3.4 | 6.4 | 3 | 0 | 0 | 17 |
| A7 | F | 0 | 265 | 61 | 81 | 3.3 | 6.2 | 2.9 | 0 | 0 | 14 |
| A8 | F | 0 | 292 | 62 | 75 | 3.4 | 6.2 | 2.8 | 0 | 0 | 20 |
| A9 | F | 0 | 176 | 52 | 77 | 3.5 | 6.2 | 2.7 | 0 | 0 | 15 |
| A10 | F | 0 | 170 | 61 | 100 | 3.4 | 6.2 | 2.8 | 0 | 0 | 25 |
| B1 | M | 0.1 | 470 | 92 | 97 | 3.4 | 6.3 | 2.9 | 0 | 0 | 18 |
| B2 | M | 0.1 | 227 | 65 | 99 | 3.2 | 5.8 | 2.6 | 0 | 0 | 18 |
| B3 | M | 0.1 | 364 | 74 | 93 | 3.1 | 5.9 | 2.8 | 0 | 0 | 21 |
| B4 | M | 0.1 | 358 | 44 | 85 | 3.1 | 6.2 | 3.1 | 0 | 0 | 14 |
| B5 | M | 0.1 | 618 | 78 | 106 | 3.4 | 6.3 | 2.9 | 0 | 0 | 19 |
| B6 | F | 0.1 | 261 | 57 | 78 | 3.6 | 6.7 | 3.1 | 0 | 0 | 21 |
| B7 | F | 0.1 | 140 | 43 | 69 | 3.5 | 6.3 | 2.8 | 0 | 0 | 19 |
| B8 | F | 0.1 | 209 | 47 | 70 | 3.6 | 6.7 | 3.1 | 0 | 0 | 21 |
| B9 | F | 0.1 | 412 | 62 | 86 | 3.8 | 6.9 | 3.1 | 0 | 0 | 19 |
| B10 | F | 0.1 | 220 | 39 | 82 | 3.6 | 6.6 | 3 | 0 | 0 | 19 |
| C1 | M | 0.75 | 479 | 75 | 103 | 3.4 | 6.2 | 2.8 | 0 | 0 | 15 |
| C2 | M | 0.75 | 297 | 57 | 66 | 3.1 | 5.7 | 2.6 | 0 | 0 | 18 |
| C3 | M | 0.75 | 325 | 65 | 97 | 3.2 | 5.9 | 2.7 | 0 | 0 | 12 |
| C4 | M | 0.75 | 337 | 50 | 90 | 3.3 | 6.1 | 2.8 | 0 | 0 | 17 |
| C5 | M | 0.75 | 416 | 87 | 92 | 3.4 | 6.3 | 2.9 | 0.1 | 0.1 | 21 |
| C6 | F | 0.75 | 123 | 63 | 95 | 3.5 | 6.3 | 2.8 | 0 | 0 | 15 |
| C7 | F | 0.75 | 142 | 49 | 69 | 3.6 | 6.3 | 2.7 | 0 | 0 | 16 |
| C8 | F | 0.75 | 235 | 55 | 76 | 3.4 | 6.2 | 2.8 | 0 | 0 | 15 |
| C9 | F | 0.75 | 130 | 49 | 54 | 3.6 | 6.6 | 3 | 0 | 0 | 16 |
| C10 | F | 0.75 | 160 | 57 | 72 | 3.5 | 6.5 | 3 | 0 | 0 | 15 |
| D1 | M | 1.5 | 389 | 107 | 144 | 3.4 | 6.2 | 2.8 | 0 | 0 | 18 |
| D2 | M | 1.5 | 644 | 61 | 83 | 3.2 | 5.7 | 2.5 | 0 | 0 | 17 |
| D3 | M | 1.5 | 304 | 65 | 91 | 3.5 | 6.3 | 2.8 | 0 | 0 | 18 |
| D4 | M | 1.5 | 357 | 58 | 77 | 3.2 | 5.8 | 2.6 | 0 | 0 | 18 |
| D5 | M | 1.5 | 363 | 61 | 90 | 3.3 | 6 | 2.7 | 0 | 0 | 15 |
| D6 | F | 1.5 | 189 | 47 | 78 | 3.7 | 6.6 | 2.9 | 0 | 0 | 18 |
| D7 | F | 1.5 | 218 | 53 | 82 | 3.5 | 6.5 | 3 | 0 | 0 | 17 |
| D8 | F | 1.5 | 181 | 43 | 70 | 3.5 | 6.5 | 3 | 0 | 0 | 15 |
| D9 | F | 1.5 | 272 | 58 | 88 | 3.4 | 6.3 | 2.9 | 0.1 | 0.1 | 18 |
| D10 | F | 1.5 | 110 | 45 | 65 | 3.3 | 5.9 | 2.6 | 0 | 0 | 13 |
| E1 | M | 0.75¹ | 241 | 54 | 89 | 3.1 | 6 | 2.9 | 0.1 | 0 | 15 |
| E2 | M | 0.75¹ | 257 | 59 | 81 | 3.1 | 6.2 | 3.1 | 0 | 0 | 18 |
| E3 | M | 0.75¹ | 313 | 45 | 72 | 3.1 | 5.8 | 2.7 | 0 | 0 | 19 |
| E5 | M | 0.75¹ | 353 | 65 | 87 | 3 | 6 | 3 | 0.1 | 0 | 18 |
| E6 | F | 0.75¹ | 274 | 97 | 133 | 3.3 | 6.4 | 3.1 | 0.1 | 0 | 20 |
| E9 | F | 0.75¹ | 138 | 54 | 75 | 3.3 | 6.6 | 3.3 | 0.1 | 0.1 | 17 |
| E10 | F | 0.75¹ | 154 | 54 | 101 | 3.3 | 6.5 | 3.2 | 0.1 | 0 | 20 |

¹Dose is 0.75 mg/kg nicotine

| | | | Clinical Chemistry Parameters | | | | | | | | |
|---|---|---|---|---|---|---|---|---|---|---|---|
| | | Anatabine | | CHOLES- | | | PHOS- | | | | |
| Animal | | Dose | CREATININE | TEROL | GLUCOSE | CALCIUM | PHORUS | CHLORIDE | POTASSIUM | SODIUM | A/G |
| ID | Sex | (mg/kg) | mg/dL | mg/dL | mg/dL | mg/dL | mg/dL | mEq/L | mEq/L | mEq/L | RATIO |
| A1 | M | 0 | 0.3 | 54 | 183 | 12.3 | 10.9 | 97 | 6.8 | 144 | 1.2 |
| A2 | M | 0 | 0.4 | 62 | 215 | 12.1 | 11 | 97 | 6.1 | 145 | 1.2 |
| A3 | M | 0 | 0.4 | 60 | 217 | 12.2 | 10.8 | 99 | 6.2 | 147 | 1.2 |
| A4 | M | 0 | 0.3 | 65 | 302 | 12.4 | 11.4 | 100 | 6.3 | 145 | 1.3 |
| A5 | M | 0 | 0.4 | 59 | 193 | 11.6 | 10 | 100 | 6.1 | 146 | 1.2 |
| A6 | F | 0 | 0.4 | 85 | 205 | 11.9 | 10 | 100 | 5.5 | 145 | 1.1 |
| A7 | F | 0 | 0.4 | 72 | 176 | 11.3 | 9 | 99 | 5.5 | 146 | 1.1 |
| A8 | F | 0 | 0.4 | 56 | 206 | 11.4 | 9.1 | 100 | 5.8 | 146 | 1.2 |
| A9 | F | 0 | 0.4 | 64 | 197 | 11.6 | 8.9 | 100 | 6.7 | 146 | 1.3 |
| A10 | F | 0 | 0.5 | 60 | 207 | 12.1 | 8.3 | 100 | 6.1 | 145 | 1.2 |
| B1 | M | 0.1 | 0.4 | 65 | 258 | 12.7 | 10.3 | 98 | 5.9 | 148 | 1.2 |
| B2 | M | 0.1 | 0.4 | 64 | 219 | 11.8 | 10.3 | 97 | 6.5 | 144 | 1.2 |
| B3 | M | 0.1 | 0.4 | 75 | 214 | 11.9 | 11.2 | 99 | 6.6 | 146 | 1.1 |
| B4 | M | 0.1 | 0.3 | 56 | 194 | 11.8 | 11.5 | 97 | 6.9 | 147 | 1 |
| B5 | M | 0.1 | 0.3 | 76 | 210 | 11.8 | 10.8 | 99 | 6.1 | 146 | 1.2 |
| B6 | F | 0.1 | 0.4 | 90 | 224 | 12 | 8.5 | 99 | 5.4 | 147 | 1.2 |
| B7 | F | 0.1 | 0.5 | 74 | 170 | 11.4 | 8.2 | 102 | 6.3 | 148 | 1.3 |
| B8 | F | 0.1 | 0.5 | 65 | 227 | 11.9 | 7.4 | 102 | 5.2 | 148 | 1.2 |
| B9 | F | 0.1 | 0.4 | 66 | 220 | 12.4 | 8.5 | 97 | 5.5 | 145 | 1.2 |
| B10 | F | 0.1 | 0.4 | 87 | 197 | 10.9 | 7.8 | 101 | 6.7 | 146 | 1.2 |
| C1 | M | 0.75 | 0.4 | 69 | 211 | 12.3 | 11 | 98 | 6.3 | 148 | 1.2 |
| C2 | M | 0.75 | 0.4 | 60 | 222 | 12.2 | 10.7 | 99 | 6.3 | 146 | 1.2 |
| C3 | M | 0.75 | 0.3 | 54 | 219 | 11.8 | 10 | 100 | 5.4 | 147 | 1.2 |
| C4 | M | 0.75 | 0.3 | 62 | 160 | 11.5 | 10.6 | 99 | 6 | 150 | 1.2 |
| C5 | M | 0.75 | 0.4 | 69 | 198 | 12.4 | 12.3 | 97 | 6.6 | 148 | 1.2 |
| C6 | F | 0.75 | 0.5 | 66 | 237 | 11.8 | 9.7 | 97 | 5.5 | 145 | 1.3 |
| C7 | F | 0.75 | 0.4 | 64 | 247 | 11.3 | 7.7 | 96 | 5.9 | 144 | 1.3 |
| C8 | F | 0.75 | 0.4 | 63 | 192 | 11.6 | 9.6 | 99 | 6 | 145 | 1.2 |
| C9 | F | 0.75 | 0.4 | 69 | 211 | 12.1 | 9.2 | 97 | 5.7 | 144 | 1.2 |
| C10 | F | 0.75 | 0.5 | 87 | 217 | 11.6 | 7 | 101 | 6.3 | 147 | 1.2 |
| D1 | M | 1.5 | 0.4 | 57 | 209 | 12.2 | 10.9 | 99 | 7.2 | 146 | 1.2 |
| D2 | M | 1.5 | 0.4 | 68 | 204 | 11 | 9.6 | 100 | 5.4 | 149 | 1.3 |
| D3 | M | 1.5 | 0.4 | 64 | 207 | 12.2 | 11.6 | 99 | 6.3 | 148 | 1.3 |
| D4 | M | 1.5 | 0.3 | 64 | 196 | 12.1 | 11.6 | 100 | 5.3 | 149 | 1.2 |
| D5 | M | 1.5 | 0.3 | 59 | 212 | 11.9 | 11.3 | 101 | 5.9 | 147 | 1.2 |
| D6 | F | 1.5 | 0.4 | 68 | 196 | 11.8 | 8.4 | 102 | 6.1 | 148 | 1.3 |
| D7 | F | 1.5 | 0.4 | 78 | 173 | 11.6 | 8.5 | 100 | 6.3 | 147 | 1.2 |
| D8 | F | 1.5 | 0.4 | 81 | 192 | 11.3 | 7.2 | 101 | 5.2 | 148 | 1.2 |
| D9 | F | 1.5 | 0.4 | 67 | 193 | 11.2 | 8.6 | 98 | 5 | 147 | 1.2 |
| D10 | F | 1.5 | 0.4 | 56 | 188 | 10.9 | 8.5 | 103 | 6.1 | 146 | 1.3 |
| E1 | M | 0.75¹ | 0.3 | 53 | 199 | 11.1 | 10.5 | 100 | 6.7 | 147 | 1.1 |
| E2 | M | 0.75¹ | 0.3 | 71 | 204 | 11.1 | 10.4 | 99 | 6 | 148 | 1 |
| E3 | M | 0.75¹ | 0.4 | 55 | 213 | 11 | 9.6 | 99 | 5.6 | 146 | 1.1 |
| E5 | M | 0.75¹ | 0.4 | 65 | 188 | 11 | 11 | 101 | 6.5 | 146 | 1 |
| E6 | F | 0.75¹ | 0.5 | 76 | 299 | 11.1 | 8.8 | 97 | 6.8 | 144 | 1.1 |
| E9 | F | 0.75¹ | 0.4 | 71 | 196 | 11.2 | 9.1 | 99 | 6.2 | 144 | 1 |
| E10 | F | 0.75¹ | 0.4 | 48 | 221 | 10.8 | 7.2 | 100 | 5.8 | 147 | 1 |

¹Dose is 0.75 mg/kg nicotine

TABLE 34
Coagulation Parameters

| | | | Coagulation Parameters | |
|---|---|---|---|---|
| | | | ACTIVE PARTIAL | PRO- |
| | | Anatabine | THROMBO- | THROMBIN |
| Animal | | Dose | PLASTIN TIME | TIME |
| ID | Sex | (mg/kg) | seconds | seconds |
| A1 | M | 0 | 33.5 | 13.3 |
| A2 | M | 0 | 31.7 | 13.3 |
| A3 | M | 0 | 29.6 | 12.5 |
| A4 | M | 0 | 30.4 | 12.3 |
| A5 | M | 0 | 34.9 | 13.1 |
| A6 | F | 0 | 36.7 | 12.8 |
| A7 | F | 0 | 36.2 | 12.7 |
| A8 | F | 0 | 40.6 | 13.2 |
| A9 | F | 0 | 34.5 | 12.5 |
| A10 | F | 0 | 29.2 | 12.5 |
| B1 | M | 0.1 | 34.9 | 13.6 |
| B2 | M | 0.1 | 36.4 | 13.4 |
| B3 | M | 0.1 | 29.6 | 12.5 |
| B4 | M | 0.1 | 28.9 | 12.3 |
| B5 | M | 0.1 | 32 | 12.1 |
| B6 | F | 0.1 | 31.1 | 12.3 |
| B7 | F | 0.1 | 34.2 | 11.3 |
| B8 | F | 0.1 | 26 | 12.2 |
| B9 | F | 0.1 | 26.7 | 12.3 |
| B10 | F | 0.1 | 28.9 | 12.3 |
| C1 | M | 0.75 | 39.7 | 12.4 |
| C2 | M | 0.75 | 43.9 | 12.1 |
| C3 | M | 0.75 | 27.9 | 12.1 |
| C4 | M | 0.75 | 50.4 | 17.9 |
| C5 | M | 0.75 | 31.7 | 12.1 |
| C6 | F | 0.75 | 31.4 | 11.4 |
| C7 | F | 0.75 | 32.5 | 12.1 |
| C8 | F | 0.75 | 31.5 | 11.6 |
| C9 | F | 0.75 | 32.7 | 11.4 |
| C10 | F | 0.75 | 38.2 | 23.8 |
| D1 | M | 1.5 | 34.1 | 13.8 |
| D2 | M | 1.5 | 33.8 | 13.1 |
| D3 | M | 1.5 | 28.3 | 12.9 |
| D4 | M | 1.5 | 27.9 | 12.9 |
| D5 | M | 1.5 | 29.1 | 13.5 |
| D6 | F | 1.5 | 37.2 | 13.2 |
| D7 | F | 1.5 | 31.7 | 13.2 |
| D8 | F | 1.5 | 29.3 | 13 |
| D9 | F | 1.5 | 32.3 | 13.6 |
| D10 | F | 1.5 | 32 | 13.3 |
| E1 | M | 0.75¹ | 15 | 13.8 |
| E2 | M | 0.75¹ | 16.3 | 13.2 |
| E3 | M | 0.75¹ | 17.6 | 14.2 |
| E5 | M | 0.75¹ | 16.2 | 13 |
| E6 | F | 0.75¹ | 18.7 | 13.9 |
| E9 | F | 0.75¹ | 15 | 14.2 |
| E10 | F | 0.75¹ | UNABLE TO | UNABLE TO |
| | | | OBTAIN | OBTAIN |
| | | | RESULTS DUE TO | RESULTS DUE TO |
| | | | FIBRIN CLOTS | FIBRIN CLOTS |

¹Dose is 0.75 mg/kg nicotine

TABLE 35
Urinalysis Results

| | | Anat- | | | | | | | | | | | | |
|---|---|---|---|---|---|---|---|---|---|---|---|---|---|---|
| | | abine | Urinalysis | | | | | | | | | | | |
| Ani- | | Dose | | | | | SPEC | | | | | URO- | | |
| mal | | (mg/ | | | | | GRAV- | | PRO- | | | BILIN- | | |
| ID | Sex | kg) | VOLUME | | COLOR | CLARITY | ITY | pH | TEIN | GLUCOSE | KETONES | OGEN | BILIRUBIN | BLOOD |
| A1 | M | 0 | <0.5 | mL | YELLOW | HAZY | 1.046 | 7 | TRACE | NEGATIVE | 1+ | NORMAL | NEGATIVE | NEGA- |
| | | | | | | | | | | | | | | TIVE |
| A2 | M | 0 | <0.5 | mL | YELLOW | HAZY | 1.046 | 7 | TRACE | NEGATIVE | 1+ | NORMAL | NEGATIVE | NEGA- |
| | | | | | | | | | | | | | | TIVE |
| A3 | M | 0 | <0.5 | mL | YELLOW | HAZY | 1.046 | 7.5 | TRACE | NEGATIVE | 1+ | NORMAL | 1+ | NEGA- |
| | | | | | | | | | | | | | | TIVE |
| A4 | M | 0 | <0.5 | mL | YELLOW | HAZY | 1.031 | 7.5 | TRACE | NEGATIVE | NEGATIVE | NORMAL | NEGATIVE | 2+ |
| A5 | M | 0 | <0.5 | mL | YELLOW | HAZY | 1.026 | 7.5 | TRACE | NEGATIVE | NEGATIVE | NORMAL | NEGATIVE | TRACE |
| A6 | F | 0 | <0.5 | mL | YELLOW | HAZY | 1.034 | 6.5 | TRACE | NEGATIVE | NEGATIVE | NORMAL | NEGATIVE | TRACE |
| A7 | F | 0 | <0.5 | mL | YELLOW | HAZY | 1.034 | 7 | TRACE | NEGATIVE | NEGATIVE | NORMAL | NEGATIVE | TRACE |
| A8 | F | 0 | <0.5 | mL | YELLOW | HAZY | 1.024 | 8.5 | A | NEGATIVE | NEGATIVE | NORMAL | NEGATIVE | NEGA- |
| | | | | | | | | | | | | | | TIVE |
| A9 | F | 0 | <0.5 | mL | YELLOW | HAZY | 1.021 | 8 | 1+ | NEGATIVE | NEGATIVE | NORMAL | NEGATIVE | NEGA- |
| | | | | | | | | | | | | | | TIVE |
| A10 | F | 0 | <0.25 | mL | YELLOW | HAZY | 1.047 | 7 | A | NEGATIVE | NEGATIVE | NORMAL | NEGATIVE | TRACE |
| B1 | M | 0.1 | <0.5 | mL | YELLOW | HAZY | 1.027 | 8.5 | TRACE | NEGATIVE | 1+ | NORMAL | NEGATIVE | TRACE |
| B2 | M | 0.1 | A | | DNR | DNR | DNR | DNR | DNR | DNR | DNR | DNR | DNR | DNR |
| B3 | M | 0.1 | <0.5 | mL | YELLOW | HAZY | 1.039 | 7.5 | TRACE | NEGATIVE | NEGATIVE | NORMAL | NEGATIVE | TRACE |
| B4 | M | 0.1 | <0.5 | mL | YELLOW | HAZY | 1.027 | 7 | NEGA- | NEGATIVE | 1+ | NORMAL | NEGATIVE | NEGA- |
| | | | | | | | | | TIVE | | | | | TIVE |
| B5 | M | 0.1 | <0.5 | mL | YELLOW | HAZY | 1.048 | 7.5 | TRACE | NEGATIVE | 1+ | NORMAL | NEGATIVE | 1+ |
| B6 | F | 0.1 | <0.5 | mL | YELLOW | HAZY | 1.039 | 7.5 | A | NEGATIVE | NEGATIVE | NORMAL | NEGATIVE | NEGA- |
| | | | | | | | | | | | | | | TIVE |
| B7 | F | 0.1 | <0.5 | mL | YELLOW | HAZY | 1.036 | 7.5 | A | NEGATIVE | 1+ | NORMAL | NEGATIVE | TRACE |
| B8 | F | 0.1 | <0.5 | mL | YELLOW | HAZY | 1.045 | 7 | NEGA- | NEGATIVE | 1+ | NORMAL | 1+ | NEGA- |
| | | | | | | | | | TIVE | | | | | TIVE |
| B9 | F | 0.1 | 0.5 | | YELLOW | HAZY | 1.017 | 8 | NEGA- | NEGATIVE | NEGATIVE | NORMAL | NEGATIVE | NEGA- |
| | | | | | | | | | TIVE | | | | | TIVE |
| B10 | F | 0.1 | A | | DNR | DNR | DNR | DNR | DNR | DNR | DNR | DNR | DNR | DNR |
| C1 | M | 0.75 | No Urine Sample Submitted | | | | | | | | | | | |
| C2 | M | 0.75 | <0.5 | mL | YELLOW | HAZY | 1.05 | 7 | TRACE | NEGATIVE | 1+ | NORMAL | NEGATIVE | TRACE |
| C3 | M | 0.75 | <0.5 | mL | YELLOW | HAZY | 1.051 | 7 | TRACE | NEGATIVE | 1+ | NORMAL | NEGATIVE | NEGA- |
| | | | | | | | | | | | | | | TIVE |
| C4 | M | 0.75 | <0.5 | mL | YELLOW | HAZY | 1.043 | 8 | TRACE | NEGATIVE | NEGATIVE | NORMAL | 1+ | NEGA- |
| | | | | | | | | | | | | | | TIVE |
| C5 | M | 0.75 | <0.5 | mL | YELLOW | HAZY | 1.049 | 7 | TRACE | NEGATIVE | 1+ | NORMAL | NEGATIVE | NEGA- |
| | | | | | | | | | | | | | | TIVE |
| C6 | F | 0.75 | <0.5 | mL | YELLOW | HAZY | 1.016 | 7.5 | A | NEGATIVE | NEGATIVE | NORMAL | NEGATIVE | 3+ |
| C7 | F | 0.75 | <0.5 | mL | YELLOW | HAZY | 1.039 | 6 | A | NEGATIVE | NEGATIVE | NORMAL | NEGATIVE | TRACE |
| C8 | F | 0.75 | <0.5 | mL | YELLOW | HAZY | 1.036 | 8 | A | NEGATIVE | 1+ | NORMAL | NEGATIVE | NEGA- |
| | | | | | | | | | | | | | | TIVE |
| C9 | F | 0.75 | <0.25 | mL | YELLOW | HAZY | A | DNR | DNR | NEGATIVE | 1+ | DNR | NEGATIVE | DNR |
| C10 | F | 0.75 | <0.5 | mL | YELLOW | HAZY | 1.014 | 8 | 1+ | NEGATIVE | NEGATIVE | NORMAL | NEGATIVE | 2+ |
| D1 | M | 1.5 | <0.25 | mL | YELLOW | HAZY | 1.031 | 8 | A | NEGATIVE | 1+ | NORMAL | NEGATIVE | 2+ |
| D2 | M | 1.5 | <0.5 | mL | STRAW | CLOUDY | 1.034 | 7.5 | A | NEGATIVE | 1+ | NORMAL | NEGATIVE | 2+ |
| D3 | M | 1.5 | <0.25 | mL | YELLOW | HAZY | 1.034 | 7.5 | A | NEGATIVE | 1+ | NORMAL | NEGATIVE | 2+ |
| D4 | M | 1.5 | <0.5 | mL | YELLOW | HAZY | 1.045 | 7.5 | 2+ | NEGATIVE | 1+ | NORMAL | NEGATIVE | 2+ |
| D5 | M | 1.5 | <0.5 | mL | YELLOW | HAZY | 1.047 | 8.5 | TRACE | NEGATIVE | 1+ | NORMAL | NEGATIVE | NEGA- |
| | | | | | | | | | | | | | | TIVE |
| D6 | F | 1.5 | <0.5 | mL | YELLOW | HAZY | 1.039 | 8 | 1+ | NEGATIVE | NEGATIVE | NORMAL | NEGATIVE | TRACE |
| D7 | F | 1.5 | <0.5 | mL | YELLOW | HAZY | 1.038 | 8.5 | NEGA- | NEGATIVE | 1+ | NORMAL | NEGATIVE | NEGA- |
| | | | | | | | | | TIVE | | | | | TIVE |
| D8 | F | 1.5 | <0.5 | mL | YELLOW | HAZY | 1.039 | 7 | NEGA- | NEGATIVE | NEGATIVE | NORMAL | NEGATIVE | NEGA- |
| | | | | | | | | | TIVE | | | | | TIVE |

| | | | |
|---|---|---|---|
| D9 | F | 1.5 | No Urine Sample Submitted |
| D10 | F | 1.5 | No Urine Sample Submitted |

DNR—did not report - insufficient sample
A. Sample quantity was not sufficient for complete testing

| | | | Urinalysis | | | | | | | | | | |
|---|---|---|---|---|---|---|---|---|---|---|---|---|---|
| | | Anatabine | | | | | SPEC | | | | | | |
| Animal | | Dose | | | | | GRAV- | | | | | | |
| ID | Sex | (mg/kg) | VOLUME | | COLOR | CLARITY | ITY | pH | PROTEIN | GLUCOSE | KETONES | UROBILINOGEN | BILI [symbol] |
| E1 | M | 0.75¹ | <0.5 | mL | YELLOW | HAZY | 1.054 | 6 | NEGATIVE | NEGATIVE | NEGATIVE | NORMAL | NEG [symbol] |
| E2 | M | 0.75¹ | <0.5 | mL | YELLOW | HAZY | 1.058 | 6.5 | TRACE | NEGATIVE | NEGATIVE | NORMAL | NEG [symbol] |
| E3 | M | 0.75¹ | No Urine Sample Submitted | | | | | | | | | | |
| E5 | M | 0.75¹ | <0.5 | mL | YELLOW | HAZY | 1.046 | 7 | NEGATIVE | NEGATIVE | NEGATIVE | NORMAL | NEG [symbol] |
| E6 | F | 0.75¹ | 10 | UL | YELLOW | HAZY | 1.047 | 6.5 | NEGATIVE | NEGATIVE | NEGATIVE | NORMAL | NEG [symbol] |
| E9 | F | 0.75¹ | <0.5 | mL | YELLOW | HAZY | 1.052 | 7.5 | NEGATIVE | NEGATIVE | NEGATIVE | NORMAL | NEG [symbol] |

| | | | |
|---|---|---|---|
| E10 | F | 0.75¹ | No Urine Sample Submitted |

¹Dose is 0.75 mg/kg nicotine
A. Sample quantity was not sufficient for complete testing; DNR did not report due to insufficient sample [symbol]
indicates data missing or illegible when filed

TABLE 36
Tissue Collection Weights (g)
Group A: Vehicle (Males)

| 1 | | 2 | | 3 | | 4 | | 5 | |
|---|---|---|---|---|---|---|---|---|---|
| Tissue | weights | Tissue | weights | Tissue | weights | Tissue | weights | Tissue | weights |
| Thymus | 0.80 | Thymus | 0.80 | Thymus | 0.80 | Thymus | 0.70 | Thymus | 0.76 |
| Heart | 1.65 | Heart | 1.78 | Heart | 1.87 | Heart | 1.39 | Heart | 1.62 |
| Lungs | 2.02 | Lungs | 1.67 | Lungs | 2.16 | Lungs | 1.70 | Lungs | 1.97 |
| Thyroid/ | Cass | Thyroid/ | Cass | Thyroid/ | Cass | Thyroid/ | Cass | Thyroid/ | Cass |
| parathyroid | | parathyroid | | parathyroid | | parathyroid | | parathyroid | |
| Liver | 17.19 | Liver | 17.01 | Liver | 15.67 | Liver | 13.61 | Liver | 15.58 |
| Adrenals | Cass | Adrenals | Cass | Adrenals | Cass | Adrenals | Cass | Adrenals | Cass |
| Kidneys | 3.86 | Kidneys | 3.51 | Kidneys | 3.67 | Kidneys | 3.06 | Kidneys | 3.54 |
| Spleen | 1.12 | Spleen | 0.86 | Spleen | 1.41 | Spleen | 0.89 | Spleen | 0.88 |
| Small | 0.53 | Small | 1.4 | Small | 0.47 | Small | 0.41 | Small | 0.41 |
| intestine | | intestine | | intestine | | intestine | | intestine | |
| Prostate | 0.31 | Prostate | 0.56 | Prostate | 0.56 | Prostate | 0.57 | Prostate | 0.43 |
| Testes | 3.40 | Testes | 3.33 | Testes | 3.31 | Testes | 3.46 | Testes | 3.47 |
| Brain | 1.82 | Brain | 2.04 | Brain | 2.04 | Brain | 2.04 | Brain | 2.14 |
| Pituitary | Cass | Pituitary | cass | Pituitary | cass | Pituitary | Cass | Pituitary | Cass |
| Marrow | ✓ | Marrow | ✓ | Marrow | ✓ | Marrow | ✓ | Marrow | ✓ |

Group A Vehicle (Females)

| 6 | | 7 | | 8 | | 9 | | 10 | |
|---|---|---|---|---|---|---|---|---|---|
| Tissue | weights | Tissue | weights | Tissue | weights | Tissue | weights | Tissue | weights |
| Thymus | 0.66 | Thymus | 0.79 | Thymus | 0.78 | Thymus | 0.55 | Thymus | 0.66 |
| Heart | 1.33 | Heart | 1.31 | Heart | 1.34 | Heart | 1.14 | Heart | 1.22 |
| Lungs | 1.67 | Lungs | 1.60 | Lungs | 1.36 | Lungs | 1.41 | Lungs | 1.56 |
| Thyroid/ | Cass | Thyroid/ | Cass | Thyroid/ | Cass | Thyroid/ | Cass | Thyroid/ | Cass |
| parathyroid | | parathyroid | | parathyroid | | parathyroid | | parathyroid | |
| Liver | 12.40 | Liver | 11.24 | Liver | 10.19 | Liver | 9.36 | Liver | 11.49 |
| Adrenals | Cass | Adrenals | Cass | Adrenals | Cass | Adrenals | Cass | Adrenals | Cass |
| Kidneys | 2.66 | Kidneys | 2.72 | Kidneys | 2.14 | Kidneys | 1.75 | Kidneys | 2.42 |
| Spleen | 0.72 | Spleen | 0.68 | Spleen | 0.56 | Spleen | 0.50 | Spleen | 0.61 |
| Small | 1.33 | Small | 0.41 | Small | 0.90 | Small | 0.44 | Small | 1.02 |
| intestine | | intestine | | intestine | | intestine | | intestine | |
| Ovaries/ | 0.76 | Ovaries/ | 1.42 | Ovaries/ | 0.83 | Ovaries/ | 1.88 | Ovaries/ | 0.61 |
| uterus | | uterus | | uterus | | uterus | | uterus | |
| Brain | 1.87 | Brain | 2.06 | Brain | 1.85 | Brain | 2.12 | Brain | 1.76 |
| Pituitary | cass | Pituitary | cass | Pituitary | cass | Pituitary | cass | Pituitary | cass |
| Marrow | ✓ | Marrow | ✓ | Marrow | ✓ | Marrow | ✓ | Marrow | ✓ |

Group B: Anatabine 0.1 mg/kg (Males)

| 1 | | 2 | | 3 | | 4 | | 5 | |
|---|---|---|---|---|---|---|---|---|---|
| Tissue | weights | Tissue | weights | Tissue | weights | Tissue | weights | Tissue | weights |
| Thymus | 0.74 | Thymus | 0.74 | Thymus | 0.76 | Thymus | 0.71 | Thymus | 0.81 |
| Heart | 1.71 | Heart | 1.59 | Heart | 1.97 | Heart | 1.60 | Heart | 1.85 |
| Lungs | 2.31 | Lungs | 1.77 | Lungs | 2.08 | Lungs | 1.91 | Lungs | 1.83 |
| Thyroid/ | Cass | Thyroid/ | Cass | Thyroid/ | Cass | Thyroid/ | Cass | Thyroid/ | Cass |
| parathyroid | | parathyroid | | parathyroid | | parathyroid | | parathyroid | |
| Liver | 17.92 | Liver | 15.45 | Liver | 16.98 | Liver | 16.32 | Liver | 17.38 |
| Adrenals | Cass | Adrenals | Cass | Adrenals | Cass | Adrenals | Cass | Adrenals | Cass |
| Kidneys | 3.55 | Kidneys | 3.33 | Kidneys | 3.70 | Kidneys | 3.27 | Kidneys | 3.99 |
| Spleen | 0.91 | Spleen | 0.66 | Spleen | 0.82 | Spleen | 0.93 | Spleen | 0.76 |
| Small | 0.61 | Small | 0.96 | Small | 0.88 | Small | 0.50 | Small | 0.35 |
| intestine | | intestine | | intestine | | intestine | | intestine | |
| Prostate | 0.72 | Prostate | 0.49 | Prostate | 0.73 | Prostate | 0.53 | Prostate | 0.48 |
| Testes | 3.69 | Testes | 3.64 | Testes | 3.14 | Testes | 3.35 | Testes | 3.29 |
| Brain | 2.35 | Brain | 2.16 | Brain | 2.24 | Brain | 2.11 | Brain | 1.88 |
| Pituitary | cass | Pituitary | cass | Pituitary | cass | Pituitary | cass | Pituitary | cass |
| Marrow | ✓ | Marrow | ✓ | Marrow | ✓ | Marrow | ✓ | Marrow | ✓ |

Group B: Anatabine 0.1 mg/kg (Females)

| 6 | | 7 | | 8 | | 9 | | 10 | |
|---|---|---|---|---|---|---|---|---|---|
| Tissue | weights | Tissue | weights | Tissue | weights | Tissue | weights | Tissue | weights |
| Thymus | 0.68 | Thymus | 0.59 | Thymus | 0.49 | Thymus | 0.71 | Thymus | 0.73 |
| Heart | 1.29 | Heart | 1.12 | Heart | 0.95 | Heart | 1.05 | Heart | 1.25 |
| Lungs | 1.42 | Lungs | 1.39 | Lungs | 1.15 | Lungs | 1.52 | Lungs | 1.59 |
| Thyroid/ | Cass | Thyroid/ | Cass | Thyroid/ | Cass | Thyroid/ | Cass | Thyroid/ | Cass |
| parathyroid | | parathyroid | | parathyroid | | parathyroid | | parathyroid | |
| Liver | 11.54 | Liver | 10.09 | Liver | 9.94 | Liver | 12.94 | Liver | 9.67 |
| Adrenals | Cass | Adrenals | Cass | Adrenals | Cass | Adrenals | Cass | Adrenals | Cass |
| Kidneys | 2.46 | Kidneys | 2.02 | Kidneys | 2.02 | Kidneys | 2.14 | Kidneys | 2.25 |
| Spleen | 0.79 | Spleen | 0.62 | Spleen | 0.51 | Spleen | 0.67 | Spleen | 0.65 |
| Small | 0.87 | Small | 0.71 | Small | 0.55 | Small | 0.49 | Small | 0.63 |
| intestine | | intestine | | intestine | | intestine | | intestine | |
| Ovaries/ | 0.96 | Ovaries/ | 1.79 | Ovaries/ | 0.84 | Ovaries/ | 1.42 | Ovaries/ | 0.70 |
| uterus | | uterus | | uterus | | uterus | | uterus | |
| Brain | 1.91 | Brain | 1.89 | Brain | 1.75 | Brain | 1.86 | Brain | 1.89 |
| Pituitary | cass | Pituitary | cass | Pituitary | cass | Pituitary | cass | Pituitary | cass |
| Marrow | ✓ | Marrow | ✓ | Marrow | ✓ | Marrow | ✓ | Marrow | ✓ |

Group C: Anatabine 0.75 mg/kg (Males)

| 1 | | 2 | | 3 | | 4 | | 5 | |
|---|---|---|---|---|---|---|---|---|---|
| Tissue | weights | Tissue | weights | Tissue | weights | Tissue | weights | Tissue | weights |
| Thymus | 1.02 | Thymus | 0.52 | Thymus | 0.67 | Thymus | 0.69 | Thymus | 0.47 |
| Heart | 1.52 | Heart | 1.38 | Heart | 1.56 | Heart | 1.39 | Heart | 1.29 |
| Lungs | 1.86 | Lungs | 1.97 | Lungs | 1.72 | Lungs | 1.95 | Lungs | 1.58 |
| Thyroid/ | Cass | Thyroid/ | Cass | Thyroid/ | Cass | Thyroid/ | Cass | Thyroid/ | Cass |
| parathyroid | | parathyroid | | parathyroid | | parathyroid | | parathyroid | |
| Liver | 14.92 | Liver | 13.91 | Liver | 14.70 | Liver | 14.01 | Liver | 14.94 |
| Adrenals | Cass | Adrenals | Cass | Adrenals | Cass | Adrenals | Cass | Adrenals | Cass |
| Kidneys | 3.45 | Kidneys | 2.77 | Kidneys | 3.49 | Kidneys | 3.16 | Kidneys | 2.89 |
| Spleen | 0.71 | Spleen | 0.63 | Spleen | 0.79 | Spleen | 0.93 | Spleen | 0.64 |
| Small | 0.54 | Small | 1.41 | Small | 0.56 | Small | 0.41 | Small | 0.47 |
| intestine | | intestine | | intestine | | intestine | | intestine | |
| Prostate | 0.49 | Prostate | 0.49 | Prostate | 0.65 | Prostate | 0.46 | Prostate | 0.45 |
| Testes | 3.19 | Testes | 3.84 | Testes | 3.03 | Testes | 3.28 | Testes | 2.70 |
| Brain | 2.01 | Brain | 2.19 | Brain | 2.12 | Brain | 2.00 | Brain | 2.02 |
| Pituitary | cass | Pituitary | cass | Pituitary | cass | Pituitary | cass | Pituitary | cass |
| Marrow | ✓ | Marrow | ✓ | Marrow | ✓ | Marrow | ✓ | Marrow | ✓ |

Group C: Anatabine 0.75 mg/kg (Females)

| 6 | | 7 | | 8 | | 9 | | 10 | |
|---|---|---|---|---|---|---|---|---|---|
| Tissue | weights | Tissue | weights | Tissue | weights | Tissue | weights | Tissue | weights |
| Thymus | 0.56 | Thymus | 0.53 | Thymus | 0.63 | Thymus | 0.52 | Thymus | 0.54 |
| Heart | 1.09 | Heart | 1.21 | Heart | 1.23 | Heart | 1.03 | Heart | 0.99 |
| Lungs | 1.46 | Lungs | 1.39 | Lungs | 1.62 | Lungs | 1.30 | Lungs | 1.49 |
| Thyroid/ | Cass | Thyroid/ | Cass | Thyroid/ | Cass | Thyroid/ | Cass | Thyroid/ | Cass |
| parathyroid | | parathyroid | | parathyroid | | parathyroid | | parathyroid | |
| Liver | 10.71 | Liver | 8.65 | Liver | 11.37 | Liver | 11.20 | Liver | 10.19 |
| Adrenals | Cass | Adrenals | Cass | Adrenals | Cass | Adrenals | Cass | Adrenals | Cass |
| Kidneys | 2.08 | Kidneys | 2.20 | Kidneys | 2.68 | Kidneys | 2.18 | Kidneys | 2.36 |
| Spleen | 0.70 | Spleen | 0.65 | Spleen | 0.65 | Spleen | 0.51 | Spleen | 0.49 |
| Small | 0.72 | Small | 0.37 | Small | 0.85 | Small | 0.47 | Small | 1.34 |
| intestine | | intestine | | intestine | | intestine | | intestine | |
| Ovaries/ | 0.81 | Ovaries/ | 1.50 | Ovaries/ | 0.72 | Ovaries/ | 1.23 | Ovaries/ | 3.65 |
| uterus | | uterus | | uterus | | uterus | | uterus | |
| Brain | 1.99 | Brain | 1.93 | Brain | 1.73 | Brain | 2.06 | Brain | 1.81 |
| Pituitary | cass | Pituitary | cass | Pituitary | cass | Pituitary | cass | Pituitary | cass |
| Marrow | ✓ | Marrow | ✓ | Marrow | ✓ | Marrow | ✓ | Marrow | ✓ |

Group D: Anatabine 1.5 mg/kg (Males)

| 1 | | 2 | | 3 | | 4 | | 5 | |
|---|---|---|---|---|---|---|---|---|---|
| Tissue | weights | Tissue | weights | Tissue | weights | Tissue | weights | Tissue | weights |
| Thymus | 0.83 | Thymus | 0.77 | Thymus | 0.54 | Thymus | 0.86 | Thymus | 0.59 |
| Heart | 1.50 | Heart | 1.53 | Heart | 1.47 | Heart | 1.61 | Heart | 1.47 |
| Lungs | 1.77 | Lungs | 2.06 | Lungs | 1.93 | Lungs | 2.02 | Lungs | 1.90 |
| Thyroid/ | Cass | Thyroid/ | Cass | Thyroid/ | Cass | Thyroid/ | Cass | Thyroid/ | Cass |
| parathyroid | | parathyroid | | parathyroid | | parathyroid | | parathyroid | |
| Liver | 15.86 | Liver | 17.03 | Liver | 17.35 | Liver | 18.27 | Liver | 14.26 |
| Adrenals | Cass | Adrenals | Cass | Adrenals | Cass | Adrenals | Cass | Adrenals | Cass |
| Kidneys | 3.48 | Kidneys | 3.54 | Kidneys | 3.02 | Kidneys | 3.79 | Kidneys | 3.43 |
| Spleen | 0.71 | Spleen | 0.83 | Spleen | 0.86 | Spleen | 0.97 | Spleen | 0.92 |
| Small | 0.43 | Small | 0.77 | Small | 0.56 | Small | 1.00 | Small | 0.66 |
| intestine | | intestine | | intestine | | intestine | | intestine | |
| Prostate | 0.50 | Prostate | 0.45 | Prostate | | Prostate | 0.49 | Prostate | 0.45 |
| Testes | 2.78 | Testes | 3.28 | Testes | 3.51 | Testes | 3.41 | Testes | 3.23 |
| Brain | 1.75 | Brain | 1.81 | Brain | 2.02 | Brain | 2.00 | Brain | 2.08 |
| Pituitary | cass | Pituitary | cass | Pituitary | cass | Pituitary | cass | Pituitary | cass |
| Marrow | ✓ | Marrow | ✓ | Marrow | ✓ | Marrow | ✓ | Marrow | ✓ |

Group D: Anatabine 1.5 mg/kg (Females)

| 6 | | 7 | | 8 | | 9 | | 10 | |
|---|---|---|---|---|---|---|---|---|---|
| Tissue | weights | Tissue | weights | Tissue | weights | Tissue | weights | Tissue | weights |
| Thymus | 0.59 | Thymus | 0.63 | Thymus | 0.52 | Thymus | 0.61 | Thymus | 0.51 |
| Heart | 1.12 | Heart | 0.88 | Heart | 1.04 | Heart | 0.97 | Heart | 0.98 |
| Lungs | 1.29 | Lungs | 1.46 | Lungs | 1.37 | Lungs | 1.60 | Lungs | 1.59 |
| Thyroid/ | Cass | Thyroid/ | Cass | Thyroid/ | Cass | Thyroid/ | Cass | Thyroid/ | Cass |
| parathyroid | | parathyroid | | parathyroid | | parathyroid | | parathyroid | |
| Liver | 9.65 | Liver | 8.83 | Liver | 11.46 | Liver | 11.05 | Liver | 9.21 |
| Adrenals | Cass | Adrenals | Cass | Adrenals | Cass | Adrenals | Cass | Adrenals | Cass |
| Kidneys | 2.17 | Kidneys | 2.08 | Kidneys | 2.52 | Kidneys | 2.64 | Kidneys | 1.97 |
| Spleen | 0.53 | Spleen | 0.65 | Spleen | 0.64 | Spleen | 0.68 | Spleen | 0.51 |
| Small | 0.49 | Small | 0.60 | Small | 1.07 | Small | 0.56 | Small | 0.77 |
| intestine | | intestine | | intestine | | intestine | | intestine | |
| Ovaries/ | 0.84 | Ovaries/ | 1.20 | Ovaries/ | 0.98 | Ovaries/ | 1.34 | Ovaries/ | 0.78 |
| uterus | | uterus | | uterus | | uterus | | uterus | |
| Brain | 1.90 | Brain | 1.91 | Brain | 1.97 | Brain | 2.08 | Brain | 1.91 |
| Pituitary | cass | Pituitary | cass | Pituitary | cass | Pituitary | cass | Pituitary | cass |
| Marrow | ✓ | Marrow | ✓ | Marrow | ✓ | Marrow | ✓ | Marrow | ✓ |

Group E: Nicotine 0.75 mg/kg (Males)

| 1 | | 2 | | 3 | | 5 | |
|---|---|---|---|---|---|---|---|
| Tissue | weights | Tissue | weights | Tissue | Weights | Tissue | weights |
| Thymus | 0.54 | Thymus | 0.46 | Thymus | 0.56 | Thymus | 0.76 |
| Heart | 1.20 | Heart | 1.17 | Heart | 1.21 | Heart | 1.42 |
| Lungs | 1.84 | Lungs | 1.49 | Lungs | 1.62 | Lungs | 1.66 |
| Thyroid/ | Cass | Thyroid/ | Cass | Thyroid/ | Cass | Thyroid/ | Cass |
| parathyroid | | parathyroid | | parathyroid | | parathyroid | |
| Liver | 11.97 | Liver | 12.52 | Liver | 12.06 | Liver | 14.20 |
| Adrenals | Cass | Adrenals | Cass | Adrenals | Cass | Adrenals | Cass |
| Kidneys | 3.19 | Kidneys | 3.04 | Kidneys | 2.87 | Kidneys | 3.01 |
| Spleen | 0.81 | Spleen | 0.62 | Spleen | 0.78 | Spleen | 0.86 |
| Small | 0.71 | Small | 0.56 | Small | 0.53 | Small | 0.40 |
| intestine | | intestine | | intestine | | intestine | |
| Prostate | 0.41 | Prostate | 0.28 | Prostate | 0.45 | Prostate | 0.28 |
| Testes | 3.05 | Testes | 3.13 | Testes | 3.02 | Testes | 2.92 |
| Brain | 2.06 | Brain | 1.90 | Brain | 1.84 | Brain | 1.94 |
| Pituitary | Cass | Pituitary | Cass | Pituitary | Cass | Pituitary | Cass |
| Marrow | ✓ | Marrow | ✓ | Marrow | ✓ | Marrow | ✓ |

Group E: Nicotine 0.75 mg/kg (Females)

| 6 | | 9 | | 10 | |
|---|---|---|---|---|---|
| Tissue | weights | Tissue | weights | Tissue | weights |
| Thymus | 0.54 | Thymus | 0.48 | Thymus | 0.48 |
| Heart | 1.09 | Heart | 1.06 | Heart | 0.83 |
| Lungs | 1.20 | Lungs | 1.48 | Lungs | 1.28 |
| Thyroid/ | Cass | Thyroid/ | Cass | Thyroid/ | Cass |
| parathyroid | | parathyroid | | parathyroid | |
| Liver | 9.48 | Liver | 10.11 | Liver | 9.18 |
| Adrenals | Cass | Adrenals | Cass | Adrenals | Cass |
| Kidneys | 2.34 | Kidneys | 2.29 | Kidneys | 2.29 |
| Spleen | 0.46 | Spleen | 0.76 | Spleen | 0.46 |
| Small | 0.41 | Small | 0.40 | Small | 0.28 |
| intestine | | intestine | | intestine | |
| Ovaries/ | 1.59 | Ovaries/ | 0.92 | Ovaries/ | 1.32 |
| uterus | | uterus | | uterus | |
| Brain | 1.77 | Brain | 1.93 | Brain | 1.87 |
| Pituitary | Cass | Pituitary | Cass | Pituitary | Cass |
| Marrow | ✓ | Marrow | ✓ | Marrow | ✓ |

TABLE 37
Dosing solutions

| | | | Test | Test | |
|---|---|---|---|---|---|
| | | | compound | compound | |
| | Percentage | Dose | concentra- | concentra- | Injection |
| Test | content of | level | tion (total) | tion (base) | volume |
| compound | anatabine | (mg/kg) | (mg/mL) | (mg/mL) | (mL/kg) |
| Anatabine | 5.18 | 0.20 | 0.772 | 0.04 | 5 |
| Anatabine | 5.18 | 2.0 | 7.72 | 0.4 | 5 |

TABLE 38
Dosing

| Test | | | Concentration | # of | Dosing times |
|---|---|---|---|---|---|
| compound | Route | Sex | mg/kg/dose | animals | (minutes) |
| Anatabine | p.o. | M | 0.2 | 4 | 0, 240, 480 |
| | | F | 0.2 | 4 | |
| | | M | 2.0 | 4 | |
| | | F | 2.0 | 4 | |

TABLE 39
Blood Collection Times

| | | | | | Blood |
|---|---|---|---|---|---|
| Test | | | Concentration | # of | collection |
| compound | Route | Sex | mg/kg/dose | animals | (minutes) |
| Anatabine | p.o. | M | 0.2 | 4 | 30, 60, 235 |
| | | F | 0.2 | 4 | (pre-dose), 270, |
| | | M | 2.0 | 4 | 300, 475 (pre- |
| | | F | 2.0 | 4 | dose), 540, 600, |
| | | | | | 720, 1440 |

TABLE 40
Calibration Curve Concentrations

| nominal concentration | |
|---|---|
| (nM) | stock concentration (μM) |
| 5000 | 250 |
| 1667 | 83.3 |
| 555.5 | 27.8 |
| 185.2 | 9.3 |
| 61.7 | 3.1 |
| 20.6 | 1.0 |
| 6.9 | 0.34 |
| 2.3 | 0.11 |
| 0.76 | 0.038 |
| 0.25 | 0.013 |

TABLE 41
LC/MS/MS ionization conditions and identity
of parent and product ions.

| | | | Pre- | Prod- | Collision |
|---|---|---|---|---|---|
| | | Polariza- | cursor | uct | energy |
| Compound | MW | tion | m/z | m/z | (V) |
| Anatabine | 160.2 | Positive | 161.1 | 115.1 | 28 |
| (R,S)-Antabine- | 164.24 | Positive | 165.1 | 148.1 | 20 |
| 2,4,5,6-d₄ | | | | | |

TABLE 42
Limits of Detection and Calibration Curves

| | | Lower Limit of | Upper Limit of |
|---|---|---|---|
| | Limit of Detection | Quantitation | Quantitation |
| Sample | (LOD) (ng/mL) | (LLQ) (ng/mL) | (ULQ) (ng/mL) |
| Anatabine in | 0.37 | 1.1 | ≧801 |
| rat plasma | | | |

TABLE 43
Dosing Solution Analysis

| | | Expected | Actual | Actual |
|---|---|---|---|---|
| | | Concen- | Concen- | Concentration |
| | Dose | tration | tration | relative to |
| Compound | (mg/kg) | (mg/mL) | (mg/mL) | Expected (%) |
| anatabine | 0.2 | 0.04 | 0.025 | 63% |
| anatabine | 2.0 | 0.4 | 0.335 | 84% |

TABLE 44
Comparison of pharmacokinetic parameters (Tₘₐₓ, C_{p, max} and C_{p, min})
between male and female rats in each of the two treatment groups.

| | Anatabine (0.6 mg/kg) | | | | | | | Anatabine (6.0 mg/kg) | | | | | | |
|---|---|---|---|---|---|---|---|---|---|---|---|---|---|---|
| | Male | | | Female | | | | Male | | | Female | | | |
| Parameter | n | Mean | SD | n | Mean | SD | p | n | Mean | SD | n | Mean | SD | p |
| T_{max (1)} (hr) | 4 | 0.75 | 0.29 | 4 | 0.63 | 0.25 | 0.537 | 4 | 0.63 | 0.25 | 4 | 0.50 | 0.00 | 0.356 |
| T_{max (2)} (hr) | 4 | 0.63 | 0.25 | 4 | 0.88 | 0.25 | 0.207 | 4 | 0.63 | 0.25 | 4 | 0.63 | 0.25 | 1.000 |
| T_{max (3)} (hr) | 4 | 1.00 | 0 | 4 | 1.25 | 0.50 | 0.356 | 4 | 2.00 | 1.41 | 4 | 1.25 | 0.50 | 0.356 |
| C_{p, max (1)} | 4 | 34.3 | 2.1 | 4 | 38.3 | 6.1 | 0.262 | 4 | 244 | 86 | 4 | 260 | 35 | 0.738 |
| (ng/mL) | | | | | | | | | | | | | | |
| C_{p, max (2)} | 4 | 30.3 | 3.8 | 4 | 31.3 | 14.2 | 0.896 | 4 | 305 | 53 | 4 | 312 | 77 | 0.889 |
| (ng/mL) | | | | | | | | | | | | | | |
| C_{p, max (3)} | 3 | 37.3 | 8.1 | 4 | 35.5 | 10.6 | 0.814 | 4 | 283 | 94 | 4 | 375 | 123 | 0.281 |
| (ng/mL) | | | | | | | | | | | | | | |
| C_{p, min (1)} | 3 | 11.0 | 2.0 | 4 | 10.3 | 6.4 | 0.856 | 4 | 75 | 26 | 4 | 52 | 26 | 0.254 |
| (ng/mL) | | | | | | | | | | | | | | |
| C_{p, min (2)} | 3 | 15.3 | 5.5 | 4 | 7.5 | 1.7 | 0.040 | 4 | 93 | 16 | 4 | 180 | 31 | 0.002 |
| (ng/mL) | | | | | | | | | | | | | | |

TABLE 45
Comparison of pharmacokinetic parameters (C_{p, max} and C_{p, min}) over
time for male and female rats in each of the two treatment groups.

| | Anatabine (0.6 mg/kg) | | | | | | Anatabine (6.0 mg/kg) | | | | | |
|---|---|---|---|---|---|---|---|---|---|---|---|---|
| | Male | | | Female | | | Male | | | Female | | |
| Parameter | Mean | SD | p | Mean | SD | p | Mean | SD | p | Mean | SD | p |
| C_{p, max (1)} (ng/mL) | 34.3 | 2.1 | 0.195 | 38.3 | 6.1 | 0.570 | 287 | 11 | 0.897 | 259.8 | 35.4 | 0.142 |
| C_{p, max (2)} (ng/mL) | 30.3 | 3.8 | | 31.3 | 14.2 | | 305 | 53 | | 312.0 | 76.8 | |
| C_{p, max (3)} (ng/mL) | 37.3 | 8.1 | | 35.5 | 10.6 | | 283 | 94 | | 374.8 | 122.9 | |
| C_{p, min (1)} (ng/mL) | 11.0 | 2.0 | 0.177 | 10.3 | 6.4 | 0.428 | 75 | 26 | 0.131 | 51.5 | 26.0 | 0.015 |
| C_{p, min (2)} (ng/mL) | 15.3 | 5.5 | | 7.5 | 1.7 | | 93 | 16 | | 180.0 | 30.7 | |

TABLE 46
Comparison of pharmacokinetic parameters (AUC_{0□□}, t_{1/2, 0□4}, t_{1/2, terminal},
MTT_{0□4} and MAT_{0□4}) between male and female rats in each of the two treatment groups.

| | Anatabine (0.6 mg/kg) | | | | | | | Anatabine (6.0 mg/kg) | | | | | | |
|---|---|---|---|---|---|---|---|---|---|---|---|---|---|---|
| | Male | | | Female | | | | Male | | | Female | | | |
| Parameter | n | Mean | SD | n | Mean | SD | p | n | Mean | SD | n | Mean | SD | p |
| AUC_{0□□} (ng · hr/mL) | 3 | 280 | 102 | 4 | 290 | 71 | 0.880 | 4 | 3257 | 480 | 4 | 3735 | 587 | 0.255 |
| t_{1/2, 0□□} (hr) | 4 | 2.05 | 0.43 | 4 | 2.06 | 1.20 | 0.986 | 3 | 1.76 | 0.39 | 4 | 1.82 | 0.81 | 0.918 |
| t_{1/2, terminal} (hr) | 3 | 1.78 | 0.68 | 4 | 1.80 | 0.72 | 0.970 | 4 | 5.07 | 2.05 | 4 | 3.99 | 1.52 | 0.430 |
| MTT_{0□□} (hr) | 3 | 3.17 | 0.51 | 4 | 2.98 | 1.84 | 0.873 | 4 | 3.18 | 1.29 | 4 | 2.76 | 1.39 | 0.668 |
| MAT_{0□□} (hr) | 3 | 0.71 | 1.34 | 4 | 0.53 | 1.84 | 0.873 | 4 | 0.73 | 1.29 | 4 | 0.30 | 1.39 | 0.668 |

TABLE 47
Comparison of pharmacokinetic parameters (AUC_{0□□}, t_{1/2, 0□4},
t_{1/2, terminal}, MTT_{0□4} and MAT_{0□4}) between treatment groups.

| | Anatabine (0.6 mg/kg) | | | Anatabine(6.0 mg/kg) | | | |
|---|---|---|---|---|---|---|---|
| Parameter | n | Mean | SD | n | Mean | SD | p |
| AUC_{0□□} | 7 | 285 | 77 | 8 | 3496 | 559 | <0.001 |
| (ng · hr/mL) | | | | | | | |
| t_{1/2, 0□□} (hr) | 8 | 2.05 | 0.83 | 7 | 1.79 | 0.62 | 0.514 |
| t_{1/2, terminal} (hr) | 7 | 1.79 | 0.64 | 8 | 4.53 | 1.77 | 0.002 |
| MTT_{0□□} (hr) | 7 | 3.06 | 1.34 | 8 | 2.97 | 1.26 | 0.898 |
| MAT_{0□□} (hr) | 7 | 0.61 | 1.34 | 8 | 0.52 | 1.26 | 0.898 |

TABLE 48
Comparison of pharmacokinetic parameters (t_{1/2, 0□4},
MTT_{0□4} and MAT_{0□4}) between male and female rats
in both treatment groups, combined, and all data combined.

| | Male | | | Female | | | | Overall | | |
|---|---|---|---|---|---|---|---|---|---|---|
| Parameter | n | Mean | SD | n | Mean | SD | p | n | Mean | SD |
| t_{1/2, 0□□} (hr) | 7 | 1.92 | 0.41 | 8 | 1.94 | 0.96 | 0.973 | 15 | 1.93 | 0.73 |
| MTT_{0□□} (hr) | 7 | 3.18 | 0.96 | 8 | 2.87 | 1.52 | 0.650 | 15 | 3.01 | 1.25 |
| MAT_{0□□} (hr) | 7 | 0.72 | 0.96 | 8 | 0.42 | 1.52 | 0.650 | 15 | 0.56 | 1.25 |

TABLE 49
Animal Weights and Dosing Times

| | | | Dose | | Time Points for Samples (hrs) - retro-orbital | | | | | | | | | | | |
|---|---|---|---|---|---|---|---|---|---|---|---|---|---|---|---|---|
| Cmpnd | Rat | B.W. (g) | volume (ml) | time | 0.5 | 1 | 4 (pre) | dose (2) | 4.5 | 5 | 8 (pre) | dose (3) | 9 | 10 | 12 | 24 |
| 1 | A | 246 | 1.2 | 6:06 | 6:36 | 7:06 | 10:01 | 10:06 | 10:36 | 11:06 | 2:01 | 2:06 | 3:06 | 4:06 | 6:06 | 6:06 |
| MALE | B | 231 | 1.2 | 6:07 | 6:37 | 7:07 | 10:02 | 10:07 | 10:37 | 11:07 | 2:02 | 2:07 | 3:07 | 4:07 | 6:07 | 6:07 |
| Anatabine | C | 244 | 1.2 | 6:08 | 6:38 | 7:08 | 10:03 | 10:08 | 10:38 | 11:08 | 2:03 | 2:08 | 3:08 | 4:08 | 6:08 | 6:08 |
| 0.2 MPK/dose | D | 242 | 1.2 | 6:09 | 6:39 | 7:09 | 10:04 | 10:09 | 10:39 | 11:09 | 2:04 | 2:09 | 3:09 | 4:09 | 6:09 | 6:09 |
| 2 | A | 203 | 1.0 | 6:10 | 6:40 | 7:10 | 10:05 | 10:10 | 10:40 | 11:10 | 2:05 | 2:10 | 3:10 | 4:10 | 6:10 | 6:10 |
| FEMALE | B | 200 | 1.0 | 6:11 | 6:41 | 7:11 | 10:06 | 10:11 | 10:41 | 11:11 | 2:06 | 2:11 | 3:11 | 4:11 | 6:11 | 6:11 |
| Anatabine | C | 212 | 1.1 | 6:12 | 6:42 | 7:12 | 10:07 | 10:12 | 10:42 | 11:12 | 2:07 | 2:12 | 3:12 | 4:12 | 6:12 | 6:12 |
| 0.2 MPK/dose | D | 201 | 1.0 | 6:13 | 6:43 | 7:13 | 10:08 | 10:13 | 10:43 | 11:13 | 2:08 | 2:13 | 3:13 | 4:13 | 6:13 | 6:13 |
| 3 | A | 240 | 1.2 | 6:14 | 6:44 | 7:14 | 10:09 | 10:14 | 10:44 | 11:14 | 2:09 | 2:14 | 3:14 | 4:14 | 6:14 | 6:14 |
| MALE | B | 239 | 1.2 | 6:15 | 6:45 | 7:15 | 10:10 | 10:15 | 10:45 | 11:15 | 2:10 | 2:15 | 3:15 | 4:15 | 6:15 | 6:15 |
| Anatabine | C | 241 | 1.2 | 6:16 | 6:46 | 7:16 | 10:11 | 10:16 | 10:46 | 11:16 | 2:11 | 2:16 | 3:16 | 4:16 | 6:16 | 6:16 |
| 2.0 MPK/dose | D | 240 | 1.2 | 6:17 | 6:47 | 7:17 | 10:12 | 10:17 | 10:47 | 11:17 | 2:12 | 2:17 | 3:17 | 4:17 | 6:17 | 6:17 |
| 4 | A | 208 | 1.0 | 6:18 | 6:48 | 7:18 | 10:13 | 10:18 | 10:48 | 11:18 | 2:13 | 2:18 | 3:18 | 4:18 | 6:18 | 6:18 |
| FEMALE | B | 215 | 1.1 | 6:19 | 6:49 | 7:19 | 10:14 | 10:19 | 10:49 | 11:19 | 2:14 | 2:19 | 3:19 | 4:19 | 6:19 | 6:19 |
| Anatabine | C | 207 | 1.0 | 6:20 | 6:50 | 7:20 | 10:15 | 10:20 | 10:50 | 11:20 | 2:15 | 2:20 | 3:20 | 4:20 | 6:20 | 6:20 |
| 2.0 MPK/dose | D | 201 | 1.00 | 6:21 | 6:51 | 7:21 | 10:16 | 10:21 | 10:51 | 11:21 | 2:16 | 2:21 | 3:21 | 4:21 | 6:21 | 6:21 |

TABLE 50
Measured Concentrations of Anatabine in
Rat Plasma Samples at Each Time Point

| | | | Time | Anatabine |
|---|---|---|---|---|
| Dose | Animal | | Point | Concentration |
| (mg/kg) | ID | Sex | (min) | (ng/mL) |
| 0.2 | 1A | male | 30 | 34 |
| | | | 60 | 26 |
| | | | 235 | 11 |
| | | | 270 | 35 |
| | | | 300 | 22 |
| | | | 475 | 9 |
| | | | 540 | 4 |
| | | | 600 | <LOQ |
| | | | 720 | <LOQ |
| | | | 1440 | <LOQ |
| | 1B | male | 30 | <LOQ |
| | | | 60 | 32 |
| | | | 235 | <LOQ |
| | | | 270 | 31 |
| | | | 300 | 26 |
| | | | 475 | <LOQ |
| | | | 540 | 28 |
| | | | 600 | 17 |
| | | | 720 | 5 |
| | | | 1440 | <LOQ |
| | 1C | male | 30 | 29 |
| | | | 60 | 34 |
| | | | 235 | 13 |
| | | | 270 | 20 |
| | | | 300 | 29 |
| | | | 475 | 18 |
| | | | 540 | 41 |
| | | | 600 | 35 |
| | | | 720 | 12 |
| | | | 1440 | <LOQ |
| | 1D | male | 30 | 37 |
| | | | 60 | 24 |
| | | | 235 | 9 |
| | | | 270 | 26 |
| | | | 300 | 22 |
| | | | 475 | 19 |
| | | | 540 | 43 |
| | | | 600 | 34 |
| | | | 720 | 19 |
| | | | 1440 | <LOQ |
| | 2A | female | 30 | 34 |
| | | | 60 | 29 |
| | | | 235 | 4 |
| | | | 270 | 14 |
| | | | 300 | 18 |
| | | | 475 | 7 |
| | | | 540 | 24 |
| | | | 600 | 12 |
| | | | 720 | 18 |
| | | | 1440 | <LOQ |
| | 2B | female | 30 | 34 |
| | | | 60 | 33 |
| | | | 235 | 18 |
| | | | 270 | 11 |
| | | | 300 | 31 |
| | | | 475 | 7 |
| | | | 540 | 44 |
| | | | 600 | 18 |
| | | | 720 | 15 |
| | | | 1440 | <LOQ |
| | 2C | female | 30 | 42 |
| | | | 60 | 47 |
| | | | 235 | 6 |
| | | | 270 | 17 |
| | | | 300 | 25 |
| | | | 475 | 6 |
| | | | 540 | 29 |
| | | | 600 | 20 |
| | | | 720 | 7 |
| | | | 1440 | <LOQ |
| | 2D | female | 30 | 38 |
| | | | 60 | 25 |
| | | | 235 | 13 |
| | | | 270 | 51 |
| | | | 300 | 44 |
| | | | 475 | 10 |
| | | | 540 | 29 |
| | | | 600 | 45 |
| | | | 720 | 15 |
| | | | 1440 | <LOQ |
| 2 | 3A | male | 30 | 298 |
| | | | 60 | 153 |
| | | | 235 | 84 |
| | | | 270 | 131 |
| | | | 300 | 312 |
| | | | 475 | 82 |
| | | | 540 | 223 |
| | | | 600 | 269 |
| | | | 720 | 133 |
| | | | 1440 | 12 |
| | 3B | male | 30 | 288 |
| | | | 60 | 106 |
| | | | 235 | 46 |
| | | | 270 | 236 |
| | | | 300 | 232 |
| | | | 475 | 79 |
| | | | 540 | 214 |
| | | | 600 | 173 |
| | | | 720 | 401 |
| | | | 1440 | <LOQ |
| | 3C | male | 30 | 269 |
| | | | 60 | 272 |
| | | | 235 | 63 |
| | | | 270 | 364 |
| | | | 300 | 116 |
| | | | 475 | 97 |
| | | | 540 | 290 |
| | | | 600 | 130 |
| | | | 720 | 137 |
| | | | 1440 | 42 |
| | 3D | male | 30 | 116 |
| | | | 60 | 116 |
| | | | 235 | 105 |
| | | | 270 | 309 |
| | | | 300 | 202 |
| | | | 475 | 114 |
| | | | 540 | 173 |
| | | | 600 | 150 |
| | | | 720 | 71 |
| | | | 1440 | 36 |
| | 4A | female | 30 | 245 |
| | | | 60 | 81 |
| | | | 235 | 75 |
| | | | 270 | 237 |
| | | | 300 | 216 |
| | | | 475 | 144 |
| | | | 540 | 231 |
| | | | 600 | 197 |
| | | | 720 | 186 |
| | | | 1440 | 42 |
| | 4B | female | 30 | 78 |
| | | | 60 | 95 |
| | | | 235 | 36 |
| | | | 270 | 324 |
| | | | 300 | 97 |
| | | | 475 | 219 |
| | | | 540 | 314 |
| | | | 600 | 207 |
| | | | 720 | 165 |
| | | | 1440 | 8 |
| | 4C | female | 30 | 218 |
| | | | 60 | 127 |
| | | | 235 | 23 |
| | | | 270 | 273 |
| | | | 300 | 191 |
| | | | 475 | 178 |
| | | | 540 | 369 |
| | | | 600 | 480 |
| | | | 720 | 244 |
| | | | 1440 | 36 |
| | 4D | female | 30 | 98 |
| | | | 60 | 165 |
| | | | 235 | 72 |
| | | | 270 | 350 |
| | | | 300 | 414 |
| | | | 475 | 179 |
| | | | 540 | 474 |
| | | | 600 | 288 |
| | | | 720 | 217 |
| | | | 1440 | <LOQ |

TABLE 51
Mean Concentrations and Descriptive Statistics of
Anatabine in Plasma Samples at Each Time Point

| | | | | | | | Combined Male and Female | | | |
|---|---|---|---|---|---|---|---|---|---|---|
| | | | | | | | Male and | | | |
| | | Time | Male or Female | | | | Female | | | |
| Dose | | Point | | Avg. Conc. | | | Avg. Conc. | | | |
| Group | Sex | (min) | N = | (ng/ml) | ±SD | ±SEM | (ng/ml) | n = | ±SD | ±SEM |
| 0.2 | male | 30 | 3 | 33 | 4.2 | 2.4 | 36 | 7 | 4.3 | 1.6 |
| | | 60 | 4 | 30 | 4.6 | 2.3 | 31 | 8 | 7.5 | 2.6 |
| | | 235 | 3 | 11 | 1.6 | 0.9 | 10 | 7 | 4.7 | 1.8 |
| | | 270 | 4 | 26 | 6.9 | 3.4 | 26 | 8 | 13.3 | 4.7 |
| | | 300 | 4 | 26 | 3.0 | 1.5 | 27 | 8 | 7.9 | 2.8 |
| | | 475 | 3 | 18 | 5.3 | 3.0 | 11 | 7 | 5.4 | 2.0 |
| | | 540 | 4 | 38 | 17.9 | 8.9 | 30 | 8 | 13.1 | 4.6 |
| | | 600 | 3 | 29 | 10.4 | 6.0 | 26 | 7 | 12.3 | 4.3 |
| | | 720 | 3 | 12 | 6.5 | 3.8 | 13 | 7 | 5.2 | 1.8 |
| | | 1440 | 0 | n/a | n/a | n/a | n/a | 0 | n/a | n/a |
| 0.2 | female | 30 | 4 | 37 | 3.9 | 2.0 | | | | |
| | | 60 | 4 | 35 | 9.7 | 4.9 | | | | |
| | | 235 | 4 | 12 | 6.4 | 3.2 | | | | |
| | | 270 | 4 | 26 | 18.7 | 9.4 | | | | |
| | | 300 | 4 | 34 | 10.9 | 5.5 | | | | |
| | | 475 | 4 | 8 | 1.7 | 0.9 | | | | |
| | | 540 | 4 | 34 | 8.7 | 4.4 | | | | |
| | | 600 | 4 | 28 | 14.7 | 7.3 | | | | |
| | | 720 | 4 | 12 | 4.8 | 2.4 | | | | |
| | | 1440 | 0 | n/a | n/a | n/a | | | | |
| 2 | male | 30 | 4 | 243 | 85.6 | 42.8 | 201 | 8 | 90.1 | 31.9 |
| | | 60 | 4 | 165 | 76.1 | 38.0 | 139 | 8 | 60.4 | 21.3 |
| | | 235 | 4 | 72 | 25.7 | 12.9 | 63 | 8 | 26.9 | 9.5 |
| | | 270 | 4 | 303 | 100.9 | 50.5 | 278 | 8 | 76.4 | 27.0 |
| | | 300 | 4 | 183 | 80.7 | 40.3 | 222 | 8 | 102.3 | 36.2 |
| | | 475 | 4 | 97 | 16.2 | 8.1 | 136 | 8 | 51.7 | 18.3 |
| | | 540 | 4 | 226 | 48.3 | 24.1 | 286 | 8 | 98.7 | 34.9 |
| | | 600 | 4 | 151 | 61.7 | 30.8 | 237 | 8 | 112.2 | 39.7 |
| | | 720 | 4 | 203 | 146.6 | 73.3 | 194 | 8 | 99.1 | 35.0 |
| | | 1440 | 3 | 39 | 15.9 | 9.2 | 29 | 6 | 15.5 | 6.3 |
| 2 | female | 30 | 4 | 160 | 83.8 | 41.9 | | | | |
| | | 60 | 4 | 129 | 37.3 | 18.6 | | | | |
| | | 235 | 4 | 44 | 25.8 | 12.9 | | | | |
| | | 270 | 4 | 316 | 50.8 | 25.4 | | | | |
| | | 300 | 4 | 234 | 133.3 | 66.6 | | | | |
| | | 475 | 4 | 192 | 30.7 | 15.3 | | | | |
| | | 540 | 4 | 386 | 102.1 | 51.1 | | | | |
| | | 600 | 4 | 325 | 131.0 | 65.5 | | | | |
| | | 720 | 4 | 208 | 34.7 | 17.3 | | | | |
| | | 1440 | 3 | 22 | 18.6 | 10.7 | | | | |

### EXAMPLE 6

### Treatment of Thyroiditis

This example illustrates administering anatabine for treating thyroiditis. A female patient, aged approximately 52, had been afflicted with Hashimoto's thyroiditis for approximately 5 years. The patient's condition had advanced to a state where the treating physician recommended a thyroid lobectomy. The patient orally ingested a tablet containing about 600 μg anatabine citrate, 20 times daily over a period of 30 days. At the conclusion of the treatment, inflammation of the thyroid was reduced to normal levels, such that the patient was no longer in need of a thyroid lobectomy. The patient continued the treatment for an additional 30 days, after which time the patient's voice distortion associated with thyroiditis was no longer present.

### EXAMPLE 7

### Use of Anatabine to Treat Epilepsy and Autism

A 10-year old male patient, who was diagnosed with autism and a seizure disorder, had brain surgery and began rehabilitation the following month. About 4 months later, in addition to continuing rehabilitation, he began a course of treatment with 1.0 mg of anatabine three times per day. Over the course of 3½ weeks the frequency of the patient's seizures decreased from one per day to approximately one per week. The patient also experienced cognitive benefits beginning approximately one week after the start of the anatabine treatment, with noticeable improvements daily. These benefits included improved communication and language skills and the ability to focus.

### EXAMPLE 8

### Effect of Nicotine and Anatabine on Nicotinic Acetylcholine Receptor Channels In Vitro

This example demonstrates the in vitro effects of nicotine (−) isomer and anatabine racemate (“test articles”) on three cloned human nicotinic acetylcholine receptor (nAChR) channels expressed in mammalian cells using a Fluo-8 calcium kit and a Fluorescence Imaging Plate Reader (FLIPR TETRA™) instrument. The following three (3) channels were evaluated:
1. nAChR α3/β4 (encoded by the human CHRNA3 and CHRNB4 gene and stably expressed in CHO cells);
2. nAChR α4/β2 (CHRNA4 and CHRNB2 gene and transiently expressed in HEK293 cells); and
3. nAChR α7 (encoded by the human nicotinic α7 gene coexpressed in CHO cells with the chaperone RIC-3 encoded by the human RIC3 gene).

The ability of each test article to act as an agonist, a positive allosteric modulator or antagonist of three nAChR receptor channels was evaluated in the presence of 0.1 μM atropine. Both test articles were evaluated for responses on each nAChR channel at eight (8) concentrations: 0.3, 1, 3, 10, 30, 100, 300, and 1000 μM (n=4 for each concentration). The results are summarized below and detailed in Tables 54, 56, and 58. The z-prime factors for each channel are presented in Tables 55, 57, and 59.

In the agonist assay, both test articles increased all three nAChR channel signals in a concentration-dependent manner indicating that the test articles were agonists of the channels. The nicotine (−) isomer showed the highest agonist activity towards the nAChR α4/β2 channel (EC₅₀=1.302 μM), followed by the nAChR α3/β4 channel (EC₅₀=27.78 μM). The EC₅₀ of nicotine for the nAChR 7 channel could not be determined as maximal stimulation was not achieved within the range of concentrations tested. The EC₅₀ for the anatabine racemate could only be determined for the nAChR α4/β2 receptor (EC₅₀=282 μM) as the maximum level of stimulation was not achieved or could not be determined for the nAChR α3/β4 and nAChR α7 channels. Therefore, anatabine displays full agonist activity towards the α4/β2 receptor and agonist activity towards the α3/β4 and α7 receptors; however, it was not possible to determine if anatabine is a partial agonist of the latter two.

In the potentiation assay, the test articles did not increase the signals with a low dose of Ach stimulation, indicating that they are not potentiators or allosteric modulators of the channels.

In the antagonist assay, after stimulation with high dose of Ach, the test articles decreased the Ach-induced signals. However, since the test articles acted as agonists, the reduction of Ach-induced signals was caused by the desensitization of the channel themselves, rather than the channel blockage. Nicotine and anatabine are not considered to be antagonists of these receptors.

Formulations All chemicals used in solution preparations were purchased from Sigma-Aldrich (St. Louis, Mo.) unless otherwise noted and were of ACS reagent grade purity or higher. Stock solutions of test and control articles were prepared in dimethyl sulfoxide (DMSO) and stored frozen. Test article and positive control concentrations were prepared fresh daily by diluting stock solutions into the appropriate solutions. The test and control article formulations were loaded in a glass-lined, 384-well compound plate, and placed in the compound plate wells of a FLIPR TETRA™ (MDS-AT) instrument.

Test Articles The effect of 8 concentrations of each test article was evaluated (n=4). The sponsor provided the anatabine racemate and nicotine (−) isomer was purchased from Sigma-Aldrich.

| | | | | | | Test |
|---|---|---|---|---|---|---|
| | | | Amount | | | Concen- |
| Test | | MW | Received | | Purity | trations |
| Article ID | Lot | (g/mol): | (mg or μl) | | (%) | (μM) |
| Anatabine | A210192 | 160.22 | 2 | g | 99.5% | 0.3, 1, 3, |
| (racemate) | | | | | | 10, 30, 100, |
| | | | | | | 300, 1000 |
| Nicotine (−) | 1425810V | 162.23 | 25 | mL | 99 | 0.3, 1, 3, |
| isomer | | | | | | 10, 30, 100, |
| | | | | | | 300, 1000 |

Positive Control Articles Stock solutions of positive control articles were prepared in DMSO and stored frozen. Acetylcholine (Ach) was prepared in distilled H₂O and stored frozen.

TABLE 52

| | | | Molecular | Rationale for |
|---|---|---|---|---|
| Purpose | Name | Source | Weight | selection |
| positive | PNU-120596 | Tocris | 311.72 g/mol | positive |
| control | | | | allosteric |
| | | | | modulator of |
| | | | | nicotinic |
| | | | | receptor |
| | | | | channel |
| positive | Epibatidine | Tocris | 208.69 g/mol | agonist and |
| control | | | | potentiator of |
| | | | | and nicotinic |
| | | | | receptor |
| | | | | channels |
| positive | Methyllycaconitine | Tocris | 874.93 g/mol | inhibits α3, |
| control | citrate (MLA) | | | α4, and α7 |
| | | | | nicotinic |
| | | | | receptor |
| | | | | channels |
| positive | Acetylcholine | Sigma- | 181.66 g/mol | activates all |
| control | (Ach) | Aldrich | | nicotinic |
| | | | | receptor |
| | | | | channels |
| test article | dimethyl sulfoxide | Sigma- | 78.13 g/mol | 0.3% DMSO |
| carrier | (DMSO) | Aldrich | | does not |
| | | | | affect ion |
| | | | | channel |
| | | | | function |

### Test Systems

Cells were maintained in tissue culture incubators per ChanTest SOP. Stocks were maintained in cryogenic storage.

### Cell Culture Procedures

HEK293 or CHO cells were transiently or stably transfected with the appropriate human ion channel cDNAs. Stable transfectants were selected by coexpression with the antibiotic-resistance gene(s) incorporated into the expression plasmid(s). Selection pressure was maintained by including selection antibiotics in the culture medium. HEK293 cells were cultured in Dulbecco's Modified Eagle Medium/Nutrient Mixture F-12 (D-MEM/F-12) supplemented with 10% fetal bovine serum, 100 U/mL penicillin G sodium, 100 g/mL streptomycin sulfate and appropriate selection antibiotics. CHO cells were cultured in Ham's F-12 supplemented with 10% fetal bovine serum, 100 U/mL penicillin G sodium, 100 g/mL streptomycin sulfate and appropriate selection antibiotics.

For FLIPR TETRA™ assay, cells were plated in 384-well black wall, flat clear bottom microtiter plates (Type: BD Biocoat Poly-D-Lysine Multiwell Cell Culture Plate) at 20,000 to 30,000 cells per well (384-well plate). Cells were incubated at 37° C. overnight or until the cells reached a sufficient density in the wells (near confluent monolayer) to use in fluorescence assays. For α4β2 assay, cells were incubated at 27° C. for at least 7 hours before use.

### Test Methods

Experiments were performed with either the FLIPR calcium sensitive dye kit (Fluo-8, ABDbioquest, for nAChR α3/β4 and nAChR α7) or FLIPR membrane potential kit (Molecular Devices, for nAChR α4/β2) according to the manufacturer's instructions. Briefly, cells were incubated with 20 l dye for 30 min at 37° C. After dye loading period, for the agonist assay, 5 μl of the test, vehicle, or control articles at concentration of 5 times of final concentration were applied to the cells without stimulation. For the positive allosteric modulation (PAM) and antagonist assay, after test article application, the cells were stimulated with either low dose (for PAM) or high dose (for antagonist) of Ach.

TABLE 53

| designation | organism | tissue | morphology | age/stage | strain source | sub-strain source |
|---|---|---|---|---|---|---|
| HEK293 | H. sapiens | kidney; Transformed | epithelial | embryo | ATCC, | ChanTest Corp., |
| | | with adenovirus 5 DNA; | | | Manassas, VA | Cleveland, OH |
| | | Transfected with human | | | | |
| | | ion channel cDNAs | | | | |
| CHO | Cricetulus | Ovary; transfected | epithelial | embryo | | |
| | griseus | with human ion | | | | |
| | | channel cDNA(s) | | | | |

### Analysis

Data was stored on the ChanTest computer network (and backed-up nightly) for off-line analysis. Data acquisition was performed via the FLIPR Control software that is supplied with the FLIPR System (MDS-AT) and data was analyzed using Microsoft Excel 2003 (Microsoft Corp., Redmond, Wash.).

Concentration-response data was fitted to a Hill equation of the following form:

$$RESPONSE = Base + \frac{Max - Base}{1 + {( \frac{xhalf}{x} )}^{rate}}$$

where Base is the response at low concentrations of test article, Max is the maximum response at high concentrations, xhalf is the EC₅₀ or IC₅₀, the concentration of test article producing either half-maximal activation or inhibition, and rate is the Hill coefficient. Nonlinear least squares fits were made assuming a simple one-to-one binding model.

Z-prime factors for the agonist, positive allosteric modulator and antagonist control assays were calculated and are indicative of assay quality. Values above 0.5 represent an excellent assay with clear separation between positive and negative control responses. Z-prime factors between 0 and 0.5 are marginal, but still useful for screening purposes.

1. nAChR α3/β4 Receptor Channel Assay

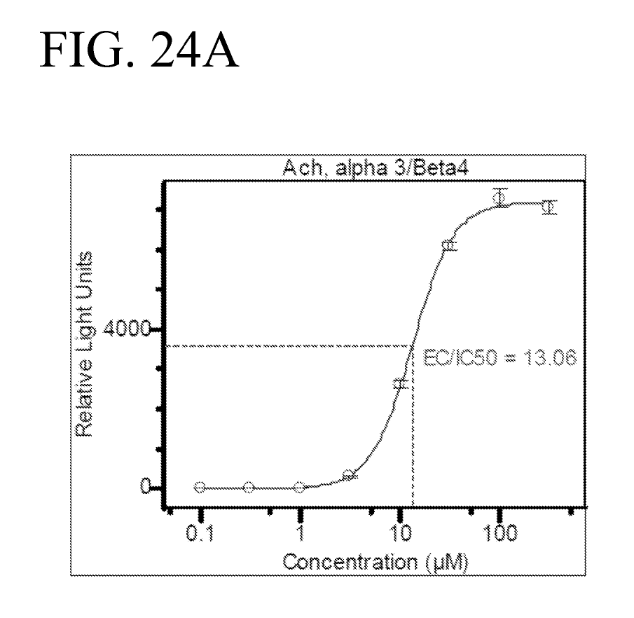

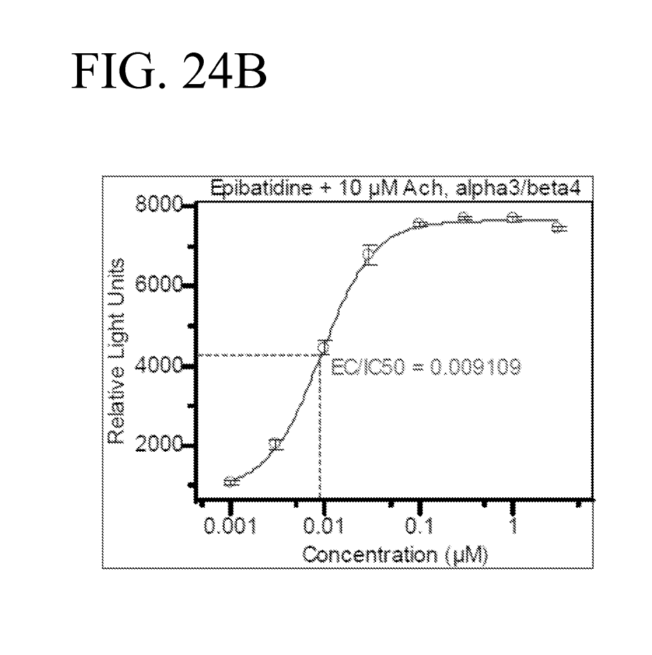

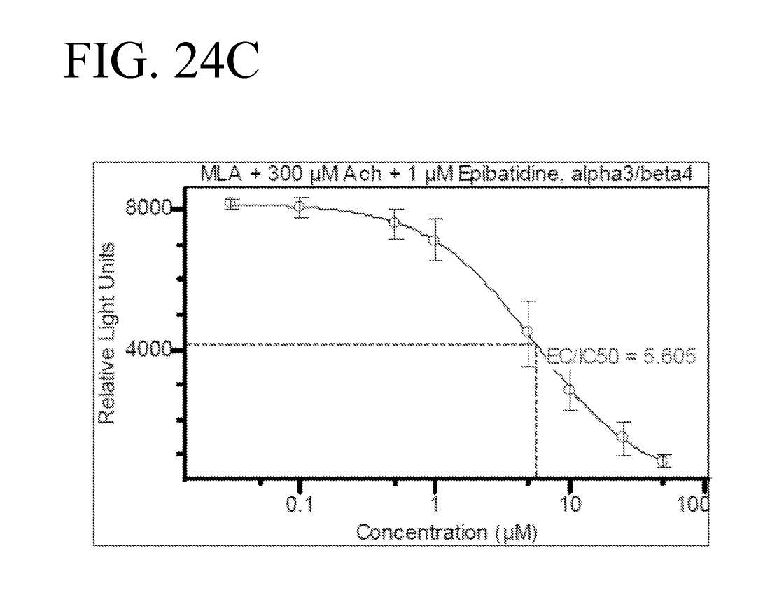

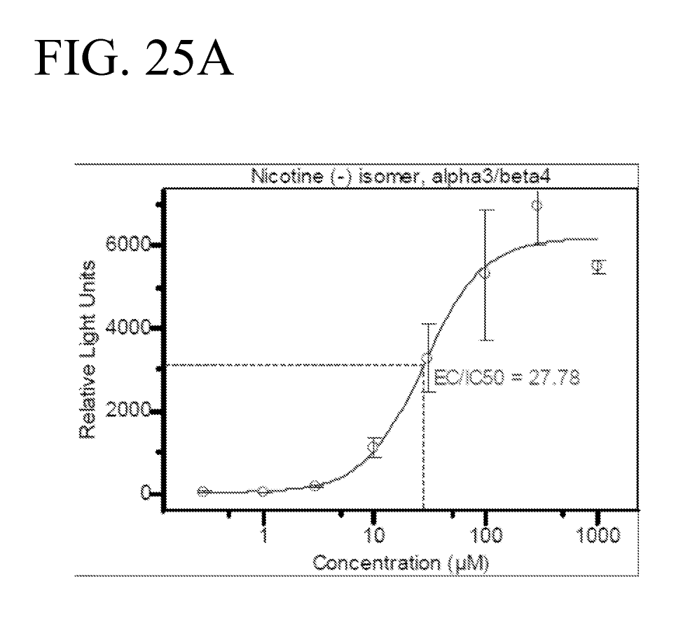

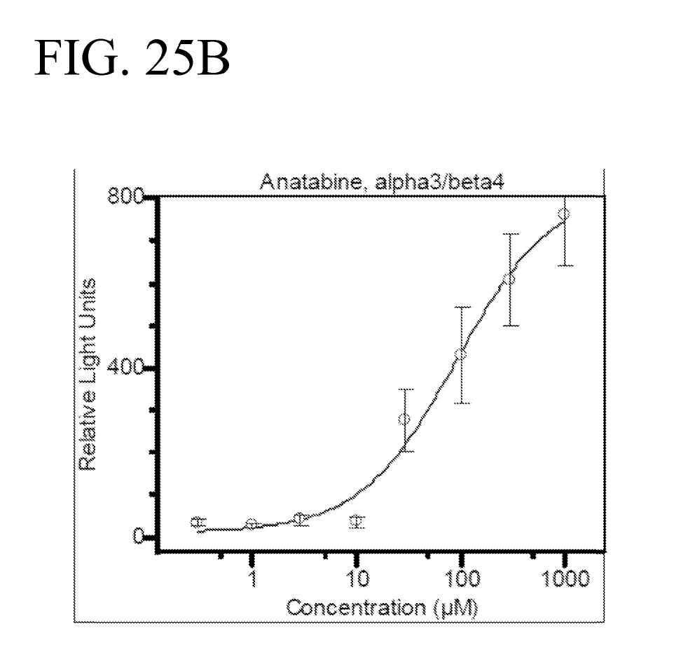

The ability of both test articles to act as an agonist of nAChR carried out in the absence of the positive control agonist. The signal elicited in the presence of the positive control agonist, 30 μM acetylcholine (Ach) was set to 100% and the signal from the vehicle control, HEPES-buffered physiological saline (HBPS) solution was set to 0%. The test article results are presented as normalized % activation and are shown in Table 54. Values were considered significant (bold font) if the test article mean was three or more standard deviations above the vehicle control mean. The concentration-response relationship of normalized % activation of Ach is shown in FIG. 24A. The EC₅₀ of nicotine (−) isomer could be determined (EC₅₀=27.78 μM) and the concentration-response relationship of normalized % activation for both test articles are presented in FIG. 25. Although racemic anatabine showed agonist activity, the EC₅₀ could not be determined as the maximal level of stimulation was not achieved within the concentration range that was evaluated.

The ability of each test article to act as a positive allosteric modulator of nAChR α3β4 was carried out in the presence of a low concentration of the positive control agonist (10 μM Ach) alone. The signal elicited in the presence of the positive control agonist and allosteric modulator (10 μM Ach+1 μM epibatidine) was set to 100% and the signal from the agonist control (10 μM Ach) was set to 0%. The test article results are presented as normalized % potentiation and are shown in Table 54. Values were considered significant (bold font) if the test article mean was three or more standard deviations above the agonist control mean. Neither nicotine nor anatabine potentiated the activity of the nAChR α3/β4 receptor. The concentration-response relationship of the normalized % potentiation by epibatidine is shown in FIG. 24B.

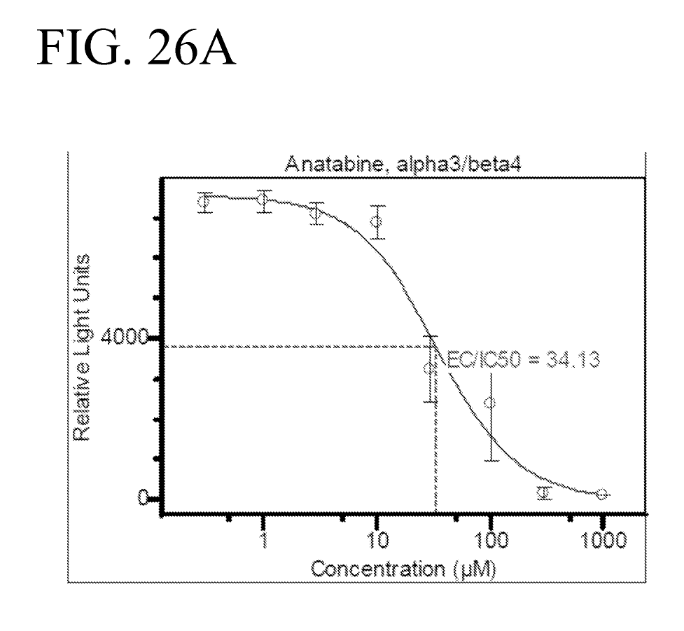

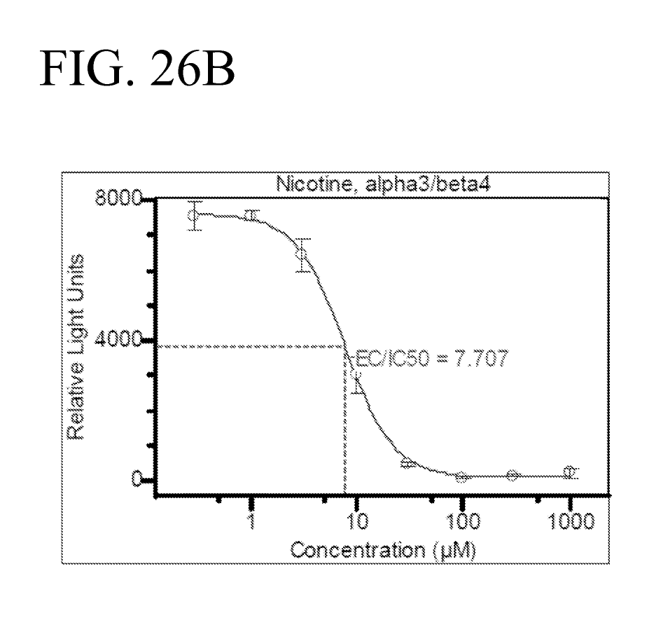

The ability of each test article to act as an antagonist of nAChR α3/β4 was carried out in the presence of a high concentration of the positive control agonist (300 μM Ach) and the positive allosteric modulator (1 μM epibatidine). The signal, elicited in the presence of the positive control agonist and the positive allosteric modulator (300 μM Ach+1 μM epibatidine), was set to 100% and the signal in the presence of the positive control antagonist (300 μM Ach+1 μM epibatidine+10 μM methyllycaconitine, MLA) was set to 0. The normalized inhibition of the test articles are shown in Table 54. Values were considered significant (bold font) if the test article mean was three or more standard deviations below the positive control agonist plus positive allosteric modulator mean. The concentration-response relationship of normalized % inhibition of MLA is shown in FIG. 24C. The IC₅₀ of both test articles could be determined (nicotine (−) isomer, IC₅₀ 7.707 μM; racemic anatabine, IC₅₀=34.13 μM) and the concentration-response relationships of normalized % inhibition are presented in FIG. 26. It should be noted that because nicotine and anatabine are both agonists of this receptor, their inhibitory effects are indicative of receptor desensitization rather than antagonism.

The Z-prime factors for the agonist, positive allosteric modulator and antagonist assays are presented in Table 55 and are indicative of assay quality.

TABLE 54
Effect of the Test Articles on nAChR α3/β4 Channel

| | | Normal- | | Normal- | | Normal- | |
|---|---|---|---|---|---|---|---|
| Test | Test | ized % | | ized % | | ized % | |
| Article | conc | Acti- | | Poten- | | Inhi- | |
| ID | (μM) | vation | SD | tiation | SD | bition | SD |
| Anatabine | 0.3 | 1.32 | 0.21 | −1.98 | 0.90 | 22.21 | 4.61 |
| (racemate) | 1 | −0.01 | 0.89 | −4.08 | 2.89 | 21.83 | 4.51 |
| | 3 | −1.53 | 0.54 | −2.88 | 0.68 | 27.15 | 4.93 |
| | 10 | −2.73 | 0.43 | −3.20 | 1.39 | 30.61 | 7.11 |
| | 30 | 4.93 | 2.71 | −8.50 | 1.70 | 99.02 | 3.43 |
| | 100 | 10.44 | 4.31 | −9.46 | 0.68 | 118.93 | 4.11 |
| | 300 | 16.97 | 4.00 | −9.38 | 0.46 | 144.56 | 2.11 |
| | 1000 | 22.70 | 4.38 | −9.17 | 0.47 | 144.81 | 0.13 |
| Nicotine | 0.3 | 0.94 | 1.49 | −1.23 | 0.69 | 19.08 | 6.99 |
| (−) | 1 | −1.28 | 1.38 | −1.19 | 0.51 | 19.01 | 2.88 |
| isomer | 3 | 1.11 | 0.65 | −3.92 | 0.43 | 37.78 | 7.71 |
| | 10 | 38.17 | 9.39 | −5.67 | 2.19 | 95.81 | 9.31 |
| | 30 | 122.59 | 32.46 | −9.28 | 0.93 | 138.96 | 0.88 |
| | 100 | 201.50 | 62.22 | −9.72 | 0.58 | 145.26 | 0.29 |
| | 300 | 264.77 | 37.00 | −8.45 | 0.23 | 143.89 | 0.62 |
| | 1000 | 208.23 | 5.74 | −9.57 | 0.94 | 142.14 | 2.34 |

Note:
Bolded values are significantly different from the respective control means
For activation: 3 × SD of vehicle control mean (HBPS) = 4.08; values > 4.08 are significant.
For positive allosteric modulation: 3 × SD of agonist mean (10 μM Ach) = 9.24; values > 9.24 are significant
For inhibition: 3 × SD of agonist plus positive allosteric modulator mean (300 μM Ach + 1 μM epibatidine) = 10.89; values > 10.89 are significant

TABLE 55
Positive Controls

| | | Normalized | |
|---|---|---|---|
| Channel | Test Article ID | Signals | SD |
| nAChR α3/β4 | 30 μM Ach | 100 | 5.07 |
| (activation) | HBPS | 0 | 1.36 |
| | Z Prime = 0.81 | | |
| nAChR α3/β4 | 10 μM Ach + 1 μM | 100 | 1.12 |
| (potentiation) | Epibatidine | | |
| | 10 μM Ach | 0 | 3.08 |
| | Z Prime = 0.87 | | |
| nAChR α3/β4 | 300 μM Ach + 1 μM | 0 | 3.63 |
| (inhibition) | Epibatidine | | |
| | 300 μM Ach + 1 μM | 100 | 10.09 |
| | Epibatidine + 10 μM MLA | | |

Z Prime = 0.56

2. nAChR α4/β2 Receptor Channel Assay

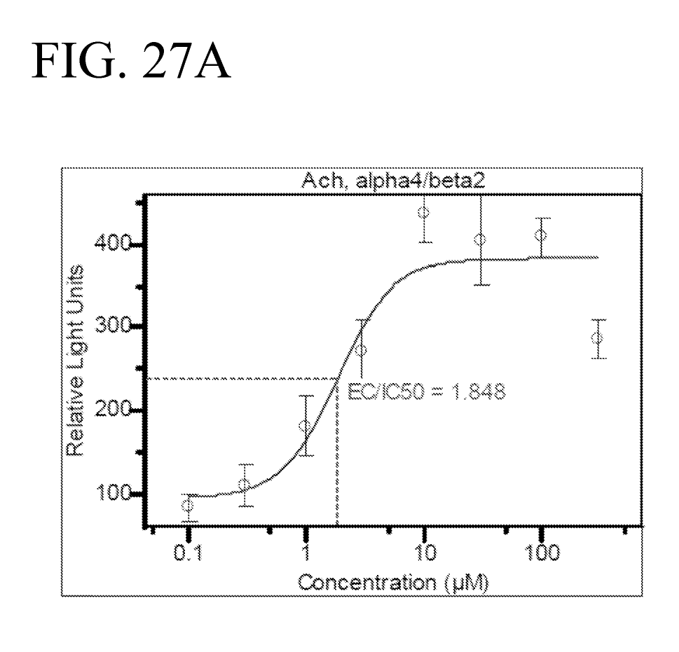

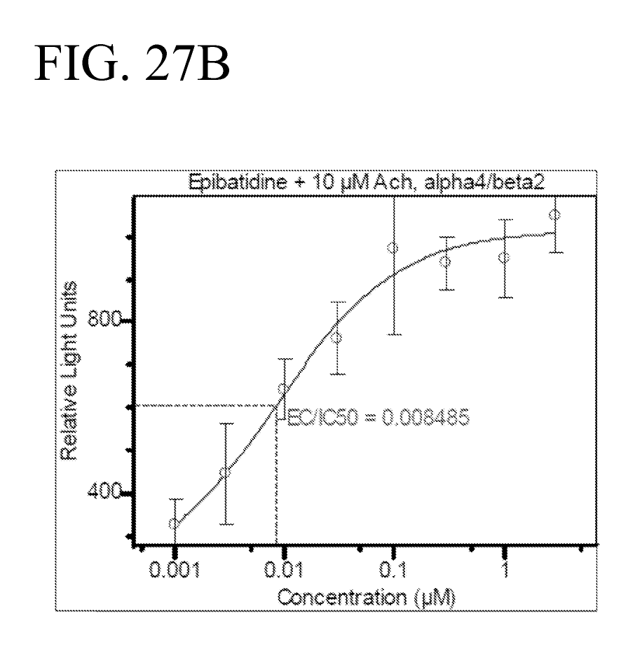

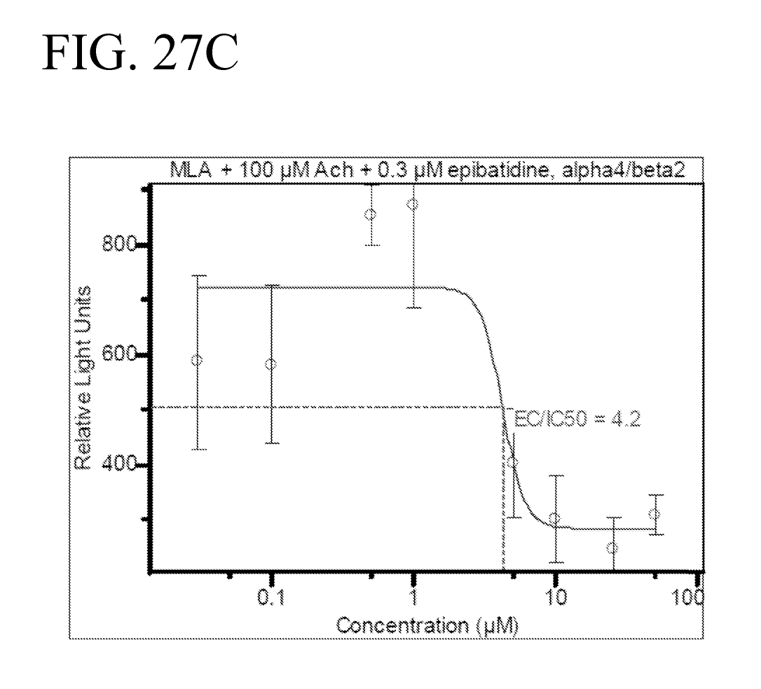

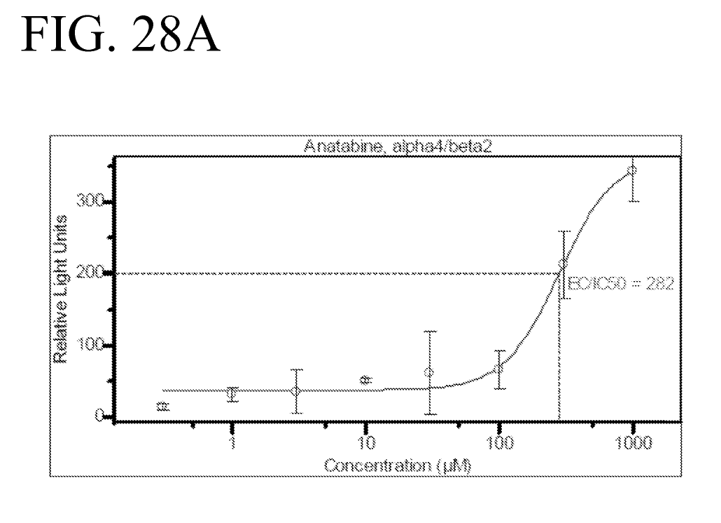

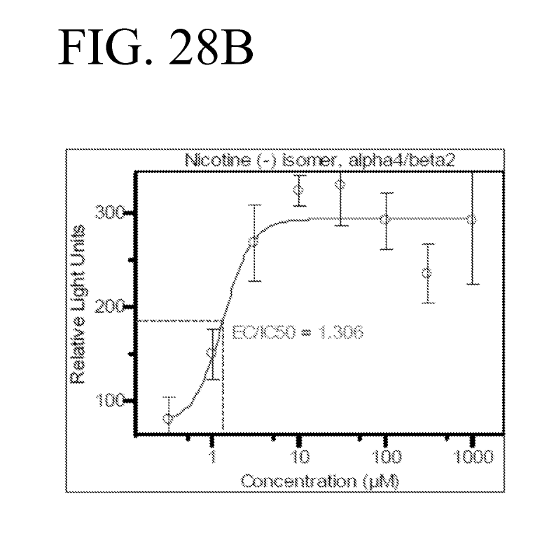

The ability of each test article to act as an agonist of the nAChR carried out in the absence of the positive control agonist. The signal elicited in the presence of the positive control agonist (30 μM Ach) was set to 100% and the signal from the vehicle control (HBPS) was set to 0%. The test article results are presented as normalized % activation and are shown in Table 56. Values were considered significant (bold font) if the test article mean was three or more standard deviations above from the vehicle control mean. The concentration-response relationship of the normalized % activation of Ach is shown in FIG. 27A. The EC₅₀ of both test articles could be determined (nicotine (−) isomer, EC₅₀=1.302 μM; racemic anatabine, EC₅₀=282 μM) and the concentration-response relationships of the normalized % activation for the test articles are presented in FIG. 28. Both test articles are full agonists of this channel.

The ability of each test article to act as a positive allosteric modulator of nAChR α4/β2 was carried out in the presence of a low concentration of the positive control agonist (10 μM Ach) alone. The signal elicited in the presence of the positive control agonist and allosteric modulator epibatidine (10 μM Ach+3 μM epibatidine) was set to 100% and the signal from the agonist control (10 μM Ach) was set to 0%. The test article results are presented as normalized % potentiation and are shown in Table 56. Values were considered significant (bold font) if the test article mean was three or more standard deviations above the agonist mean. The concentration-response relationship of the normalized % potentiation of epibatidine is shown in FIG. 27B. Neither test article potentiated the activity of the nAChR α4/β2 receptor.

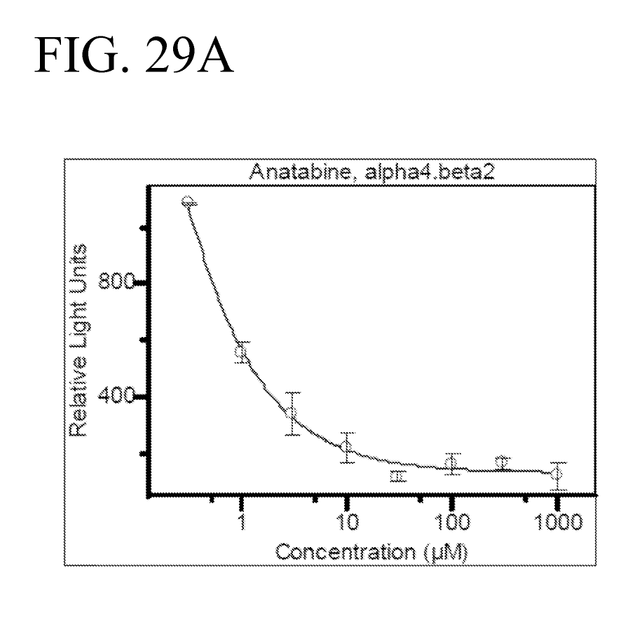

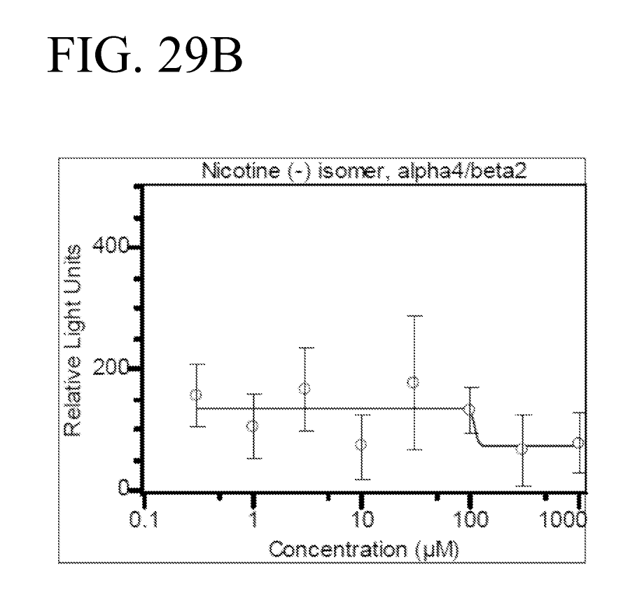

The ability of each test article to act as an antagonist of nAChR α4/β2 was carried out in the presence of a high concentration of the positive control agonist (100 μM Ach) and the positive allosteric modulator, 0.3 μM epibatidine. The signal, elicited in the presence of the positive control agonist and the positive allosteric modulator (100 μM Ach+0.3 μM epibatidine), was set to 100% and the signal in the presence of the positive control antagonist MLA (100 μM Ach+0.3 μM epibatidine+10 μM MLA) was set to 0. The normalized inhibition of the test articles are shown in Table 56. Values were considered significant (bold font) if the test article mean was three or more standard deviations below the positive control agonist mean. The concentration-response relationship of the normalized % inhibition of MLA is shown in FIG. 27C. The concentration-response relationships of the normalized % inhibition for the test articles are presented in FIG. 29. Although both nicotine and anatabine inhibited receptor activity, the IC₅₀s could not be determined for either test article since the inhibitions were not concentration-dependent. Nicotine and anatabine are both agonists of this receptor and therefore their inhibitory effects are indicative of receptor desensitization rather than antagonism.

The Z-prime factors for the agonist, positive allosteric modulator and antagonist assays are presented in Table 57.

TABLE 56
Effect of the Test Articles on nAChR α4/β2 Channel

| | | Normal- | | Normal- | | Normal- | |
|---|---|---|---|---|---|---|---|
| Test | Test | ized % | | ized % | | ized % | |
| Article | conc | Acti- | | Poten- | | Inhi- | |
| ID | (μM) | vation | SD | tiation | SD | bition | SD |
| Anatabine | 0.3 | 0.28 | 3.08 | −18.94 | 5.64 | −20.93 | 0.41 |
| (racemate) | 1 | 5.36 | 5.14 | −27.03 | 19.93 | 49.23 | 4.59 |
| | 3 | 10.41 | 8.66 | −35.59 | 2.81 | 59.57 | 10.42 |
| | 10 | 12.65 | 1.79 | −42.34 | 7.10 | 92.94 | 7.83 |
| | 30 | 9.07 | 4.24 | −39.48 | 6.15 | 84.03 | 4.80 |
| | 100 | 16.96 | 5.70 | −38.34 | 12.74 | 97.26 | 8.27 |
| | 300 | 50.60 | 2.87 | −41.07 | 9.18 | 84.96 | 4.98 |
| | 1000 | 85.29 | 10.49 | −35.30 | 2.53 | 96.88 | 3.70 |
| Nicotine | 0.3 | 16.27 | 3.95 | −46.00 | 8.03 | 85.93 | 8.84 |
| (−) | 1 | 34.05 | 7.15 | −46.34 | 5.03 | 107.14 | 6.98 |
| isomer | 3 | 61.88 | 9.61 | −40.54 | 8.72 | 85.74 | 10.24 |
| | 10 | 74.31 | 1.54 | −42.16 | 6.73 | 109.33 | 9.09 |
| | 30 | 75.53 | 7.12 | −43.70 | 8.78 | 107.14 | 4.81 |
| | 100 | 71.02 | 7.25 | −43.40 | 2.35 | 91.94 | 8.93 |
| | 300 | 58.23 | 8.27 | −34.56 | 4.78 | 95.26 | 3.36 |
| | 1000 | 57.05 | 2.03 | −39.66 | 5.99 | 108.16 | 7.40 |

Note:
Bolded values are significantly different from the respective control means
For activation: 3 × SD of vehicle control mean (HBPS) = 12.74; values > 12.74 are significant
For positive allosteric modulation: 3 × SD of agonist mean (10 μM Ach) = 20.04; values > 20.04 are significant
For inhibition: 3 × SD of agonist plus positive allosteric modulator mean (100 μM Ach + 0.3 μM epibatidine) = 43.00; values > 43.00 are significant

TABLE 57
Positive Controls

| | | Normalized | |
|---|---|---|---|
| Channel | Test Article ID | Signals | SD |
| nAChR α4/β2 | 30 μM Ach | 100 | 8.87 |
| (activation) | HBPS | 0 | 4.25 |
| | Z Prime = 0.61 | | |
| nAChR α4/β2 | 10 μM Ach + 0.3 μM | 100 | 8.24 |
| (potentiation) | Epibatidine | | |
| | 10 μM Ach | 0 | 6.68 |
| | Z Prime = 0.55 | | |
| nAChR α4/β2 | 100 μM Ach + 0.3 μM | 0 | 14.33 |
| (inhibition) | Epibatidine | | |
| | 100 μM Ach + 0.3 μM | 100 | 5.38 |
| | Epibatidine + 10 μM MLA | | |

Z Prime = 0.41

3. nAChR α7 Receptor Channel Assay

The ability of each test article to act as an agonist of nAChR α7 receptor channel was carried out in the absence of the positive control agonist, Ach. The signal elicited in the presence of the positive control agonist and allosteric modulator PNU-120596 (30 μM Ach+10 μM PNU-120596) was set to 100% and the signal from the vehicle control (HBPS) was set to 0%. In the absence of PNU-120596, the nAChR α7 receptor became desensitized very quickly before any agonist effect of Ach could be observed. Therefore, the assay of agonist activity for either Ach or for the test articles was conducted in the presence of the positive allosteric modulator.

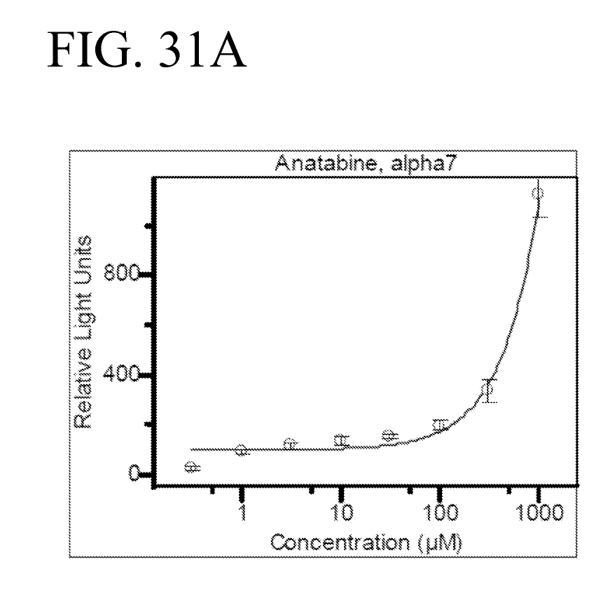

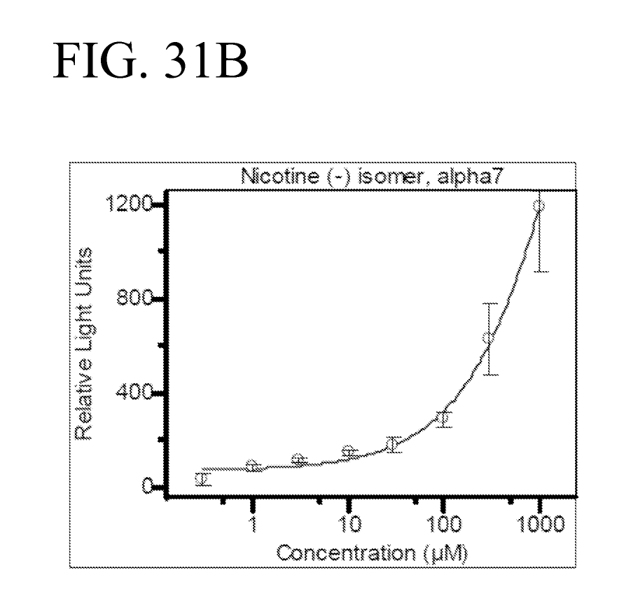

The test article results are presented as the normalized % activation and are shown in Table 58. Values were considered significant (bold font) if the test article mean was three or more standard deviations above the vehicle control mean. The concentration-response relationships of the normalized % activation for the test articles are presented in FIG. 31. Although both nicotine and anatabine showed agonist activity, maximal stimulation was not achieved within the range of concentrations tested and therefore, EC₅₀s could not be determined.

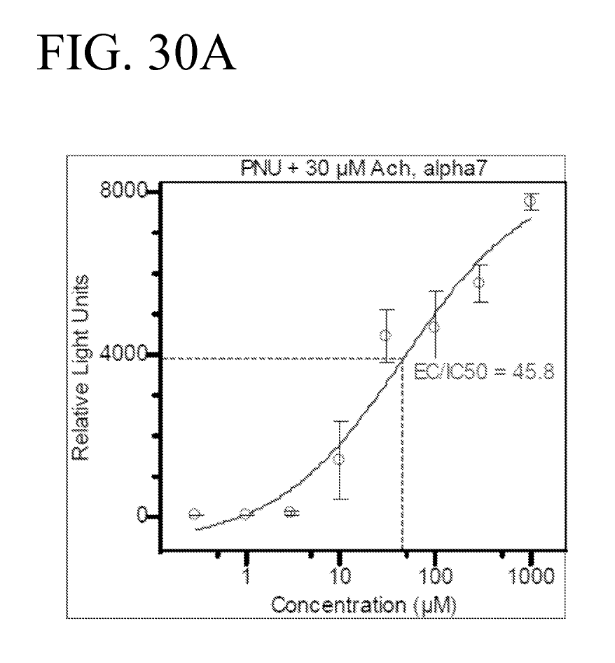

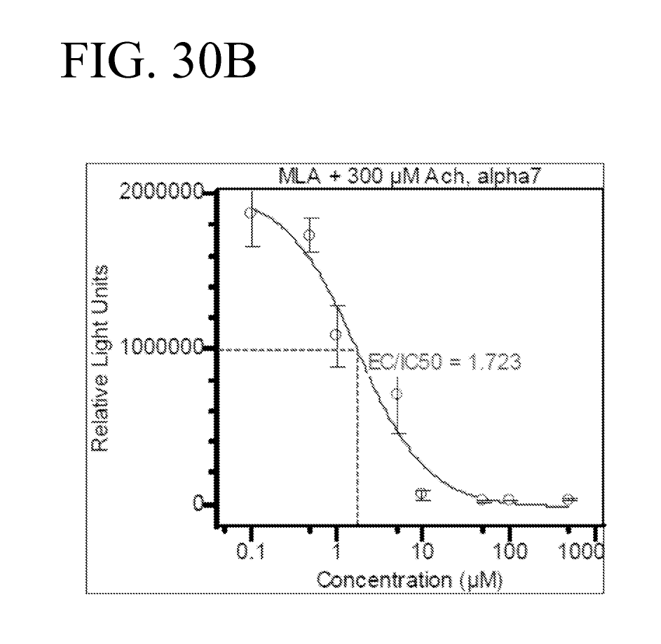

The ability of each test article to act as a positive allosteric modulator of nicotinic α7 was carried out in the presence of the positive control agonist (30 μM Ach) alone. The signal elicited in the presence of the positive control agonist and allosteric modulator PNU-120596 (30 μM Ach+10 μM PNU-120596) was set to 100% and the signal from the agonist control (30 μM Ach) was set to 0%. The test article results are presented as the normalized % potentiation and are shown in Table 58. Values were considered significant and in bold font if the test article mean was three or more standard deviations above the agonist control mean. The concentration-response relationship of the normalized % potentiation of PNU-120596 is shown in FIG. 30A. Neither test article potentiated the activity of the nAChR α7 receptor.

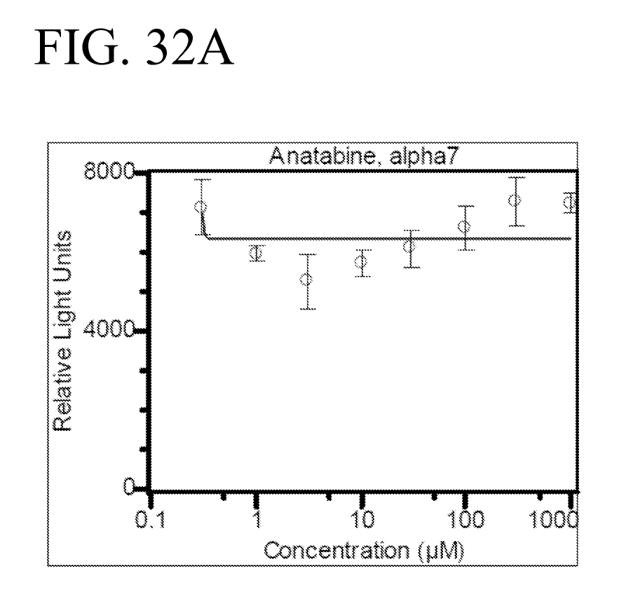

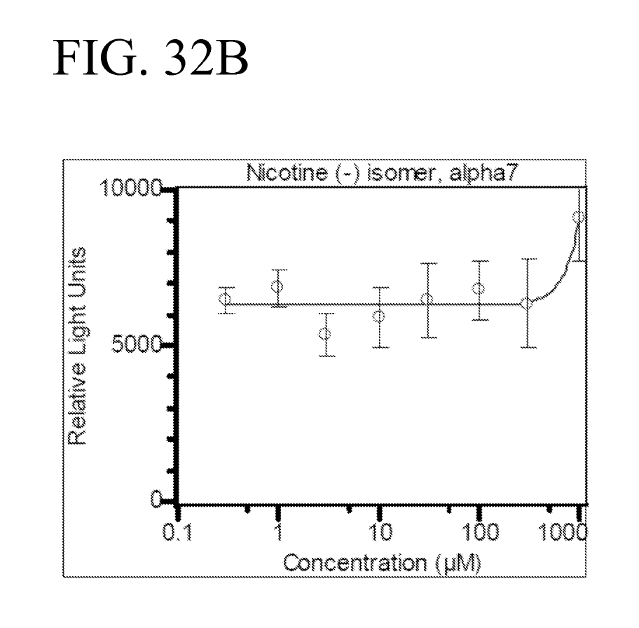

The ability of each test article to act as an antagonist of nAChR α7 was carried out in the presence of the high concentration of a positive control agonist (300 μM Ach) and the positive allosteric modulator (10 μM PNU-120596). The signal, elicited in the presence of the positive control agonist and the positive allosteric modulator (300 μM Ach+10 μM PNU-120596), was set to 100% and the signal in the presence of the positive control antagonist (300 μM Ach+10 μM PNU-120596+10 μM MLA) was set to 0. The normalized inhibition of the test articles are shown in Table 58. Values were considered significant (bold font) if the test article mean was three or more standard deviations below the positive control agonist plus positive allosteric modulator mean. The concentration-response relationship of the normalized % inhibition of MLA is shown in FIG. 30B. The concentration-response relationships of the normalized % inhibition for the test articles are presented in FIG. 32. Inhibition was not concentration-dependent and the IC₅₀s could not be determined for either test article.

The Z-prime factors for the agonist, positive allosteric modulator and antagonist assays are presented in Table 59.

TABLE 58
Effect of the Test Articles on nAChR α7 Channel

| | | Normal- | | Normal- | | Normal- | |
|---|---|---|---|---|---|---|---|
| Test | Test | ized % | | ized % | | ized % | |
| Article | conc | Acti- | | Poten- | | Inhi- | |
| ID | (μM) | vation | SD | tiation | SD | bition | SD |
| Anatabine | 0.3 | −1.81 | 0.31 | −0.94 | 0.09 | 22.38 | 2.91 |
| (racemate) | 1 | 0.34 | 0.42 | −1.05 | 0.06 | 22.23 | 9.43 |
| | 3 | 0.78 | 0.43 | −0.82 | 0.07 | 45.19 | 5.32 |
| | 10 | 1.65 | 0.57 | −0.51 | 0.24 | 33.42 | 4.55 |
| | 30 | 2.27 | 0.31 | −0.57 | 0.11 | 22.88 | 8.05 |
| | 100 | 5.69 | 0.45 | −0.64 | 0.04 | 21.99 | 8.18 |
| | 300 | 14.45 | 2.30 | −0.74 | 0.09 | 7.51 | 5.17 |
| | 1000 | 60.41 | 5.29 | −1.05 | 0.15 | 10.84 | 4.92 |
| Nicotine | 0.3 | −1.03 | 1.21 | −0.95 | 0.07 | 2.78 | 7.04 |
| (−) | 1 | 0.15 | 0.31 | −1.03 | 0.11 | 11.83 | 7.05 |
| isomer | 3 | 0.72 | 0.45 | −0.81 | 0.14 | 41.24 | 9.79 |
| | 10 | 2.44 | 0.64 | −0.61 | 0.10 | 32.54 | 1.79 |
| | 30 | 5.19 | 1.24 | −0.47 | 0.10 | 39.20 | 11.28 |
| | 100 | 11.60 | 1.79 | −0.46 | 0.08 | 18.65 | 5.90 |
| | 300 | 27.98 | 7.59 | −0.34 | 0.20 | 14.10 | 10.79 |
| | 1000 | 48.47 | 8.31 | −0.51 | 0.27 | −37.01 | 7.09 |

Note:
Bolded values are significantly different from the respective control means
For activation: 3 × SD of vehicle control mean (HBPS) = 2.65; values > 2.65 are significant
For positive allosteric modulation: 3 × SD of agonist mean (30 μM Ach) = 19.00; values > 19.00 are significant
For inhibition: 3 × SD of agonist with the positive allosteric modulation mean (300 μM Ach + 10 μM PNU) = 28.86; values > 28.86 are significant

TABLE 59
Positive Controls

| | | Normalized % | |
|---|---|---|---|
| Channel | Test Article ID | signals | SD |
| Nicotinic α7 | 30 μM Ach + 10 μM | 100 | 1.10 |
| (activation) | PNU-120596 | | |
| | HBPS | 0 | 0.88 |
| | Z Prime = 0.50 | | |
| Nicotinic α7 | 30 μM Ach + 10 μM | 100 | 6.33 |
| (potentiation) | PNU-120596 | | |
| | 30 μM Ach | 0 | 0.21 |
| | Z Prime = 0.80 | | |
| Nicotinic α7 | 300 μM Ach + 10 μM | 0 | 9.62 |
| (inhibition) | PNU-120596 | | |
| | 300 μM Ach + 10 μM | 100 | 7.46 |
| | PNU-120596 + 10 μM MLA | | |

Z Prime = 0.49

### Discussion and Conclusions

In this study, the ability of nicotine (−) isomer and anatabine racemate to act as agonists, positive allosteric modulators or antagonists of three nAChR receptor channels was evaluated. Both test articles were evaluated for responses on the α3/β4, α4/β2 and α7 nAChR channels at eight (8) concentrations with four (4) replicates for each concentration.

In the agonist assay, both test articles increased all three nAChR channel signals in a concentration-dependent manner indicating that the test articles were agonists. The nicotine (−) isomer showed the highest activity towards the nAChR α4/β2 channel (EC₅₀=1.302 μM), followed by the nAChR α3/β4 channel (EC₅₀=27.78 μM). The EC₅₀ of nicotine for the nAChR 7 channel could not be determined as maximal stimulation was not achieved within the range of concentrations tested.

The EC₅₀ for the anatabine racemate could only be determined for the nAChR (EC₅₀=282 μM) as the maximum level of stimulation was not achieved or could not be determined for the nAChR α3/β4 and nAChR α7 receptor. From these results it can be concluded that the nicotine (−) isomer is a more potent agonist than the anatabine racemate of both the α4/β2 and α3/β4 nAChR channels. The relative agonist potency of the two test articles towards the α7 receptor could not be established. Nevertheless, it is possible to conclude that anatabine is an agonist of all three channels, and in particular of the α4/β2 subtype. It is not possible to determine if anatabine has partial agonist activity towards the α3/β4 and α7 channels as a higher concentration range would need to be evaluated so that the level of maximal stimulation can be clearly identified.

In the potentiation assay, the test articles did not increase the signals with a low dose of Ach stimulation indicating that the test articles were not potentiators or allosteric modulators of the channels.

In the antagonist assay, after stimulation with high dose of Ach, the test articles decreased the Ach-induced signals. However, since the test articles acted as agonists, the reduction of Ach-induced signals was caused by the desensitization of the channels themselves, rather than the channel blockage. Nicotine and anatabine are not considered to be antagonists of these receptors.

### EXAMPLE 9

### Anatabine Reduces BACE-1 mRNA Levels In Vitro

The effect of anatabine (“RCP006”) on BACE-1 mRNA levels in SHSY cells was measured by RTPCR quantification using standard methodologies.

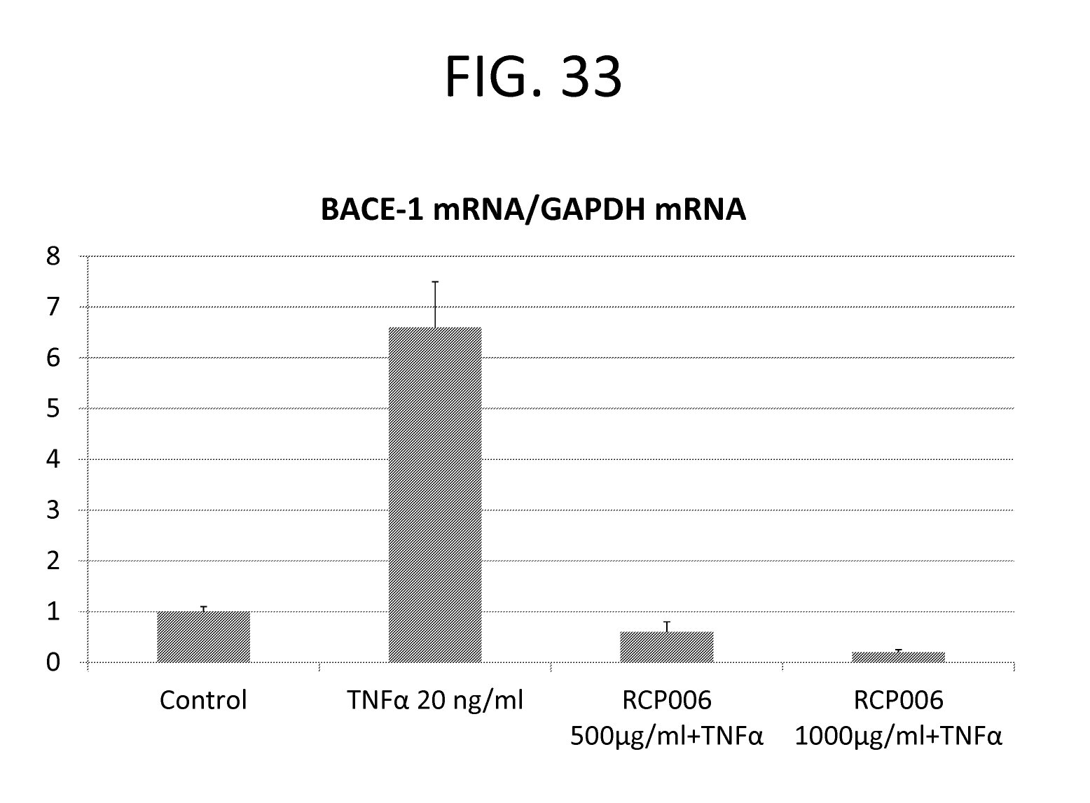

The effect of anatabine (30 minutes) on BACE-1 mRNA expression in human neuronal SHSY cells is shown in FIG. 33. TNF-α was used to increase BACE expression. Anatabine reduced this signal in a concentration-dependent manner.

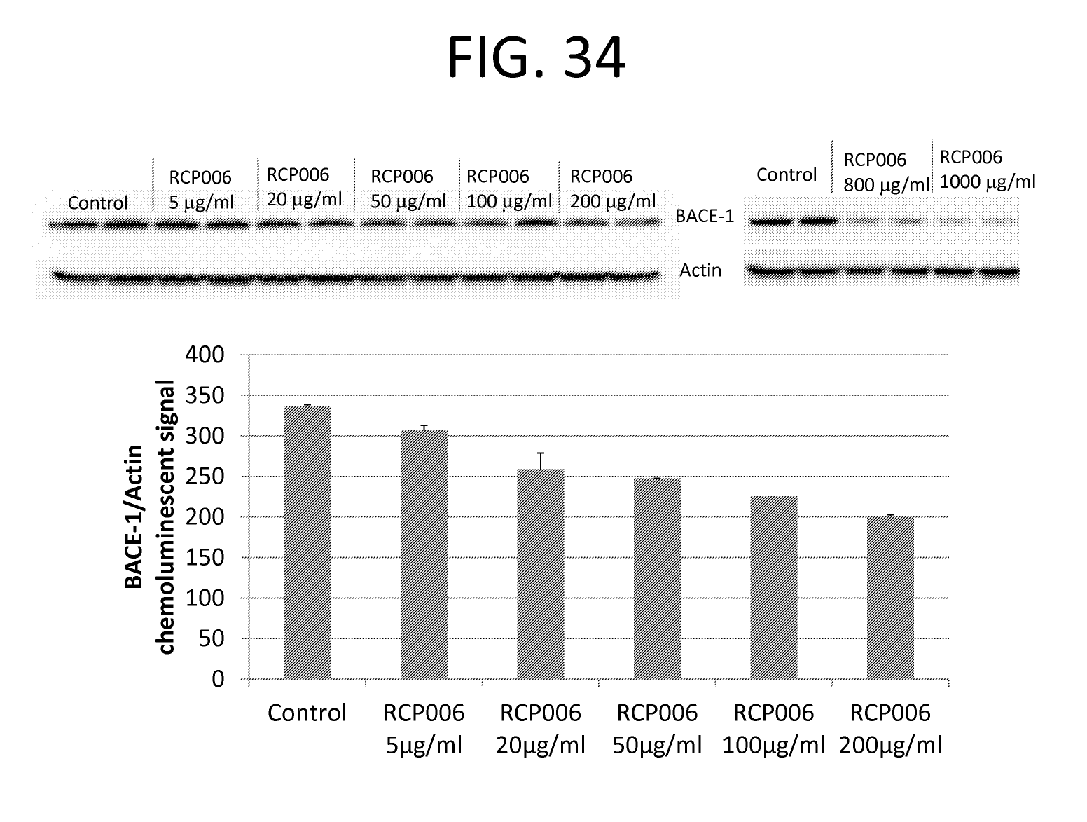

FIG. 34 shows the effect of anatabine (24 hours) on BACE-1 protein expression in human neuronal SHSY cells, corrected for actin expression. Increasing concentrations of anatabine reduced BACE expression.

These results demonstrate that anatabine can reduce BACE expression levels and suggest a mechanism by which anatabine could lower Aβ production.

### EXAMPLE 10

### Anatabine Reduces Aβ Production In Vitro

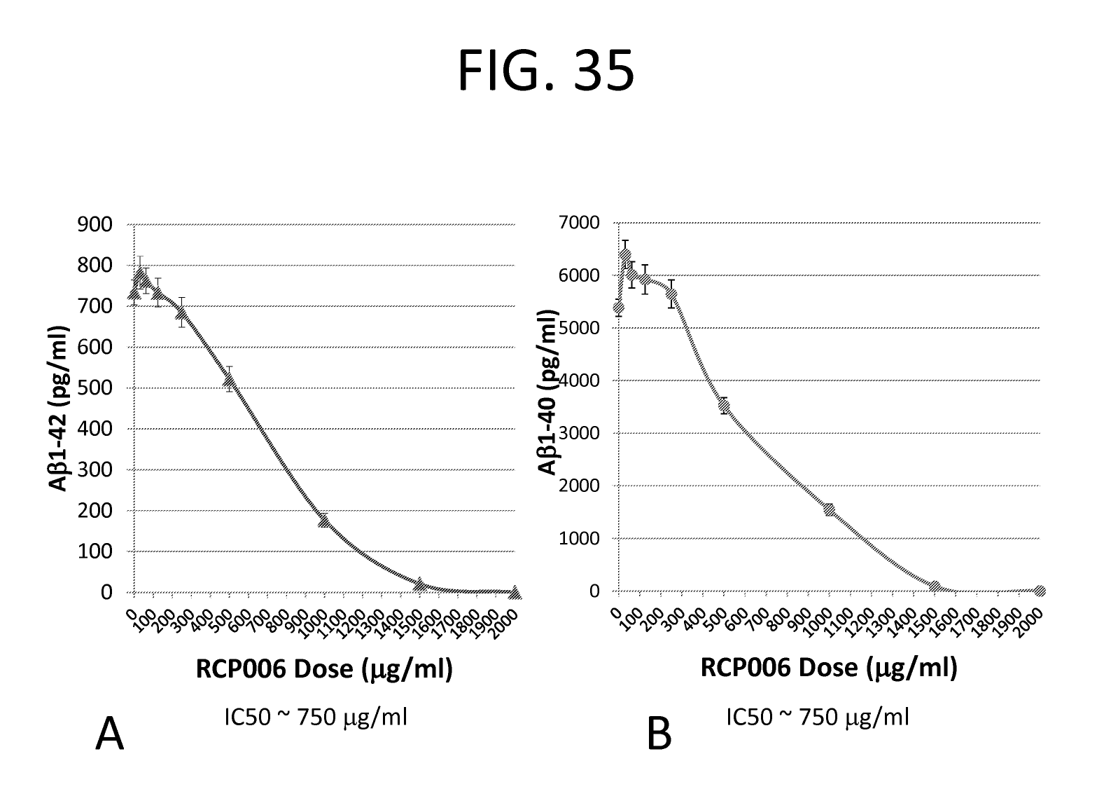

The effect of anatabine (“RCP006”) on Aβ production in vitro in 7W CHO cells was measured. The results are shown in FIG. 35A and FIG. 35B. Both graphs show a concentration-dependent reduction of Aβ production by anatabine.

### EXAMPLE 11

### Anatabine Reduces β-Cleavage of APP In Vitro

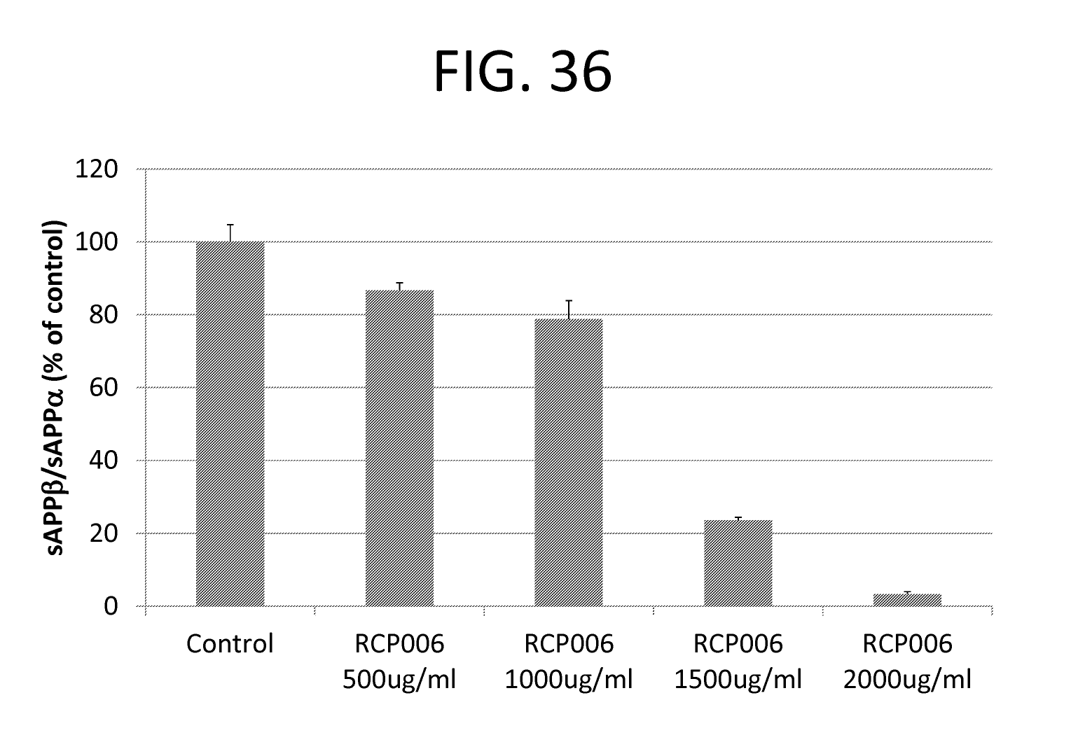

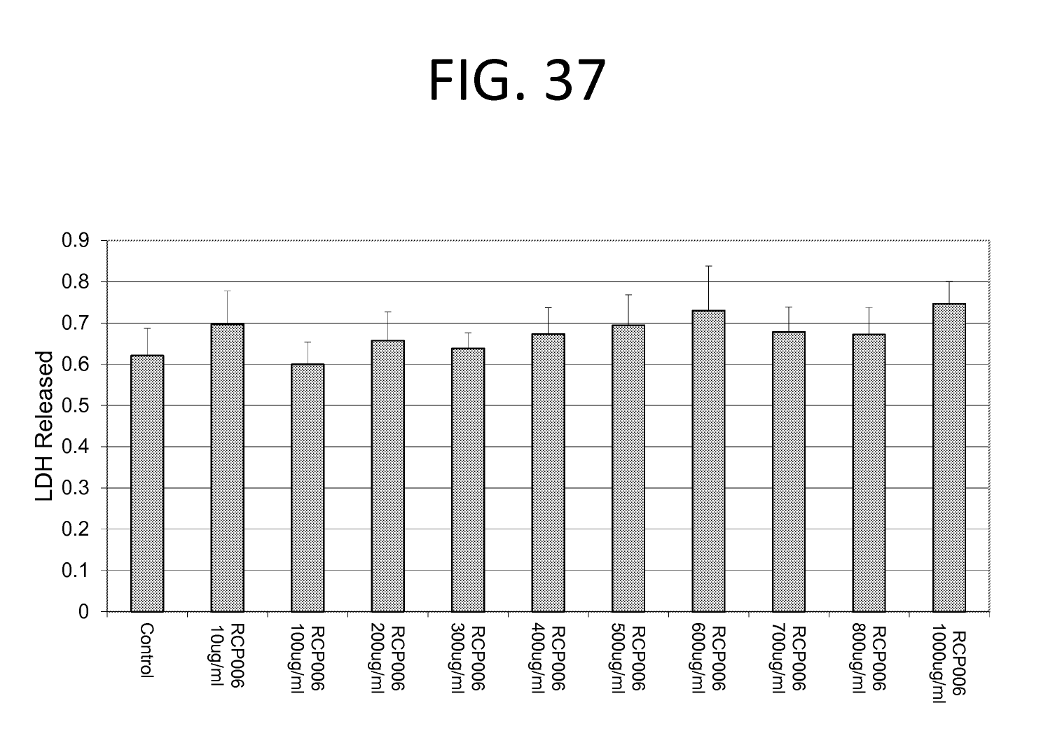

The effect of anatabine (“RCP006”) on sAPPβ/sAPPα production in vitro was measured in 7W CHO cells. The results are shown in FIG. 36. These results demonstrate that anatabine reduces the sAPPβ/sAPPα ratio, which suggests that anatabine can lower the activity of BACE to cleave APP. No toxicity was observed; see FIG. 37.

### EXAMPLE 12

### Anatabine Reduces p65 Phosphorylation In Vivo

Wild-type mice (B6/SJL), 75 weeks of age, were injected intraperitoneally with PBS or with 2 mg/kg of anatabine (“RCP006”). After 5 minutes, mice were intracranially injected with 0.5 mg of TNFα. Mice were euthanized ten minutes after the intracranial injection. The portion of the brain surrounding the intracranial site of injection was collected, and proteins were extracted. Phosphorylation of p65 was measured with an antibody towards phosphorylated p65.

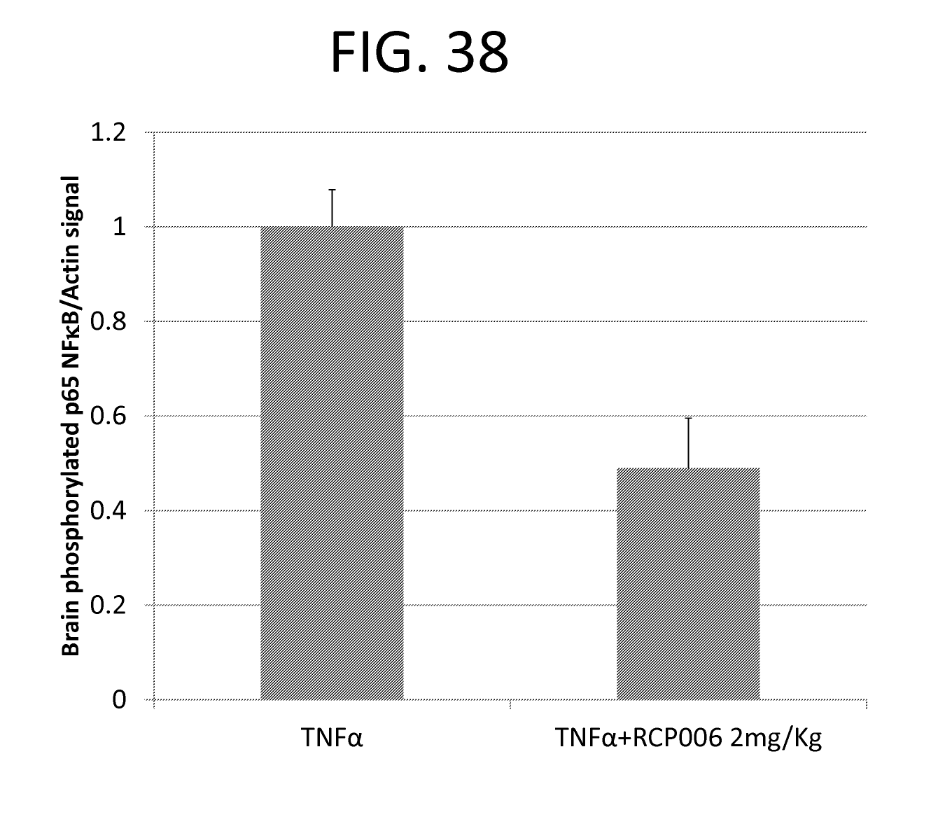

The results are shown in FIG. 38, normalized to an actin signal. These results demonstrate that anatabine treatment results in reduced p65 phosphorylation in mouse brain.

### EXAMPLE 13

### Anatabine Inhibits LPS-Induced IL-1β Release in Whole Human Blood

Whole human blood was treated with LPS to stimulate inflammatory responses. LPS treatment was also accompanied by treatment with LIPITOR® or with anatabine (“RCP006”). The inflammatory molecule IL-1β was measured after 16 hours.

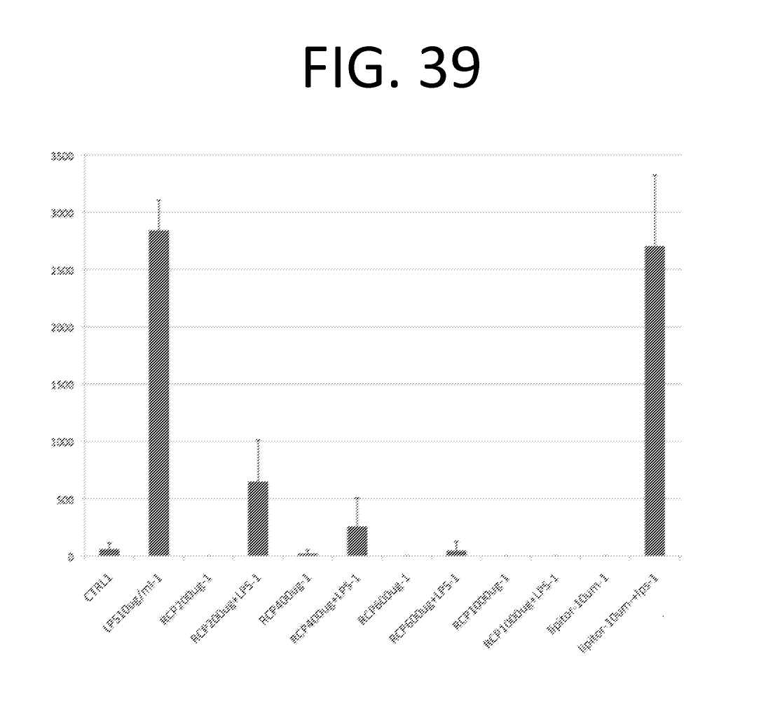

The results are shown in FIG. 39. A reduced accumulation of IL-1β was observed in anatabine-treated blood compared to untreated blood, whereas LIPITOR® had no effect at the dose shown.

### EXAMPLE 14

### Anatabine Reduces LPS-Induced IL-1β Release in Whole Human Blood

Whole human blood was treated with LPS to stimulate inflammatory responses. LPS treatment was also accompanied by treatment with known anti-inflammatory compounds or with anatabine (“RCP006”). The inflammatory molecule IL-1β was measured after 16 hours.

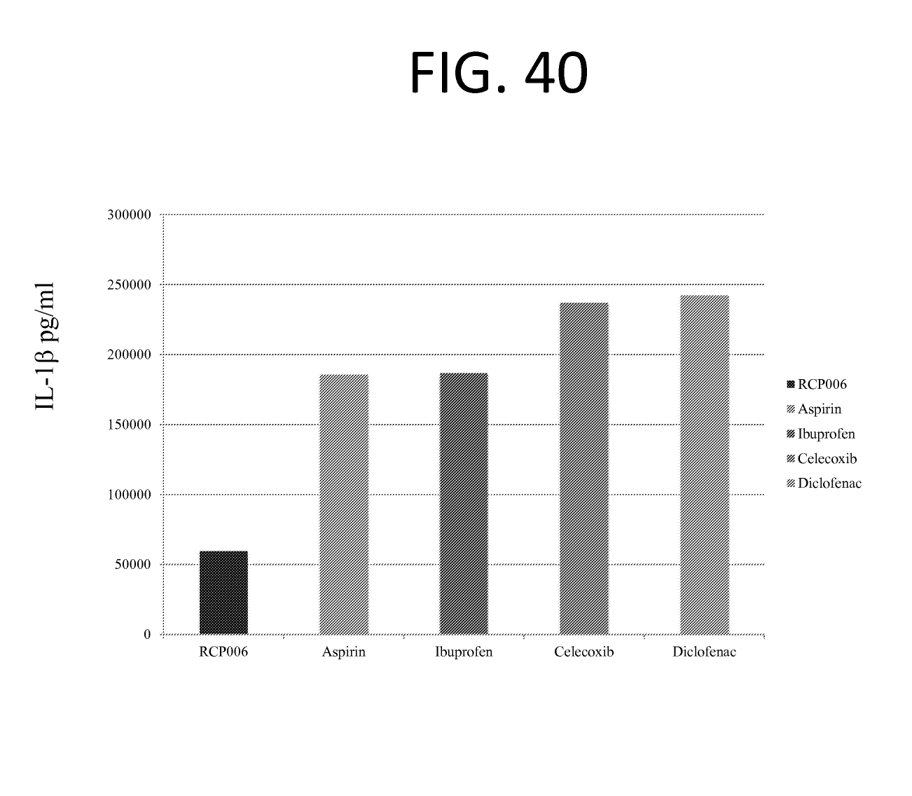

The results are shown in FIG. 40. The data shown were generated by summing the area under the curve for the dose response (measured in IL-1β levels) from zero to the treatment where full inhibition was achieved for each compound.

A reduced accumulation of IL-1β in anatabine-treated blood was observed, whereas the commonly used anti-inflammatory agents all triggered an increase in IL-1β production at lower doses prior to declines at higher doses. These data are consistent with anatabine having anti-inflammatory effects in human blood.

### EXAMPLE 15

### Anatabine Rapidly and Continuously Suppresses IL-1β Production in Human Blood

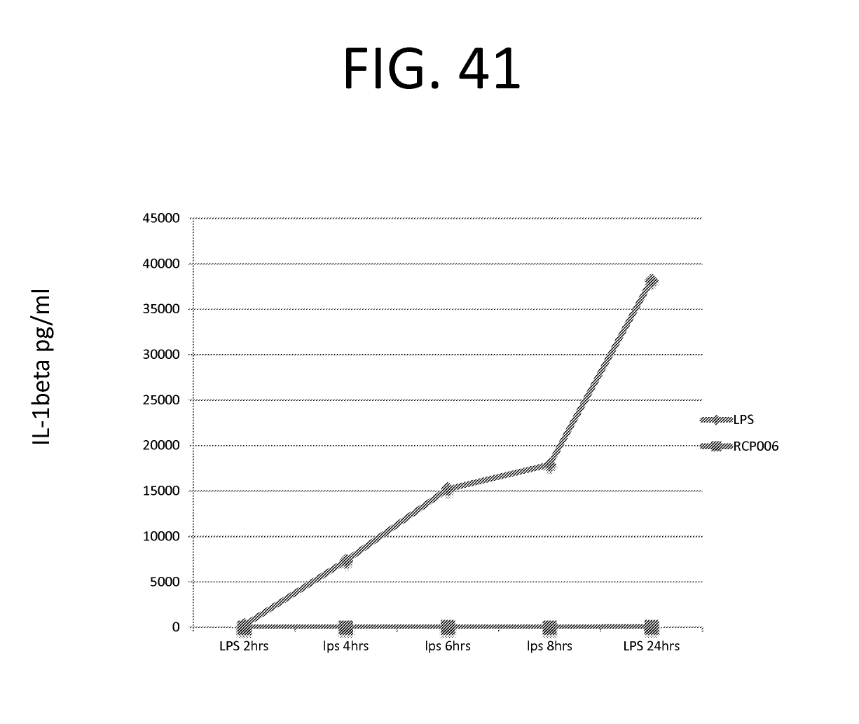

Accumulation of IL-1β was measured repeatedly over time with and without anatabine (“RCP006”) treatment, using methods of Examples 13 and 14. The results are shown in FIG. 41.

These results demonstrate that anatabine has a rapid and continuous effect on the suppression if IL-1β production after LPS stimulation of human blood.

### EXAMPLE 16A

### Effects of Anatabine in a Mouse Model of Autoimmune Thyroiditis

Effects of anatabine were studied in a mouse model of autoimmune thyroiditis (Experimental Autoimmune Thyroiditis; EAT). Thyroiditis was induced by injection of thyroglobulin emulsified in complete Freund's adjuvant (CFA). On days 0 and 7, female mice received subcutaneous injection of 100 μg thyroglobulin (2 injections of 50 μg each). Control mice drank water. Anatabine-treated mice were provided with water containing anatabine (0.05 mg/ml; approximately 12.5 mg/kg body weight/day). Mice were sacrificed on day 21.

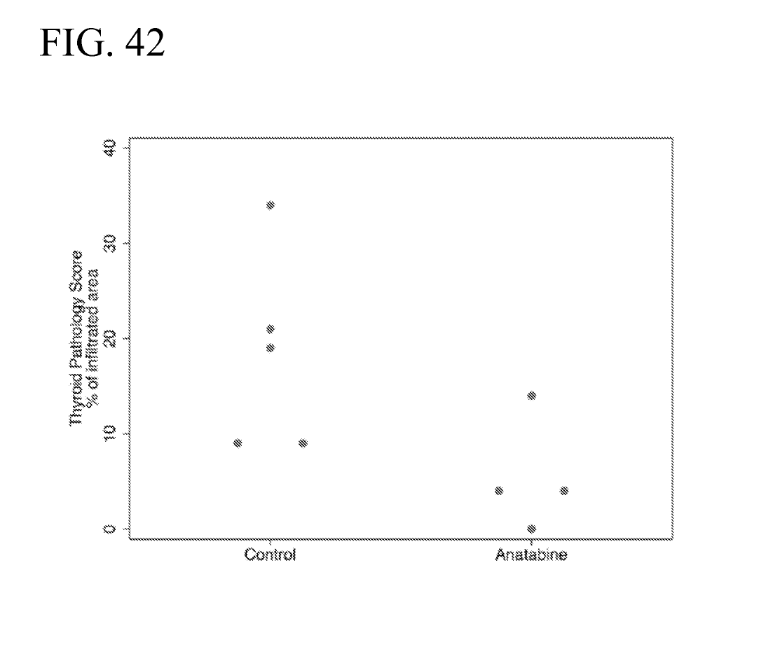

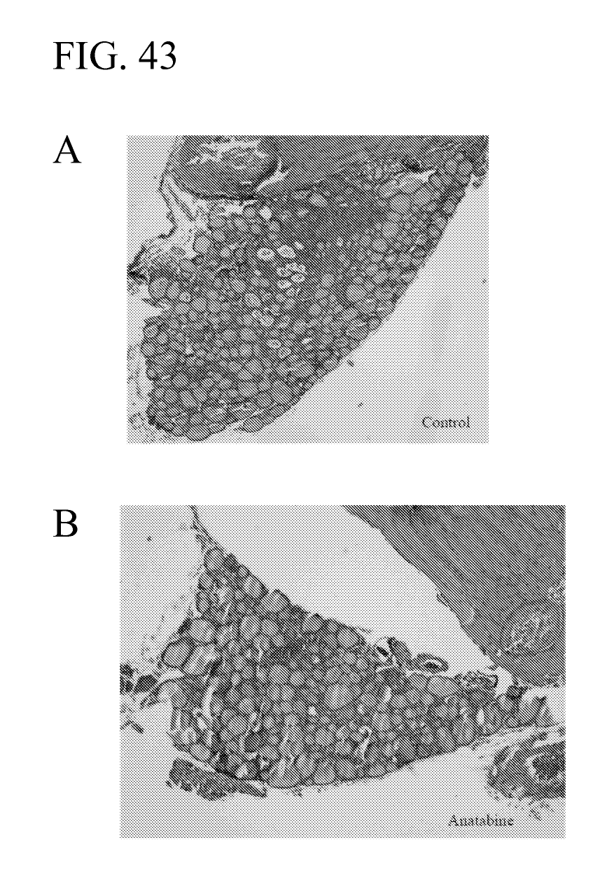

Thyroid histopathology. Thyroid glands were removed. One lobe was fixed in formalin for histopathology. One lobe was frozen for immunohistochemistry. The extent of lymphocytic infiltration and destruction of the thyroid gland was assessed digitally. Initially, the entire thyroid lobe was examined. Then, all regions that showed pathological damage were selected. The final score was expressed as the percent of the thyroid area infiltrated by lymphocytes and damaged. The results are shown graphically in FIG. 42. Anatabine-treated mice developed less severe thyroiditis than control mice (p=0.05). Photomicrographs of hematoxylin- and eosin-stained thyroids from control and anatabine-treated mice are shown in FIG. 43A (control) and FIG. 43B (anatabine-treated).

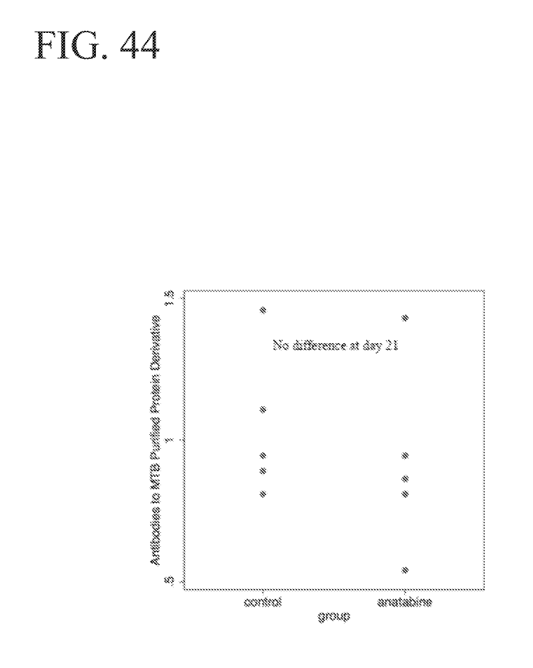

Immunochemistry. Serum from control and anatabine-treated mice was examined to determine levels of antibodies to a foreign antigen (PPD). The results are shown in FIG. 44. There was no significant difference between the control and anatabine-treated mice.

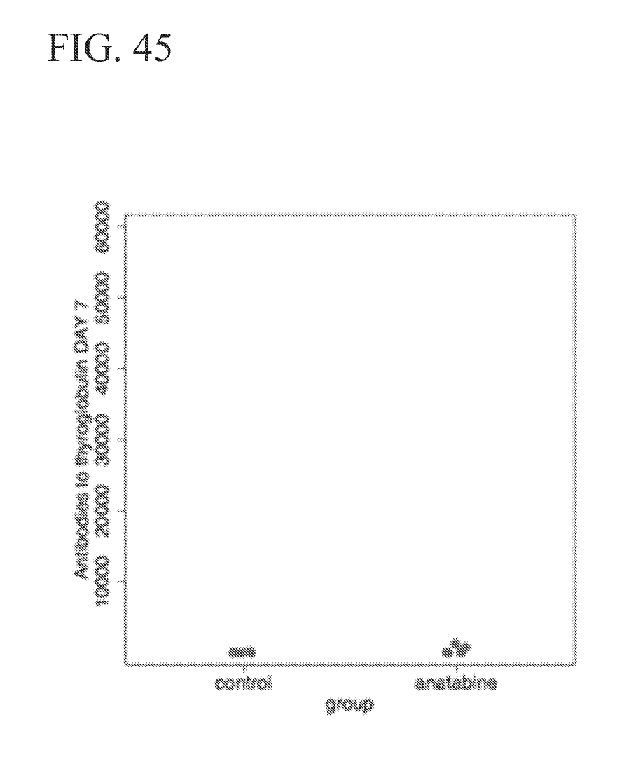

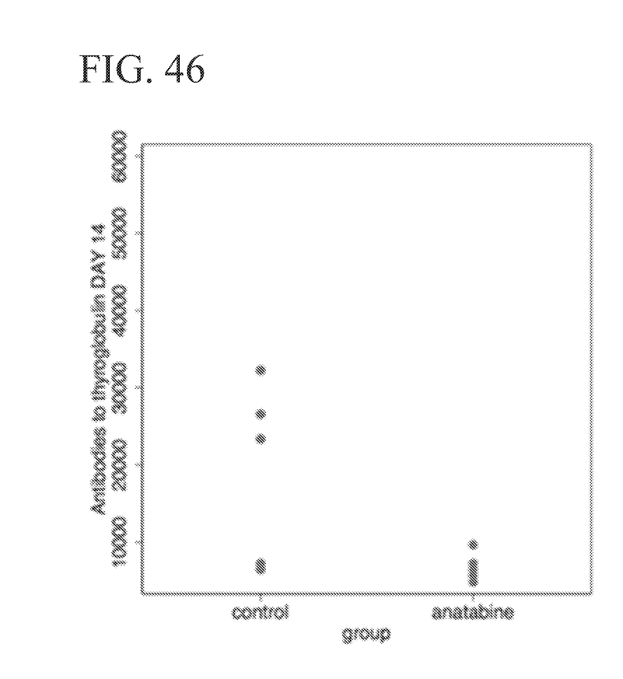

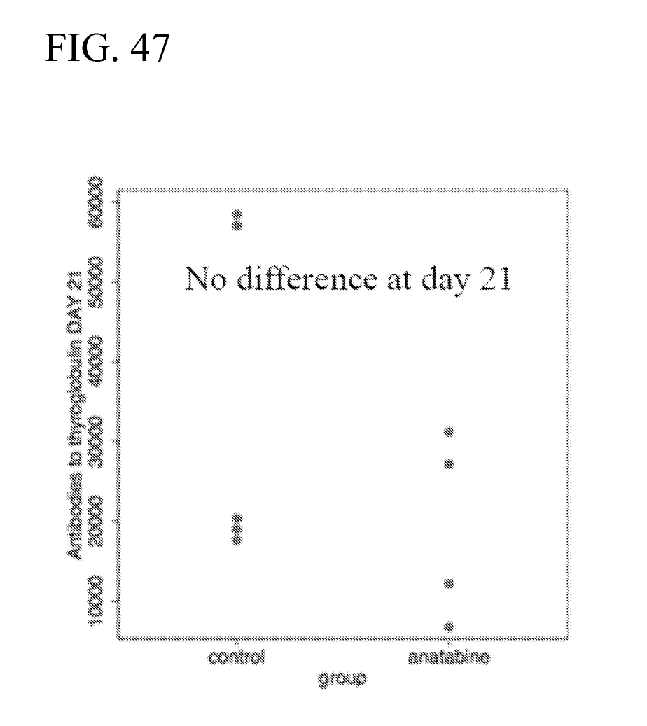

Levels of antibodies to thyroglobulin were examined on days 7, 14, and 21 after immunization. There was no significant difference between the control and anatabine-treated mice at day 7 (FIG. 45). Anatabine-treated mice tended to have lower thyroglobulin antibodies by day 14 (FIG. 46; p=0.086); by day 21, there was no significant difference between the control and the anatabine-treated mice (FIG. 47).

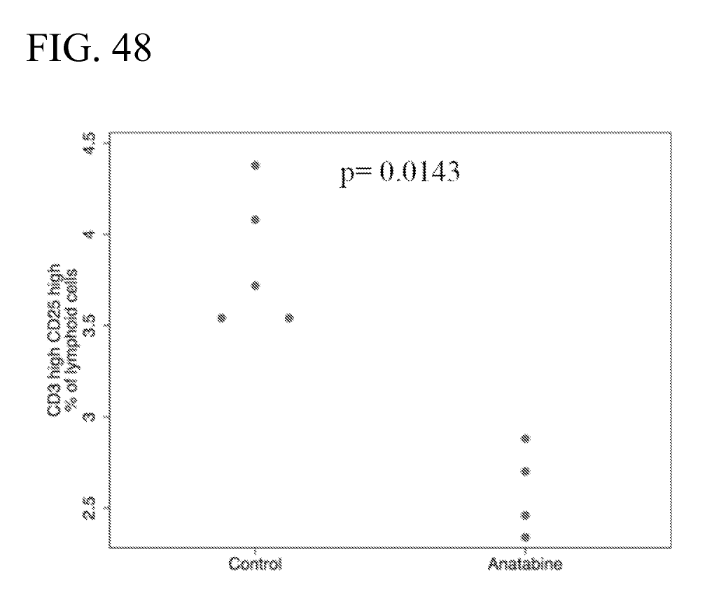

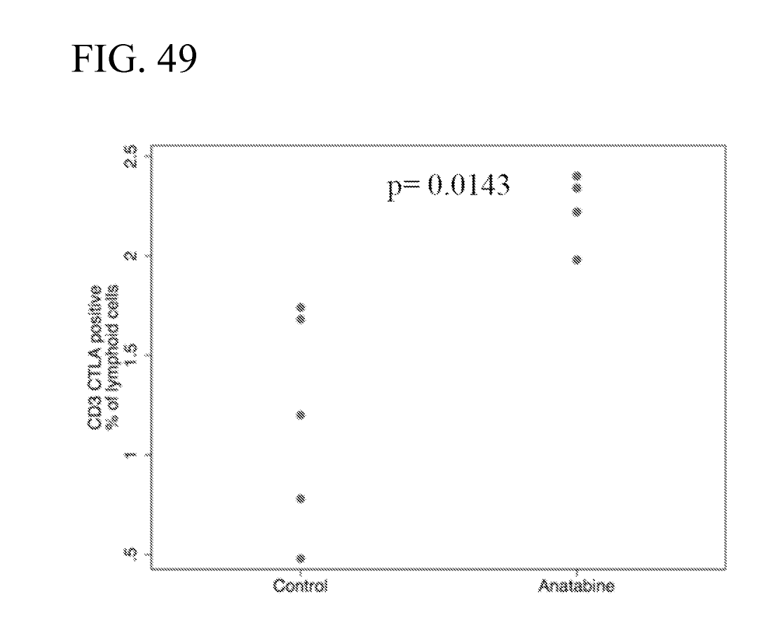

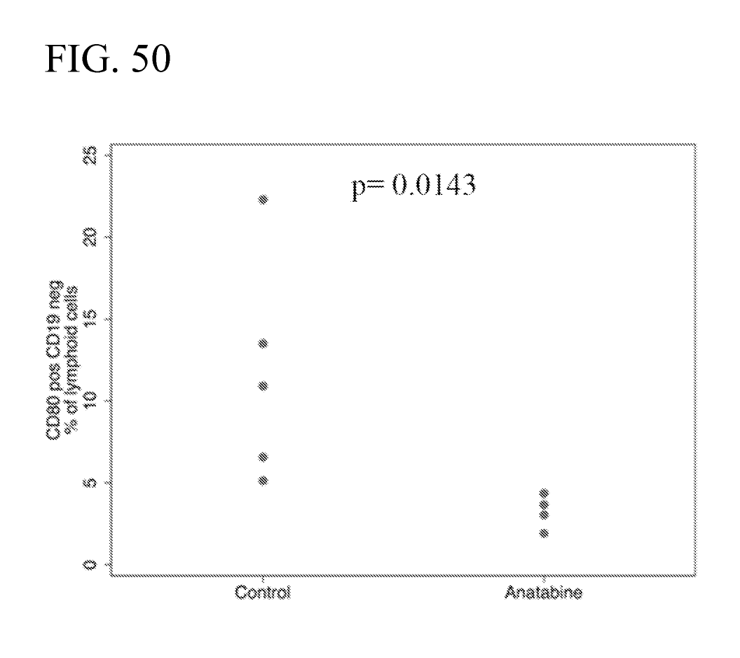

Lymphoid typing. Cervical lymph nodes were removed for lymphoid typing by flow cytometry. Spleen and peritoneal macrophages were removed for ex vivo stimulation. Anatabine-treated mice seemed to have fewer activated T cells (FIG. 48; p=0.0143), more regulatory T cells (FIG. 49; p=0.0143), and a lower ability to present antigens (FIG. 50; p=0.0143).

### EXAMPLE 16B

### Effects of Anatabine in a Mouse Model of Autoimmune Thyroiditis

In another experiment, eighteen CBA/J female mice were immunized with mouse thyroglobulin, emulsified in complete Freund's adjuvant, on day 0 and day 7. One group of mice (n=10) drank regular water. The other group drank water supplemented with anatabine (0.05 mg/ml; approximately 12.5 mg/kg body weight/day). Mice were sacrificed 21 days after the first immunization to collect the thyroid gland and the blood. The thyroid was analyzed for the presence of infiltrating mononuclear cells. The blood was analyzed for the levels of antibodies against thyroglobulin.

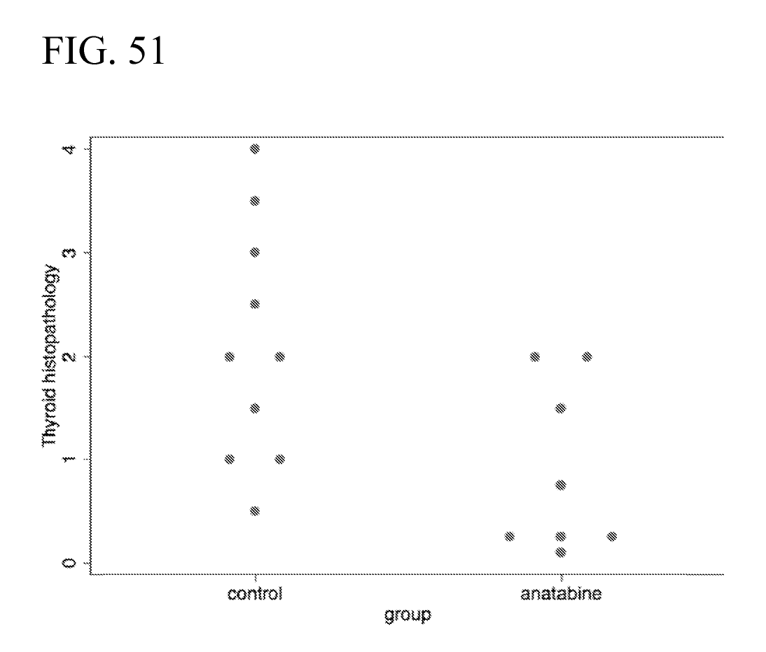

The results are shown in FIG. 51. Mice that drank anatabine developed thyroiditis less frequently and of lower severity (p=0.023 by Wilcoxon rank sum test). Anatabine had no effect on the levels of thyroglobulin antibodies, which increased in both groups after immunization.

### EXAMPLE 17

### Effect of S-(−)-anatabine on TNFα-Induced NFκB Activity In Vitro

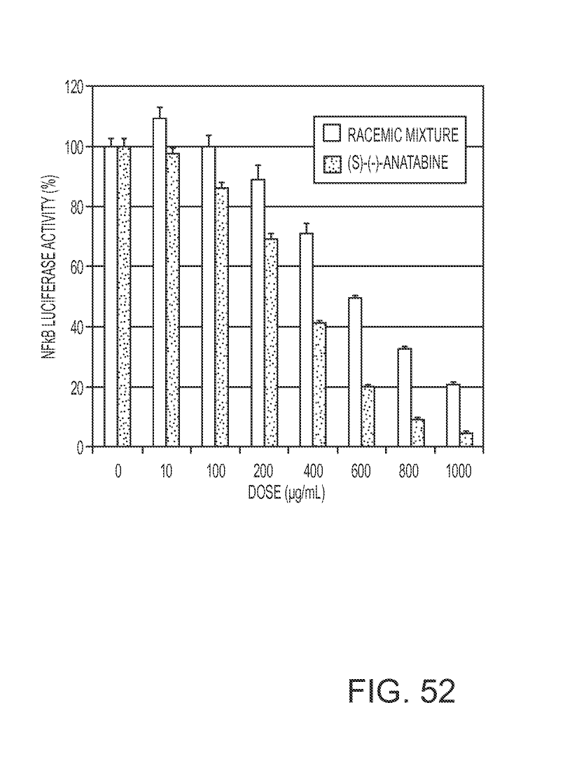

The effect of S-(−)-anatabine on TNFα-induced NFκB activity in vitro was determined as described in Example 1. NFκB activity was stimulated with 20 ng/ml of TNFα, then varying doses of a racemic mixture of anatabine or S(−)-anatabine were applied to the challenged cells. The data were plotted as a percentage of the TNFα-induced NFκB activity and are shown in FIG. 52. In this assay the IC₅₀ for the racemic mixture of anatabine is approximately 600 μg/ml, whereas the IC₅₀ for the (S)-enantiomer is approximately 330 μg/ml.

1. A method of reducing a symptom in an individual of a disorder comprising an NFκB-mediated inflammatory component, comprising administering to the individual a composition comprising a therapeutically effective dose of an isolated form of a compound of Formula I 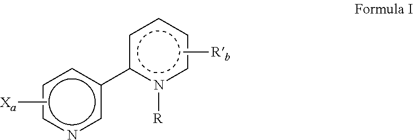
wherein:
R represents hydrogen or C₁-C₅ alkyl;
R′ represents hydrogen or C₁-C₇ alkyl; and
X represents halogen or C₁-C₇ alkyl,
or a salt thereof.

2. The method of claim 1 wherein the compound is anatabine or S-(−)-anatabine.

3. The method of claim 1 wherein the composition comprises a salt of anatabine or S-(−)-anatabine.

4. The method of claim 1 wherein the NFκB-mediated inflammatory component is chronic inflammation.

5. The method of claim 1 wherein the disorder is selected from the group consisting of an autoimmune disorder, a vascular inflammatory disorder, a gastrointestinal inflammatory disorder, a neurodegenerative disorder, cancer, an upper respiratory tract infection, a joint disorder, seizure disorder, and an autism spectrum disorder.

6. The method of claim 5 wherein the disorder is an autoimmune disorder and the autoimmune disorder is selected from the group consisting of thyroiditis, rheumatoid arthritis, and diabetes.

7. The method of claim 5 wherein the disorder is a vascular inflammatory disorder and the vascular inflammatory disorder is selected from the group consisting of atherosclerosis, coronary artery disease, cerebrovascular disease, peripheral vascular disease, and inflammation-induced myocarditis.

8. The method of claim 5 wherein the disorder is a neurodegenerative disorder and the neurodegenerative disorder is selected from the group consisting of Alzheimer's disease and Parkinson's disease, and wherein the compound of Formula I is S-(−)-anatabine.

9. The method of claim 5 wherein the disorder is a cancer.

10. The method of claim 5 wherein the disorder is an upper respiratory tract infection and the upper respiratory tract infection is a cold.

11. The method of claim 5 wherein the disorder is a joint disorder and the joint disorder is selected from the group consisting of osteoarthritis and degenerative joint disease.

12. The method of claim 5 wherein the disorder is a seizure disorder or an autism spectrum disorder.

13. The method of claim 1 wherein the dose is in an extended release formulation.

14. The method of claim 1 wherein the dose is from about 0.1 to about 1.5 mg/kg body weight.

15. The method of claim 1 further comprising administration of a second therapeutic agent.

16. A method of reducing a risk in an individual of developing a disorder comprising an NFκB-mediated inflammatory component, comprising administering to the individual a composition comprising a therapeutically effective dose of an isolated form of a compound of Formula I or a salt thereof.

17. A beverage product suitable for human consumption comprising a beverage container containing a liquid medium and an isolated form of a compound of Formula I 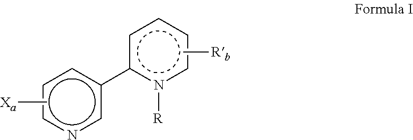
wherein:
R represents hydrogen or C₁-C₅ alkyl;
R′ represents hydrogen or C₁-C₇ alkyl; and
X represents halogen or C₁-C₇ alkyl,
or a salt thereof.

18. The beverage product of claim 17 wherein the compound is anatabine or S-(−)-anatabine.

19. A consumer product selected from the group consisting of paste, cream, lotion, moisturizer, sunscreen, toothpaste, mouthwash, and an inhaler, wherein the product contains an isolated form of a compound of Formula I: 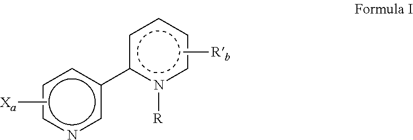
wherein:
R represents hydrogen or C₁-C₅ alkyl;
R′ represents hydrogen or C₁-C₇ alkyl; and
X represents halogen or C₁-C₇ alkyl,
or a salt thereof.

20. The consumer product of claim 19 wherein the compound is anatabine or S-(−)-anatabine.
